(19) United States
(12) Patent Application Publication
(10) Pub. No.: US 2026/0200893 A1
(43) Pub. Date: Jul. 16, 2026

(54) INHIBITORS OF PROCASPASE-6 ACTIVATION AND USES THEREOF

(71) Applicants: THE REGENTS OF THE UNIVERSITY OF CALIFORNIA, Oakland, CA (US); ELGIA THERAPEUTICS, INC., La Jolla, CA (US)

(72) Inventors: Adam R. Renslo, Oakland, CA (US); Stacie S. CANAN, La Jolla, CA (US); M. Katharine HOLLOWAY, Austin, TX (US)

(21) Appl. No.: 19/138,034

(22) PCT Filed: Dec. 13, 2023
(86) PCT No.: PCT/US2023/083892

§ 371 Date: Jun. 11, 2025

Related U.S. Application Data
(60) Provisional application No. 63/432,560, filed on Dec. 14, 2022.

(51) Int. Cl. C07D 401/14; A61K 31/4439; A61K 31/444; A61K 31/501; C07D 405/14; C07D 409/14; C07D 413/14; C07D 417/14

(52) U.S. Cl. CPC C07D 401/14; A61K 31/4439; A61K 31/444; A61K 31/501; C07D 405/14; C07D 409/14; C07D 413/14; C07D 417/14

(57) ABSTRACT

Described herein, inter alia, are inhibitors of procaspase-6 activation and uses thereof.

### CROSS-REFERENCES TO RELATED APPLICATIONS

This application claims the benefit of U.S. Provisional Application No. 63/432,560, filed Dec. 14, 2022, which is incorporated herein by reference in its entirety and for all purposes.

### REFERENCE TO AN ELECTRONIC SEQUENCE LISTING

The contents of the electronic sequence listing (048536-754001WO_Sequence_Listing_ST26.xml; Size: 2,608 bytes; and Date of Creation: Nov. 29, 2023) is hereby incorporated by reference in its entirety.

### BACKGROUND

Caspase-6 is implicated in several neurodegenerative conditions, including Huntington's and Alzheimer's disease. Expressed as an inactive zymogen form, procaspase-6, its conversion to proteolytically active caspase-6 is observed during all stages of AD. Additionally, the activation of Casp6 correlates with a lower cognitive score in normal aged individuals. Disclosed herein, inter alia, are solutions to these and other problems in the art.

### BRIEF SUMMARY

In an aspect is provided a compound, or a pharmaceutically acceptable salt thereof, having the formula:

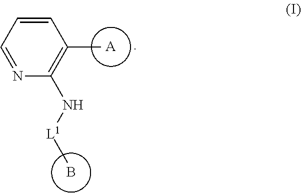

Ring A is a substituted or unsubstituted 5 to 6 membered heteroaryl or substituted or unsubstituted 8 to 10 membered fused ring heteroaryl.

Ring B is a substituted or unsubstituted 9 to 10 membered fused ring aryl or substituted or unsubstituted 9 to 10 membered fused ring heteroaryl.

L¹ is a bond or substituted or unsubstituted C₁-C₃ alkylene.

In an aspect is provided a pharmaceutical composition including a compound described herein, or a pharmaceutically acceptable salt thereof, and a pharmaceutically acceptable excipient.

In an aspect is provided a method of treating a neurodegenerative disease in a subject in need thereof, the method including administering to the subject in need thereof a therapeutically effective amount of a compound described herein, or a pharmaceutically acceptable salt thereof.

In an aspect is provided a method of treating a liver disease in a subject in need thereof, the method including administering to the subject in need thereof a therapeutically effective amount of a compound described herein, or a pharmaceutically acceptable salt thereof.

In an aspect is provided a method of treating a fibrotic disease in a subject in need thereof, the method including administering to the subject in need thereof a therapeutically effective amount of a compound described herein, or a pharmaceutically acceptable salt thereof.

In an aspect is provided a method of treating a coronavirus infection in a subject in need thereof, the method including administering to the subject in need thereof a therapeutically effective amount of a compound described herein, or a pharmaceutically acceptable salt thereof.

In an aspect is provided a method of reducing the level of activity of caspase-6 protein in a cell, the method including contacting the cell with an effective amount of a compound as described herein, or a pharmaceutically acceptable salt thereof.

In an aspect is provided a method of reducing the level of activation of procaspase-6 protein in a cell, the method including contacting the cell with an effective amount of a compound as described herein, or a pharmaceutically acceptable salt thereof.

### BRIEF DESCRIPTION OF THE DRAWINGS

FIG. 1. Structure and bound conformation of compound 1, a molecular glue that binds the dimer interface of procaspase-6. The pyrimidine ring of 1 stacks between tyrosine 198^{A} and 198^{B} from the respective halves of the C2 symmetric dimer (PDB: 4NBL).

FIG. 2. Compounds 1-30 synthesized to study heteroarene-aryl stacking of diverse probe heterocycles. The grey sphere indicates the site of connection to the shared ligand scaffold present in progenitor ligand 1.

FIG. 3. The aligned complex crystal structures of five-membered ring analogs 7, 8, 10, 11, 19, 20, and 21 bound to procaspase-6 are shown on the left. The aligned complex crystal structures of six-membered ring analogs 1, 3, and 5 bound to procaspase-6 are on the right. Tyrosine residues are from the procaspase-6 complex structures of 11 (left) and 3 (right).

FIG. 4. Computed interaction energies (Eᵢₙₜ) and experimental binding free energies (ΔG; kcal/mol) for test compounds bearing probe heteroarenes with either one or two heteroatoms. Data points are labelled with compound numbers from FIG. 2.

FIG. 5. Computed interaction energies (Eᵢₙₜ) and experimental binding free energies (ΔG; kcal/mol) for test compounds bearing probe heteroarenes with either three or four heteroatoms. Data points are labelled with compound numbers from FIG. 2.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —S—CH₂—CH₂, —S(O)—CH₃. —CH₂—CH₂—S(O)₂—CH₃. —CH═CHO—CH₃, —Si(CH₃)₃. —CH₂—CH═N—OCH₃. —CH═CH—N(CH₃)—CH₃, —O—CH₃. —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., 0, N. S, Si, or P). A heteroalkyl moiety may include two optionally different heteroatoms (e.g., O, N, S, Si, or P). A heteroalkyl moiety may include three optionally different heteroatoms (e.g., O, N, S. Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl.” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl.” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′—and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom.

Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “-” denotes the point of attachment of a chemical moiety to the remainder of a molecule or chemical formula.

The term “oxo,” as used herein, means an oxygen that is double bonded to a carbon atom.

The term “alkylarylene” as an arylene moiety covalently bonded to an alkylene moiety (also referred to herein as an alkylene linker). In embodiments, the alkylarylene group has the formula:

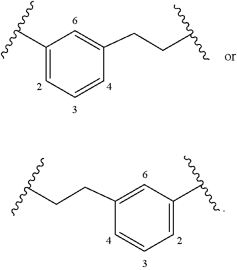 [SMILES: *CCc1cccc(*)c1 ; *CCc1cccc(*)c1]

An alkylarylene moiety may be substituted (e.g., with a substituent group) on the alkylene moiety or the arylene linker (e.g., at carbons 2, 3, 4, or 6) with halogen, oxo, —N₃. —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, =NR′, =N—OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R″, —NR″C(O)₂R′, —NRC(NR′R″R′″)=NR″″, —NRC(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen, —SiR′R″R′″, —OC(O)R′, —C(O)R′, —CO₂R′, —CONR′R″, —OC(O)NR′R″, —NR″C(O)R′, —NR′C(O)NR″R″, —NR″C(O)₂R′, —NR—C(NR′R″R′″)=NR″″, —NR—C(NR′R″)=NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl. —NR′SO₂R″, —NR′C(O)R″, —NR′C(O)OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -A-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), selenium (Se), and silicon (Si). In embodiments, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH. —NH₂, —COOH, —CONH₂, —NO₂. —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
(i) oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl. C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted anil (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (a) oxo, halogen, —CCl₃, —CBr₃, —CF₃, —CI₃, —CHCl₂, —CHBr₂, —CHF₂, —CH₁₂, —CH₂Cl, —CH₂Br, —CH₂F, —CH₂I, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —CN, —OH, —NH₂. —COOH, —CONH₂, —NO₂, —SH. —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, —SF₅, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group.” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group.” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on an R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}. R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1}. As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g., R^{1.1}, R^{2.1}, R^{3.1}, R^{4.1}, R^{5.1} . . . R^{100.1}; R^{1A.1}, R^{2A.1}, R^{3A.1}, R^{4A.1}, R^{5A.1} . . . R^{100A.1}; R^{L1.1}, R^{L2.1}, R^{L3.1}, R^{L4.1}, R^{L5.1} . . . R^{L100.1}) may be further substituted with one or more second substituent groups (e.g., R^{1.2}, R^{2.2}, R^{3.2}, R^{4.2}, R^{5.2} . . . R^{100.2}. R^{1A.2}, R^{2A.2}, R^{3A.2}, R^{4A.2}, R^{5A.2} . . . R^{100A.2}; R^{L1.2}, R^{L2.2}, R^{L3.2}, R^{L4.2}, R^{L5.2} . . . R^{L1000.2} respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g., R^{1.2}, R^{2.2}, R^{3.2}, R^{4.2}, R^{5.2} . . . R^{100.2}, R^{1A.2}, R^{2A.2}, R^{3A.2}, R^{4A2}, R^{5A2} . . . R^{100A.2}; R^{L1.2}, R^{L2.2}, R^{L3.2}, R^{L4.2}, R^{L5.2} . . . R^{L100.2}) may be further substituted with one or more third substituent groups (e.g., R^{1.3}, R^{2.3}, R^{3.3}, R^{4.3}, R^{5.3} . . . R^{100.3}; R^{1A.3}, R^{2A.3}, R^{3A.3}, R^{4A.3}, R^{5A.3} . . . R^{100A.3}; R^{L1.3}, R^{L2.3}, R^{L3.3}, R^{L4.3}, R^{L5.3} . . . R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein. R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW.2}; may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g., when R^{WW.1} is R^{WW.2}-substituted or unsubstituted alkyl, examples of groups so formed include but are not limited to itself optionally substituted by 1 or more R^{WW.2}, which R^{WW.2} is optionally substituted by one or more R^{WW.3}. By way of example when the R^{WW} group is phenyl substituted by R^{WW.1}, which is methyl, the methyl group may be further substituted to form groups including but not limited to:

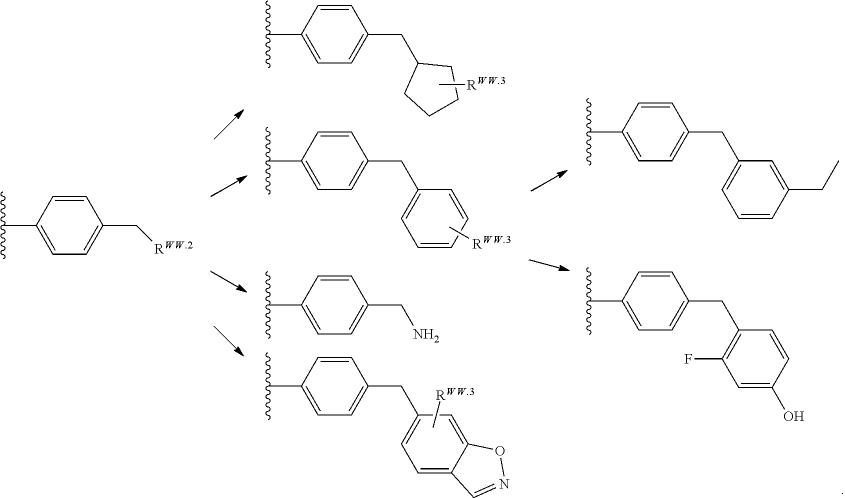

R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{WW.2}-substituted or unsubstituted alkyl (e.g., C₁-C₈, C₁-C₆, C₁-C₄, or C₁-C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃-C₈, C₃-C₆, C₄-C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆-C₁₂, C₆-C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH. —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆. C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃. R^{WW.3}-substituted or unsubstituted alkyl (e.g., C₁-C₈, C₁-C₆, C₁-C₄, or C₁-C₂), R^{WW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{WW.3}-substituted or unsubstituted heterocycloalkyl (e.g., 3 to 8 membered, 3 to 6 membered, 4 to 6 membered, 4 to 5 membered, or 5 to 6 membered). R^{WW.3}-substituted or unsubstituted aryl (e.g., C₆-C₁₂, C₆-C₁₀, or phenyl), or R^{WW.3}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂. —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.3} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —OSO₃H, —SO₂NH₂, —NHNH₂. —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆. C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g., substituted cycloalkyl, substituted heterocycloalkyl, substituted aryl or substituted heteroaryl), in embodiments the openly substituted ring may be independently substituted with one or more first substituent groups, referred to herein as R^{WW.1} each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.3}; and each third substituent group, R^{WW.3}, is unsubstituted.

Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.3} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.3} is R^{100A.3}. Alternatively, in embodiments where R^{100A} and R^{100B} are optionally joined together to form an openly substituted ring, R^{WW.1} is R^{100B.1}, R^{WW.2} is R^{100B.2} and R^{WW.3} is R^{100B.3}·R^{WW.1}, R^{WW.2} and R^{WW.3} in this paragraph are as defined in the preceding paragraphs.

R^{LWW.1} is independently oxo, halogen, —CX^{LWW.1}₃—CHX^{LWW.1}₂—CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.2}-substituted or unsubstituted alkyl (e.g., C₁-C₈, C₁-C₆, C₁-C₄, or C₁-C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃-C₈, C₃-C₆, C₄-C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆-C₁₂, C₆-C₁₀, or phenyl), or R^{LWW.2}-substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂. —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH, —NHOH, —N₃, R^{LWW.3}-substituted or unsubstituted alkyl (e.g., C₁-C₈, C₁-C₆, C₁-C₄, or C₁-C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.3}-substituted or unsubstituted cycloalkyl (e.g., C₃-C₈, C₃-C₆, C₄-C₆, or C₅-C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆-C₁₂, C₆-C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₃, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, —NHC(NH)NH₂, —NHSO₂H, —NHC(O)H, —NHC(O)OH. —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —OSO₃H, —SO₂NH₂, —NHNH₂, —ONH₂, —NHC(O)NHNH₂, —NHC(O)NH₂, NHC(NH)NH₂, —NHSO₂H, —NHC(O)H, —NHC(O)OH, —NHOH, —N₃, unsubstituted alkyl (e.g., C₁-C₈, C₁-C₆, C₁-C₄, or C₁-C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃-C₈, C₃-C₆, C₄-C₆, or C₅-C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆-C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH. —CONH₂, —NO₂, —SH, —SO₃H, —OSO₃H, —SO₂NH₂, —NHNH₂, —ONH₂, —NHC(O)NHNH₂, —NHC(O)NH₂, NHC(NH)NH₂, —NHSO₂H, —NHC(O)H, —NHC(O)OH, —NHOH, —N₃, R^{WW.1}-substituted or unsubstituted alkyl (e.g., C₁-C₈, C₁-C₆, C₁-C₄, or C₁-C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃-C₈, C₃-C₆, C₄-C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆-C₁₂, C₆-C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g., 1, 2, 3, 1A, 2A, 3A, 1B, 2B, 3B, etc.). R^{WW.1}, R^{WW.2}, and R^{WW.3} are as defined above.

In the event that any L linker group recited in a claim or chemical formula description set forth herein (i.e., an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —NHC(NH)NH—, —C(O)O—, —OC(O)—, —S—, —SO₂—, —SO₂NH—, R^{LWW.1}-substituted or unsubstituted alkylene (e.g., C₁-C₈, C₁-C₆, C₁-C₄, or C₁-C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃-C₈, C₃-C₆, C₄-C₆, or C₅-C₆). R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆-C₁₂, C₆-C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{L}w, as well as R^{LWW.2} and R^{LWW.3} are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “Isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the terms “bioconjugate” and “bioconjugate linker” refer to the resulting association between atoms or molecules of bioconjugate reactive groups or bioconjugate reactive moieties. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson. BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized: (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an”, as used in herein means one or more. In addition, the phrase “substituted with a [n]”, as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl”, the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Co-administer” is meant that a composition described herein is administered at the same time, just prior to, or just after the administration of one or more additional therapies.

The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

The terms “treating” or “treatment” refers to any indicia of success in the treatment or amelioration of an injury, disease, pathology or condition, including any objective or subjective parameter such as abatement: remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters: including the results of a physical examination, neuropsychiatric exams, and/or a psychiatric evaluation. The term “treating” and conjugations thereof, include prevention of an injury, pathology, condition, or disease. In embodiments, treating is preventing. In embodiments, treating does not include preventing. In embodiments, the treating or treatment is no prophylactic treatment.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount” when referred to in this context. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms.

The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. An “activity increasing amount,” as used herein, refers to an amount of agonist required to increase the activity of an enzyme relative to the absence of the agonist. A “function increasing amount,” as used herein, refers to the amount of agonist required to increase the function of an enzyme or protein relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003. Gennaro. Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., signaling pathway) of a protein in the absence of a compound as described herein (including embodiments, examples, figures, or Tables).

“Contacting” is used in accordance with its plain ordinary meaning and refers to the process of allowing at least two distinct species (e.g., chemical compounds including biomolecules, or cells) to become sufficiently proximal to react, interact or physically touch.

It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a cellular component (e.g., protein, ion, lipid, nucleic acid, nucleotide, amino acid, protein, particle, organelle, cellular compartment, microorganism, virus, lipid droplet, vesicle, small molecule, protein complex, protein aggregate, or macromolecule). In some embodiments contacting includes allowing a compound described herein to interact with a cellular component (e.g., protein, ion, lipid, nucleic acid, nucleotide, amino acid, protein, particle, virus, lipid droplet, organelle, cellular compartment, microorganism, vesicle, small molecule, protein complex, protein aggregate, or macromolecule) that is involved in a signaling pathway.

As defined herein, the term “activation,” “activate,” “activating” and the like in reference to a protein refers to conversion of a protein into a biologically active derivative from an initial inactive or deactivated state. The terms reference activation, or activating, sensitizing, or up-regulating signal transduction or enzymatic activity or the amount of a protein decreased in a disease.

The terms “agonist,” “activator,” “upregulator,” etc. refer to a substance capable of detectably increasing the expression or activity of a given gene or protein. The agonist can increase expression or activity by at least 10%, 20%, 30%, 40%, 50%, 60%, 70%, 80%, 90%, 95%, 96%, 97%, 98%, or 99% in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition,” “inhibit,” “inhibiting” and the like in reference to a cellular component-inhibitor interaction means negatively affecting (e.g., decreasing) the activity or function of the cellular component (e.g., decreasing the signaling pathway stimulated by a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)), relative to the activity or function of the cellular component in the absence of the inhibitor. In embodiments inhibition means negatively affecting (e.g., decreasing) the concentration or levels of the cellular component relative to the concentration or level of the cellular component in the absence of the inhibitor. In some embodiments, inhibition refers to reduction of a disease or symptoms of disease. In some embodiments, inhibition refers to a reduction in the activity of a signal transduction pathway or signaling pathway (e.g., reduction of a pathway involving the cellular component). Thus, inhibition includes, at least in part, partially or totally blocking stimulation, decreasing, preventing, or delaying activation, or inactivating, desensitizing, or down-regulating the signaling pathway or enzymatic activity or the amount of a cellular component.

The terms “inhibitor,” “repressor,” “antagonist,” or “downregulator” interchangeably refer to a substance capable of detectably decreasing the expression or activity of a given gene or protein. The antagonist can decrease expression or activity by at least 10%, 20%, 30%, 40%, 50%, 60%, 70%, 80%, 90%, 95%, 96%, 97%, 98%, or 99% in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule (e.g., a target may be a cellular component (e.g., protein, ion, lipid, virus, lipid droplet, nucleic acid, nucleotide, amino acid, protein, particle, organelle, cellular compartment, microorganism, vesicle, small molecule, protein complex, protein aggregate, or macromolecule)) relative to the absence of the composition.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometrv, immunofluorescence, immunohistochemistry, etc.).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

“Patient”, “patient in need thereof”, “subject”, or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a patient is human. In embodiments, a patient in need thereof is human. In embodiments, a subject is human. In embodiments, a subject in need thereof is human.

“Disease” or “condition” refer to a state of being or health status of a patient or subject capable of being treated with the compounds or methods provided herein. In some embodiments, the disease is a disease related to (e.g., caused by) a cellular component (e.g., protein, ion, lipid, nucleic acid, nucleotide, amino acid, protein, particle, organelle, cellular compartment, microorganism, vesicle, small molecule, protein complex, protein aggregate, or macromolecule). In embodiments, the disease is a neurodegenerative disease. In embodiments, the disease is a liver disease. In embodiments, the disease is a fibrotic disease. In embodiments, the disease is a coronavirus infection.

As used herein, the term “neurodegenerative disease” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease. Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease. HIV-associated dementia, Kennedy's disease, Krabbe's disease, kuru, Lewy body dementia. Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

As used herein, the term “tauopathy” refers to a neurodegenerative disease characterized by tau deposits in the brain. Examples of tauopathies include, but are not limited to, Alzheimer's disease, primary age-related tauopathy, chronic traumatic encephalopathy, progressive supranuclear palsy, corticobasal degeneration, frontotemporal dementia, vacuolar tauopathy, Lytico-bodig disease, ganglioglioma, gangliocytoma, meningioangiomatosis, postencephalitic parkinsonism, subacute sclerosing panencephalitis, lead encephalopathy, tuberous sclerosis. Pantothenate kinase-associated neurodegeneration, and lipofuscinosis.

As used herein, the term “liver disease” refers to a disease or condition characterized by liver problems (e.g., an increased level of liver problems compared to a control such as a healthy person not suffering from a disease). Examples of liver diseases include, but are not limited to, Alagille syndrome, autoimmune hepatitis, biliary atresia, cirrhosis, endoscopic retrograde cholangiopancreatography (ERCP), hemochromatosis, hepatitis A, hepatitis B, hepatitis C, hepatitis D, hepatitis E, viral hepatitis, liver cancer, nonalcoholic fatty liver disease (NAFLD), nonalcoholic steatohepatitis (NASH), porphyria, primary biliary cholangitis, primary sclerosing cholangitis (PSC), and Wilson disease.

The term “fibrosis” or “fibrotic disease” is used in accordance with its plain ordinary meaning and refers to any disease or condition characterized by the formation of excess fibrous connective tissue. The formation of excess fibrous connective tissue may be in response to a reparative or reactive process. Fibrosis may be pulmonary fibrosis, liver fibrosis, myelofibrosis, skin fibrosis (e.g., nephrogenic systemic fibrosis and keloid fibrosis), mediastinal fibrosis, cardiac fibrosis, kidney fibrosis, stromal fibrosis, epidural fibrosis, epithelial fibrosis, or idiopathic fibrosis.

The term “coronavirus” is used in accordance with its plain ordinary meaning and refers to an RNA virus that in humans causes respiratory tract infections. Coronaviruses constitute the subfamily Orthocoronavirinae, in the family Coronaviridae, order Nidovirales, and realm Riboviria. In embodiments, the coronavirus is an enveloped viruses with a positive-sense single-stranded RNA genome.

The term “severe acute respiratory syndrome coronavirus” or “SARS-CoV” or “SARS-CoV-1” refers to the strain of coronavirus that causes severe acute respiratory syndrome (SARS). In embodiments, SARS-CoV-1 is an enveloped, positive-sense, single-stranded RNA virus that infects the epithelial cells within the lungs. In embodiments, the virus enters the host cell by binding to the angiotensin-converting enzyme 2 (ACE2) receptor.

The term “severe acute respiratory syndrome coronavirus 2” or “SARS-CoV-2” refers to the strain of coronavirus that causes coronavirus disease 2019 (COVID-19). In embodiments, SARS-CoV-2 is a positive-sense single-stranded RNA virus.

As used herein, the term “cancer” refers to all types of cancer, neoplasm or malignant tumors found in mammals (e.g., humans), including leukemia, lymphoma, carcinomas and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus medulloblastoma, colorectal cancer, or pancreatic cancer. Additional examples include Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma. Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifomi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor. e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The term “drug” is used in accordance with its common meaning and refers to a substance which has a physiological effect (e.g., beneficial effect, is useful for treating a subject) when introduced into or to a subject (e.g., in or on the body of a subject or patient).

A drug moiety is a radical of a drug.

A “detectable agent,” “detectable compound,” “detectable label,” or “detectable moiety” is a substance (e.g., element), molecule, or composition detectable by spectroscopic, photochemical, biochemical, immunochemical, chemical, magnetic resonance imaging, or other physical means. For example, detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y ⁸⁹Sr, ⁸⁹Zr, ⁹⁴Tc, ⁹⁴Tc, ⁹⁹mTc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g., fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g., carbon-11, nitrogen-13, oxygen-15, fluorine-18, rubidium-82), fluorodeoxyglucose (e.g., fluorine-18 labeled), any gamma ray emitting radionuclides, positron-emitting radionuclide, radiolabeled glucose, radiolabeled water, radiolabeled ammonia, biocolloids, microbubbles (e.g., including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ⁹⁹mTc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶HO, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb, and Lu.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the term “administering” is used in accordance with its plain and ordinary meaning and includes oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, orjust after the administration of one or more additional therapies. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compounds described herein can be used in combination with one another, with other active agents known to be useful in treating a disease associated with cells expressing a disease associated cellular component, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In therapeutic use for the treatment of a disease, compound utilized in the pharmaceutical compositions of the present invention may be administered at the initial dosage of about 0.001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound or drug being employed. For example, dosages can be empirically determined considering the type and stage of disease (e.g., neurodegenerative disease, liver disease, fibrotic disease, or coronavirus infection) diagnosed in a particular patient. The dose administered to a patient, in the context of the present invention, should be sufficient to affect a beneficial therapeutic response in the patient over time. The size of the dose will also be determined by the existence, nature, and extent of any adverse side effects that accompany the administration of a compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired.

The term “associated” or “associated with” in the context of a substance or substance activity or function associated with a disease (e.g., a protein associated disease, disease associated with a cellular component) means that the disease (e.g., neurodegenerative disease, liver disease, fibrotic disease, or coronavirus infection) is caused by (in whole or in part), or a symptom of the disease is caused by (in whole or in part) the substance or substance activity or function or the disease or a symptom of the disease may be treated by modulating (e.g., inhibiting or activating) the substance (e.g., cellular component). As used herein, what is described as being associated with a disease, if a causative agent, could be a target for treatment of the disease.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The term “electrophilic” as used herein refers to a chemical group that is capable of accepting electron density. An “electrophilic substituent,” “electrophilic chemical moiety,” or “electrophilic moiety” refers to an electron-poor chemical group, substituent, or moiety (monovalent chemical group), which may react with an electron-donating group, such as a nucleophile, by accepting an electron pair or electron density to form a bond.

“Nucleophilic” as used herein refers to a chemical group that is capable of donating electron density.

The term “isolated.” when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

An amino acid residue in a protein “corresponds” to a given residue when it occupies the same essential structural position within the protein as the given residue. For example, a selected residue in a selected protein corresponds to Y198^{A} of procaspase-6 when the selected residue occupies the same essential spatial or other structural relationship as Y198^{A} of procaspase-6. In some embodiments, where a selected protein is aligned for maximum homology with the procaspase-6 protein, the position in the aligned selected protein aligning with Y198^{A} is said to correspond to Y198^{A}. Instead of a primary sequence alignment, a three dimensional structural alignment can also be used, e.g., where the structure of the selected protein is aligned for maximum correspondence with the procaspase-6 protein and the overall structures compared. In this case, an amino acid that occupies the same essential position as Y198^{A} in the structural model is said to correspond to the Y198^{A} residue.

The term “protein complex” is used in accordance with its plain ordinary meaning and refers to a protein which is associated with an additional substance (e.g., another protein, protein subunit, or a compound). Protein complexes typically have defined quaternary structure. The association between the protein and the additional substance may be a covalent bond. In embodiments, the association between the protein and the additional substance (e.g., compound) is via non-covalent interactions. In embodiments, a protein complex refers to a group of two or more polypeptide chains. Proteins in a protein complex are linked by non-covalent protein-protein interactions. A non-limiting example of a protein complex is the proteasome.

The term “protein aggregate” is used in accordance with its plain ordinary meaning and refers to an aberrant collection or accumulation of proteins (e.g., misfolded proteins).

Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In embodiments, protein aggregates are termed aggresomes.

The term “procaspase 6” or “procaspase-6” refers to an inactive precursor form of caspase 6. In embodiments, procaspase-6 exhibits a dimeric structure.

The term “caspase 6” or “caspase-6” or “Casp6” refers to a protein (including homologs, isoforms, and functional fragments thereof) that is a member of the cysteine-aspartic acid protease (caspase) family. Caspase-6 cleaves substrates (e.g., HTT in Huntington's, APP in Alzheimer's disease, tau in Alzheimer's disease), which may result in protein aggregation of the fragments. In embodiments, caspase 6 cleaves substrates that lead to inflammation (e.g., neuroinflammation), and to cell death. In embodiments, cell death leads to cirrhosis and fibrosis (e.g., in liver or other organs). In embodiments, caspase-6 is involved in axonal degradation. The term includes any recombinant or naturally-occurring form of caspase-6 variants thereof that maintain caspase-6 activity (e.g., within at least 30%, 40%, 50%, 60%, 70%, 80%, 90%, 95%, or 100% activity compared to wildtype caspase-6). In embodiments, caspase-6 is encoded by the CASP6 gene. In embodiments, caspase-6 has the amino acid sequence set forth in or corresponding to Entrez 839, UniProt P55212, RefSeq (protein) NP_001217.2, or RefSeq (protein) NP_116787.1. In embodiments, caspase-6 has the sequence:

(SEQ ID NO: 1)
MSSASGLRRGHPAGGEENMTETDAFYKREMFDPAEKYKMDHRRRGIALI
FNHERFFWHLTLPERRGTCADRDNLTRRFSDLGFEVKCFNDLKAEELLL
KIHEVSTVSHADADCFVCVFLSHGEGNHIYAYDAKIEIQTLTGLFKGDK
CHSLVGKPKIFIIQACRGNQHDVPVIPLDVVDNQTEKLDTNITEVDAAS
VYTLPAGADFLMCYSVAEGYYSHRETVNGSWYIQDLCEMLGKYGSSLEF
TELLTLVNRKVSQRRVDFCKDPSAIGKKQVPCFASMLTKKLHFFPKSN.

### II. Compounds

In an aspect is provided a compound, or a pharmaceutically acceptable salt thereof, having the formula:

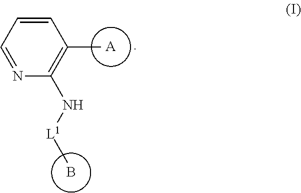

Ring A is a substituted or unsubstituted 5 to 6 membered heteroaryl or substituted or unsubstituted 8 to 10 membered fused ring heteroaryl.

Ring B is a substituted or unsubstituted 9 to 10 membered fused ring aryl or substituted or unsubstituted 9 to 10 membered fused ring heteroaryl.

L¹ is a bond or substituted or unsubstituted C₁-C₃ alkylene.

In embodiments, a substituted Ring B (e.g., substituted 9 to 10 membered fused ring aryl and/or substituted 9 to 10 membered fused ring heteroaryl) is substituted with at least one substituent group, size-limited substituent group, or lower substituent group; wherein if the substituted Ring B is substituted with a plurality of groups selected from substituent groups, size-limited substituent groups, and lower substituent groups: each substituent group, size-limited substituent group, and/or lower substituent group may optionally be different. In embodiments, when Ring B is substituted, it is substituted with at least one substituent group.

In embodiments, when Ring B is substituted, it is substituted with at least one size-limited substituent group. In embodiments, when Ring B is substituted, it is substituted with at least one lower substituent group.

In embodiments, Ring B is substituted or unsubstituted benzoisoxazolyl, substituted or unsubstituted benzoisothiazolyl, substituted or unsubstituted benzofuranyl, substituted or unsubstituted indazolyl, substituted or unsubstituted benzoxazolyl, substituted or unsubstituted benzothiazolyl, substituted or unsubstituted benzothiophenyl, or substituted or unsubstituted indolyl. In embodiments, Ring B is substituted or unsubstituted benzoisoxazolyl. In embodiments, Ring B is substituted or unsubstituted benzoisothiazolyl.

In embodiments. Ring B is substituted or unsubstituted benzofuranyl. In embodiments, Ring B is substituted or unsubstituted indazolyl. In embodiments, Ring B is substituted or unsubstituted benzoxazolyl. In embodiments, Ring B is substituted or unsubstituted benzothiazolyl. In embodiments, Ring B is substituted or unsubstituted benzothiophenyl. In embodiments, Ring B is substituted or unsubstituted indolyl.

In embodiments, Ring B is

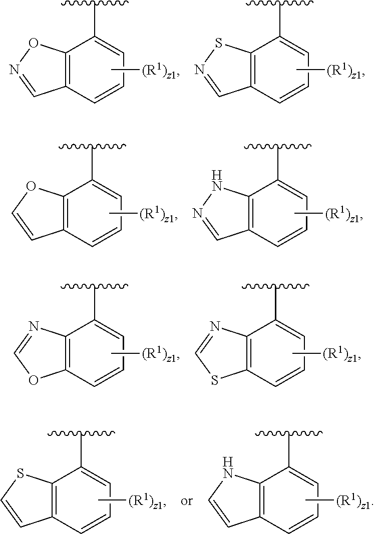

R¹ is independently halogen. —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —B(OR^{1C})(OR^{1D}), —SF₅, —N₃, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁-C₈, C₁-C₆, C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, 4 to 6 membered, 4 to 5 membered, or 5 to 6 membered) or substituted or unsubstituted heteroaryl (e.g., 5 to 10 membered, 5 to 9 membered, or 5 to 6 membered).

Each X¹ is independently —F, —Cl, —Br, or —I.

The symbol n1 is an integer from 0 to 4.

The symbols m1 and v1 are independently 1 or 2.

The symbol z1 is an integer from 0 to 5.

In embodiments, Ring B is

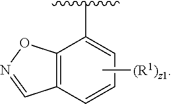

In embodiments, Ring B is

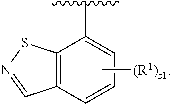

In embodiments, Ring B is

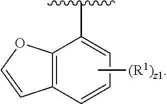

In embodiments, Ring B is

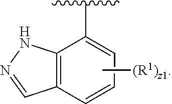

In embodiments, Ring B is

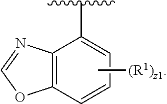

In embodiments, Ring B is

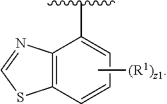

In embodiments, Ring B is

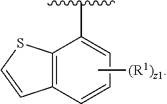

In embodiments, Ring B is

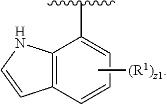

In embodiments, Ring B is

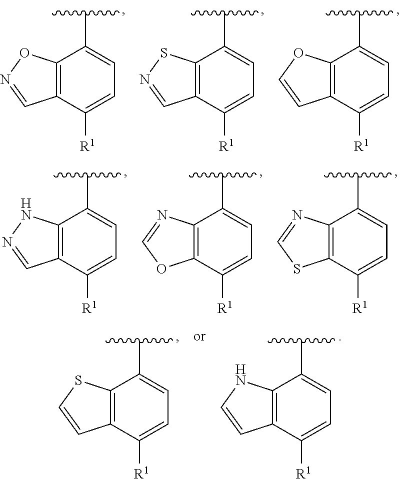

In embodiments, Ring B is

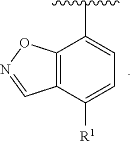

In embodiments, Ring B is

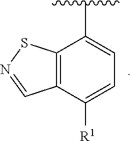

In embodiments, Ring B is

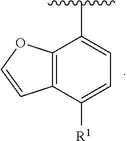

In embodiments, Ring B is

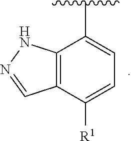

In embodiments, Ring B is

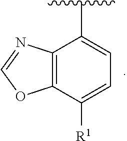

In embodiments, Ring B is

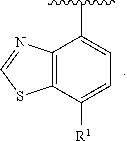

In embodiments, Ring B is

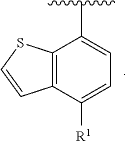

In embodiments, Ring B is

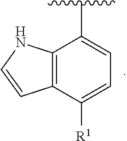

In embodiments, Ring B is

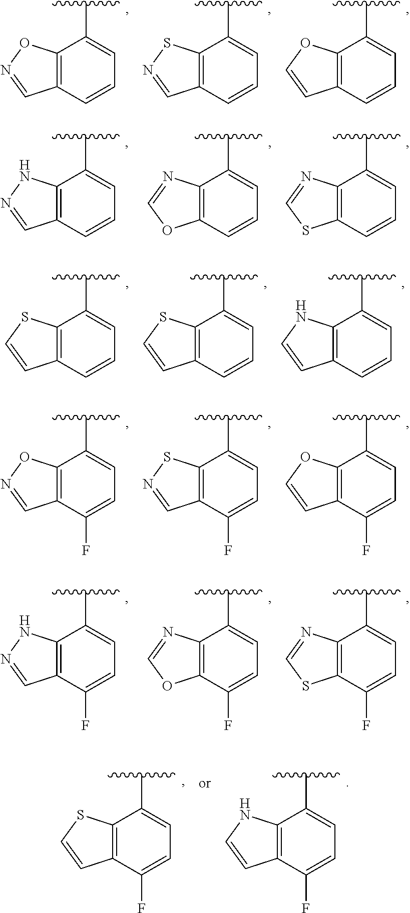

In embodiments, Ring B is

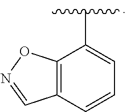 [SMILES: *c1cccc2cnoc12]

In embodiments, Ring B is

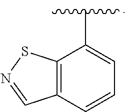 [SMILES: *c1cccc2cnsc12]

In embodiments, Ring B is

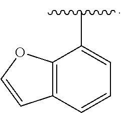 [SMILES: *c1cccc2ccoc12]

In embodiments, Ring B is

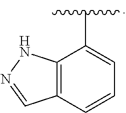 [SMILES: *c1cccc2cn[nH]c12]

In embodiments, Ring B is

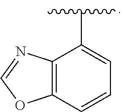 [SMILES: *c1cccc2ocnc12]

In embodiments, Ring B is

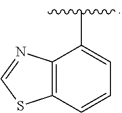 [SMILES: *c1cccc2scnc12]

In embodiments, Ring B is

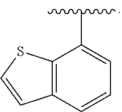 [SMILES: *c1cccc2ccsc12]

In embodiments, Ring B is

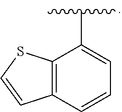 [SMILES: *c1cccc2ccsc12]

In embodiments, Ring B is

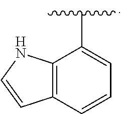 [SMILES: *c1cccc2cc[nH]c12]

In embodiments, Ring B is

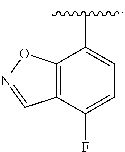 [SMILES: *c1ccc(F)c2cnoc12]

In embodiments, Ring B is

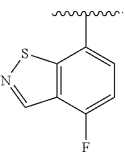 [SMILES: *c1ccc(F)c2cnsc12]

In embodiments, Ring B is

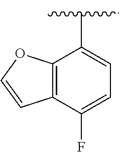 [SMILES: *c1ccc(F)c2ccoc12]

In embodiments, Ring B is

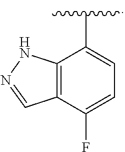 [SMILES: *c1ccc(F)c2cn[nH]c12]

In embodiments, Ring B is

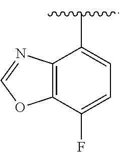 [SMILES: *c1ccc(F)c2ocnc12]

In embodiments, Ring B is

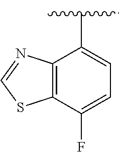 [SMILES: *c1ccc(F)c2scnc12]

In embodiments, Ring B is

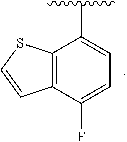 [SMILES: *c1ccc(F)c2ccsc12]

In embodiments, Ring B is

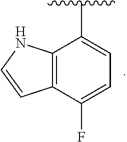 [SMILES: *c1ccc(F)c2cc[nH]c12]

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when Ria is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups: each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups: each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups: each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H. —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F, —OCH₂I, —OCHCl₂, —OCHBr₂, —OCHF₂, —OCHI₂, —B(OH)₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is independently halogen. —CCl₃, —CBr₃, —CF₃, —CI₃. —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, ONH₂, —NHC(O)NH₂, —NHSO₂H, —NHC(O)H, —NHC(O)OH, —NHOH, —OCCl₃, —OCBr₃, —OCF₃, —OCl₃, —OCH₂Cl, —OCH₂Br, —OCH₂F. —OCH₂I, —OCHCl₂. —OCHBr₂, —OCHF₂, —OCHL₂, —B(OH)₂, —SF₅. —N₃, substituted or unsubstituted C₁-C₆ alkyl, substituted or unsubstituted 2 to 6 membered heteroalkyl, substituted or unsubstituted C₃-C₈ cycloalkyl, substituted or unsubstituted 3 to 8 membered heterocycloalkyl, substituted or unsubstituted phenyl, or substituted or unsubstituted 5 to 6 membered heteroaryl.

In embodiments, R¹ is independently halogen. In embodiments, R¹ is independently —F. In embodiments, R¹ is independently —Cl. In embodiments, R¹ is independently —Br. In embodiments, R¹ is independently —I. In embodiments, R¹ is independently —OR^{1D}, wherein R^{1D} is as described herein, including in embodiments. In embodiments, R¹ is independently —OH. In embodiments, R¹ is independently —NH₂. In embodiments, R¹ is independently —B(OR^{1C})(OR^{1D}), wherein R^{1C} and R^{1D} are as described herein, including in embodiments. In embodiments, R¹ is independently —B(OR^{1C})(OH), wherein R^{1C} is as described herein, including in embodiments. In embodiments, R¹ is independently —B(OH)₂. In embodiments, R¹ is independently unsubstituted C₁-C₄ alkyl. In embodiments, R¹ is independently unsubstituted methyl. In embodiments, R¹ is independently unsubstituted ethyl. In embodiments, R¹ is independently unsubstituted propyl. In embodiments, R¹ is independently unsubstituted n-propyl. In embodiments, R¹ is independently unsubstituted isopropyl. In embodiments, R¹ is independently unsubstituted butyl. In embodiments, R¹ is independently unsubstituted n-butyl. In embodiments, R¹ is independently unsubstituted isobutyl. In embodiments, R¹ is independently unsubstituted tert-butyl. In embodiments, R¹ is independently unsubstituted 2 to 6 membered heteroalkyl. In embodiments, R¹ is independently unsubstituted methoxy. In embodiments, R¹ is independently unsubstituted ethoxy. In embodiments, R¹ is independently unsubstituted propoxy. In embodiments, R¹ is independently unsubstituted n-propoxy. In embodiments, R¹ is independently unsubstituted isopropoxy. In embodiments, R¹ is independently unsubstituted butoxy.

In embodiments, R^{1A} is independently hydrogen. In embodiments, R^{1A} is independently unsubstituted C₁-C₄ alkyl. In embodiments, R^{1A} is independently unsubstituted methyl. In embodiments, R^{1A} is independently unsubstituted ethyl. In embodiments, R^{1A} is independently unsubstituted propyl. In embodiments, R^{1A} is independently unsubstituted n-propyl. In embodiments, R^{1A} is independently unsubstituted isopropyl. In embodiments, R^{1A} is independently unsubstituted butyl. In embodiments, R^{1A} is independently unsubstituted n-butyl. In embodiments, R^{1A} is independently unsubstituted isobutyl. In embodiments. R^{1A} is independently unsubstituted tert-butyl.

In embodiments, R^{1B} is independently hydrogen. In embodiments, R^{1B} is independently unsubstituted C₁-C₄ alkyl. In embodiments, R^{1B} is independently unsubstituted methyl. In embodiments, R^{1B} is independently unsubstituted ethyl. In embodiments, R^{1B} is independently unsubstituted propyl. In embodiments. R^{1B} is independently unsubstituted n-propyl. In embodiments, R^{1B} is independently unsubstituted isopropyl. In embodiments, R^{1B} is independently unsubstituted butyl. In embodiments, R^{1B} is independently unsubstituted n-butyl. In embodiments, R^{1B} is independently unsubstituted isobutyl. In embodiments. R^{1B} is independently unsubstituted tert-butyl.

In embodiments, R^{1C} is independently hydrogen. In embodiments, R^{1C} is independently unsubstituted C₁-C₄ alkyl. In embodiments, R^{1C} is independently unsubstituted methyl. In embodiments, R^{1C} is independently unsubstituted ethyl. In embodiments, R^{1C} is independently unsubstituted propyl. In embodiments, RIC is independently unsubstituted n-propyl. In embodiments, RC is independently unsubstituted isopropyl. In embodiments, R^{1C} is independently unsubstituted butyl. In embodiments, R^{1C} is independently unsubstituted n-butyl. In embodiments, R^{1C} is independently unsubstituted isobutyl. In embodiments, R^{1C} is independently unsubstituted tert-butyl.

In embodiments, R^{1D} is independently hydrogen. In embodiments, R^{1D} is independently unsubstituted C₁-C₄ alkyl. In embodiments, R^{1D} is independently unsubstituted methyl. In embodiments, R^{1D} is independently unsubstituted ethyl. In embodiments, R^{1D} is independently unsubstituted propyl. In embodiments, R^{1D} is independently unsubstituted n-propyl. In embodiments, R^{1D} is independently unsubstituted isopropyl. In embodiments, R^{1D} is independently unsubstituted butyl. In embodiments, R^{1D} independently unsubstituted n-butyl. In embodiments, R^{1D} is independently unsubstituted isobutyl. In embodiments, R^{1D} is independently unsubstituted tert-butyl.

In embodiments, z1 is 0. In embodiments, z1 is 1. In embodiments, z1 is 2. In embodiments, z1 is 3. In embodiments, z1 is 4. In embodiments, z1 is 5.

In embodiments, a substituted Ring A (e.g., substituted 5 to 6 membered heteroaryl and/or substituted 8 to 10 membered fused ring heteroaryl) is substituted with at least one substituent group, size-limited substituent group, or lower substituent group; wherein if the substituted Ring A is substituted with a plurality of groups selected from substituent groups, size-limited substituent groups, and lower substituent groups; each substituent group, size-limited substituent group, and/or lower substituent group may optionally be different. In embodiments, when Ring A is substituted, it is substituted with at least one substituent group. In embodiments, when Ring A is substituted, it is substituted with at least one size-limited substituent group. In embodiments, when Ring A is substituted, it is substituted with at least one lower substituent group.

In embodiments, Ring A is a substituted or unsubstituted nitrogen-containing 5 to 6 membered heteroaryl or substituted or unsubstituted nitrogen-containing 8 to 10 membered fused ring heteroaryl. In embodiments, Ring A is a substituted or unsubstituted nitrogen-containing 5 to 6 membered heteroaryl. In embodiments, Ring A is a substituted or unsubstituted nitrogen-containing 8 to 10 membered fused ring heteroaryl.

In embodiments, Ring A is substituted or unsubstituted pyridyl, substituted or unsubstituted pyridazinyl, substituted or unsubstituted pyrimidinyl, substituted or unsubstituted pyrazinyl, substituted or unsubstituted triazinyl, substituted or unsubstituted imidazolyl, substituted or unsubstituted pyrazolyl, substituted or unsubstituted oxazolyl, substituted or unsubstituted isoxazolyl, substituted or unsubstituted furanyl, substituted or unsubstituted thienyl, substituted or unsubstituted thiazolyl, substituted or unsubstituted isothiazolyl, substituted or unsubstituted triazolyl, substituted or unsubstituted oxadiazolyl, substituted or unsubstituted tetrazolyl, substituted or unsubstituted imidazopyridinyl, substituted or unsubstituted benzothiazolyl, or substituted or unsubstituted benzoimidazolyl. In embodiments, Ring A is substituted or unsubstituted pyridyl. In embodiments, Ring A is substituted or unsubstituted pyridazinyl. In embodiments, Ring A is substituted or unsubstituted pyrimidinyl. In embodiments, Ring A is substituted or unsubstituted pyrazinyl. In embodiments, Ring A is substituted or unsubstituted triazinyl. In embodiments, Ring A is substituted or unsubstituted imidazolyl. In embodiments, Ring A is substituted or unsubstituted pyrazolyl. In embodiments. Ring A is substituted or unsubstituted oxazolyl. In embodiments, Ring A is substituted or unsubstituted isoxazolyl. In embodiments, Ring A is substituted or unsubstituted furanyl. In embodiments, Ring A is substituted or unsubstituted thienyl. In embodiments, Ring A is substituted or unsubstituted thiazolyl. In embodiments, Ring A is substituted or unsubstituted isothiazolyl. In embodiments, Ring A is substituted or unsubstituted triazolyl. In embodiments, Ring A is substituted or unsubstituted oxadiazolyl. In embodiments, Ring A is substituted or unsubstituted tetrazolyl. In embodiments, Ring A is substituted or unsubstituted imidazopyridinyl. In embodiments, Ring A is substituted or unsubstituted benzothiazolyl. In embodiments, Ring A is substituted or unsubstituted benzoimidazolyl.

In embodiments, Ring A is

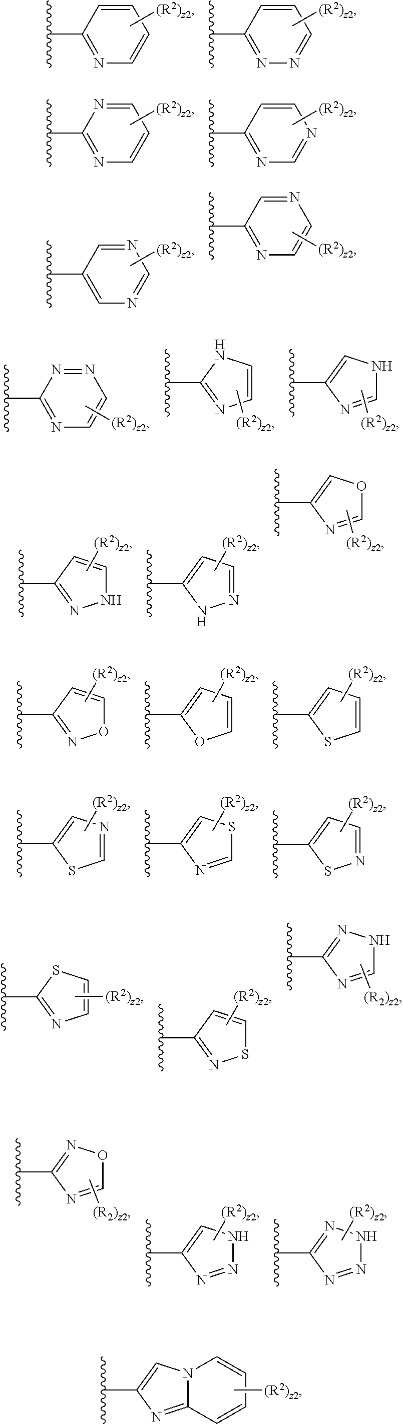

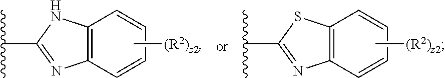

R² is independently halogen, —CCl₃, —CBr₃, —CF₃. —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br. —OCH₂F, —OCH₂I, —OCHCl₂, —OCHBr₂, —OCHF₂, —OCHI₂, —SF₅, —N₃, substituted or unsubstituted alkyl (e.g., C₁-C₈, C₁-C₆. C₁-C₄, or C₁-C₂), substituted or unsubstituted heteroalkyl (e.g., 2 to 8 membered, 2 to 6 membered, 4 to 6 membered, 2 to 3 membered, or 4 to 5 membered), substituted or unsubstituted cycloalkyl (e.g., C₃-C₈, C₃-C₆, C₄-C₆, or C₅-C₆), substituted or unsubstituted heterocycloalkyl (e.g., 3 to 8 membered, 3 to 6 membered, 4 to 6 membered, 4 to 5 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered).

The symbol z2 is an integer from 0 to 5.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, R² is independently —NH₂ or unsubstituted C₁-C₄ alkyl. In embodiments, R² is independently —NH₂ or unsubstituted methyl. In embodiments, R² is independently —NH₂. In embodiments, R² is independently unsubstituted C₁-C₄ alkyl. In embodiments, R² is independently unsubstituted methyl. In embodiments, R² is independently unsubstituted ethyl. In embodiments, R² is independently unsubstituted propyl. In embodiments, R² is independently unsubstituted n-propyl. In embodiments, R² is independently unsubstituted isopropyl. In embodiments, R² is independently unsubstituted butyl. In embodiments, R² is independently unsubstituted n-butyl. In embodiments, R² is independently unsubstituted isobutyl. In embodiments, R² is independently unsubstituted tert-butyl.

In embodiments, z2 is 0. In embodiments, z2 is 1. In embodiments, z2 is 2. In embodiments, z2 is 3. In embodiments, z2 is 4. In embodiments, z2 is 5. In embodiments, Ring A is

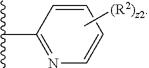

In embodiments, Ring A is

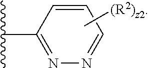

In embodiments, Ring A is

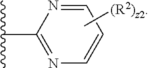

In embodiments, Ring A is

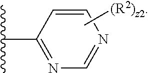

In embodiments, Ring A is

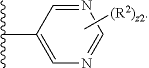

In embodiments, Ring A is

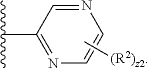

In embodiments, Ring A is

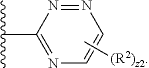

In embodiments, Ring A is

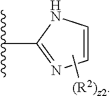

In embodiments, Ring A is

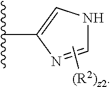

In embodiments, Ring A is

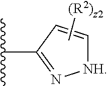

In embodiments, Ring A is

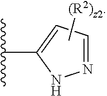

In embodiments, Ring A is

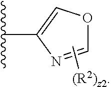

In embodiments, Ring A is

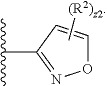

In embodiments, Ring A is

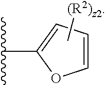

In embodiments, Ring A is

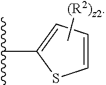

In embodiments, Ring A is

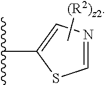

In embodiments, Ring A is

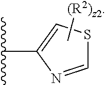

In embodiments, Ring A is

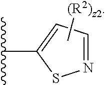

In embodiments, Ring A is

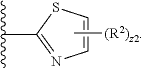

In embodiments, Ring A is

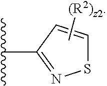

In embodiments, Ring A is

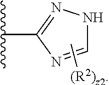

In embodiments, Ring A is

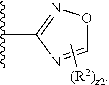

In embodiments, Ring A is

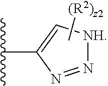

In embodiments, Ring A is

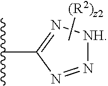

In embodiments, Ring A is

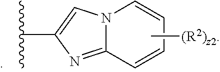

In embodiments, Ring A is

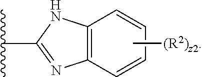

In embodiments, Ring A is

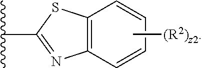

In embodiments, Ring A is

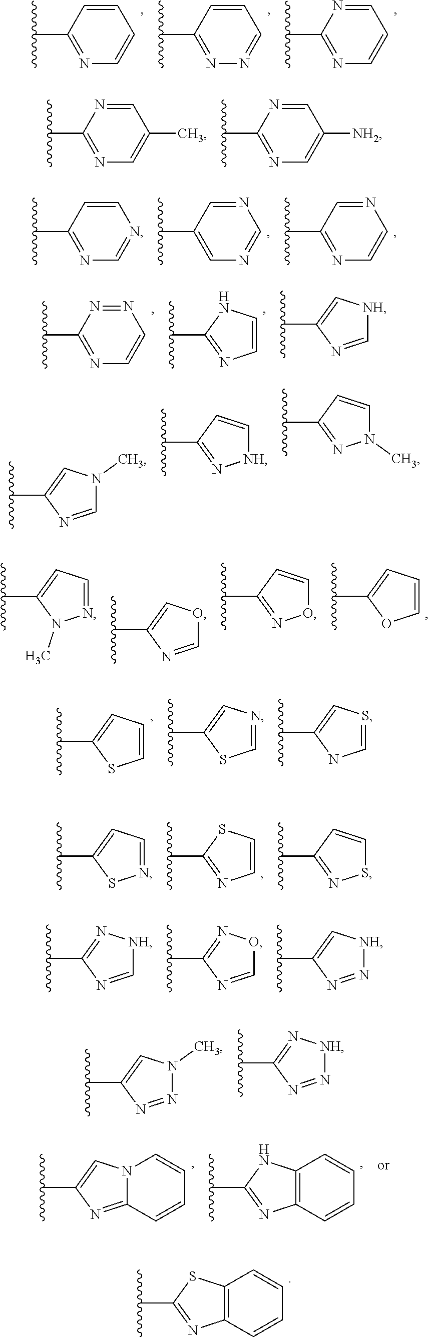

In embodiments, Ring A is

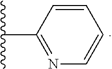 [SMILES: *c1ccccn1]

In embodiments, Ring A is

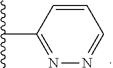 [SMILES: *c1cccnn1]

In embodiments, Ring A is

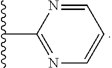 [SMILES: *c1ncccn1]

In embodiments, Ring A is

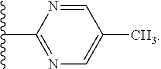 [SMILES: *c1ncc(C)cn1]

In embodiments, Ring A is

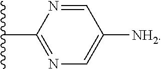 [SMILES: *c1ncc(N)cn1]

In embodiments, Ring A is

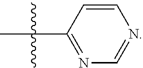 [SMILES: *c1ccncn1]

In embodiments, Ring A is

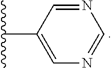 [SMILES: *c1cncnc1]

In embodiments, Ring A is

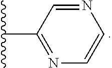 [SMILES: *c1cnccn1]

In embodiments, Ring A is

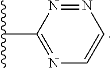 [SMILES: *c1nccnn1]

In embodiments, Ring A is

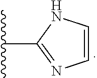 [SMILES: *c1ncc[nH]1]

In embodiments, Ring A is

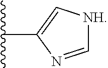 [SMILES: *c1c[nH]cn1]

In embodiments, Ring A is

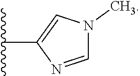 [SMILES: *c1cn(C)cn1]

In embodiments, Ring A

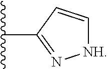 [SMILES: *c1cc[nH]n1]

In embodiments, Ring A is

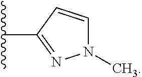 [SMILES: *c1ccn(C)n1]

In embodiments, Ring A is

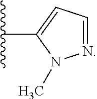 [SMILES: *c1ccnn1C]

In embodiments, Ring A is

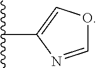 [SMILES: *c1cocn1]

In embodiments, Ring A is

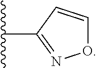 [SMILES: *c1ccon1]

In embodiments, Ring A is

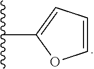 [SMILES: *c1ccco1]

In embodiments, Ring A is

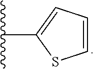 [SMILES: *c1cccs1]

In embodiments, Ring A is

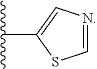 [SMILES: *c1cncs1]

In embodiments, Ring A is

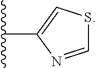 [SMILES: *c1cscn1]

In embodiments, Ring A is

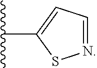 [SMILES: *c1ccns1]

In embodiments. Ring A is

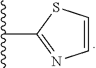 [SMILES: *c1nccs1]

In embodiments, Ring A is

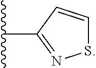 [SMILES: *c1ccsn1]

In embodiments, Ring A is

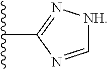 [SMILES: *c1nc[nH]n1]

In embodiments, Ring A is

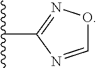 [SMILES: *c1ncon1]

In embodiments, Ring A is

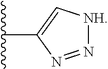 [SMILES: *c1c[nH]nn1]

In embodiments, Ring A is

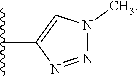 [SMILES: *c1cn(C)nn1]

In embodiments, Ring A is

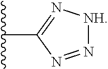 [SMILES: *c1nn[nH]n1]

In embodiments, Ring A is

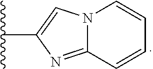 [SMILES: *c1cn2ccccc2n1]

In embodiments, Ring A is

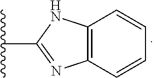 [SMILES: *c1nc2ccccc2[nH]1]

In embodiments, Ring A is

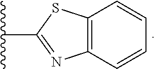 [SMILES: *c1nc2ccccc2s1]

In embodiments, a substituted L¹ (e.g., substituted C₁-C₃ alkylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, L¹ is unsubstituted C₁-C₃ alkylene. In embodiments, L¹ is unsubstituted methylene. In embodiments, L¹ is unsubstituted ethylene. In embodiments, L¹ is unsubstituted propylene. In embodiments, L¹ is unsubstituted n-propylene. In embodiments, L¹ is unsubstituted isopropylene.

In embodiments, when Ring A is substituted, Ring A is substituted with one or more first substituent groups denoted by R^{A} as explained in the definitions section above in the description of “first substituent group(s)”. In embodiments, when an R^{AA} substituent group is substituted, the R^{A.1} substituent group is substituted with one or more second substituent groups denoted by R^{A.2} as explained in the definitions section above in the description of “first substituent group(s)”. In embodiments, when an R^{A.2} substituent group is substituted, the R^{A.2} substituent group is substituted with one or more third substituent groups denoted by R^{A.3} as explained in the definitions section above in the description of “first substituent group(s)”. In the above embodiments, Ring A, R^{A.1}, R^{A.2}, and R^{A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to Ring A, R^{A.1}, R^{A.2}, and R^{A.3}, respectively.

In embodiments, when Ring B is substituted, Ring B is substituted with one or more first substituent groups denoted by R^{B.1} as explained in the definitions section above in the description of “first substituent group(s)”. In embodiments, when an R^{B.1} substituent group is substituted, the R^{B.1} substituent group is substituted with one or more second substituent groups denoted by R^{B.2} as explained in the definitions section above in the description of “first substituent group(s)”. In embodiments, when an R^{B.2} substituent group is substituted, the R^{B.2} substituent group is substituted with one or more third substituent groups denoted by R^{B.3} as explained in the definitions section above in the description of “first substituent group(s)”. In the above embodiments, Ring B, R^{B.1}, R^{B.2}, and R^{B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to Ring B, R^{B.1}, R^{B.2}, and R^{B.3}, respectively.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2} and R^{WW.3} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1A}, R^{1A.1}, R^{1A.2} and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2}, and R^{1B.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1B}, R^{1B.1}, R^{1B.2}, and R^{1B.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A.2} as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments. R^{1A.1}, R^{1A.2}, and R^{1A.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1A.1}. R^{1A.2}, and R^{1A.3}, respectively.

In embodiments, when R^{1A} and R^{1B} substituents bonded to the same nitrogen atom are optionally joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B.1}, R^{1B.2}, and R^{1B.3} have values corresponding to the values of R^{WW.1}, R^{WW.2}, and R^{WW.3}, respectively, as explained in the definitions section above in the description of “first substituent group(s)”, wherein R^{WW.1}, R^{WW.2}, and R^{WW.3} correspond to R^{1B.1}, R^{1B.2}, and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C2}, and R^{1C3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1C}, R^{1C.1}, R^{1C.2}, and R^{1C.3} respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2} and R^{1D.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.3}, respectively, as explained in the definitions section above in the description of “first substituent group(s)”, wherein R^{WW}, R^{WW.1}, R^{WW.2}, and R^{WW.3} correspond to R², R^{2.1}, R^{2.2}, and R^{2.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3} respectively, as explained in the definitions section above in the description of “first substituent group(s)”, wherein L^{WW}, R^{LWW.1}, R^{LWW.2}, and R^{LWW.3} are L¹, R^{L1.1}, R^{L1.2}, and R^{L1.3}, respectively.

In embodiments, the compound has the formula:

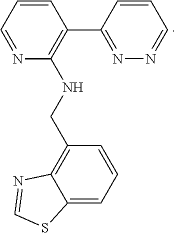 [SMILES: c1cnnc(-c2cccnc2NCc2cccc3scnc23)c1]

In embodiments, the compound has the formula:

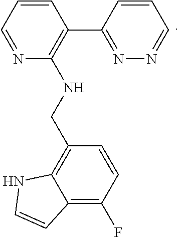 [SMILES: Fc1ccc(CNc2ncccc2-c2cccnn2)c2[nH]ccc12]

In embodiments, the compound has the formula:

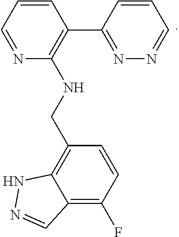 [SMILES: Fc1ccc(CNc2ncccc2-c2cccnn2)c2[nH]ncc12]

In embodiments, the compound has the formula:

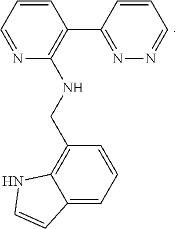 [SMILES: c1cnnc(-c2cccnc2NCc2cccc3cc[nH]c23)c1]

In embodiments, the compound has the formula:

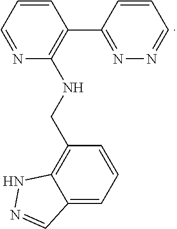 [SMILES: c1cnnc(-c2cccnc2NCc2cccc3cn[nH]c23)c1]

In embodiments, the compound has the formula:

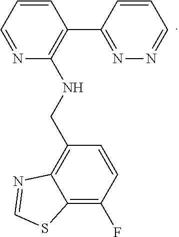 [SMILES: Fc1ccc(CNc2ncccc2-c2cccnn2)c2ncsc12]

In embodiments, the compound has the formula:

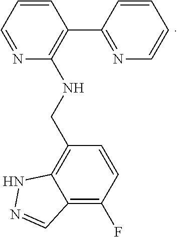 [SMILES: Fc1ccc(CNc2ncccc2-c2ccccn2)c2[nH]ncc12]

In embodiments, the compound has the formula:

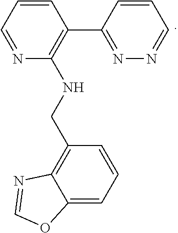 [SMILES: c1cnnc(-c2cccnc2NCc2cccc3ocnc23)c1]

In embodiments, the compound has the formula:

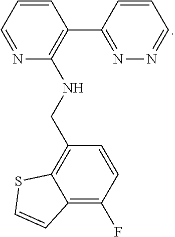 [SMILES: Fc1ccc(CNc2ncccc2-c2cccnn2)c2sccc12]

In embodiments, the compound has the formula:

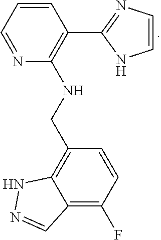 [SMILES: Fc1ccc(CNc2ncccc2-c2ncc[nH]2)c2[nH]ncc12]

In embodiments, the compound has the formula:

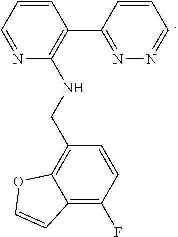 [SMILES: Fc1ccc(CNc2ncccc2-c2cccnn2)c2occc12]

In embodiments, the compound has the formula:

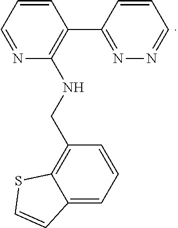 [SMILES: c1cnnc(-c2cccnc2NCc2cccc3ccsc23)c1]

In embodiments, the compound has the formula:

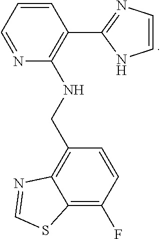 [SMILES: Fc1ccc(CNc2ncccc2-c2ncc[nH]2)c2ncsc12]

In embodiments, the compound has the formula:

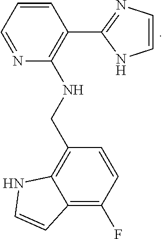 [SMILES: Fc1ccc(CNc2ncccc2-c2ncc[nH]2)c2[nH]ccc12]

In embodiments, the compound has the formula:

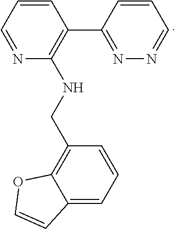 [SMILES: c1cnnc(-c2cccnc2NCc2cccc3ccoc23)c1]

In embodiments, the compound has the formula:

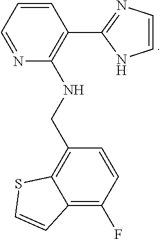 [SMILES: Fc1ccc(CNc2ncccc2-c2ncc[nH]2)c2sccc12]

In embodiments, the compound has the formula:

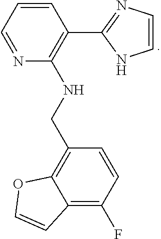 [SMILES: Fc1ccc(CNc2ncccc2-c2ncc[nH]2)c2occc12]

In embodiments, the compound has the formula:

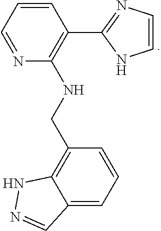 [SMILES: c1cnc(NCc2cccc3cn[nH]c23)c(-c2ncc[nH]2)c1]

In embodiments, the compound has the formula:

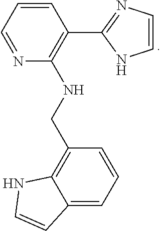 [SMILES: c1cnc(NCc2cccc3cc[nH]c23)c(-c2ncc[nH]2)c1]

In embodiments, the compound has the formula:

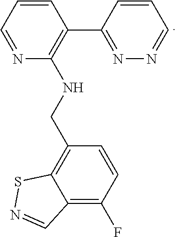 [SMILES: Fc1ccc(CNc2ncccc2-c2cccnn2)c2sncc12]

In embodiments, the compound has the formula:

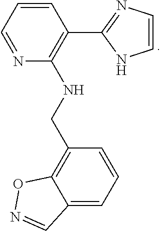 [SMILES: c1cnc(NCc2cccc3cnoc23)c(-c2ncc[nH]2)c1]

In embodiments, the compound has the formula:

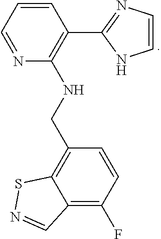 [SMILES: Fc1ccc(CNc2ncccc2-c2ncc[nH]2)c2sncc12]

In embodiments, the compound has the formula:

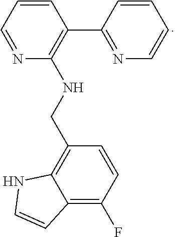 [SMILES: Fc1ccc(CNc2ncccc2-c2ccccn2)c2[nH]ccc12]

In embodiments, the compound has the formula:

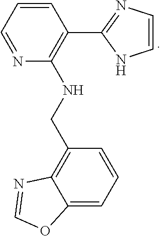 [SMILES: c1cnc(NCc2cccc3ocnc23)c(-c2ncc[nH]2)c1]

In embodiments, the compound has the formula:

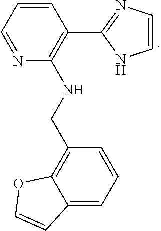 [SMILES: c1cnc(NCc2cccc3ccoc23)c(-c2ncc[nH]2)c1]

In embodiments, the compound has the formula:

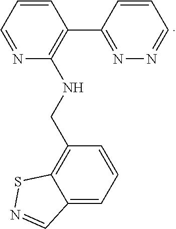 [SMILES: c1cnnc(-c2cccnc2NCc2cccc3cnsc23)c1]

In embodiments, the compound has the formula:

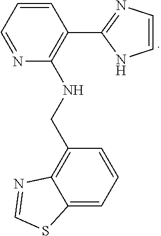 [SMILES: c1cnc(NCc2cccc3scnc23)c(-c2ncc[nH]2)c1]

In embodiments, the compound has the formula:

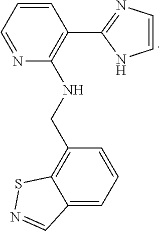 [SMILES: c1cnc(NCc2cccc3cnsc23)c(-c2ncc[nH]2)c1]

In embodiments, the compound has the formula:

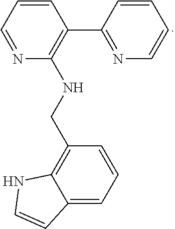 [SMILES: c1ccc(-c2cccnc2NCc2cccc3cc[nH]c23)nc1]

In embodiments, the compound has the formula:

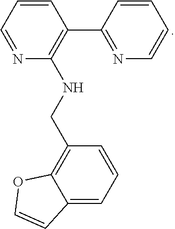 [SMILES: c1ccc(-c2cccnc2NCc2cccc3ccoc23)nc1]

In embodiments, the compound has the formula:

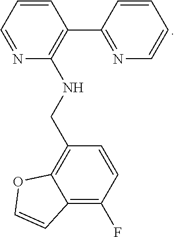 [SMILES: Fc1ccc(CNc2ncccc2-c2ccccn2)c2occc12]

In embodiments, the compound has the formula:

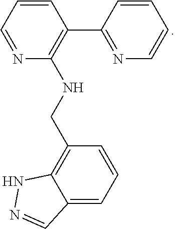 [SMILES: c1ccc(-c2cccnc2NCc2cccc3cn[nH]c23)nc1]

In embodiments, the compound has the formula:

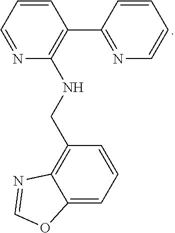 [SMILES: c1ccc(-c2cccnc2NCc2cccc3ocnc23)nc1]

In embodiments, the compound has the formula:

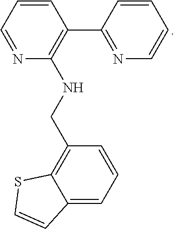 [SMILES: c1ccc(-c2cccnc2NCc2cccc3ccsc23)nc1]

In embodiments, the compound has the formula:

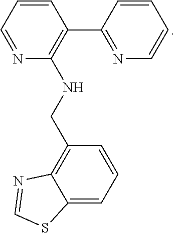 [SMILES: c1ccc(-c2cccnc2NCc2cccc3scnc23)nc1]

In embodiments, the compound has the formula:

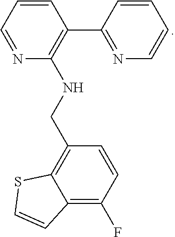 [SMILES: Fc1ccc(CNc2ncccc2-c2ccccn2)c2sccc12]

In embodiments, the compound has the formula:

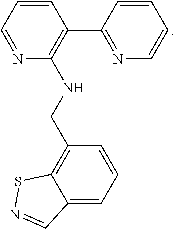 [SMILES: c1ccc(-c2cccnc2NCc2cccc3cnsc23)nc1]

In embodiments, the compound has the formula:

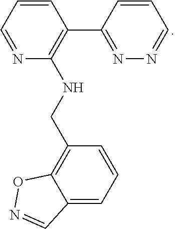 [SMILES: c1cnnc(-c2cccnc2NCc2cccc3cnoc23)c1]

In embodiments, the compound has the formula:

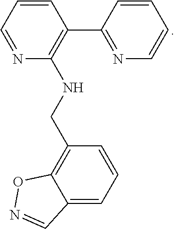 [SMILES: c1ccc(-c2cccnc2NCc2cccc3cnoc23)nc1]

In embodiments, the compound has the formula:

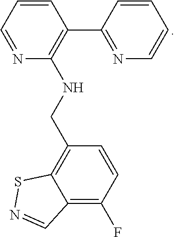 [SMILES: Fc1ccc(CNc2ncccc2-c2ccccn2)c2sncc12]

In embodiments, the compound has the formula:

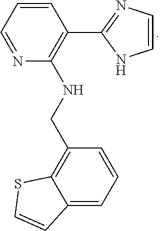 [SMILES: c1cnc(NCc2cccc3ccsc23)c(-c2ncc[nH]2)c1]

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound as described herein, including in embodiments. In embodiments the compound is a compound described herein (e.g., in the examples section, figures, tables, or claims).

### III. Pharmaceutical Compositions

In an aspect is provided a pharmaceutical composition including a compound described herein, or a pharmaceutically acceptable salt thereof, and a pharmaceutically acceptable excipient.

In embodiments, the pharmaceutical composition includes an effective amount of the compound. In embodiments, the pharmaceutical composition includes a therapeutically effective amount of the compound.

In embodiments, the compound is a compound of formula (I), including all embodiments thereof.

### IV. Methods of Use

In an aspect is provided a method of treating a neurodegenerative disease in a subject in need thereof, the method including administering to the subject in need thereof a therapeutically effective amount of a compound described herein, or a pharmaceutically acceptable salt thereof.

In embodiments, the neurodegenerative disease is a tauopathy. In embodiments, the neurodegenerative disease is Alzheimer's disease. Huntington's disease, amyotrophic lateral sclerosis, Lewy body disease, progressive supranuclear palsy, or Parkinson's disease. In embodiments, the neurodegenerative disease is Alzheimer's disease. In embodiments, the neurodegenerative disease is Huntington's disease. In embodiments, the neurodegenerative disease is amyotrophic lateral sclerosis. In embodiments, the neurodegenerative disease is Lewy body disease. In embodiments, the neurodegenerative disease is progressive supranuclear palsy. In embodiments, the neurodegenerative disease is Parkinson's disease.

In an aspect is provided a method of treating a liver disease in a subject in need thereof, the method including administering to the subject in need thereof a therapeutically effective amount of a compound described herein, or a pharmaceutically acceptable salt thereof.

In embodiments, the liver disease is nonalcoholic steatohepatitis.

In an aspect is provided a method of treating a fibrotic disease in a subject in need thereof, the method including administering to the subject in need thereof a therapeutically effective amount of a compound described herein, or a pharmaceutically acceptable salt thereof.

In an aspect is provided a method of treating a coronavirus infection in a subject in need thereof, the method including administering to the subject in need thereof a therapeutically effective amount of a compound described herein, or a pharmaceutically acceptable salt thereof.

In embodiments, the coronavirus infection is a SARS-CoV infection. In embodiments, the coronavirus infection is Severe Acute Respiratory Disease (SARS). In embodiments, the coronavirus infection is a SARS-CoV-2 infection. In embodiments, the coronavirus infection is coronavirus disease 2019 (COVID-19). In embodiments, the coronavirus infection is a MERS-CoV infection. In embodiments, the coronavirus infection is an HCoV-NL63 infection. In embodiments, the coronavirus infection is an HCoV-229E infection. In embodiments, the coronavirus infection is an HCoV—OC43 infection. In embodiments, the coronavirus infection is an HKU1 infection.

In embodiments, the compound is a compound of formula (I), including all embodiments thereof.

In an aspect is provided a method of reducing the level of activity of caspase-6 protein in a cell, the method including contacting the cell with an effective amount of a compound as described herein, or a pharmaceutically acceptable salt thereof.

In embodiments, the level of activity of the caspase-6 protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the caspase-6 protein is reduced by about 1.5-fold relative to a control (e.g., absence of the compound). In embodiments, the level of activity of the caspase-6 protein is reduced by about 2-fold relative to a control (e.g., absence of the compound). In embodiments, the level of activity of the caspase-6 protein is reduced by about 5-fold relative to a control (e.g., absence of the compound). In embodiments, the level of activity of the caspase-6 protein is reduced by about 10-fold relative to a control (e.g., absence of the compound). In embodiments, the level of activity of the caspase-6 protein is reduced by about 25-fold relative to a control (e.g., absence of the compound). In embodiments, the level of activity of the caspase-6 protein is reduced by about 50-fold relative to a control (e.g., absence of the compound). In embodiments, the level of activity of the caspase-6 protein is reduced by about 100-fold relative to a control (e.g., absence of the compound). In embodiments, the level of activity of the caspase-6 protein is reduced by about 250-fold relative to a control (e.g., absence of the compound). In embodiments, the level of activity of the caspase-6 protein is reduced by about 500-fold relative to a control (e.g., absence of the compound). In embodiments, the level of activity of the caspase-6 protein is reduced by about 1000-fold relative to a control (e.g., absence of the compound).

In embodiments, the level of activity of the caspase-6 protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activity of the caspase-6 protein is reduced by at least 1.5-fold relative to a control (e.g., absence of the compound). In embodiments, the level of activity of the caspase-6 protein is reduced by at least 2-fold relative to a control (e.g., absence of the compound). In embodiments, the level of activity of the caspase-6 protein is reduced by at least 5-fold relative to a control (e.g., absence of the compound). In embodiments, the level of activity of the caspase-6 protein is reduced by at least 10-fold relative to a control (e.g., absence of the compound). In embodiments, the level of activity of the caspase-6 protein is reduced by at least 25-fold relative to a control (e.g., absence of the compound). In embodiments, the level of activity of the caspase-6 protein is reduced by at least 50-fold relative to a control (e.g., absence of the compound). In embodiments, the level of activity of the caspase-6 protein is reduced by at least 100-fold relative to a control (e.g., absence of the compound). In embodiments, the level of activity of the caspase-6 protein is reduced by at least 250-fold relative to a control (e.g., absence of the compound). In embodiments, the level of activity of the caspase-6 protein is reduced by at least 500-fold relative to a control (e.g., absence of the compound). In embodiments, the level of activity of the caspase-6 protein is reduced by at least 1000-fold relative to a control (e.g., absence of the compound).

In an aspect is provided a method of reducing the level of activation of procaspase-6 protein in a cell, the method including contacting the cell with an effective amount of a compound as described herein, or a pharmaceutically acceptable salt thereof.

In embodiments, the level of activation of the procaspase-6 protein is reduced by about 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activation of the procaspase-6 protein is reduced by about 1.5-fold relative to a control (e.g., absence of the compound). In embodiments, the level of activation of the procaspase-6 protein is reduced by about 2-fold relative to a control (e.g., absence of the compound). In embodiments, the level of activation of the procaspase-6 protein is reduced by about 5-fold relative to a control (e.g., absence of the compound). In embodiments, the level of activation of the procaspase-6 protein is reduced by about 10-fold relative to a control (e.g., absence of the compound). In embodiments, the level of activation of the procaspase-6 protein is reduced by about 25-fold relative to a control (e.g., absence of the compound). In embodiments, the level of activation of the procaspase-6 protein is reduced by about 50-fold relative to a control (e.g., absence of the compound). In embodiments, the level of activation of the procaspase-6 protein is reduced by about 100-fold relative to a control (e.g., absence of the compound). In embodiments, the level of activation of the procaspase-6 protein is reduced by about 250-fold relative to a control (e.g., absence of the compound). In embodiments, the level of activation of the procaspase-6 protein is reduced by about 500-fold relative to a control (e.g., absence of the compound). In embodiments, the level of activation of the procaspase-6 protein is reduced by about 1000-fold relative to a control (e.g., absence of the compound).

In embodiments, the level of activation of the procaspase-6 protein is reduced by at least 1.5-, 2-, 3-, 4-, 5-, 6-, 7-, 8-, 9-, 10-, 15-, 20-, 25-, 30-, 35-, 40-, 45-, 50-, 60-, 70-, 80-, 90-, 100-, 150-, 200-, 250-, 300-, 350-, 400-, 450-, 500-, 600-, 700-, 800-, 900-, or 1000-fold relative to a control (e.g., absence of the compound). In embodiments, the level of activation of the procaspase-6 protein is reduced by at least 1.5-fold relative to a control (e.g., absence of the compound). In embodiments, the level of activation of the procaspase-6 protein is reduced by at least 2-fold relative to a control (e.g., absence of the compound). In embodiments, the level of activation of the procaspase-6 protein is reduced by at least 5-fold relative to a control (e.g., absence of the compound). In embodiments, the level of activation of the procaspase-6 protein is reduced by at least 10-fold relative to a control (e.g., absence of the compound). In embodiments, the level of activation of the procaspase-6 protein is reduced by at least 25-fold relative to a control (e.g., absence of the compound). In embodiments, the level of activation of the procaspase-6 protein is reduced by at least 50-fold relative to a control (e.g., absence of the compound). In embodiments, the level of activation of the procaspase-6 protein is reduced by at least 100-fold relative to a control (e.g., absence of the compound). In embodiments, the level of activation of the procaspase-6 protein is reduced by at least 250-fold relative to a control (e.g., absence of the compound). In embodiments, the level of activation of the procaspase-6 protein is reduced by at least 500-fold relative to a control (e.g., absence of the compound). In embodiments, the level of activation of the procaspase-6 protein is reduced by at least 1000-fold relative to a control (e.g., absence of the compound).

In embodiments, the compound binds to Y198^{A} of the procaspase-6 (e.g., human procaspase-6) protein. In embodiments, the compound binds noncovalently to Y198^{A} of the procaspase-6 (e.g., human procaspase-6) protein. In embodiments, the compound binds to Y198^{B} of the procaspase-6 (e.g., human procaspase-6) protein. In embodiments, the compound binds noncovalently to Y198^{B} of the procaspase-6 (e.g., human procaspase-6) protein.

### V. Embodiments

Embodiment P1. A compound, or a pharmaceutically acceptable salt thereof, having the formula:

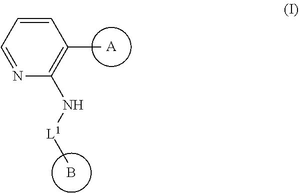

wherein
Ring A is a substituted or unsubstituted 5 to 6 membered heteroaryl or substituted or unsubstituted 8 to 10 membered fused ring heteroaryl:
Ring B is a substituted or unsubstituted 9 to 10 membered fused ring aryl or substituted or unsubstituted 9 to 10 membered fused ring heteroaryl;
L¹ is a bond or substituted or unsubstituted C₁-C₃ alkylene.

Embodiment P2. The compound of embodiment P1, wherein Ring B is substituted or unsubstituted benzoisoxazolyl, substituted or unsubstituted benzoisothiazolyl, substituted or unsubstituted benzofuranyl, substituted or unsubstituted indazolyl, substituted or unsubstituted benzoxazolvl, substituted or unsubstituted benzothiazolyl, substituted or unsubstituted benzothiophenyl, or substituted or unsubstituted indolyl.

Embodiment P3. The compound of embodiment P1, wherein Ring B is

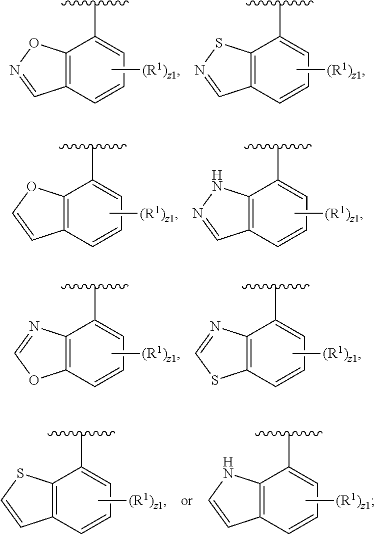

wherein
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —B(OR^{1C})(OR^{1D}), —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl:
R^{1A}, R^{1B}, R^{1C} and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂. —OCHI₂, —OCHF₂, —OCH₂Cl. —OCH₂Br, —OCH₂T, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} substituents bonded to the same nitrogen atom may optionally be joined to form a substituted or unsubstituted heterocycloalkyl or substituted or unsubstituted heteroaryl;
each X¹ is independently —F, —Cl, —Br, or —I;
n1 is an integer from 0 to 4;
m1 and v1 are independently 1 or 2; and
z1 is an integer from 0 to 5.

Embodiment P4. The compound of embodiment P3, wherein Ring B is

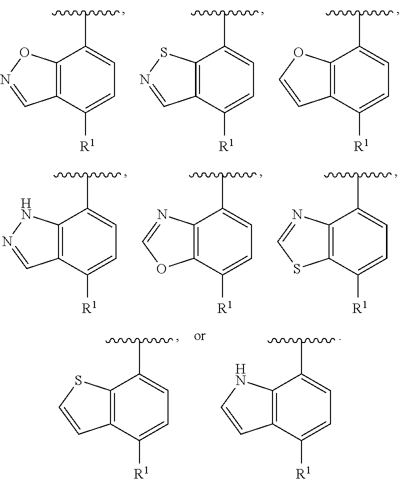

Embodiment P5. The compound of one of embodiments P3 to P4, wherein R¹ is independently halogen, —CCl₃, —CBr₃, —CF₃, —Cl₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —B(OH)₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P6. The compound of one of embodiments P3 to P4, wherein R¹ is independently halogen.

Embodiment P7. The compound of one of embodiments P3 to P4, wherein R¹ is independently —F.

Embodiment P8. The compound of embodiment P3, wherein z1 is 0.

Embodiment P9. The compound of one of embodiments P3 and P5 to P7, wherein z1 is 1.

Embodiment P10. The compound of embodiment P1, wherein Ring B is

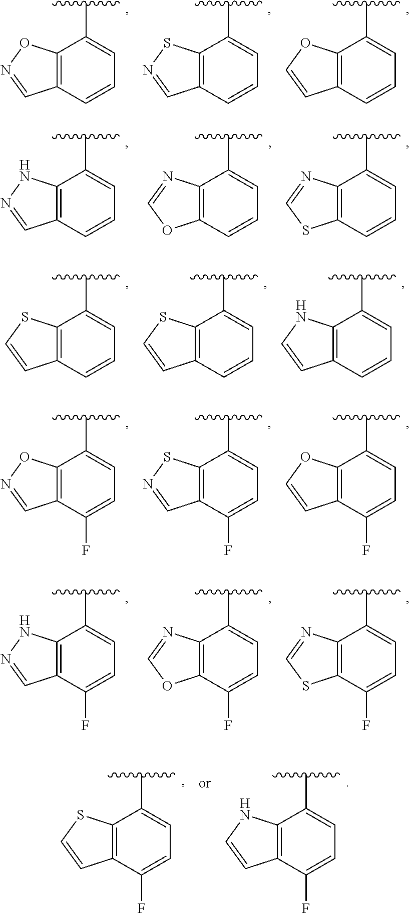

Embodiment P11. The compound of one of embodiments P1 to P10, wherein Ring A is a substituted or unsubstituted nitrogen-containing 5 to 6 membered heteroaryl or substituted or unsubstituted nitrogen-containing 8 to 10 membered fused ring heteroaryl.

Embodiment P12. The compound of one of embodiments Pt to P10, wherein Ring A is substituted or unsubstituted pyridyl, substituted or unsubstituted pyridazinyl, substituted or unsubstituted pyrimidinyl, substituted or unsubstituted pyrazinyl, substituted or unsubstituted triazinyl, substituted or unsubstituted imidazolyl, substituted or unsubstituted pyrazolyl, substituted or unsubstituted oxazolyl, substituted or unsubstituted isoxazolyl, substituted or unsubstituted furanyl, substituted or unsubstituted thienyl, substituted or unsubstituted thiazolyl, substituted or unsubstituted isothiazolyl, substituted or unsubstituted triazolyl, substituted or unsubstituted oxadiazolyl, substituted or unsubstituted tetrazolyl, substituted or unsubstituted imidazopyridinyl, substituted or unsubstituted benzothiazolyl, or substituted or unsubstituted benzoimidazolyl.

Embodiment Pt3. The compound of one of embodiments Pt to P10, wherein Ring A is

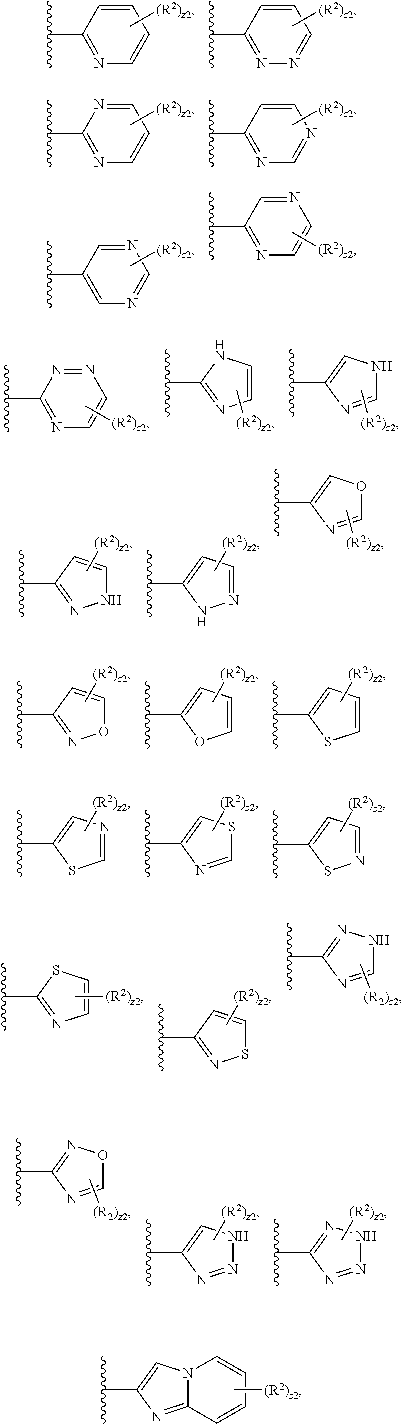

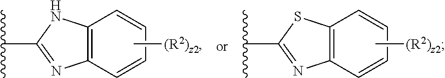

wherein
R² is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl. —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and z2 is an integer from 0 to 5.

Embodiment P14. The compound of embodiment P13, wherein R² is independently —NH₂ or unsubstituted C₁-C₄ alkyl.

Embodiment P15. The compound of embodiment P13, wherein R² is independently —NH₂ or unsubstituted methyl.

Embodiment P16. The compound of embodiment P13, wherein z2 is 0.

Embodiment P17. The compound of one of embodiments P13 to P15, wherein z2 is 1.

Embodiment P18. The compound of embodiment P1, wherein Ring A is

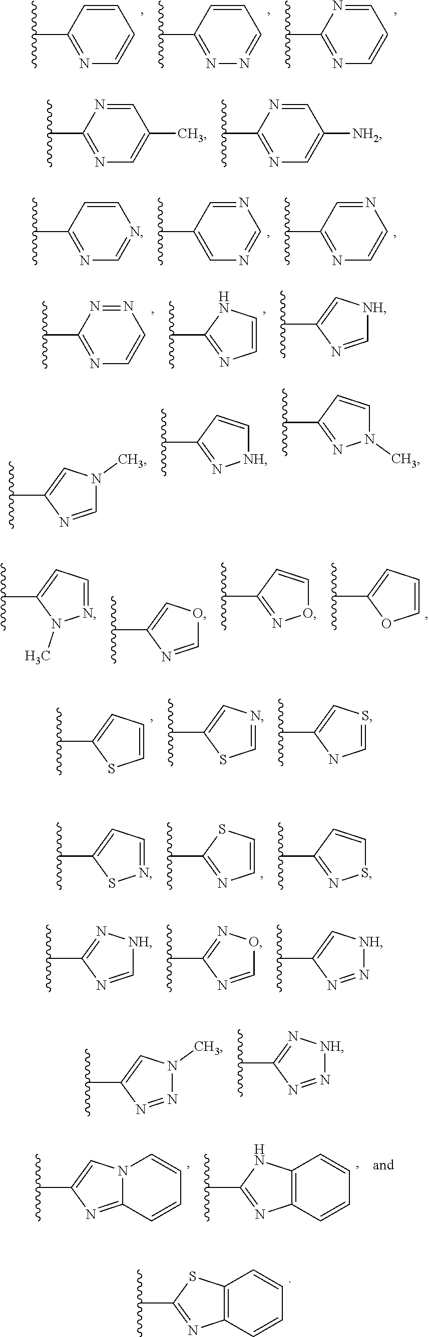

Embodiment P19. The compound of one of embodiments P1 to P18, wherein L¹ is unsubstituted C₁-C₃ alkylene.

Embodiment P20. The compound of one of embodiments P1 to P18, wherein L¹ is unsubstituted methylene.

Embodiment P21. A pharmaceutical composition comprising a compound of one of embodiments P1 to P20, or a pharmaceutically acceptable salt thereof, and a pharmaceutically acceptable excipient.

Embodiment P22. A method of treating a neurodegenerative disease in a subject in need thereof, said method comprising administering to the subject in need thereof a therapeutically effective amount of a compound of one of embodiments P1 to P20, or a pharmaceutically acceptable salt thereof.

Embodiment P23. The method of embodiment P22, wherein the neurodegenerative disease is a tauopathy.

Embodiment P24. The method of embodiment P22, wherein the neurodegenerative disease is Alzheimer's disease. Huntington's disease, amyotrophic lateral sclerosis, Lewy body disease, progressive supranuclear palsy, or Parkinson's disease.

Embodiment P25. The method of embodiment P22, wherein the neurodegenerative disease is Alzheimer's disease.

Embodiment P26. A method of treating a liver disease in a subject in need thereof, said method comprising administering to the subject in need thereof a therapeutically effective amount of a compound of one of embodiments I to P20, or a pharmaceutically acceptable salt thereof.

Embodiment P27. The method of embodiment P26, wherein the liver disease is nonalcoholic steatohepatitis.

Embodiment P28. A method of treating a fibrotic disease in a subject in need thereof, said method comprising administering to the subject in need thereof a therapeutically effective amount of a compound of one of embodiments P1 to P20, or a pharmaceutically acceptable salt thereof.

Embodiment P29. A method of treating a coronavirus infection in a subject in need thereof, said method comprising administering to the subject in need thereof a therapeutically effective amount of a compound of one of embodiments P1 to P20, or a pharmaceutically acceptable salt thereof.

Embodiment P30. A method of reducing the level of activity of caspase-6 protein in a cell, said method comprising contacting the cell with an effective amount of a compound of one of embodiments P1 to P20, or a pharmaceutically acceptable salt thereof.

Embodiment P31. A method of reducing the level of activation of procaspase-6 protein in a cell, said method comprising contacting the cell with an effective amount of a compound of one of embodiments P1 to P20, or a pharmaceutically acceptable salt thereof.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Examples

### Example 1: A Protein-Ligand Model System for the Study of Heteroarene-Aryl Stacking Under Physiological Conditions

The empirical study of (hetero)aryl-aryl stacking proteins and in ligand-protein interaction has largely been approached through bioinformatic analyses of large crystallographic databases. When taken in the aggregate, these large data sets provide useful insight into preferred stacking orientations and geometries. Such analyses are qualitative in nature, since absolute (or even relative) interaction energies cannot be extracted from purely structural information. Filling this void have been computational approaches involving various levels of theory, from the early work of Hunter and Sanders on the optimal geometry of these interactions, to an improved understanding of substituent effects using the methods of Houk and Wheeler. Recently, Wheeler described molecular descriptors that enable predictions of the strength of diverse heteroarene stacking interactions without computationally costly ab initio computations.

Various experimental systems have been employed to study heteroarene-aryl stacking, including synthetic host-guest systems, intramolecular arene stacking in 1,8-diarylnaphthlenes, and various molecular “torsion balance”. Applications of the latter to heteroarene stacking include Shimizu's cleft-like N-aryl imides, Gung's triptycenes, and Gellman's tertiary amide foldamers. Study of the conformational equilibrium between ‘closed’ and ‘open’ states, typically by NMR, is used to determine the binding enthalpy of the stacking interaction (closed state). On the other hand, the architecture of these systems imposes constraints on the orientation and approach of the interacting arenes, such that stacking interactions may form with sub-optimal geometries and distances. With a few exceptions, prior studies with torsion balances are performed in organic or polar aprotic solvent, due to a lack of sufficient aqueous solubility.

The use of a protein-ligand interaction to study intermolecular interactions is attractive in principle, but also fraught with experimental challenges. Comparisons of binding affinities are confounded by disparate contributions of desolvation/hydrophobic effects in even very similar ligands. It can also be difficult to disentangle the contribution of the binding enthalpies for the interaction under investigation from effects on distal ligand-protein interactions. The challenge is correspondingly greater when studying interactions with precise geometric and spatial requirements. Thus, ligand-protein systems were used successfully by both Dougherty and Diederich to study cation-7c interactions, while attempts by Diederich to use factor Xa and cathepsin B ligands to interrogate heteroarene-amide stacking largely failed.

Inspired by such seminal efforts, we have sought to identify new protein-ligand model systems of utility for the study of intermolecular interactions. We judge as key criteria for the protein component, a highly shape-persistent binding site that can be readily assessed crystallographically. Criteria for the ligand component include adoption of a highly conserved binding mode and ability to modify the group/moiety of interest without impacting global ligand conformation or the interactions of distal groups. Illustrative of this approach is our recent report of the bacterial serine hydrolase CTX-M and a cognate ligand to study heteroarene-amide stacking interactions. Thus, we found with congeneric analogs differing only in a terminal heteroarene substitution (e.g., all possible regioisomers of pyridine and pyrimidine), that ligand binding affinities could be correlated with the predicted favorable effect of opposing dipole-dipole interaction of the amide and heteroarene substituents. Thus, the first experimental validation of computational predictions concerning these interactions was achieved.

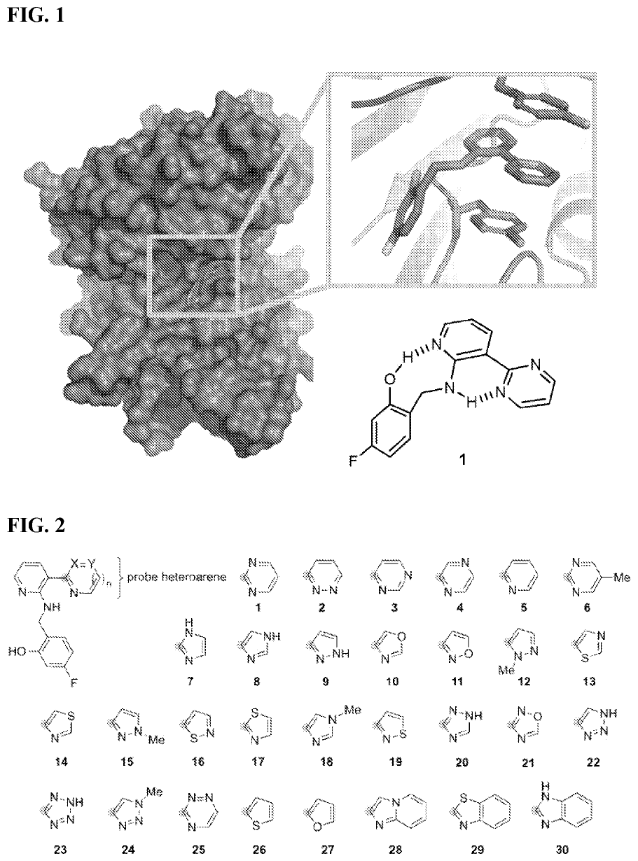

We explore herein, inter alia, heteroarene-tyrosine stacking using congeneric analogs of compound 1, a “molecular glue” that binds and stabilizes procaspase-6 toward proteolytic processing and activation. Relevant to its application in the current work is that the distal pyrimidine ring of 1 was previously shown to form a nearly ideal stacking interaction between tyrosine residues 198^{A} and 198B, each of which project from one subunit of the dimeric zymogen (FIG. 1). We synthesized more than two dozen new analogs of 1 bearing terminal five-membered, six-membered, or bicyclic heteroarenes as “probe heterocycles” and evaluated their binding affinities by surface plasmon resonance (SPR) methods, after first confirming a conserved binding mode and stacking interaction regardless of the nature of the probe heterocycle. We find that differences in binding affinity are largely attributable to the relative strength of the stacking interaction. To our knowledge, the current study represents the most comprehensive examination of heteroarene-aryl stacking interactions in a protein-ligand system, and validates a new model system for future studies of diverse intermolecular interactions under physiologically relevant conditions.

A set of test ligands (2-30, FIG. 2) was synthesized wherein the pyrimidine ring of 1 is replaced with diverse heterocycles commonly employed in materials, supramolecular, and medicinal chemistry. With a few exceptions, we selected probe heteroarenes with an ortho heteroatom (N or O) such that intramolecular hydrogen bond formation (as in 1) should promote a co-planar pyridine-heteroarene conformation optimal for stacking (FIG. 1). Pyrazole 12, bearing an ortho methyl substituent, was prepared as a negative control that was unlikely to form a productive stacking interaction. For many of the new compounds, the probe heteroarene could be introduced via a late-stage Suzuki coupling employing a common intermediate. Other heteroarene systems required bespoke syntheses from acyclic precursors. All test compounds were purified to >95% purity and fully characterized prior to crystallographic and SPR studies.

The expectation that probe heteroarenes would stack between two symmetry-related tyrosine residues was viewed as an important feature of this system, since the double-stack significantly reduces the wetted surface of probe heteroarene and was thus expected to reduce the contribution of desolvation energies in the binding of the diverse heterocycles. To confirm that binding poses and stacking interactions were conserved across compounds 1-30, we solved the complex crystal structures of fully a third of the analogs bound to procaspase-6. All ten structures revealed binding poses nearly identical to that of 1, and all placed their probe heterocycles in productive stacking interactions with tyrosines 198^{A} and 198^{B} (FIG. 3).

We used SPR to assess the binding affinity of test compounds, using an avi-tagged (biotinylated) procaspase-6 construct that was captured onto a neutravidin-coated sensor chip in a Biacore 8K instrument. Preliminary sensorgrams were recorded for all compounds at a top concentration of 50 μM to assess binding behavior and estimate preliminary K_{D} values when possible. Next, a definitive data set of K_{D} values, determined in triplicate, was generated for all compounds, using the most appropriate concentration range based on the preliminary K_{D} values obtained (see Table 1). The average of the triplicate K_{D} values for each analog was furthermore used to calculate the experimental binding energies (ΔG) provided in the figures that follow. For three weak-binding analogs (11, 12, and 26), a K_{D} was estimated based on the SPR response measured at the highest concentration and the theoretical maximum response (Rmax) for the series. The higher K_{D} value of compound 12 was not unexpected, as we had predicted a priori that this analog would be unable to form productive stacking interactions (vide supra). We thus excluded analog 12 from the analysis below since its experimental binding affinity is likely disconnected from the stacking interaction under study.

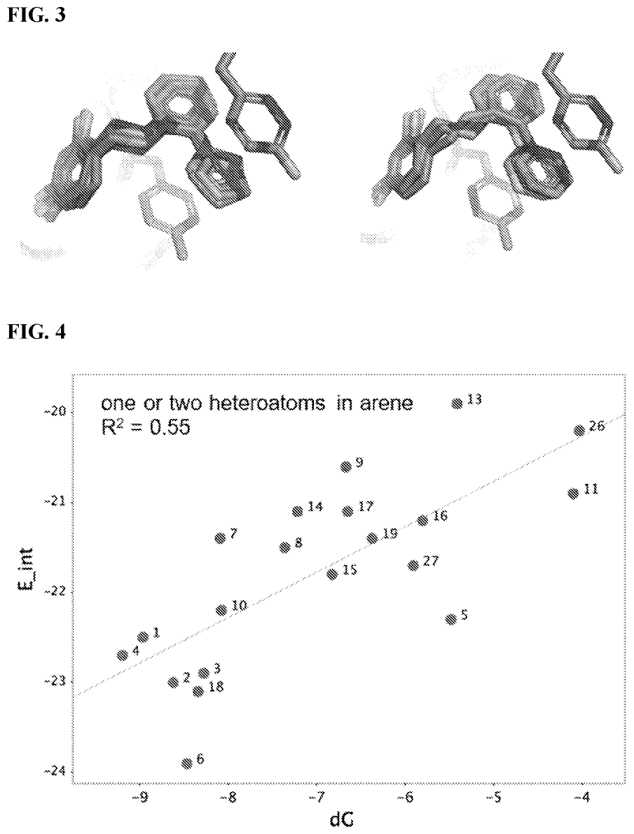

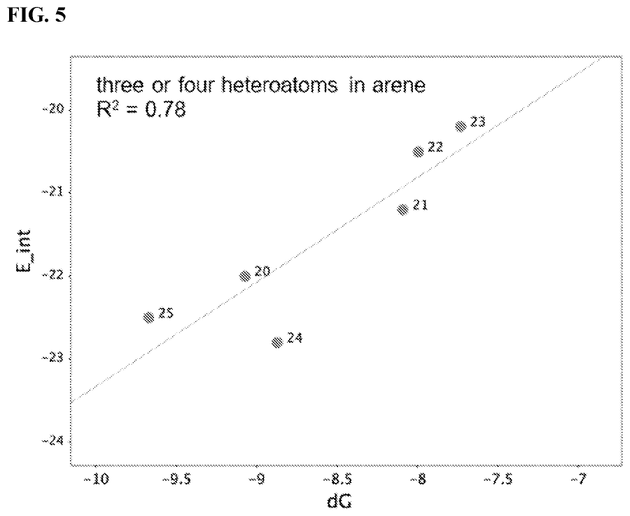

We next compared predicted E_int values to experimental ΔG values, regardless of the ring size or the number of heteroatoms in the probe arene and observed only a modest correlation (R²=0.24). Considering this, we speculated that differences in desolvation energies could be a root cause of the poor correlation. For example, the four analogs for which E_int most overpredicted binding affinties (6, and 28-30) all possessed hydrophobic functionality expected to extend beyond the periphery of the tyrosine stacking surface. Conversely, among analogs for which E_int underpredicted binding affinities were analogs 20-23 with hydrophilic probe arenes possessing a high heteroatom count relative to ring size. When the data were reevaluated taking into account the number of heteroatoms in the probe arene (a surrogate measure of desolvation penalties), a more robust correlation of the experimental and computed data was observed. Thus, among analogs bearing one or two heteroatoms in a five- or six-membered probe arene, the correlation of experimental and computed data was quite reasonable (R²=0.55; FIG. 4). The correlation among the set of analogs bearing three or four heteroatoms was even better (R²=0.78; FIG. 5). Finally, analyzing the bicyclic analogs 28-30 separately on account of their much greater wetted surface compared to monocyclic analogs returned the strongest correlation of all (R²=0.86) albeit with a limited number of data points.

Overall, the correlation of computed and experimental data suggested that experimental ΔG values determined with this ligand-protein model system are largely reflective of differences in the relative strength of the stacking interaction involving the respective probe heteroarene. Thus, comparing pyridine analog 5 with isosteric diazenes (1-4) and triazene (25) analogs, we observe the diazenes and triazine to all bind significantly more potently than the pyridine (FIG. 4 and FIG. 5). This result conforms well with the expectation that more electron deficient heteroarenes should form stronger stacking interactions with an electron-rich tyrosine side chain. The trend of increasing binding affinities when moving from thiophene (26) to furan (27), to indazole (9) and imidazole (7) is also consistent with expectations (FIG. 4). Finally, the favorable effect of N-methylation of nitrogen heterocycles is a striking and consistent effect across indazole (cf. 9 vs. 15), imidazole (cf 8 vs 18), and triazole (cf 22 vs. 24) comparators. These data suggest the potential utility of this model system for studies of substituent effects in heteroarene stacking, and area of considerable recent interest in the computational arena.

Other findings of this study may surprise the empiricist medicinal chemist, such as the dramatically improved ΔG value (nearly 4 kcal/mol) for oxazole 10 as compared to isoxazole 11. Notably, this effect is reasonably well predicted by the computed E_int values, and likely reflects the greater basicity of the oxygen atom in oxazole, combined with its proximity to, and possible interaction with, the polar OH function of the tyrosine ring.

### Example 2: Experimental Procedures and Characterization Data

### Route for ELG-000468

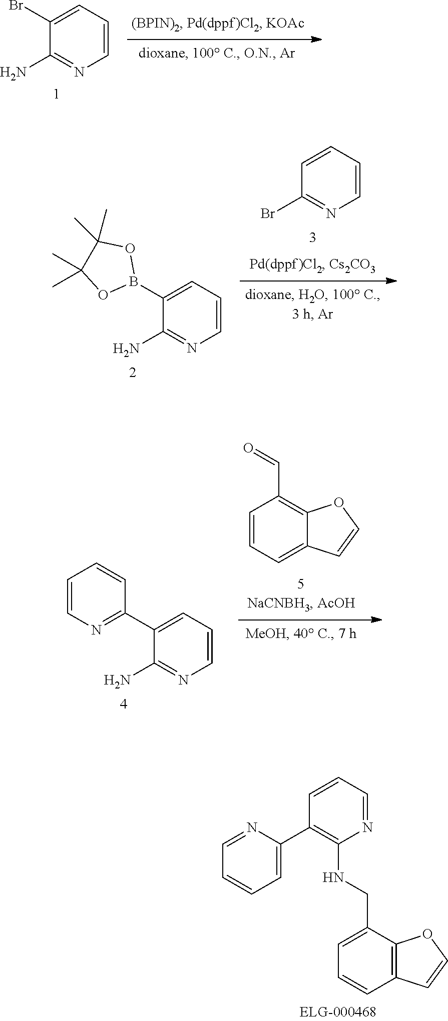 [SMILES: Nc1ncccc1Br ; CC1(C)OB(c2cccnc2N)OC1(C)C ; Brc1ccccn1 ; O=Cc1cccc2ccoc12 ; Nc1ncccc1-c1ccccn1 ; c1ccc(-c2cccnc2NCc2cccc3ccoc23)nc1]

### The Synthesis of 3-(4,4,5,5-tetramethyl-1,3,2-dioxaborolan-2-yl)pyridin-2-amine (2)

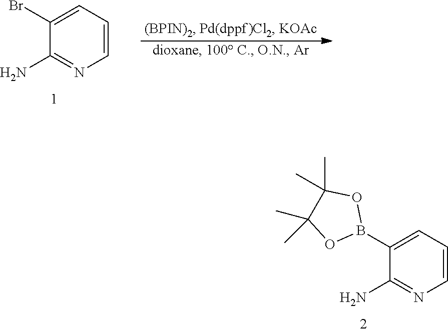 [SMILES: Nc1ncccc1Br ; "dioxane, 100° C., O.N., Ar" ; "(BPIN)2, Pd(dppf)Cl2, KOAc" ; CC1(C)OB(c2cccnc2N)OC1(C)C]

To a 250 mL round bottom flask were added 1 (2.5 g, 14.50 mmol). Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

The synthesis of [2,3′-bipyridin]-2′-amine (4)

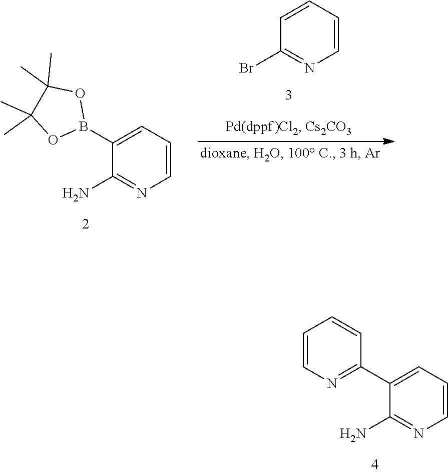 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol). Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.0 g, yield: 40%).

### The Synthesis of N-(benzofuran-7-ylmethyl)-[2,3′-bipyridin]-2′-amine (ELG-000468)

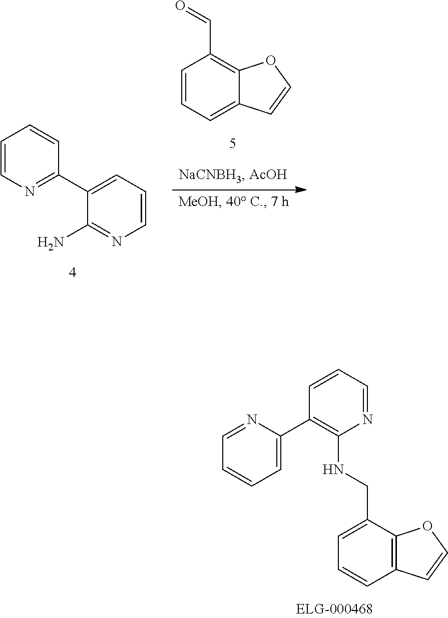 [SMILES: O=Cc1cccc2ccoc12 ; Nc1ncccc1-c1ccccn1 ; c1ccc(-c2cccnc2NCc2cccc3ccoc23)nc1]

To a 100 mL round bottom flask were added 4 (40.0 mg, 0.23 mmol), MeOH (3 mL), 5 (34.1 mg, 0.23 mmol) and AcOH (3 drops), the mixture was stirred at 40° C. until 4 consumed, about 2 hours. Then added NaCNBH₃ (72.3 mg, 1.15 mmol), the reaction mixture was stirred at room temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000468 (15.0 mg, yield: 21%) as a yellow oil. LC-MS (Agilent LCMS 1260-6125B, Column: Boltimate EXT C₁₈ CoreShell (50 mm*4.6 mm*2.7 μm); Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 96.28%, Rt=1.220 min; MS Calcd: 301.12; MS Found: 302.1 [M+H]⁺. HPLC (Waters UPLC, Column: ACQUITY UPLC BEH C18 1.7 μm*2.1 mm*150 mm); Column Temperature: 40° C.: Flow Rate: 0.3 mL/mm; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 93.89%, Rt=4.424 min. ¹H NMR (400 MHz, DMSO-d₆) δ 10.38 (s, 1H), 8.69-8.54 (m, 1H), 8.30-8.22 (m, 1H), 8.12-8.04 (m, 3H), 7.98 (td, J=8.0, 1.8 Hz, 1H), 7.57 (dd, J=7.2, 1.5 Hz, 1H), 7.42 (dd, J=6.9, 5.2 Hz, 1H), 7.27-7.16 (m, 2H), 7.00 (d, J=2.2 Hz, 1H), 6.89-6.73 (m, 1H), 5.06 (s, 2H). Chemical Formula: C₁₉H₁₅N₃O. Molecular Weight: 301.35.

### Route for ELG-000469

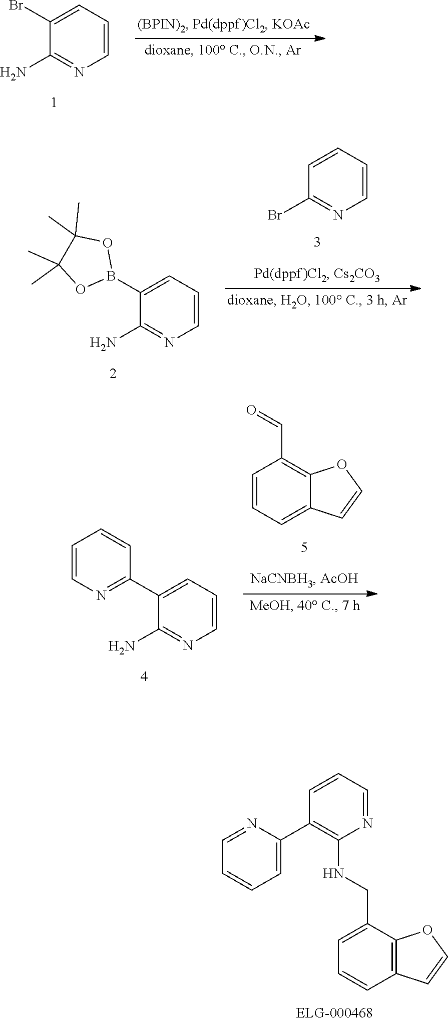 [SMILES: Nc1ncccc1Br ; CC1(C)OB(c2cccnc2N)OC1(C)C ; Brc1ccccn1 ; O=Cc1cccc2ccoc12 ; Nc1ncccc1-c1ccccn1 ; c1ccc(-c2cccnc2NCc2cccc3ccoc23)nc1]

### The Synthesis of 3-(4,4,5,5-tetramethyl-1,3,2-dioxaborolan-2-yl)pyridin-2-amine (2)

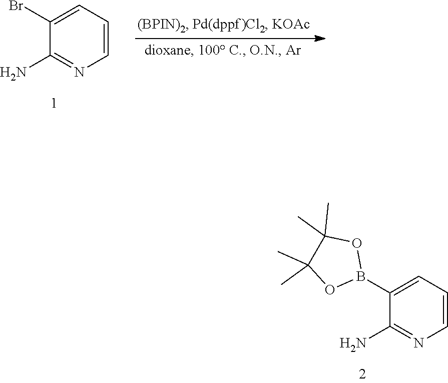 [SMILES: Nc1ncccc1Br ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of ┌2,3′-bipyridin┐-2′-amine (4)

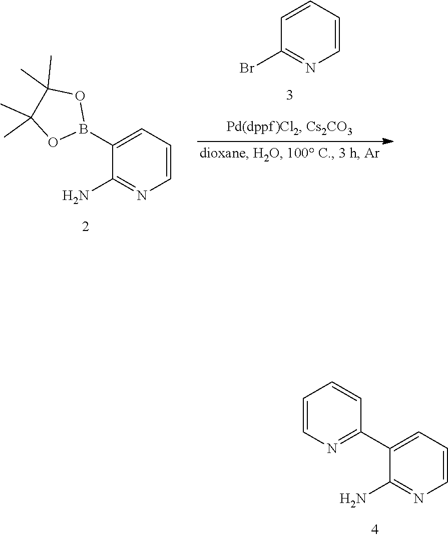 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol). Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.0 g, yield: 40%).

### The Synthesis of N-((4-fluorobenzofuran-7-yl)methyl)-[2,3′-bipyridin]-2′-amine (ELG-000469)

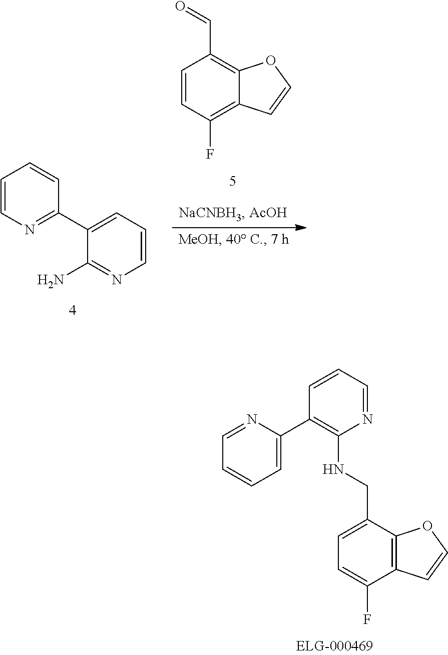 [SMILES: O=Cc1ccc(F)c2ccoc12 ; Nc1ncccc1-c1ccccn1 ; Fc1ccc(CNc2ncccc2-c2ccccn2)c2occc12]

To a 50 mL round bottom flask were added 4 (64 mg, 0.37 mmol), 5 (61.4 mg, 0.37 mmol). AcOH (5 drops) and MeOH (5 mL), the mixture was heated to 40° C. and stirred for 2 hours. Then the reaction solution was cooled to room temperature with an ice water bath. The NaBH₃CN (117.5 mg, 1.87 mmol) was added, keep stir at room temperature for another 5 hours. The reaction solution was quenched by water (0.5 mL), concentrated to dryness, purified by prep. TLC to afford ELG-000469 (35.9 mg, yield: 30%) as a white solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%. Rt=1.269 min; MS Calcd: 319.11; MS Found: 320.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 100%, Rt=4.497 min. ¹H NMR (400 MHz, DMSO-d₆) δ 9.79 (t, J=5.8 Hz, 1H), 8.62 (ddd, J=4.9, 1.9, 0.9 Hz, 1H), 8.11 (dd, J=3.9, 2.0 Hz, 2H), 8.10-8.06 (m, 1H), 8.01 (dt, J=8.3, 1.1 Hz, 1H), 7.94 (ddd. J=8.3, 7.4, 1.9 Hz, 1H), 7.36 (ddd. J=7.4, 5.0, 1.1 Hz, 1H), 7.23 (dd. J=8.3, 5.3 Hz, 1H), 7.10 (d, J=2.3 Hz, 1H), 7.06-6.99 (m, 1H), 6.70 (dd, J=7.0, 5.4 Hz, 1H), 4.99 (d, J=5.8 Hz, 2H). Chemical Formula: C₁₉H₁₄N₃OF. Molecular Weight: 319.34.

### Route for ELG-000472

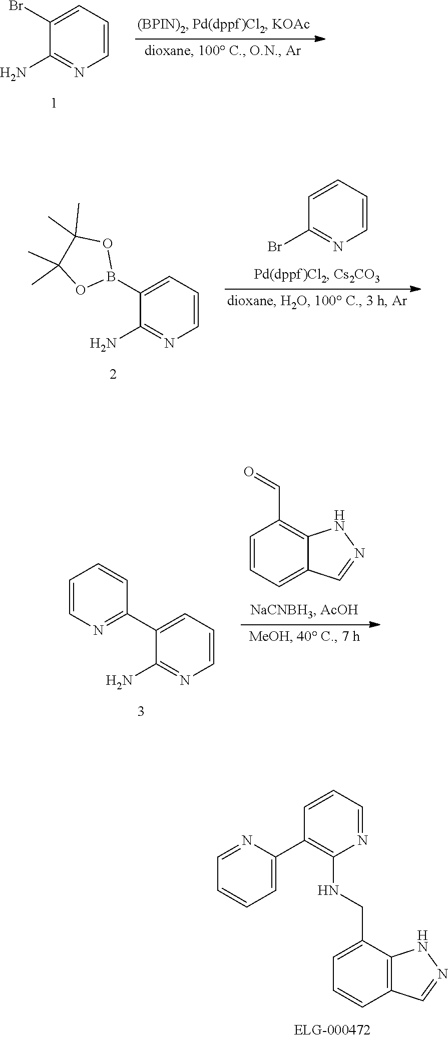 [SMILES: Nc1ncccc1Br ; CC1(C)OB(c2cccnc2N)OC1(C)C ; Brc1ccccn1 ; Nc1ncccc1-c1ccccn1 ; O=Cc1cccc2cn[nH]c12 ; c1ccc(-c2cccnc2NCc2cccc3cn[nH]c23)nc1]

### The Synthesis of 3-(4,4,5,5-tetramethyl-1,3,2-dioxaborolan-2-yl)pyridin-2-amine (2)

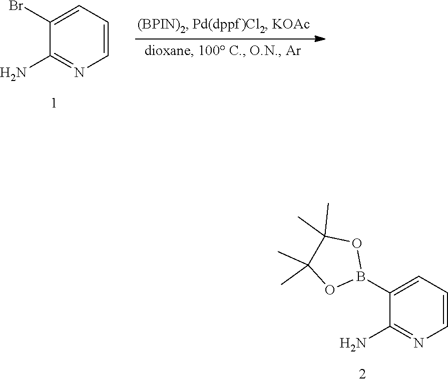 [SMILES: Nc1ncccc1Br ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (3)

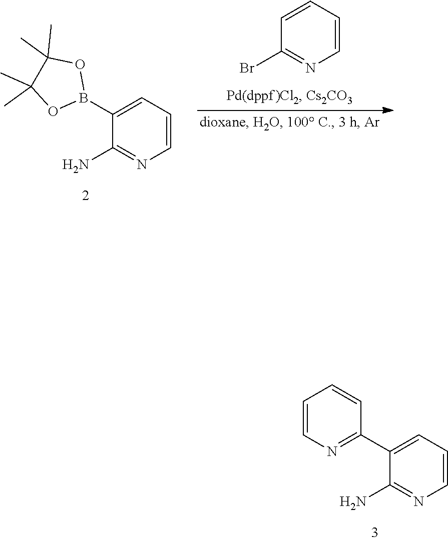 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 2-bromopyridine (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 3 (1.0 g, yield: 40%).

### The Synthesis of N-((1H-indazol-7-yl)methyl)-[2,3′-bipyridin]-2′-amine (ELG-000472)

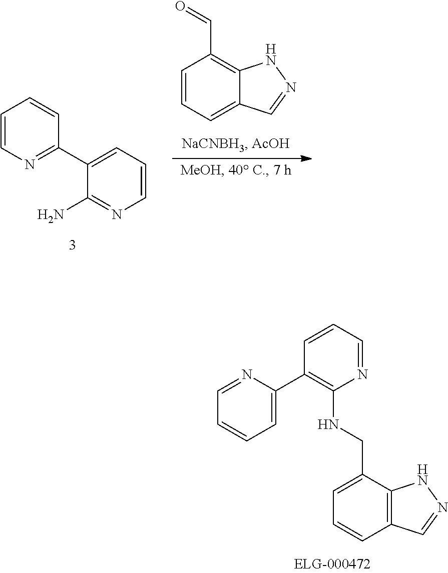 [SMILES: Nc1ncccc1-c1ccccn1 ; O=Cc1cccc2cn[nH]c12 ; c1ccc(-c2cccnc2NCc2cccc3cn[nH]c23)nc1]

### 3 ELG-000472

To a 100 mL round bottom flask were added 3 (78.0 mg, 0.46 mmol). MeOH (3 mL), 1H-indazole-7-carbaldehyde (67.0 mg, 0.46 mmol) and AcOH (3 drops), the mixture was stirred at 40° C. until start materials consumed, about 2 hours. Then added NaCNBH₃ (58.0 mg, 0.92 mmol), the reaction mixture was stirred at the same temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000472 (5 mg, yield: 4%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.114 min; MS Calcd: 301.13: MS Found: 302.1 [M+H]⁺. HPLC (Waters UPLC. Column: ACQUITY UPLC BEH C18 1.7 μm*2.1 mm*150 mm): Column Temperature: 40° C.; Flow Rate: 0.3 mL/min: Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 97.65%, Rt=4.110 min. ¹H NMR (400 MHz, DMSO-d₆) δ 13.24 (s, 1H), 9.72 (d, J=5.7 Hz, 1H), 8.73-8.50 (m, 1H), 8.13-8.07 (m, 3H), 8.02-7.91 (m, 2H), 7.62 (d, J=8.0 Hz, 1H), 7.42-7.32 (m, 1H), 7.25 (d, J=6.8 Hz, 1H), 7.11-6.98 (m, 1H), 6.69 (dd, J=7.6, 4.8 Hz, 1H), 5.02 (d, J=5.7 Hz, 2H). Chemical Formula: C18H₁₅N₅. Molecular Weight: 301.35.

### Route for ELG-000480

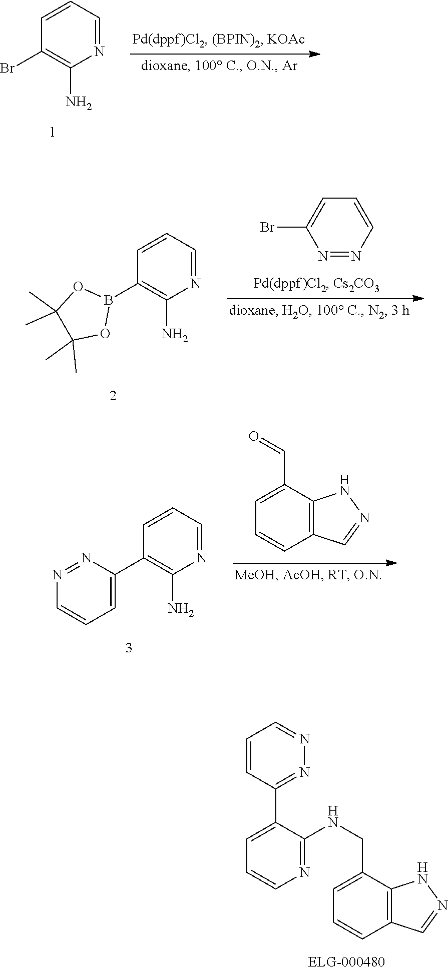 [SMILES: Nc1ncccc1Br ; CC1(C)OB(c2cccnc2N)OC1(C)C ; Brc1cccnn1 ; Nc1ncccc1-c1cccnn1 ; O=Cc1cccc2cn[nH]c12 ; c1cnnc(-c2cccnc2NCc2cccc3cn[nH]c23)c1]

### The Synthesis of 3-(4,4,5,5-tetramethyl-1,3,2-dioxaborolan-2-yl)pyridin-2-amine (2)

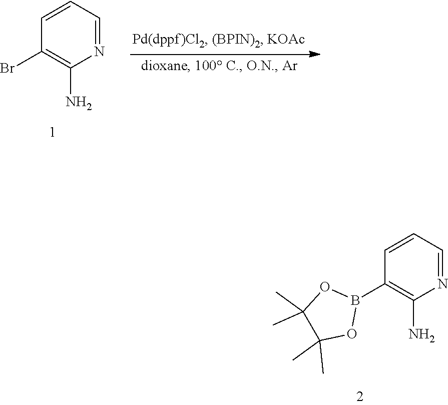 [SMILES: Nc1ncccc1Br ; CC1(C)OB(c2cccnc2N)OC1(C)C]

To a 250 mL round bottom flask were added 1 (500 mg, 2.89 mmol), (BPIN)₂ (881 mg, 3.47 mmol), AcOK (850 mg, 8.67 mmol) and Pd(dppf)Cl₂ (64 mg, 0.09 mmol). The mixture was dewatered by heated oil pump for 10 min and then the dry dioxane (20 mL) was added. The reaction mixture was degassed and refilled with Ar five times, then reaction mixture was stirred at 100° C. overnight. The reaction mixture was used directly for the next step without additional purification.

### The Synthesis of 3-(pyridazin-3-yl)pyridin-2-amine (3)

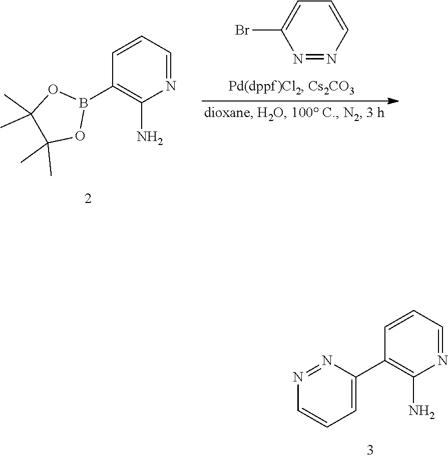 [SMILES: CC1(C)OB(c2cccnc2N)OC1(C)C ; Brc1cccnn1 ; Nc1ncccc1-c1cccnn1]

To the above reaction mixture (2) were added 3-bromopyridazine (0.53 g, 3.34 mmol), Cs₂CO₃ (2.07 g, 6.36 mmol), Pd(dppf)Cl₂ (0.07 g, 0.10 mmol) and H₂O (5 mL). The reaction mixture was degassed and refilled with N₂ five times, then reaction mixture was stirred at 100° C. for 3 hours. The reaction mixture was concentrated in vacuo, extracted with EA (40 mL×3) and H₂O (60 mL). The combined organic layers were washed with brine, dried over anhydrous Na₂SO₄, filtered and the filtrate was concentrated. The crude product was purified by silica gel chromatography using a mixture of petroleum ether-ethyl acetate (0:100, v/v) as eluent to afford 3 (260 mg, yield: 48%).

### The Synthesis of N-((1H-indazol-7-yl)methyl)-3-(pyridazin-3-yl)pyridin-2-amine (ELG-000480)

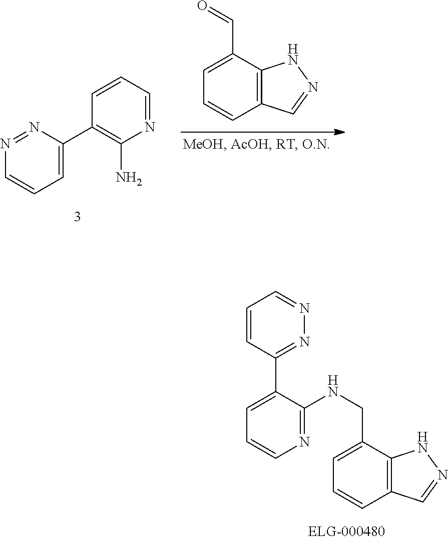 [SMILES: Nc1ncccc1-c1cccnn1 ; O=Cc1cccc2cn[nH]c12 ; c1cnnc(-c2cccnc2NCc2cccc3cn[nH]c23)c1]

To a 100 mL round bottom flask were added 3 (40 mg, 0.23 mmol), MeOH (3 mL) and AcOH (3 drops), and then add 1H-indazole-7-carbaldehyde (32 mg, 0.22 mmol) to the reaction mixture, the mixture was stirred at room temperature for 2 hours, then add NaBH₃CN (70 mg, 1.11 mmol) to the reaction mixture after the start materials disappears. The reaction mixture was stirred at room temperature overnight. The reaction mixture was quenched with H₂O and concentrated in vacuo, the crude product was purified by silica gel chromatography using a mixture of petroleum ether-ethyl acetate (50:50, v/v) as eluent to afford a green solid (20 mg, crude), then the crude was further purified by prep-HPLC (a mixture of CH₃CN and H₂O with 0.5% HCO₂H as eluent) to afford ELG-000480 (3.2 mg, yield: 4%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.004 min; MS Calcd: 302.13; MS Found: 303.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min: Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 95.90%, Rt=3.546 min. ¹H NMR (400 MHz. DMSO-d₆) δ 13.25 (s, 1H), 9.58 (t, J=5.7 Hz, 1H), 9.19 (dd, J=4.8, 1.5 Hz, 1H), 8.33 (dd, J=8.9, 1.4 Hz, 1H), 8.18 (ddd, J=9.4, 6.3, 1.7 Hz, 2H), 8.11 (d, J=17.5 Hz, 1H), 7.84 (dd, J=8.9, 4.8 Hz, 1H), 7.63 (d, J=8.0 Hz, 1H), 7.28 (d, J=7.0 Hz, 1H), 7.04 (dd, J=8.0, 7.1 Hz, 1H), 6.77 (dd, J=7.7, 4.8 Hz, 1H), 5.06 (d. J=5.7 Hz, 2H). Chemical Formula: C₁₇H₁₄N₆. Molecular Weight: 302.34.

### Route for ELG-000481

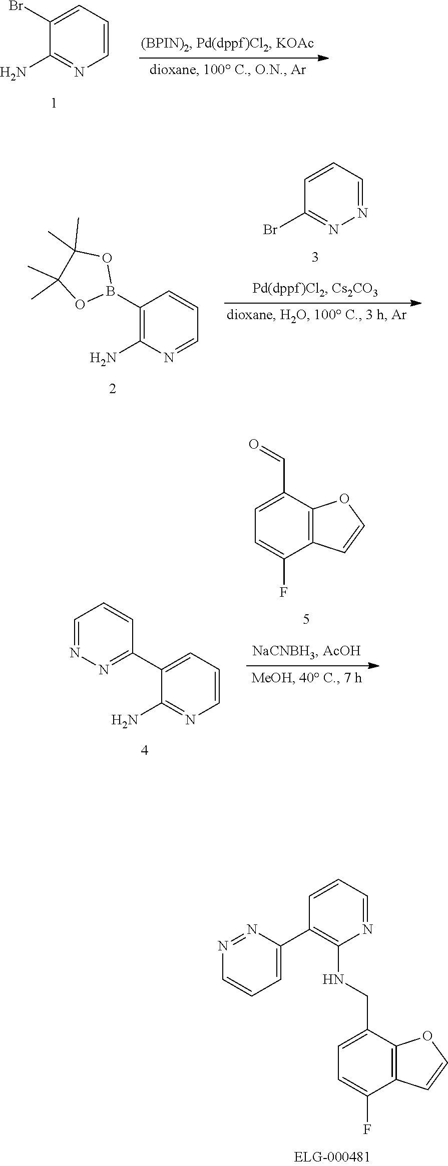 [SMILES: Nc1ncccc1Br ; CC1(C)OB(c2cccnc2N)OC1(C)C ; Brc1cccnn1 ; O=Cc1ccc(F)c2ccoc12 ; Nc1ncccc1-c1cccnn1 ; Fc1ccc(CNc2ncccc2-c2cccnn2)c2occc12]

### The Synthesis of 3-(4,4,5,5-tetramethyl-1,3,2-dioxaborolan-2-yl)pyridin-2-amine (2)

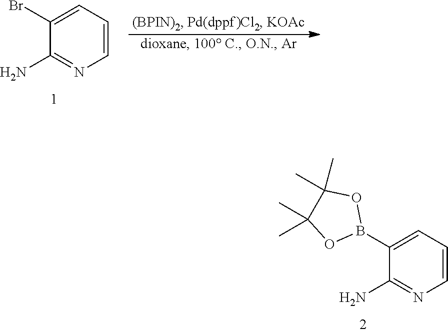

To a 250 mL round bottom flask were added 1 (2.5 g, 14.50 mmol). Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (4)

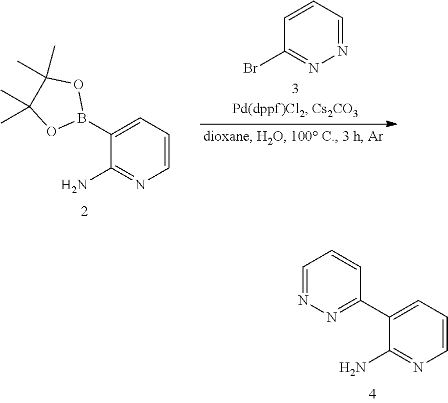 [SMILES: CC1(C)OB(c2cccnc2N)OC1(C)C ; Brc1cccnn1 ; Nc1ncccc1-c1cccnn1]

To a 250 mL round bottom flask were added 2 (3.19 g, 14.50 mmol). Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.1 g, yield: 44%).

### The Synthesis of N-((4-fluorobenzofuran-7-yl)methyl)-3-(pyridazin-3-yl)pyridin-2-amine (ELG-000481)

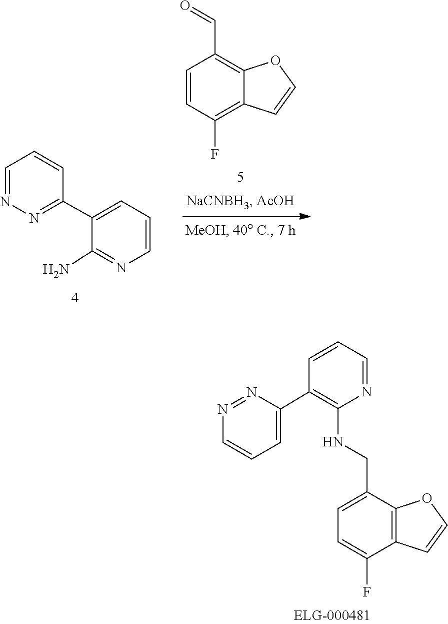 [SMILES: Nc1ncccc1-c1cccnn1 ; O=Cc1ccc(F)c2ccoc12 ; Fc1ccc(CNc2ncccc2-c2cccnn2)c2occc12]

To a 100 mL round bottom flask were added 4 (40.0 mg, 0.23 mmol). MeOH (3 mL), 5 (36.4 mg, 0.22 mmol) and AcOH (5 drops), the mixture was stirred at 40° C. until 5 consumed, about 2 hours. Then added NaCNBH₃ (29.0 mg, 0.46 mmol), the reaction mixture was stirred at room temperature for another 5 hours. The reaction mixture was quenched by water (0.5 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000481 (25.7 mg, yield: 36%) as a yellow solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 98.92%, Rt=1.163 min; MS Calcd: 320.11: MS Found: 321.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 100%, Rt=4.256 min. ¹H NMR (400 MHz, DMSO-d₆) δ 9.98 (brs, 1H), 9.21 (dd. J=4.9, 1.4 Hz, 1H), 8.39 (dd. J=8.9, 1.4 Hz, 1H), 8.31 (d, J=7.6 Hz, 1H), 8.19 (dd, J=5.1, 1.7 Hz, 1H), 8.10 (d, J=2.2 Hz, 1H), 7.87 (dd, J=8.9, 4.9 Hz, 1H), 7.27 (dd, J=8.2, 5.3 Hz, 1H), 7.11 (d, J=2.3 Hz, 1H), 7.05 (dd, J=9.6, 8.3 Hz, 1H), 6.86 (dd, J=7.6, 5.2 Hz, 1H), 5.06 (s, 2H). Chemical Formula: C18H₁₃N₄OF. Molecular Weight: 320.33.

### Route for ELG-000483

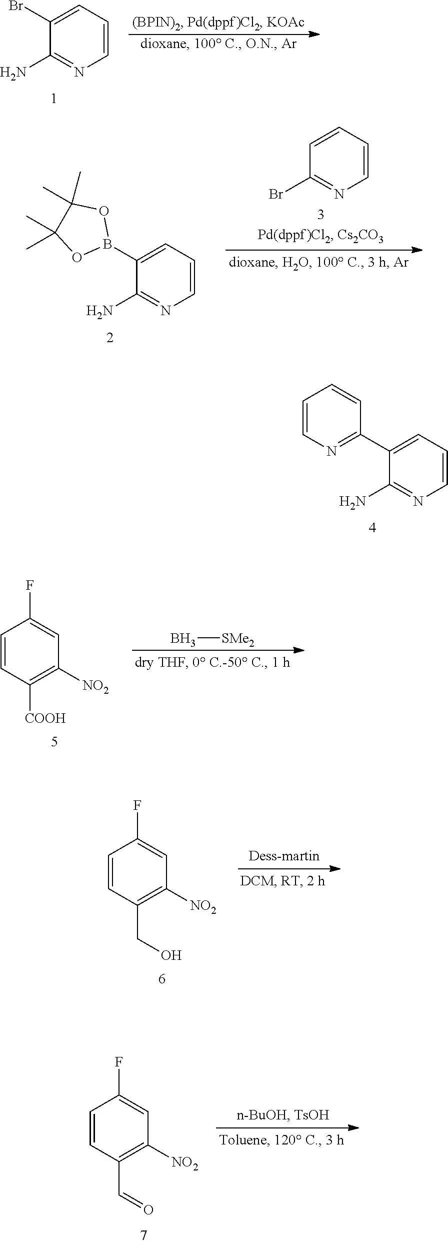

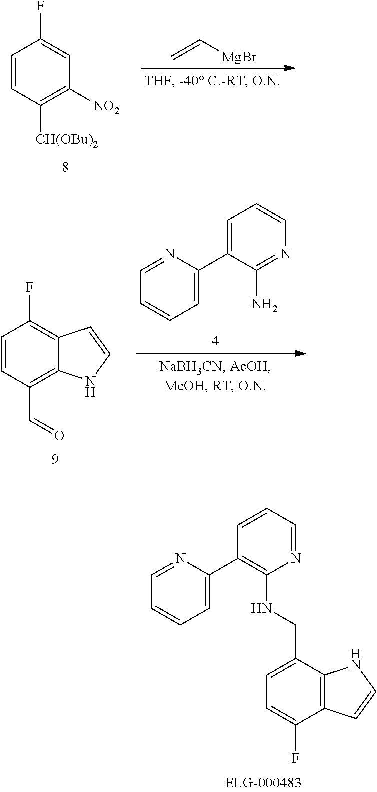

### The Synthesis of 3-(4,4,5,5-tetramethyl-1,3,2-dioxaborolan-2-yl)pyridin-2-amine (2)

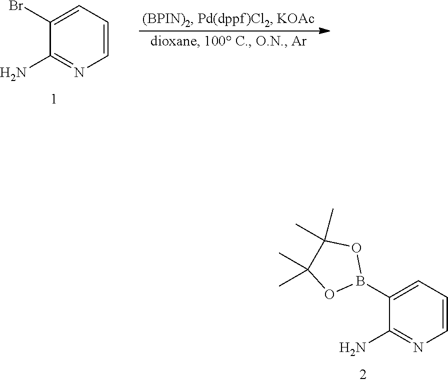 [SMILES: Nc1ncccc1Br ; "dioxane, 100° C., O.N., Ar" ; "(BPIN)2, Pd(dppf)Cl2, KOAc" ; CC1(C)OB(c2cccnc2N)OC1(C)C]

To a 250 mL round bottom flask were added 1 (2.5 g, 14.50 mmol). Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (4)

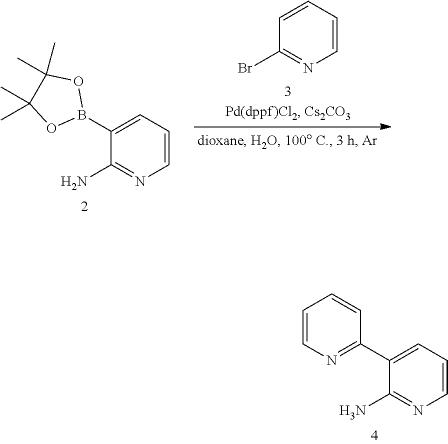 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.0 g, yield: 40%).

### The Synthesis of (4-fluoro-2-nitrophenyl) methanol (6)

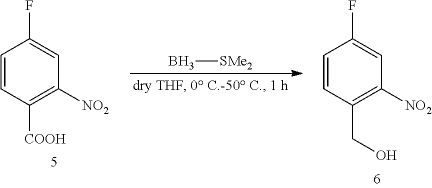

To a 100 mL three-necked flask were added 5 (2.0 g, 10.8 mmol) and THF (30 mL). The mixture was cooled to 0° C. in an ice-water bath, slowly added BH₃—SMe₂ (16.2 mL, 32.0 mmol) dropwise, then heated to 50° C. and stirred for 1 hour. After the reaction is complete, MeOH (20 mL) and water (50 mL) were added. The mixture was concentrated to remove organic solvents, extracted by EA (30 mL×5), combined organic layer, washed with brine (30 mL×3), dried over anhydrous sodium sulfate, filtered and concentrated the filtrate to afford a light yellow solid 6 (1.9 g, yield: 98%) without further purification.

### The Synthesis of 4-fluoro-2-nitrobenzaldehyde (7)

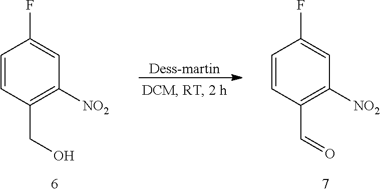 [SMILES: O=[N+]([O-])c1cc(F)ccc1CO ; "Dess-martin" ; O=Cc1ccc(F)cc1[N+](=O)[O-]]

To a 250 mL round bottom flask were added 6 (1.9 g, 11.1 mmol), DCM (40 mL) and Dess-Martin (7.1 g, 16.7 mmol) at 0° C., the mixture was stirred at room temperature for 2 hours. After the reaction is complete, water (50 mL) was added. The mixture was concentrated to remove organic solvents, extracted by EA (30 mL×3), combined organic layer, washed with brine (30 mL×2), dried over anhydrous sodium sulfate, filtered and concentrated the filtrate, purified by silica gel chromatography using a mixture of PE-EA (95:5, v/v) as eluent to afford to afford 7 (1.3 g, yield: 69%).

### The Synthesis of 1-(dibutoxymethyl)-4-fluoro-2-nitrobenzene (8)

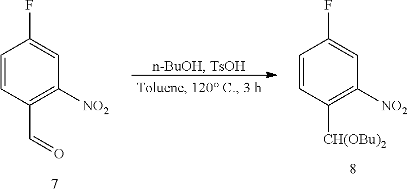

To a 100 mL round bottom flask were added 7 (500.0 mg, 2.96 mmol), n-butanol (548.2 mg, 7.39 mmol), p-toluenesulfonic acid monohydrate (11.0 mg, 0.06 mmol) and toluene (20 mL). The mixture was stirred at 120° C. and equipped with Dean-Stark equipment for 3 hours. Once the reaction completed, the reaction mixture was concentrated and purify by silica gel chromatography using a mixture of PE-EA (95:5, v/v) as eluent to afford to afford 8 (679.5 mg, yield: 76%).

### The Synthesis of 4-fluoro-1H-indole-7-carbaldehyde (9)

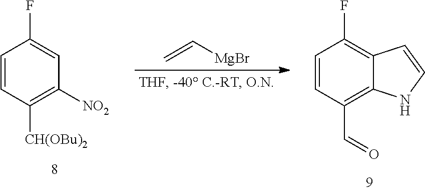

To a 100 mL three-necked flask were added 8 (450.0 mg, 1.5 mmol) and THF (15 mL), the solution was degassed and refilled Ar three times, then cooled to −40° C. and slowly added vinylmagnesium bromide (7 mL, 1 M in THF) by syringe over 1 hour in stirring. The reaction was warmed slowly to room temperature and stirred overnight. The reaction solution was quenched carefully by saturated aqueous ammonium chloride solution (20 mL) and water (30 mL), extracted by EA (30 mL×3), combined the organic layer, washed with brine (30 mL×2), dried over anhydrous sodium sulfate, filtered, concentrated the filtrate to give a yellow oil. The residue was dissolved in THF (5 mL) and hydrochloric acid (5 mL, 0.5 M in water), stirred at room temperature for 1 hour, then basified by saturated sodium bicarbonate solution till pH~8. The mixture was extracted by EA (20 mL×3), combined the organic layer, washed with brine (20 mL×2), dried over anhydrous sodium sulfate, filtered, concentrated the filtrate and purified by silica gel chromatography using a mixture of PE-EA (60:40, v/v) as eluent to afford 9 (185 mg, yield: 66%).

### The Synthesis of N-((4-fluoro-1H-indol-7-yl) methyl)-[2, 3′-bipyridin]-2-amine (ELG-000483)

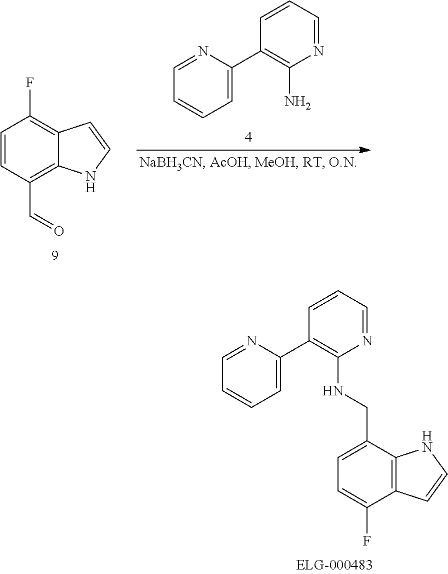 [SMILES: O=Cc1ccc(F)c2cc[nH]c12 ; Nc1ncccc1-c1ccccn1 ; Fc1ccc(CNc2ncccc2-c2ccccn2)c2[nH]ccc12]

To a 100 mL round bottom flask were added 9 (47.6 mg, 0.29 mmol), 4 (50.0 mg, 0.29 mmol), AcOH (5 drops) and methanol (4 mL), the mixture was stirred at 40° C. for 2 hours. Then the reaction solution was cooled to room temperature, added NaBH₃CN (91.7 mg, 1.46 mmol) and stirred overnight. The reaction solution was quenched by water (1 mL), purified by prep-TLC, to afford ELG-000483 (18.9 mg, yield: 20%) as a yellow solid. LC-MS (Agilent LCMS 1260-6125B, Column: Boltimate EXT C18 CoreShell (50 mm*4.6 mm*2.7 μm): Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 98.20%, Rt=1.311 min; MS Calcd: 318.13; MS Found: 319.1 [M+H]⁺. HPLC (Waters UPLC. Column: ACQUITY UPLC BEH C18 1.7 μm*2.1 mm*150 mm); Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 95.91%, Rt=4.684 min. ¹H NMR (400 MHz, DMSO-d₆) δ 11.55 (s, 1H), 9.70-9.65 (m, 1H), 8.61-8.57 (m, 1H), 8.14 (dd, J=4.8, 1.8 Hz, 1H), 8.08 (dd, J=7.7, 1.8 Hz, 1H), 7.99 (dt, J=8.4, 1.2 Hz, 1H), 7.95-7.90 (m, 1H), 7.40 (t, J=2.8 Hz, 1H), 7.35 (ddd, J=7.4, 4.9, 1.3 Hz, 1H), 7.01 (dd, J=8.0, 5.0 Hz, 1H), 6.73-6.67 (m, 2H), 6.51 (dd, J=3.2, 1.9 Hz, 1H), 4.94 (d. J=5.8 Hz, 2H). Chemical Formula: C₁₉H₁₅N₄F. Molecular Weight: 318.36.

### Route for ELG-000488

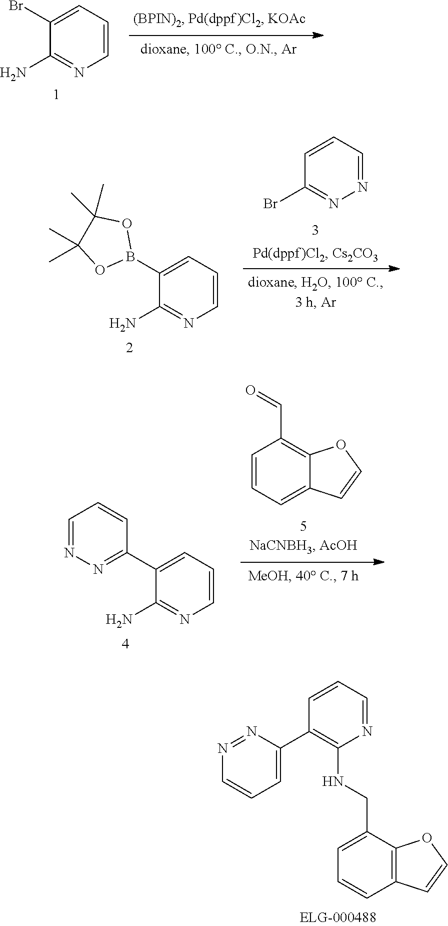 [SMILES: Nc1ncccc1Br ; CC1(C)OB(c2cccnc2N)OC1(C)C ; Brc1cccnn1 ; Nc1ncccc1-c1cccnn1 ; O=Cc1cccc2ccoc12 ; c1cnnc(-c2cccnc2NCc2cccc3ccoc23)c1]

### The Synthesis of 3-(4,4,5,5-tetramethyl-1,3,2-dioxaborolan-2-yl)pyridin-2-amine (2)

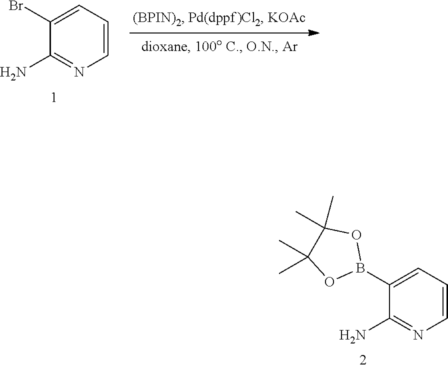 [SMILES: Nc1ncccc1Br ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (4)

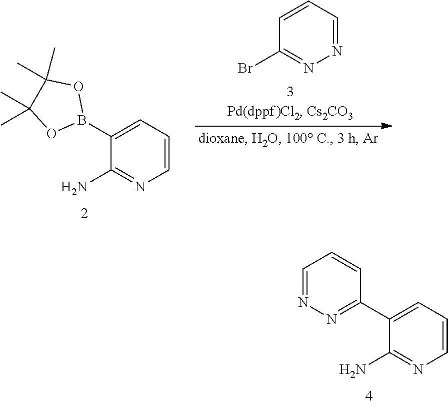 [SMILES: CC1(C)OB(c2cccnc2N)OC1(C)C ; Brc1cccnn1 ; Nc1ncccc1-c1cccnn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.1 g, yield: 44%).

### The Synthesis of N-(benzofuran-7-ylmethyl)-3-(pyridazin-3-yl) pyridin-2-amine (ELG-000488)

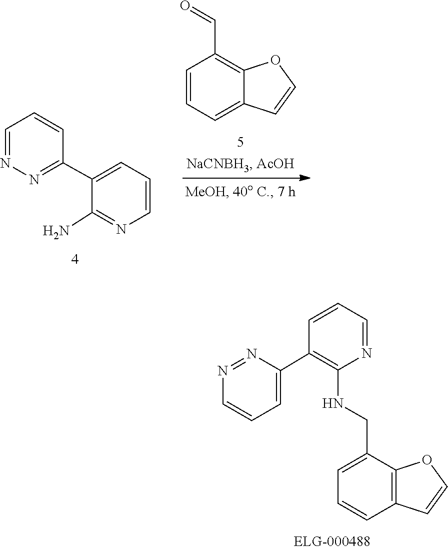 [SMILES: Nc1ncccc1-c1cccnn1 ; O=Cc1cccc2ccoc12 ; c1cnnc(-c2cccnc2NCc2cccc3ccoc23)c1]

To a 100 mL round bottom flask were added 4 (50.0 mg, 0.29 mmol), MeOH (3 mL), 5 (42.4 mg, 0.29 mmol) and AcOH (5 drops), the mixture was stirred at 40° C. until 5 consumed, about 2 hours. Then added NaCNBH₃ (29.0 mg, 0.46 mmol), the reaction mixture was stirred at room temperature for another 5 hours. The reaction mixture was quenched by water (0.5 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000488 (20.0 mg, yield: 22%) as a yellow solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.110 min: MS Calcd: 302.12; MS Found: 303.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 98.31%, Rt=3.977 min. ¹H NMR (400 MHz, DMSO-d₆) δ 9.69 (t, J=5.7 Hz, 1H), 9.17 (dd, J=4.8, 1.5 Hz, 1H), 8.36 (dd, J=8.9, 1.5 Hz, 1H), 8.25-8.12 (m, 2H), 8.01 (d, J=2.2 Hz, 1H), 7.83 (dd, J=8.9, 4.8 Hz, 1H), 7.54 (dd, J=7.6, 1.2 Hz, 1H), 7.25 (d, J=6.6 Hz, 1H), 7.18 (t, J=7.5 Hz, 1H), 6.98 (d, J=2.2 Hz, 1H), 6.77 (dd, J=7.3, 5.1 Hz, 1H), 5.07 (d, J=5.7 Hz, 2H). Chemical Formula: C18H₁₄N₄₀. Molecular Weight: 302.34.

### Route for ELG-000491

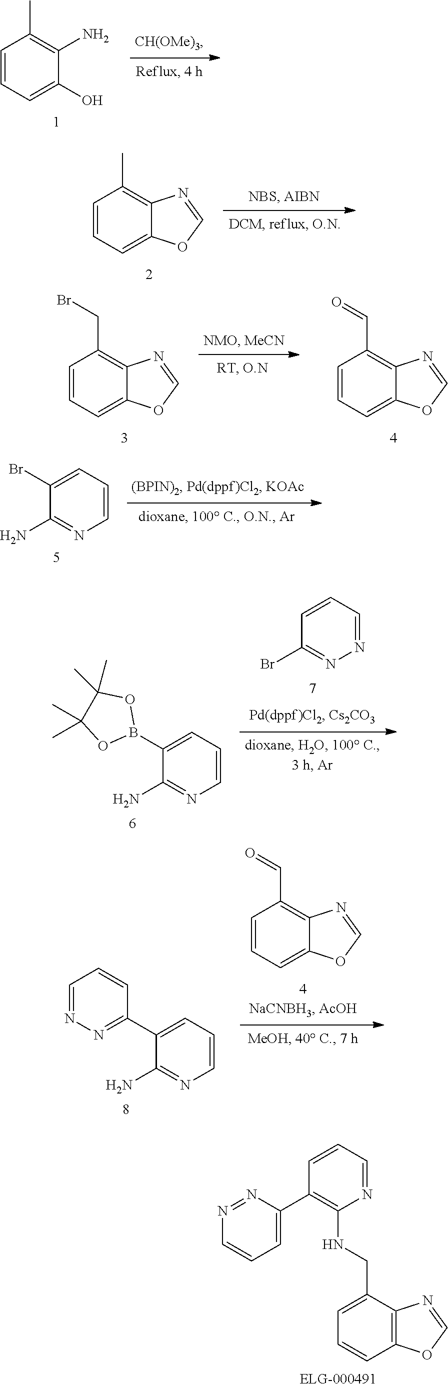

### The Synthesis of 4-methylbenzo[d]oxazole (2)

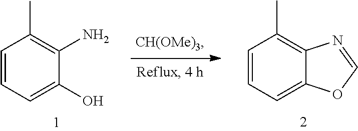

To a 100 mL round bottom flask were added 1 (616 mg, 5.0 mmol) and trimethoxymethane (15 mL), the reaction mixture was stirred at 110° C. for 4 hours. The reaction mixture was concentrated in vacuo and purified by silica gel chromatography using a mixture of petroleum ether-ethyl acetate (80:20, v/v) as eluent to afford 2 (483 mg, yield: 73%).

### The Synthesis of 4-(bromomethyl)benzo[d]oxazole (3)

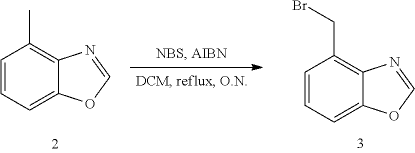

To a 100 mL round bottom flask were added 2 (483 mg, 3.6 mmol), NBS (840 mg, 4.7 mmol), AIBN (12 mg, 0.1 mmol) and DCM (40 mL), the reaction mixture was stirred at 45° C. overnight. Once the reaction completed, the reaction mixture was concentrated in vacuo and extracted with EA (20 mL×3), the organic phase was washed with saturated sodium bicarbonate aqueous solution (20 mL×3), the organic phase was combined and dried over anhydrous Na₂SO₄ and concentrated in vacuo. The crude product was purified by silica gel chromatography using a mixture of petroleum ether-ethyl acetate (95:5, v/v) as eluent to afford 3 (571 mg, yield: 74%).

### The Synthesis of benzo[d]oxazole-4-carbaldehyde (4)

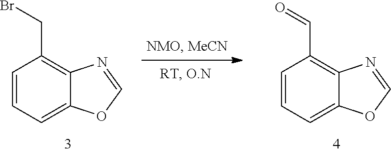 [SMILES: BrCc1cccc2ocnc12 ; O=Cc1cccc2ocnc12]

To a 100 mL round bottom flask were added 3 (250 mg, 1.18 mmol), NMO (213 mg, 1.81 mmol), acetonitrile (4 mL), the reaction mixture was stirred at room temperature for 1 hour. Once the reaction completed, water (20 mL) was added in the reaction mixture and extracted with EA (15 mL×3), the organic phase was combined, dried over anhydrous Na₂SO₄ and concentrated in vacuo. The crude product was purified by silica gel chromatography using a mixture of petroleum ether-ethyl acetate (90:10, v/v) as eluent to afford 4 (80 mg, yield: 42%).

### The Synthesis of 3-(4,4,5,5-tetramethyl-1,3,2-dioxaborolan-2-yl)pyridin-2-amine (6)

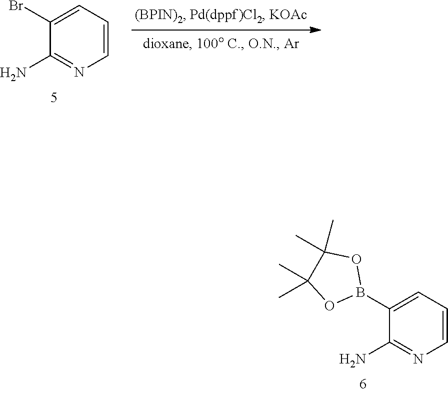 [SMILES: Nc1ncccc1Br ; CC1(C)OB(c2cccnc2N)OC1(C)C]

To a 250 mL round bottom flask were added 5 (2.5 g, 14.50 mmol). Pd(dppf)Cl₂ (530.6 mg, 0.73 mmol), (BPIN)₂ (4.4 g, 17.40 mmol). KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (8)

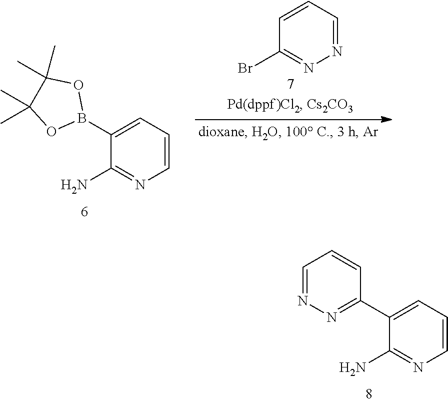 [SMILES: CC1(C)OB(c2cccnc2N)OC1(C)C ; Brc1cccnn1 ; Nc1ncccc1-c1cccnn1]

To a 250 mL round bottom flask were added 6 (3.19 g, 14.50 mmol), Pd(dppf)Cl₂ (530.6 mg, 0.73 mmol), Cs₂CO₃ (9.5 g, 29.0 mmol), 7 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 8 (1.1 g, yield: 44%).

### The Synthesis of N-(benzo[d]oxazol-4-ylmethyl)-3-(pyridazin-3-yl)pyridin-2-amine (ELG-000491)

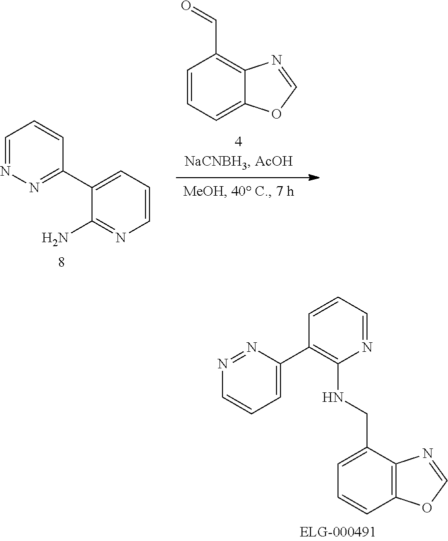 [SMILES: O=Cc1cccc2ocnc12 ; Nc1ncccc1-c1cccnn1 ; c1cnnc(-c2cccnc2NCc2cccc3ocnc23)c1]

To a 100 mL round bottom flask were added 8 (60.2 mg, 0.35 mmol), MeOH (3 mL), 4 (42.9 mg, 0.26 mmol) and AcOH (5 drops), the mixture was stirred at 40° C. until 4 consumed, about 2 hours. Then added NaCNBH₃ (54.7 mg, 0.87 mmol), the reaction mixture was stirred at room temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000491 (8.0 mg, yield: 10%) as a yellow solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 89.48%. Rt=0.979 min; MS Calcd: 303.11; MS Found: 304.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 87.62%, Rt=3.711 min. ¹H NMR (400 MHz, DMSO-d₆) δ 9.86 (brs, 1H), 9.20 (dd, J=4.8, 1.4 Hz, 1H), 8.76 (s, 1H), 8.38 (dd, J=8.9, 1.4 Hz, 1H), 8.25 (d, J=7.4 Hz, 1H), 8.19 (dd, J=5.0, 1.7 Hz, 1H), 7.86 (dd, J=8.9, 4.9 Hz, 1H), 7.66 (dd, J=7.8, 1.1 Hz, 1H), 7.41-7.28 (m, 2H), 6.82 (dd, J=7.5, 5.1 Hz, 1H), 5.14 (s, 2H). Chemical Formula: C₁₇H₁₃N₅₀. Molecular Weight: 303.33.

### Route for ELG-000496

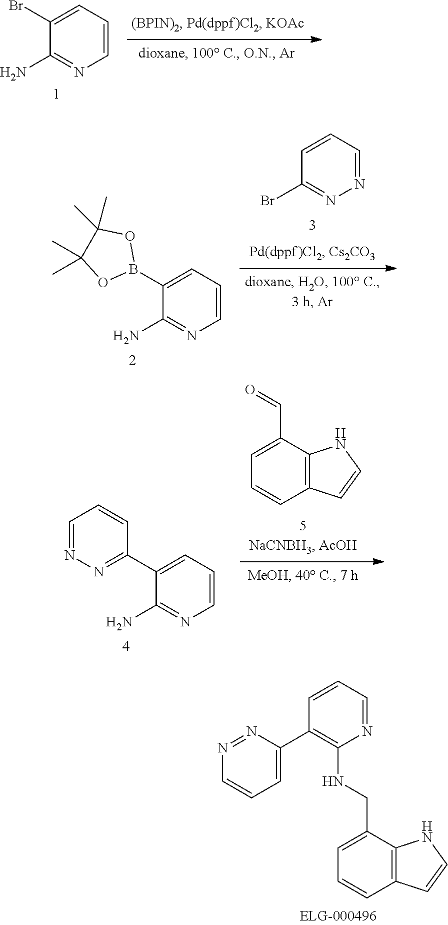 [SMILES: Nc1ncccc1Br ; CC1(C)OB(c2cccnc2N)OC1(C)C ; Brc1cccnn1 ; O=Cc1cccc2cc[nH]c12 ; Nc1ncccc1-c1cccnn1 ; c1cnnc(-c2cccnc2NCc2cccc3cc[nH]c23)c1]

### The Synthesis of 3-(4,4,5,5-tetramethyl-1,3,2-dioxaborolan-2-yl)pyridin-2-amine (2)

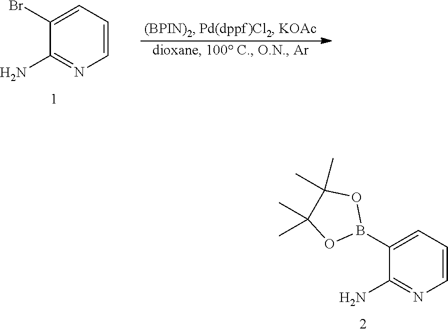 [SMILES: Nc1ncccc1Br ; "dioxane, 100° C., O.N., Ar" ; "(BPIN)2, Pd(dppf)Cl2, KOAc" ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (4)

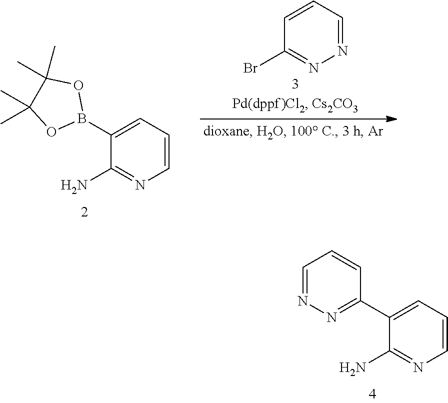 [SMILES: CC1(C)OB(c2cccnc2N)OC1(C)C ; Brc1cccnn1 ; Nc1ncccc1-c1cccnn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.1 g, yield: 44%).

### The Synthesis of N-((1H-indol-7-yl)methyl)-3-(pyridazin-3-yl)pyridin-2-amine (ELG-000496)

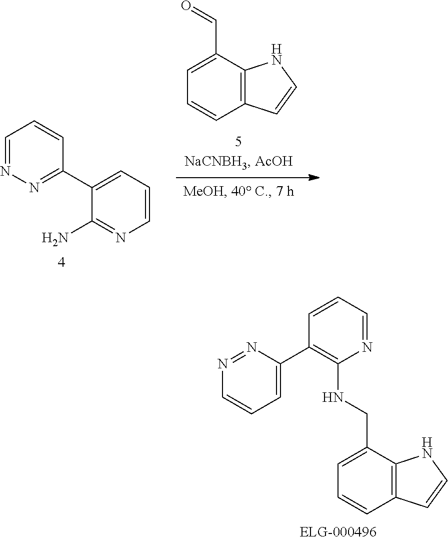 [SMILES: O=Cc1cccc2cc[nH]c12 ; Nc1ncccc1-c1cccnn1 ; c1cnnc(-c2cccnc2NCc2cccc3cc[nH]c23)c1]

To a 100 mL round bottom flask were added 4 (50.0 mg, 0.29 mmol). MeOH (3 mL), 5 (42.1 mg, 0.29 mmol) and AcOH (5 drops), the mixture was stirred at 40° C. until 5 consumed, about 2 hours. Then added NaCNBH₃ (91.1 mg, 1.45 mmol), the reaction mixture was stirred at room temperature for another 5 hours. The reaction mixture was quenched by water (0.5 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000496 (5.2 mg, yield: 6%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.145 min; MS Calcd: 301.13; MS Found: 302.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 96.20%, Rt=4.038 min. ¹H NMR (400 MHz, DMSO-d₆) δ 11.21 (brs, 1H), 9.57 (t, J=5.5 Hz, 1H), 9.16 (dd, J=4.8, 1.4 Hz, 1H), 8.33 (dd, J=8.9, 1.4 Hz, 1H), 8.24 (dd. J=4.8, 1.7 Hz, 1H), 8.17 (dd, J=7.7, 1.7 Hz, 1H), 7.82 (dd, J=8.9, 4.8 Hz, 1H), 7.44 (d, J=7.8 Hz, 1H), 7.34 (t, J=2.8 Hz, 1H), 7.08 (d, J=7.0 Hz, 1H), 6.96-6.89 (m, 1H), 6.77 (dd, J=7.7, 4.8 Hz, 1H), 6.46 (dd, J=3.0, 1.9 Hz, 1H), 5.02 (d, J=5.6 Hz, 2H). Chemical Formula: C18H₁₅N₅. Molecular Weight: 301.35.

### Route for ELG-000499

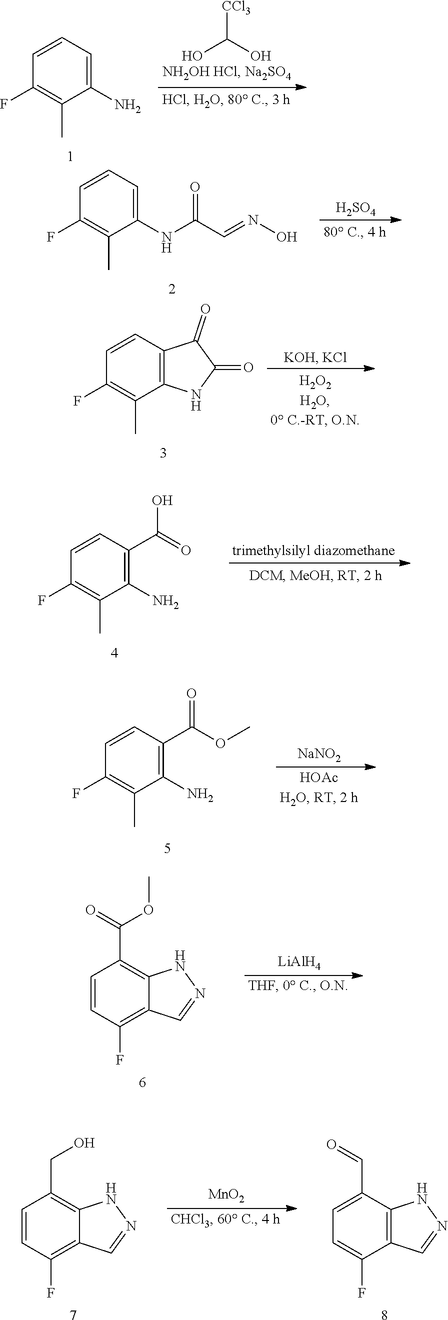

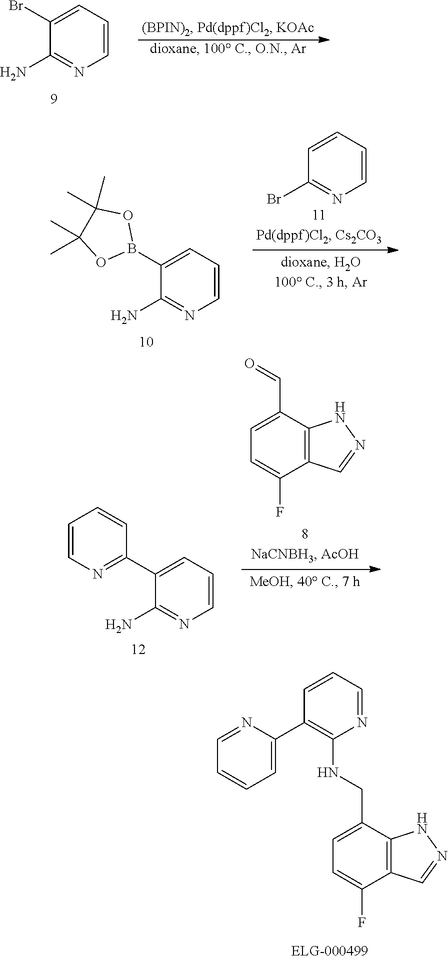

### The Synthesis of (E)-N-(3-fluoro-2-methylphenyl)-2-(hydroxyimino)acetamide (2)

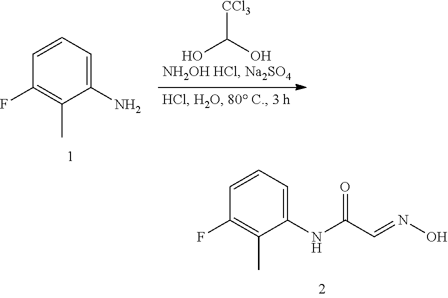

To a 1 L round bottom flask were added Na₂SO₄ (227 g, 1.59 mol), H₂O (400 mL) and 2,2,2-trichloroethane-1,1-diol (29 g, 175.6 mmol), then 1 (20.0 g, 159.8 mmol) in HCl (200 mL, 1 M in water) solution was added into the mixture, after stirred for 10 mins, NH₂OH HCl (44.4 g, 619.2 mmol) was added into the above solution and the mixture was stirred at 80° C. for 3 hours. After reaction was completed, reaction mixture was cooled down to room temperature, filtered, the filter cake was dried in vacuo to afford 2 (59 g) as crude product.

### The Synthesis of 6-fluoro-7-methylindoline-2,3-dione (3)

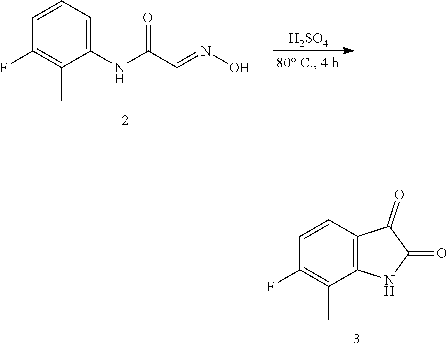

To a 1 L round bottom flask were added 2 (59 g, 310 mmol) and conc. H₂SO₄ (300 mL), the mixture was stirred at 80° C. for 4 hours. After reaction was completed, the reaction mixture was poured carefully into the ice water, extracted with EtOAc (200 mL×4), the organic phases was separated and combined, dried over anhydrous Na₂SO₄, concentrated in vacuo to afford 3 (5.34 g, yield: 17%).

### The Synthesis of 2-amino-4-fluoro-3-methylbenzoic acid (4)

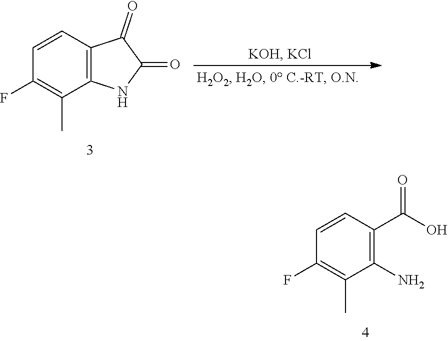

To a 250 mL three-necked flask were added 3 (5.34 g, 29.83 mmol) and H₂O (100 mL), KOH (1.84 g, 32.83 mmol) and KCl (4.66 mg, 62.52 mmol) was added at 0° C., and then slowly add H₂O₂ (5.07 g, 44.08 mmol) about 10 mins under protection of N₂, the mixture was stirred at room temperature overnight. The mixture was acidified carefully with TFA to pH=5 and diluted with H₂O (100 mL), extracted with EtOAc (100 mL×3), the organic phases was separated and combined, dried over anhydrous Na₂SO₄, concentrated in vacuo to afford 4 (4.41 g, yield: 90%).

### The Synthesis of methyl 2-amino-4-fluoro-3-methylbenzoate (5)

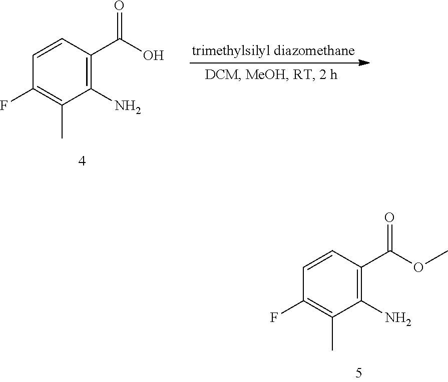

To a 250 mL round bottom flask were added 4 (4.41 g, 26.08 mmol), DCM (50 mL), MeOH (10 mL), trimethylsilyl diazomethane (4.47 g, 39.19 mmol) was added into the mixture at ice bath. Mixture was stirred at room temperature for 2 hours. Water (100 mL) was added and extracted with DCM (50 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 5 (1.36 g, yield: 84%).

### The Synthesis of methyl 4-fluoro-1H-indazole-7-carboxylate (6)

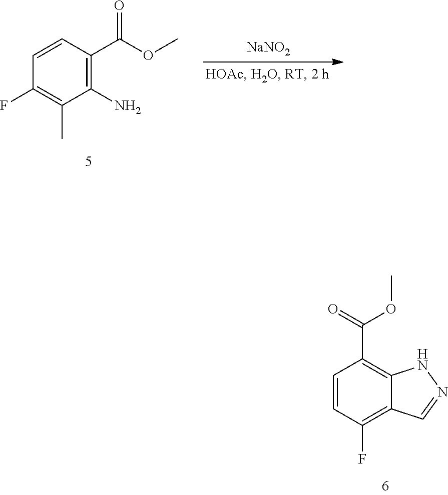

To a 100 mL round bottom flask were added 5 (1.36 g, 7.40 mmol), HOAc (15 mL), NaNO₂ (566 mg, 8.16 mmol) and H₂O (5 mL), mixture was stirred at room temperature for 2 hours. After reaction was completed, mixture was concentrated and residue was treated with a.q. NaHCO₃ solution to pH=7, mixture was extracted with EtOAc (30 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 6 (757 mg, yield: 90%).

### The Synthesis of (4-fluoro-1H-indazol-7-yl)methanol (7)

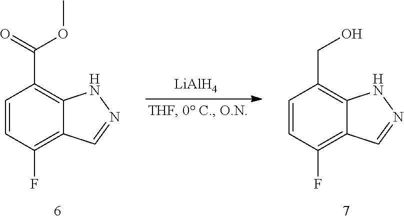

To a 100 mL three-necked flask were added 6 (757 mg, 3.94 mmol) and THF (15 mL), LiAlH₄ (2.27 mL, 5.91 mmol in THF) was added into the mixture by dropwise at 0° C., mixture was stirred at same temperature overnight. After reaction was completed, reaction mixture was quenched with 15% NaOH solution, and filtered. The filtrate was extracted with H₂O (30 mL) and EtOAc (20 mL×3), the organic phases was separated and combined, dried over anhydrous Na₂SO₄, organic phase was concentrated and purified by silica gel chromatography using a mixture of DCM-MeOH (90:10, v/v) as eluent to afford 7 (655 mg, yield: 78%).

### The Synthesis of 4-fluoro-1H-indazole-7-carbaldehyde (8)

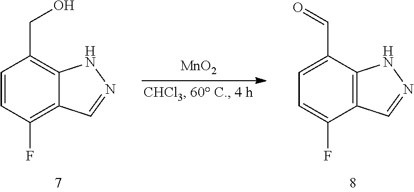 [SMILES: OCc1ccc(F)c2cn[nH]c12 ; "MnO2" ; O=Cc1ccc(F)c2cn[nH]c12]

To a 100 mL three-necked flask were added 7 (655 mg, 3.93 mmol), MnO₂ (1.7 g, 19.49 mmol) and CHCl₃ (15 mL), the mixture was stirred at 60° C. for 4 hours. The mixture was filtered and filter cake was washed with DCM, the filtrate was concentrated in vacuo and purified by silica gel chromatography using a mixture of petroleum ether-ethyl acetate (80:20, v/v) as eluent to afford 8 (450 mg, yield: 90%).

### The Synthesis of 3-(4,4,5,5-tetramethyl-1,3,2-dioxaborolan-2-yl)pyridin-2-amine (10)

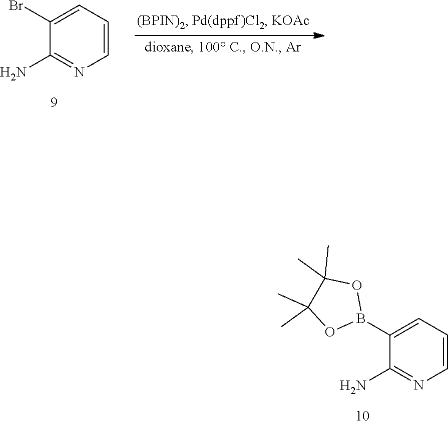 [SMILES: Nc1ncccc1Br ; CC1(C)OB(c2cccnc2N)OC1(C)C]

To a 250 mL round bottom flask were added 9 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (12)

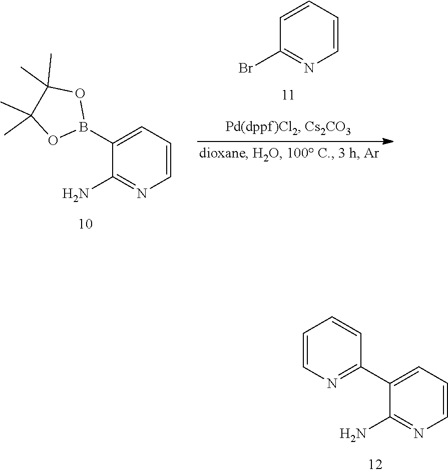 [SMILES: CC1(C)OB(c2cccnc2N)OC1(C)C ; Brc1ccccn1 ; Nc1ncccc1-c1ccccn1]

To a 250 mL round bottom flask were added 10 (3.19 g 14.50 mmol), Pd(dppf)Cl₂ (530.6 mg, 0.73 mmol), Cs₂CO₃ (9.5 g, 29.0 mmol), 11 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 12 (1.0 g. yield: 40%).

### The Synthesis of N-((4-fluoro-1H-indazol-7-yl)methyl)-[2,3′-bipyridin]-2′-amine (ELG-000499)

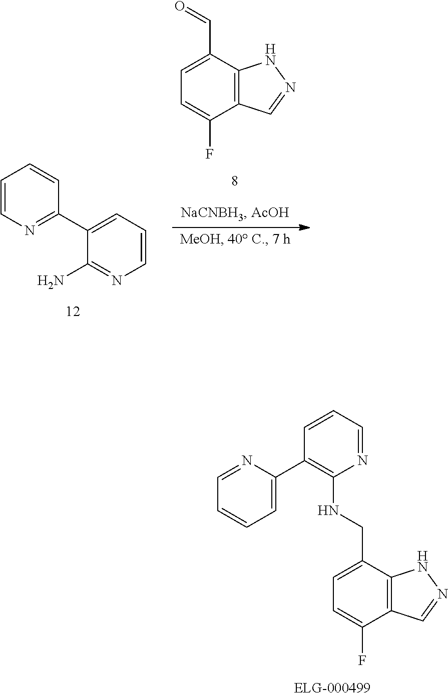 [SMILES: O=Cc1ccc(F)c2cn[nH]c12 ; Nc1ncccc1-c1ccccn1 ; Fc1ccc(CNc2ncccc2-c2ccccn2)c2[nH]ncc12]

To a 100 mL bottom flask were added 12 (50.0 mg, 0.29 mmol), 8 (48.0 mg, 0.29 mmol), AcOH (3 drops) and methanol (5 mL), the mixture stirred at 40° C. for 2 hours. Then the reaction mixture was added NaBH₃CN (91.7 mg, 1.46 mmol) and stirred at the same temperature for another 5 hours. The mixture was directly purified by prep-TLC (a mixture of petroleum ether and ethyl acetate, 1:1, v/v) to afford ELG-000499 (22.8 mg, yield: 24%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min. then under this condition for 1.0 min, finally changed to 90% [water+0.05% FA] and 10% [CH₃CN+0.05% FA] in 0.5 min), Purity: 98.79%, Rt=1.191 min; MS Calcd: 319.12; MS Found: 320.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min. then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 96.37%, Rt=4.372 min. ¹H NMR (400 MHz, DMSO-d₆) δ 13.60 (s, 1H), 9.71 (t, J=5.4 Hz, 1H), 8.65-8.60 (m, 1H), 8.19 (d, J=1.1 Hz, 1H), 8.12-8.06 (m, 2H), 8.01-7.91 (m, 2H), 7.37 (ddd, J=7.2, 4.9, 1.1 Hz, 1H), 7.23 (dd, J=7.7, 4.9 Hz, 1H), 6.81 (dd, J=10.3, 7.8 Hz, 1H), 6.70 (dd, J=7.6, 4.8 Hz, 1H), 4.98 (d, J=5.7 Hz, 2H). Chemical Formula: C18H₁₄FN₅. Molecular Weight: 319.34.

### Route for ELG-000502

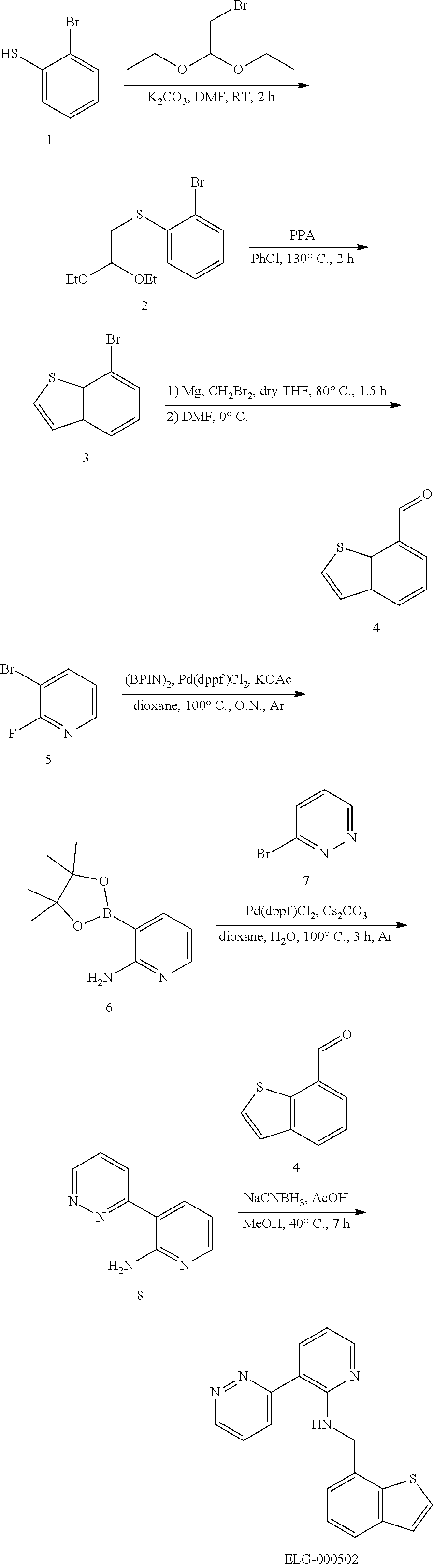

### The Synthesis of (2-bromophenyl)(2,2-diethoxyethyl)sulfane (2)

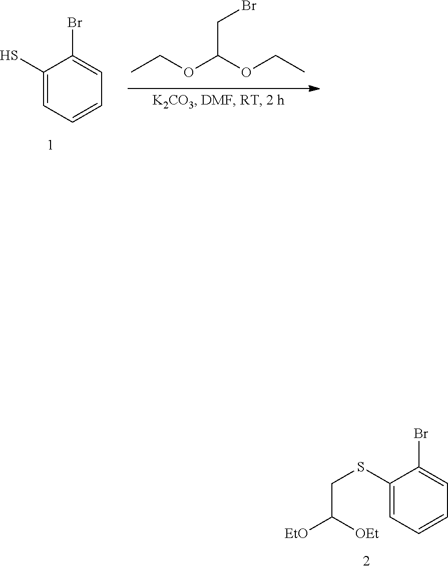

To a 100 mL round bottom flask were added 1 (4.4 g, 23.3 mmol), potassium carbonate (5.1 g, 36.9 mmol), Bromoacetaldehyde diethyl acetal (5.0 g, 25.4 mmol) and DMF (40 mL), the reaction mixture was stirred at room temperature for 4 hours. Once the reaction completed, water (100 mL) was added in the reaction and extracted with EtOAc (30 mL×4), the organic phase was washed with saturated brine (30 mL×4) and dried over Na₂SO₄ and concentrated in vacuo. The crude product was purified by silica gel chromatography using a mixture of petroleum ether-ethyl acetate (90:10, v/v) as eluent to afford 2 (6.6 g, yield: 94%).

### The Synthesis of 7-bromobenzo[b]thiophene (3)

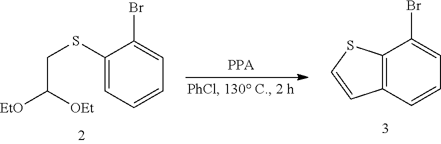 [SMILES: CCOC(CSc1ccccc1Br)OCC ; Brc1cccc2ccsc12]

To a 100 mL round bottom flask were added paraformaldehyde (15.0 g, 166.5 mmol) and chlorobenzene (50 mL) and 2 (6.6 g, 21.7 mmol). The reaction mixture was stirred at 130° C. for 2 hours. The reaction mixture was concentrated in vacuo, the residue was diluted with water (100 mL), extracted with EtOAc (30 mL×5), the combined organic phase was washed with saturated sodium bicarbonate aqueous solution (30 mL×3), dried over anhydrous Na₂SO₄ and concentrated in vacuo. The crude product was purified by silica gel chromatography using petroleum ether as eluent to afford 3 (3.7 g, yield: 79%).

### The Synthesis of benzo[b]thiophene-7-carbaldehyde (4)

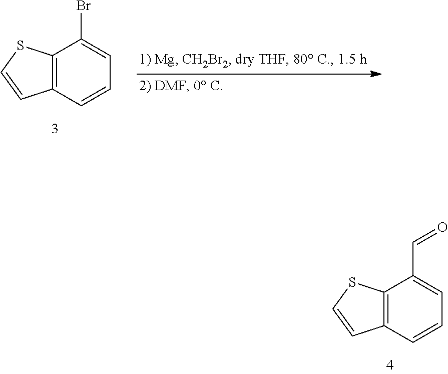 [SMILES: Brc1cccc2ccsc12 ; O=Cc1cccc2ccsc12]

To a 100 mL round bottom flask were added magnesium bars (616.8 mg, 25.7 mmol) and THF (14 mL), the reaction mixture was degassed and refilled with Ar three times, then stirred at 80° C. for 1.5 hours. The reaction mixture was cooled to 0° C., DMF (2.8 mL, 32.8 mmol) was slowly added and stirred at room temperature overnight. Once the reaction completed, the reaction mixture was concentrated in vacuo and purified by silica gel chromatography using a mixture of petroleum ether-ethyl acetate (95:5, v/v) as eluent to afford 4 (1.6 g. yield: 59%).

### The Synthesis of 3-(4,4,5,5-tetramethyl-1,3,2-dioxaborolan-2-yl)pyridin-2-amine (6)

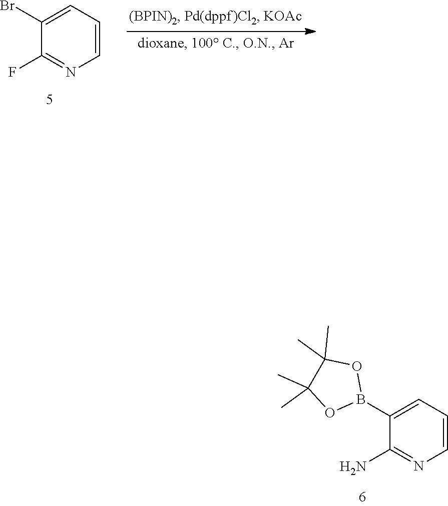 [SMILES: Fc1ncccc1Br ; CC1(C)OB(c2cccnc2N)OC1(C)C]

To a 250 mL round bottom flask were added 5 (2.5 g, 14.50 mmol). Pd(dppf)Cl₂ (530.6 mg, 0.73 mmol), (BPIN)₂ (4.4 g, 17.40 mmol). KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (8)

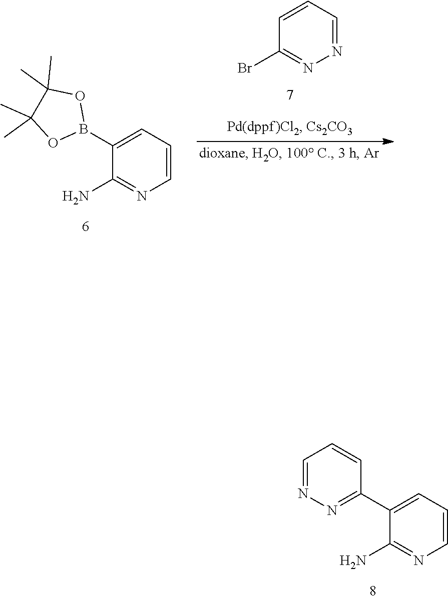 [SMILES: CC1(C)OB(c2cccnc2N)OC1(C)C ; Brc1cccnn1 ; Nc1ncccc1-c1cccnn1]

To a 250 mL round bottom flask were added 6 (3.19 g, 14.50 mmol), Pd(dppf)Cl₂ (530.6 mg, 0.73 mmol), Cs₂CO₃ (9.5 g, 29.0 mmol), 7 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 8 (1.1 g, yield: 44%).

### The Synthesis of N-(benzo[b]thiophen-7-ylmethyl)-3-(pyridazin-3-yl)pyridin-2-amine (ELG-000502)

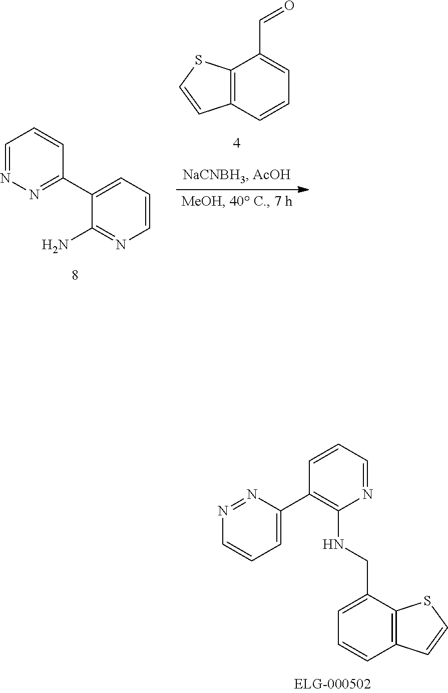 [SMILES: O=Cc1cccc2ccsc12 ; Nc1ncccc1-c1cccnn1 ; c1cnnc(-c2cccnc2NCc2cccc3ccsc23)c1]

To a 100 mL bottom flask were added 4 (59.0 mg, 0.36 mmol), 8 (75.2 mg, 0.44 mmol), AcOH (3 drops) and methanol (10 mL), the mixture stirred at 40° C. for 2 hours. Then the reaction mixture was added NaBH₃CN (114.4 mg, 1.82 mmol) and stirred at the same temperature for another 5 hours. The mixture was directly purified by prep-TLC (a mixture of petroleum ether and ethyl acetate, 5:1, v/v) to afford ELG-000502 (21.4 mg, yield: 18%) as a white solid. LC-MS (Agilent LCMS 1260-6125B. Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.196 min; MS Calcd: 318.09: MS Found: 319.00 [M+H]⁺. HPLC (Waters UPLC, Column: ACQUITY UPLC BEH C18 1.7 μm*2.1 mm*150 mm); Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 97.72%, Rt=4.340 min. ¹H NMR (400 MHz. DMSO-d₆) δ 9.76 (t, J=5.6 Hz, 1H), 9.17 (dd, J=4.8, 1.4 Hz, 1H), 8.37 (dd, J=8.9, 1.4 Hz, 1H), 8.22-8.17 (m, 2H), 7.84 (dd, J=8.9, 4.8 Hz, 1H), 7.79 (dd, J=6.7, 2.3 Hz, 1H), 7.73 (d, J=5.4 Hz, 1H), 7.49 (d, J=5.4 Hz, 1H), 7.38-7.33 (m, 2H), 6.78 (dd, J=7.6, 4.8 Hz, 1H), 5.02 (s, 2H). Chemical Formula: C18H₁₄N₄S. Molecular Weight: 318.40.

### Route for ELG-000504

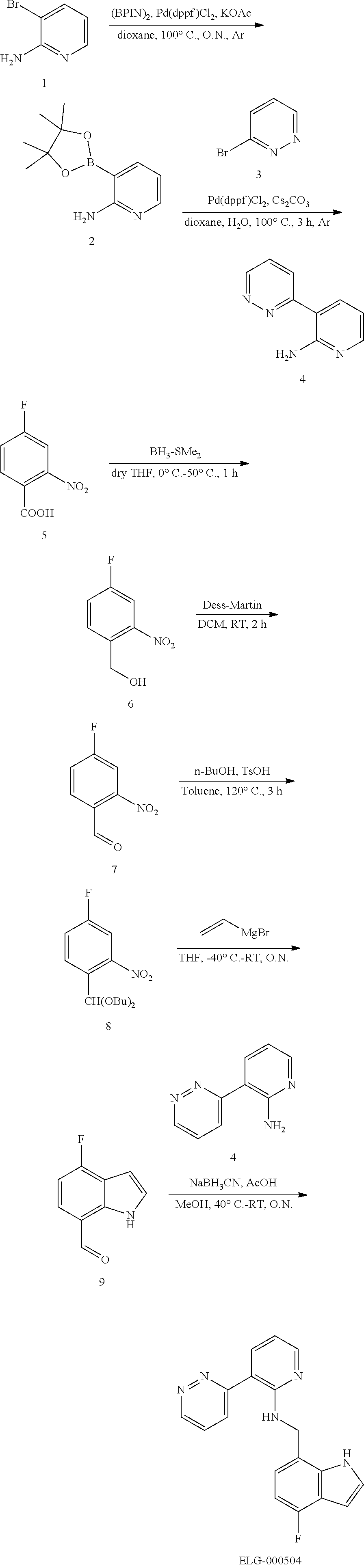

### The Synthesis of 3-(4,4,5,5-tetramethyl-1,3,2-dioxaborolan-2-yl)pyridin-2-amine (2)

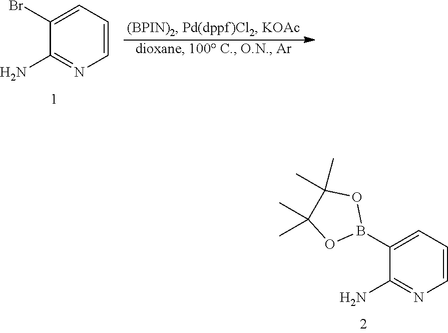

To a 250 mL round bottom flask were added 1 (2.5 g, 14.50 mmol). Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (4)

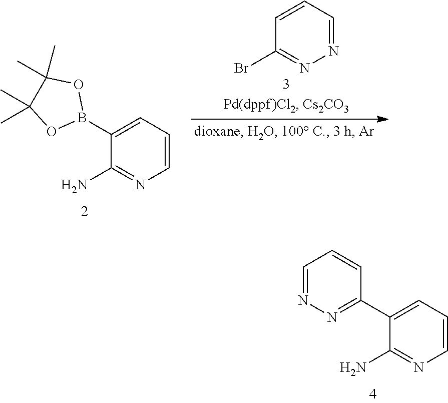 [SMILES: CC1(C)OB(c2cccnc2N)OC1(C)C ; Brc1cccnn1 ; Nc1ncccc1-c1cccnn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 3 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 4 (1.1 g, yield, 44%).

### The Synthesis of (4-fluoro-2-nitrophenyl) methanol (6)

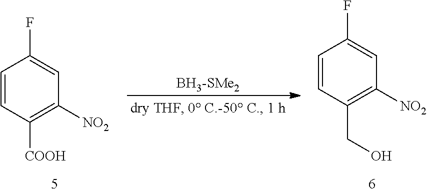 [SMILES: O=C(O)c1ccc(F)cc1[N+](=O)[O-] ; O=[N+]([O-])c1cc(F)ccc1CO ; "BH3-SMe2"]

To a 100 mL three-necked flask were added 5 (2.0 g, 10.8 mmol) and THF (30 mL). The mixture was cooled to 0° C. in an ice-water bath, slowly added BH₃—SMe₂ (16.2 mL, 32.0 mmol) dropwise, then heated to 50° C. and stirred for 1 hour. After the reaction is complete, MeOH (20 mL) and water (50 mL) were added. The mixture was concentrated to remove organic solvents, extracted by EA (30 mL×5), combined organic layer, washed with brine (30 mL×3), dried over anhydrous sodium sulfate, filtered and concentrated the filtrate to afford a light yellow solid 6 (1.9 g, yield: 98%) without further purification.

### The Synthesis of 4-fluoro-2-nitrobenzaldehyde (7)

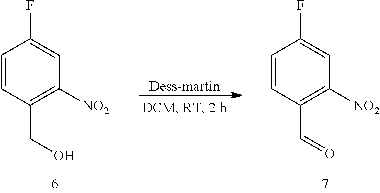 [SMILES: O=[N+]([O-])c1cc(F)ccc1CO ; "Dess-martin" ; O=Cc1ccc(F)cc1[N+](=O)[O-]]

To a 250 mL round bottom flask were added 6 (1.9 g, 11.1 mmol), DCM (40 mL) and Dess-Martin (7.1 g, 16.7 mmol) at 0° C., the mixture was stirred at room temperature for 2 hours. After the reaction is complete, water (50 mL) was added. The mixture was concentrated to remove organic solvents, extracted by EA (30 mL×3), combined organic layer, washed with brine (30 mL×2), dried over anhydrous sodium sulfate, filtered and concentrated the filtrate, purified by silica gel chromatography using a mixture of PE-EA (95:5, v/v) as eluent to afford to afford 7 (1.3 g, yield: 69%).

### The Synthesis of 1-(dibutoxymethyl)-4-fluoro-2-nitrobenzene (8)

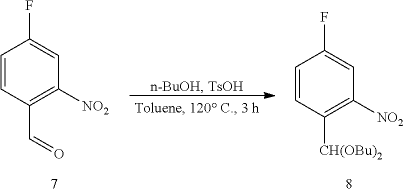

To a 100 mL round bottom flask were added 7 (500.0 mg, 2.96 mmol), n-butanol (548.2 mg, 7.39 mmol), p-toluenesulfonic acid monohydrate (11.0 mg, 0.06 mmol) and toluene (20 mL). The mixture was stirred at 120° C. and equipped with Dean-Stark equipment for 3 hours. Once the reaction completed, the reaction mixture was concentrated and purify by silica gel chromatography using a mixture of PE-EA (95:5, v/v) as eluent to afford to afford 8 (679.5 mg, yield: 76%).

### The Synthesis of 4-fluoro-1H-indole-7-carbaldehyde (9)

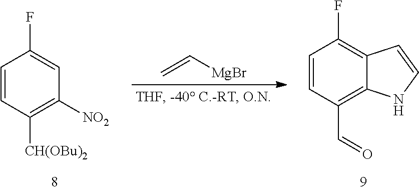

To a 100 mL three-necked flask were added 8 (450.0 mg, 1.5 mmol) and THF (15 mL), the solution was degassed and refilled Ar three times, then cooled to −40° C. and slowly added vinylmagnesium bromide (7 mL, 1 M in THF) by syringe over 1 hour in stirring. The reaction was warmed slowly to room temperature and stirred overnight. The reaction solution was quenched carefully by saturated aqueous ammonium chloride solution (20 mL) and water (30 mL), extracted by EA (30 mL×3), combined the organic layer, washed with brine (30 mL×2), dried over anhydrous sodium sulfate, filtered, concentrated the filtrate to give a yellow oil. The residue was dissolved in THF (5 mL) and hydrochloric acid (5 mL, 0.5 M in water), stirred at room temperature for 1 hour, then basified by saturated sodium bicarbonate solution till pH~8. The mixture was extracted by EA (20 mL×3), combined the organic layer, washed with brine (20 mL×2), dried over anhydrous sodium sulfate, filtered, concentrated the filtrate and purified by silica gel chromatography using a mixture of PE-EA (60:40, v/v) as eluent to afford 9 (185 mg, yield: 66%).

### The Synthesis of N-((4-fluoro-1H-indol-7-yl)methyl)-3-(pyridazin-3-yl)pyridin-2-amine (ELG-000504)

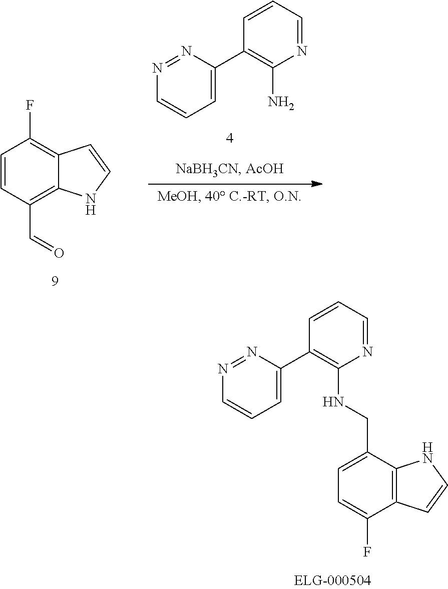 [SMILES: O=Cc1ccc(F)c2cc[nH]c12 ; Nc1ncccc1-c1cccnn1 ; Fc1ccc(CNc2ncccc2-c2cccnn2)c2[nH]ccc12]

To a 100 mL round bottom flask were added 9 (40.0 mg, 0.25 mmol), 4 (51.7 mg, 0.30 mmol), AcOH (5 drops) and methanol (4 mL), the mixture was stirred at 40° C. for 2 hours. Then the reaction solution was cooled to room temperature, added NaBH₃CN (78.6 mg, 1.25 mmol) and stirred overnight. The reaction solution was quenched by water (1 mL), purified by prep-TLC (a mixture of petroleum ether and ethyl acetate, 5:1, v/v), to afford ELG-000504 (3.8 mg, yield: 4%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.189 min; MS Calcd: 319.12; MS Found: 320.1 [M+H]⁺. HPLC (Waters UPLC. Column: ACQUITY UPLC BEH C18 1.7 μm*2.1 mm*150 mm): Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 99.46%, Rt=4.353 min. ¹H NMR (400 MHz, DMSO-d₆) δ 11.61 (s, 1H), 9.59 (t, J=5.5 Hz, 1H), 9.21 (dd, J=4.8, 1.4 Hz, 1H), 8.37 (dd, J=8.9, 1.4 Hz, 1H), 8.29 (dd, J=4.8, 1.7 Hz, 1H), 8.20 (dd, J=7.7, 1.7 Hz, 1H), 7.86 (dd, J=8.9, 4.8 Hz, 1H), 7.47-7.43 (m, 1H), 7.10 (dd, J=7.9, 5.1 Hz, 1H), 6.79 (ddd, J=18.5, 9.1, 6.4 Hz, 2H), 6.57 (dd, J=3.0, 1.5 Hz, 1H), 5.04 (d, J=5.3 Hz, 2H). Chemical Formula: C18H₁₄N₅F. Molecular Weight: 319.34.

### Route for ELG-000506

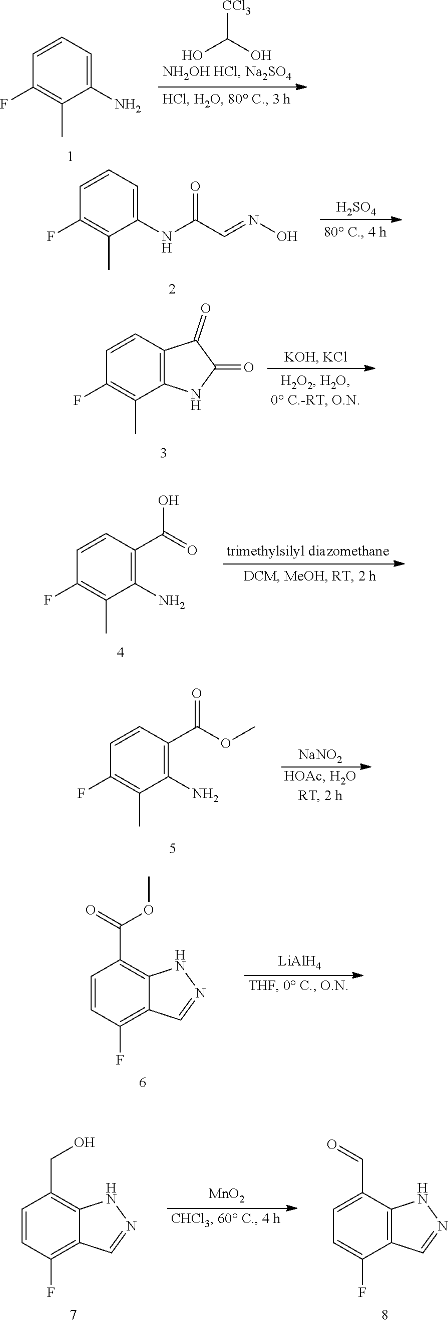

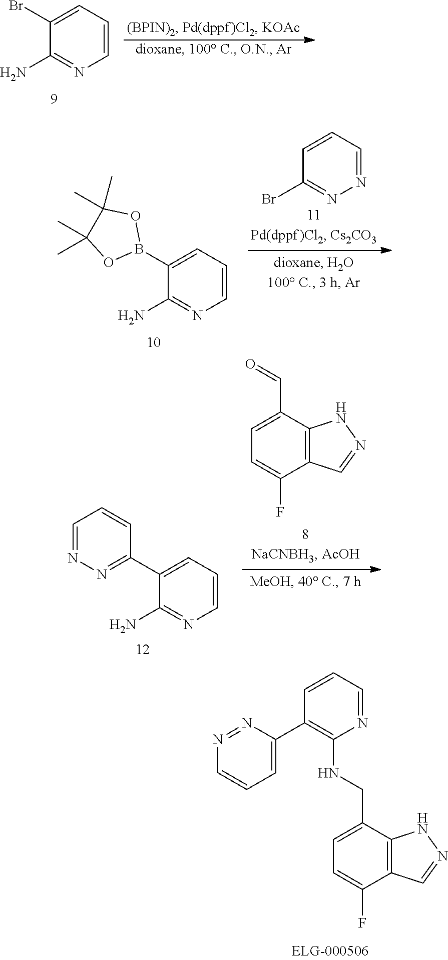

### The Synthesis of (E)-N-(3-fluoro-2-methylphenyl)-2-(hydroxyimino)acetamide (2)

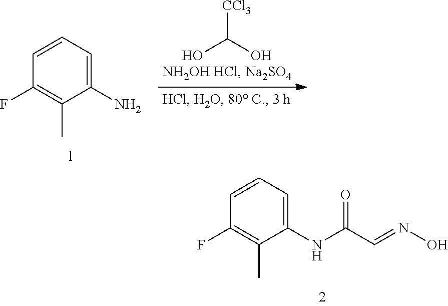

To a 1 L round bottom flask were added Na₂SO₄ (227 g, 1.59 mol), H₂O (400 mL) and 2,2,2-trichloroethane-1,1-diol (29 g, 175.6 mmol), then 1 (20.0 g, 159.8 mmol) in HCl (200 mL, 1 M in water) solution was added into the mixture, after stirred for 10 mins, NH₂OH HCl (44.4 g, 619.2 mmol) was added into the above solution and the mixture was stirred at 80° C. for 3 hours. After reaction was completed, reaction mixture was cooled down to room temperature, filtered, the filter cake was dried in vacuo to afford 2 (59 g) as crude product.

### The Synthesis of 6-fluoro-7-methylindoline-2,3-dione (3)

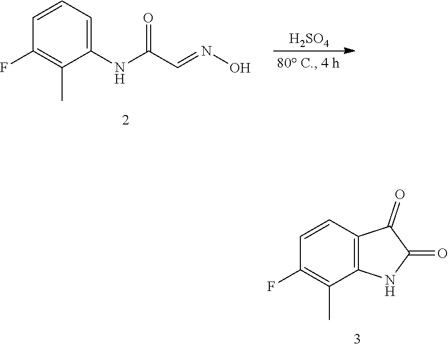

To a 1 L round bottom flask were added 2 (59 g, 310 mmol) and conc. H₂SO₄ (300 mL), the mixture was stirred at 80° C. for 4 hours. After reaction was completed, the reaction mixture was poured carefully into the ice water, extracted with EtOAc (200 mL×4), the organic phases was separated and combined, dried over anhydrous Na₂SO₄, concentrated in vacuo to afford 3 (5.34 g, yield: 17%).

### The Synthesis of 2-amino-4-fluoro-3-methylbenzoic acid (4)

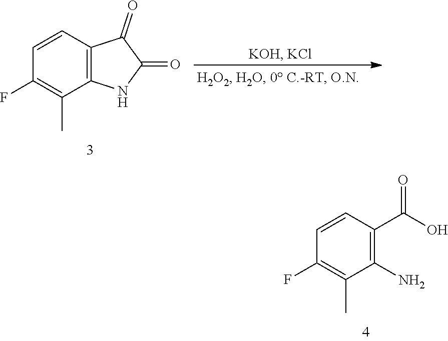

To a 250 mL three-necked flask were added 3 (5.34 g, 29.83 mmol) and H₂O (100 mL), KOH (1.84 g, 32.83 mmol) and KCl (4.66 mg, 62.52 mmol) was added at 0° C., and then slowly add H₂O₂ (5.07 g, 44.08 mmol) about 10 mins under protection of N₂, the mixture was stirred at room temperature overnight. The mixture was acidified carefully with TFA to pH=5 and diluted with H₂O (100 mL), extracted with EtOAc (100 mL×3), the organic phases was separated and combined, dried over anhydrous Na₂SO₄, concentrated in vacuo to afford 4 (4.41 g, yield: 90%).

### The Synthesis of methyl 2-amino-4-fluoro-3-methylbenzoate (5)

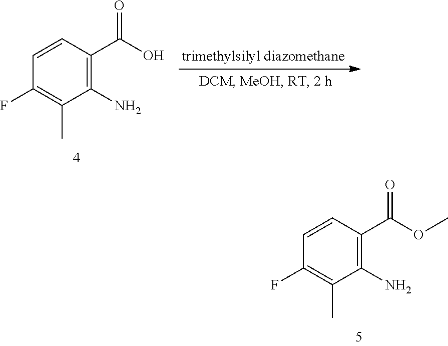

To a 250 mL round bottom flask were added 4 (4.41 g, 26.08 mmol), DCM (50 mL), MeOH (10 mL), trimethylsilyl diazomethane (4.47 g, 39.19 mmol) was added into the mixture at ice bath. Mixture was stirred at room temperature for 2 hours. Water (100 mL) was added and extracted with DCM (50 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 5 (1.36 g, yield: 84%).

### The Synthesis of methyl 4-fluoro-1H-indazole-7-carboxylate (6)

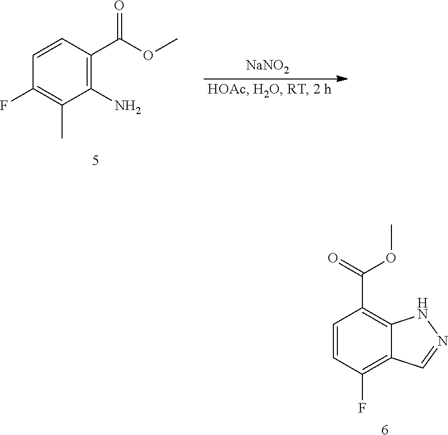

To a 100 mL round bottom flask were added 5 (1.36 g, 7.40 mmol), HOAc (15 mL), NaNO₂ (566 mg, 8.16 mmol) and H₂O (5 mL), mixture was stirred at room temperature for 2 hours. After reaction was completed, mixture was concentrated and residue was treated with a.q. NaHCO₃ solution to pH=7, mixture was extracted with EtOAc (30 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 6 (757 mg, yield: 90%).

### The Synthesis of (4-fluoro-1H-indazol-7-yl)methanol (7)

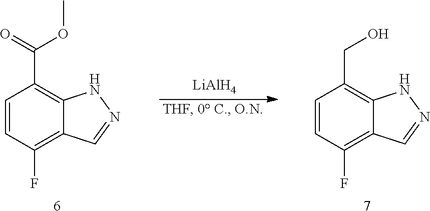

To a 100 mL three-necked flask were added 6 (757 mg, 3.94 mmol) and THF (15 mL), LiAlH₄ (2.27 mL, 5.91 mmol in THF) was added into the mixture by dropwise at 0° C., mixture was stirred at same temperature overnight. After reaction was completed, reaction mixture was quenched with 15% NaOH solution, and filtered. The filtrate was extracted with H₂O (30 mL) and EtOAc (20 mL×3), the organic phases was separated and combined, dried over anhydrous Na₂SO₄, organic phase was concentrated and purified by silica gel chromatography using a mixture of DCM-MeOH (90:10, v/v) as eluent to afford 7 (655 mg, yield: 78%).

### The Synthesis of 4-fluoro-1H-indazole-7-carbaldehyde (8)

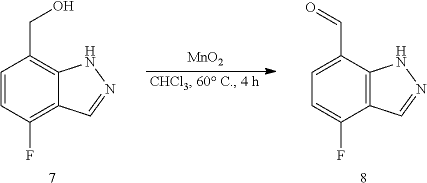

To a 100 mL three-necked flask were added 7 (655 mg, 3.93 mmol), MnO₂ (1.7 g, 19.49 mmol) and CHCl₃ (15 mL), the mixture was stirred at 60° C. for 4 hours. The mixture was filtered and filter cake was washed with DCM, the filtrate was concentrated in vacuo and purified by silica gel chromatography using a mixture of petroleum ether-ethyl acetate (80:20, v/v) as eluent to afford 8 (450 mg, yield: 90%).

### The Synthesis of 3-(4,4,5,5-tetramethyl-1,3,2-dioxaborolan-2-yl)pyridin-2-amine (10)

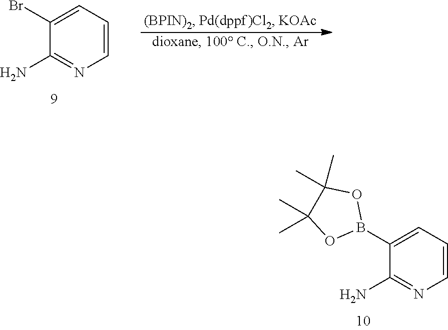 [SMILES: Nc1ncccc1Br ; "dioxane, 100° C., O.N., Ar" ; "(BPIN)2, Pd(dppf)Cl2, KOAc" ; CC1(C)OB(c2cccnc2N)OC1(C)C]

To a 250 mL round bottom flask were added 9 (2.5 g, 14.50 mmol). Pd(dppf)Cl₂ (530.6 mg, 0.73 mmol), (BPIN)₂ (4.4 g, 17.40 mmol). KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (12)

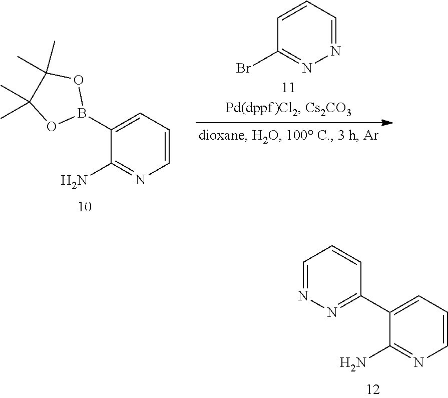 [SMILES: CC1(C)OB(c2cccnc2N)OC1(C)C ; Brc1cccnn1 ; Nc1ncccc1-c1cccnn1]

To a 250 mL round bottom flask were added 10 (3.19 g, 14.50 mmol), Pd(dppf)Cl₂ (530.6 mg, 0.73 mmol), Cs₂CO₃ (9.5 g, 29.0 mmol), 11 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 12 (1.1 g, yield: 44%).

### The Synthesis of N-((4-fluoro-1H-indazol-7-yl)methyl)-3-(pyridazin-3-yl)pyridin-2-amine (ELG-000506)

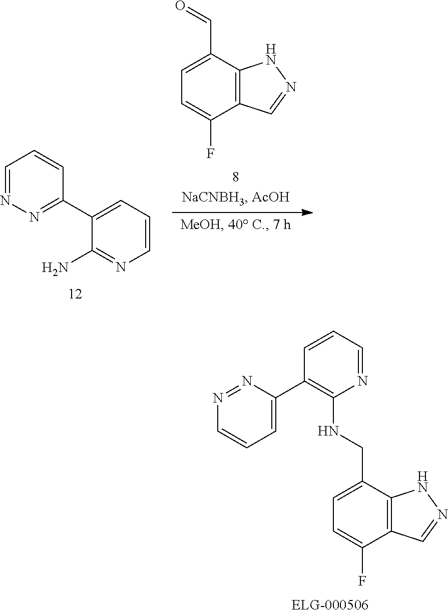 [SMILES: Nc1ncccc1-c1cccnn1 ; O=Cc1ccc(F)c2cn[nH]c12 ; Fc1ccc(CNc2ncccc2-c2cccnn2)c2[nH]ncc12]

To a 100 mL bottom flask were added 12 (54.6 mg, 0.32 mmol), 8 (40 mg, 0.24 mmol), AcOH (3 drops) and methanol (5 mL), the mixture stirred at 40° C. for 2 hours. Then the reaction mixture was added NaBH₃CN (91.7 mg, 1.46 mmol) and stirred at the same temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000506 (14.8 mg, yield: 14%) as a white solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min). Purity: 100%, Rt=1.150 min: MS Calcd: 320.12: MS Found: 321.4 [M+H]⁺. HPLC (Waters UPLC. Column: ACQUITY UPLC BEH C18 1.7 μm*2.1 mm*150 mm); Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min. finally changed to 90% [water+0.02% FA] and 10% [CH₃CN+0.02% FA] in 1.5 min and under this condition for 2 min), Purity: 98.70%, Rt=3.776 min. ¹H NMR (400 MHz, DMSO-d₆) δ 13.60 (s, 1H), 9.54 (t, J=5.6 Hz, 1H), 9.19 (dd, J=4.8, 1.4 Hz, 1H), 8.32 (d. J=8.9 Hz, 1H), 8.20 (dd, J=4.8, 1.7 Hz, 2H), 8.17-8.11 (m, 1H), 7.83 (dd, J=8.8, 4.8 Hz, 1H), 7.25 (dd, J=7.7, 4.9 Hz, 1H), 6.85-6.73 (m, 2H), 5.02 (d, J=5.5 Hz, 2H). Chemical Formula: C₁₇H₁₃N₆F. Molecular Weight: 320.33.

### Route for ELG-000507

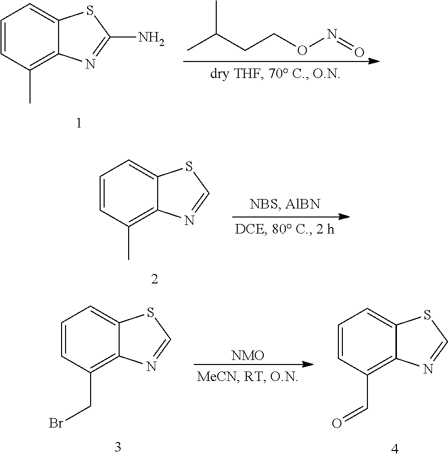

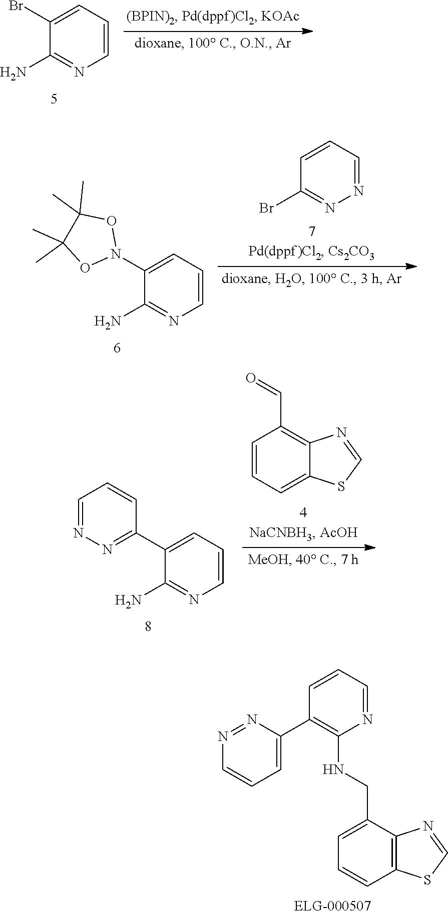 [SMILES: Nc1ncccc1Br ; CC1(C)ON(c2cccnc2N)OC1(C)C ; Brc1cccnn1 ; Nc1ncccc1-c1cccnn1 ; O=Cc1cccc2scnc12 ; c1cnnc(-c2cccnc2NCc2cccc3scnc23)c1]

### The Synthesis of 4-methylbenzo[d]thiazole (2)

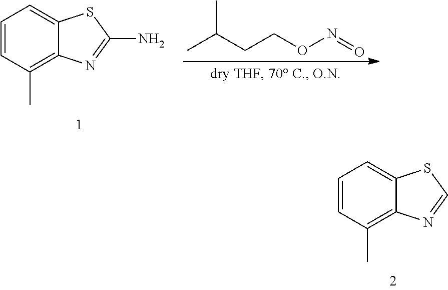

To a 250 mL round bottom flask were added 1 (2.0 g, 12.18 mmol) and dry THF(80 mL), the mixture was degassed and refilled N₂ three times, then i-AmONO (2.8 g, 24.36 mmol) was added to the mixture and stirred at 70° C. overnight. The reaction mixture was concentrated and added 50 mL water to quench the reaction. The aqueous layer was extracted with EA (8×20 mL), the combined organic phase was washed with brine (3×30 mL), dried over anhydrous Na₂SO₄, concentrated under reduced pressure and purified by silica gel chromatography using a mixture of petroleum ether-ethyl acetate (95:5, v/v) as eluent to afford 2 (1.5 g, yield: 84%).

### The Synthesis of 4-(bromomethyl)benzo[d]thiazole (3)

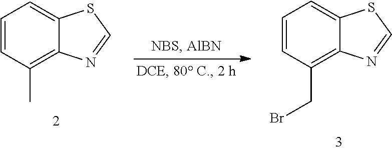

To a 250 mL round bottom flask were added 2 (1.4 g, 9.38 mmol), NBS (1.9 g, 10.32 mmol), AIBN (154.4 mg, 0.94 mmol) and DCE (40 mL), the mixture was stirred at 80° C. for 2 hours. The reaction mixture was concentrated and purified by silica gel chromatography using a mixture of petroleum ether-ethyl acetate (98:2, v/v) as eluent to afford 3 (1.5 g, yield: 71%).

### The Synthesis of benzo[d]thiazole-4-carbaldehyde (4)

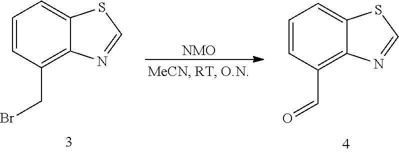

To a 100 mL round bottom flask were added 3 (700.0 mg, 3.07 mmol), NMO (1.4 g, 12.33 mmol) and MeCN (25 mL), the mixture was stirred at room temperature overnight. The reaction mixture was concentrated and purified by silica gel chromatography using a mixture of petroleum ether-ethyl acetate (97:3, v/v) as eluent to afford 4 (122.0 mg, yield: 25%).

### The Synthesis of 3-(4,4,5,5-tetramethyl-1,3,2-dioxaborolan-2-yl)pyridin-2-amine (6)

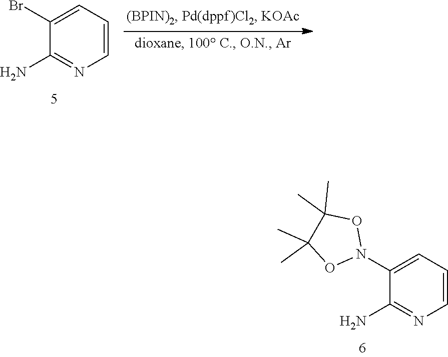 [SMILES: Nc1ncccc1Br ; CC1(C)ON(c2cccnc2N)OC1(C)C]

To a 250 mL round bottom flask were added 5 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (8)

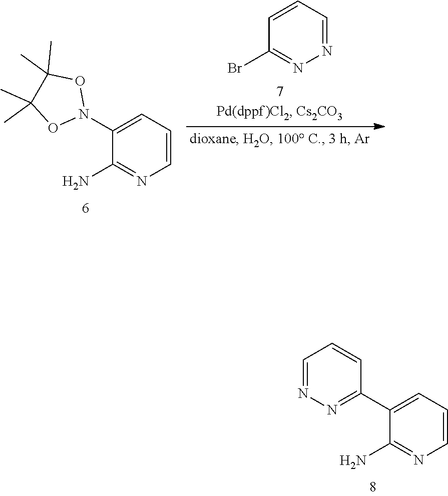 [SMILES: CC1(C)ON(c2cccnc2N)OC1(C)C ; Brc1cccnn1 ; Nc1ncccc1-c1cccnn1]

To a 250 mL round bottom flask were added 6 (3.19 g, 14.50 mmol), Pd(dppf)Cl₂ (530.6 mg, 0.73 mmol), Cs₂CO₃ (9.5 g, 29.0 mmol), 7 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 8 (1.1 g, yield: 44%).

### The Synthesis of N-(benzo[d]thiazol-4-ylmethyl)-3-(pyridazin-3-yl)pyridin-2-amine (ELG-000507)

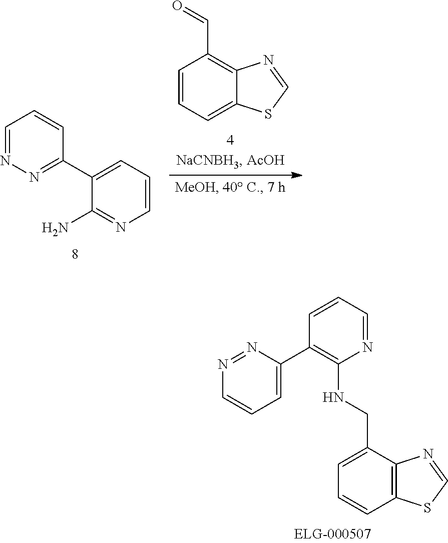 [SMILES: Nc1ncccc1-c1cccnn1 ; O=Cc1cccc2scnc12 ; c1cnnc(-c2cccnc2NCc2cccc3scnc23)c1]

To a 100 mL round bottom flask were added 4 (45.0 mg, 0.25 mmol), 8 (40.0 mg, 0.24 mmol), AcOH (3 drops) and MeOH (3 mL), the mixture stirred at 40° C. for 2 hours. Then the reaction mixture was added NaBH₃CN (45.2 mg, 0.72 mmol) and stirred at the same temperature for another 5 hours. The reaction mixture was quenched by water (0.5 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000507 (4.0 mg, yield: 5%) as a yellow solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 97.23%, Rt=1.053 min; MS Calcd: 319.09; MS Found: 320.1 [M+H]⁺. HPLC (Waters UPLC. Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 99.29%, Rt=3.864 min. ¹H NMR (400 MHz, DMSO-d₆) δ 9.74 (t, J=5.8 Hz, 1H), 9.43 (s, 1H), 9.18 (dd, J 4.8, 1.4 Hz, 1H), 8.35 (dd, J 8.9, 1.4 Hz, 1H), 8.21-8.13 (m, 2H), 8.05 (dd, J=7.7, 1.1 Hz, 1H), 7.83 (dd, J=8.9, 4.8 Hz, 1H), 7.48-7.35 (m, 2H), 6.76 (dd, J=7.2, 5.3 Hz, 1H), 5.29 (d, J=5.7 Hz, 2H). Chemical Formula: C₁₇H₁₃N₅S. Molecular Weight: 319.39.

### Route for ELG-000510

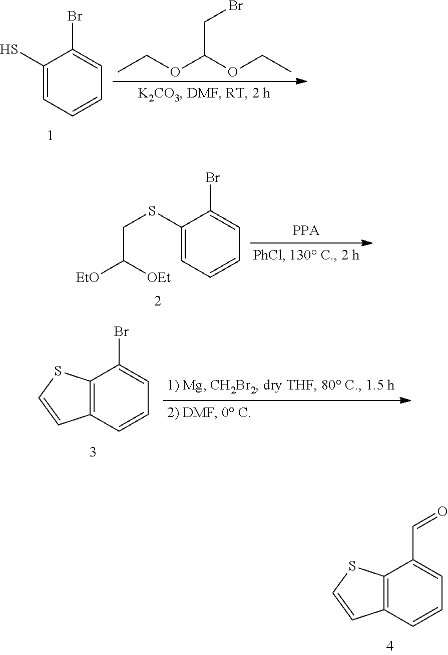

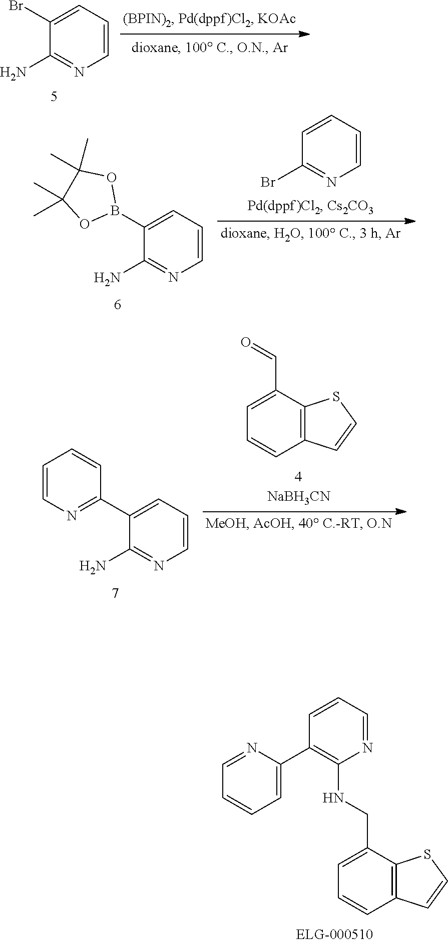 [SMILES: Nc1ncccc1Br ; CC1(C)OB(c2cccnc2N)OC1(C)C ; Brc1ccccn1 ; Nc1ncccc1-c1ccccn1 ; O=Cc1cccc2ccsc12 ; c1ccc(-c2cccnc2NCc2cccc3ccsc23)nc1]

### The Synthesis of (2-bromophenyl)(2,2-diethoxyethyl)sulfane (2)

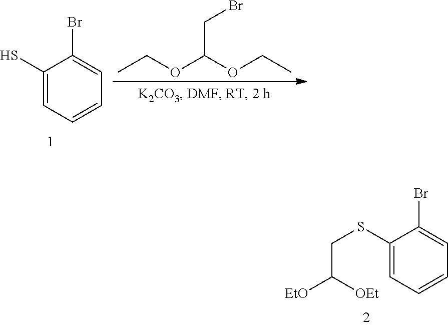

To a 100 mL round bottom flask were added 1 (4.4 g, 23.3 mmol), potassium carbonate (5.1 g, 36.9 mmol), Bromoacetaldehyde diethyl acetal (5.0 g, 25.4 mmol) and DMF (40 mL), the reaction mixture was stirred at room temperature for 4 hours. Once the reaction completed, water (100 mL) was added in the reaction and extracted with EtOAc (30 mL×4), the organic phase was washed with saturated brine (30 mL×4) and dried over Na₂SO₄ and concentrated in vacuo. The crude product was purified by silica gel chromatography using a mixture of petroleum ether-ethyl acetate (90:10, v/v) as eluent to afford 2 (6.6 g, yield: 94%).

### The Synthesis of 7-bromobenzo[b]thiophene (3)

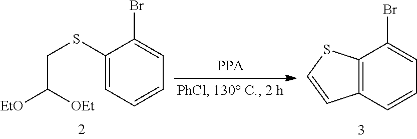 [SMILES: CCOC(CSc1ccccc1Br)OCC ; Brc1cccc2ccsc12]

To a 100 mL round bottom flask were added paraformaldehyde (15.0 g, 166.5 mmol) and chlorobenzene (50 mL) and 2 (6.6 g, 21.7 mmol). The reaction mixture was stirred at 130° C. for 2 hours. The reaction mixture was concentrated in vacuo, the residue was diluted with water (100 mL), extracted with EtOAc (30 mL×5), the combined organic phase was washed with saturated sodium bicarbonate aqueous solution (30 mL×3), dried over anhydrous Na₂SO₄ and concentrated in vacuo. The crude product was purified by silica gel chromatography using petroleum ether as eluent to afford 3 (3.7 g, yield: 79%).

### The Synthesis of benzo[b]thiophene-7-carbaldehyde (4)

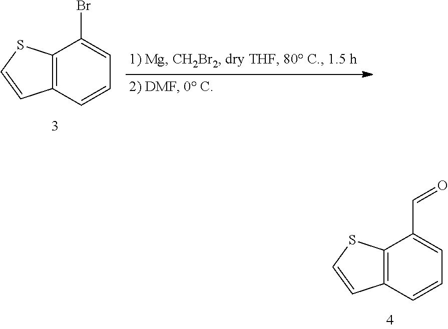 [SMILES: Brc1cccc2ccsc12 ; O=Cc1cccc2ccsc12]

To a 100 mL round bottom flask were added magnesium bars (616.8 mg, 25.7 mmol) and THF (14 mL), the reaction mixture was degassed and refilled with Ar three times, then stirred at 80° C. for 1.5 hours. The reaction mixture was cooled to 0° C., DMF (2.8 mL, 32.8 mmol) was slowly added and stirred at room temperature overnight. Once the reaction completed, the reaction mixture was concentrated in vacuo and purified by silica gel chromatography using a mixture of petroleum ether-ethyl acetate (95:5, v/v) as eluent to afford 4 (1.6 g, yield: 59%).

### The Synthesis of 3-(4,4,5,5-tetramethyl-1,3,2-dioxaborolan-2-yl)pyridin-2-amine (6)

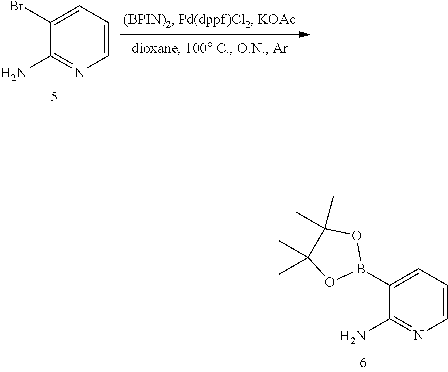 [SMILES: Nc1ncccc1Br ; CC1(C)OB(c2cccnc2N)OC1(C)C]

To a 250 mL round bottom flask were added 5 (2.5 g, 14.50 mmol), Pd(dppf)Cl₂ (531 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (7)

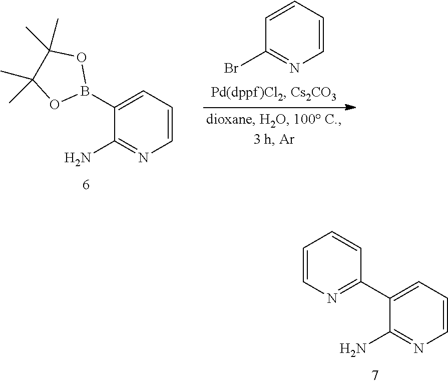 [SMILES: CC1(C)OB(c2cccnc2N)OC1(C)C ; Brc1ccccn1 ; Nc1ncccc1-c1ccccn1]

To a 250 mL round bottom flask were added 6 (3.19 g, 14.50 mmol), Pd(dppf)Cl₂ (531 mg, 0.73 mmol), Cs₂CO₃ (9.5 g, 29.0 mmol), 2-bromopyridine (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 7 (1.0 g, yield: 40%).

### The Synthesis of N-(benzo[b]thiophen-7-ylmethyl)-[2,3′-bipyridin]-2′-amine (ELG-00510)

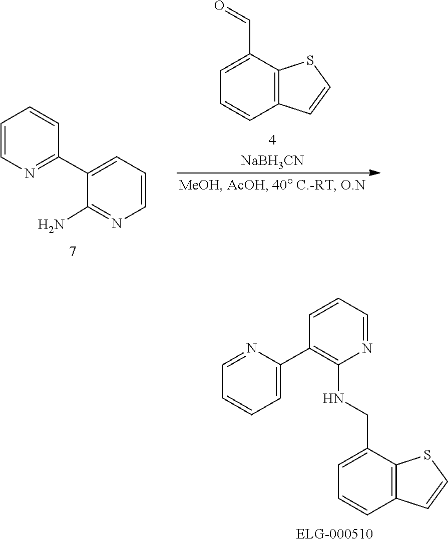

To a 100 mL round bottom flask were added 7 (60 mg, 0.35 mmol), 4 (59 mg, 0.36 mmol). MeOH (5 mL) and AcOH (5 drops), the mixture was stirred at 40° C. for 2 hours, and then add NaBH₃CN (110 mg, 1.75 mmol) to the mixture at room temperature. The reaction mixture was stirred at same temperature overnight. The reaction mixture was quenched with H₂O (20 mL), then the mixture was extracted with EtOAc (10 mL×3), the combined organic layers were washed with brine, dried over anhydrous Na₂SO₄, filtered and the filtrate was concentrated. The crude product was purified by prep-TLC to afford ELG-000510 (23.5 mg, yield: 22%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%. Rt=1.295 min; MS Calcd: 317.10; MS Found: 318.1 [M+H]⁺. HPLC (Waters UPLC, Column: ACQUITY UPLC BEH C18 1.7 μm*2.1 mm*150 mm): Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 99.41%, Rt=4.854 min. ¹H NMR (400 MHz, DMSO-d₆) δ 9.90 (t, J=5.7 Hz, 1H), 8.62 (d, J=4.1 Hz, 1H), 8.12 (dd, J=7.6, 1.6 Hz, 1H), 8.08 (dd, J=4.8, 1.6 Hz, 1H), 8.04 (d, J=8.3 Hz, 1H), 7.94 (td, J=7.9, 1.8 Hz, 1H), 7.78 (d, J=6.5 Hz, 1H), 7.73 (d. J=5.4 Hz, 1H), 7.49 (d, J=5.4 Hz, 1H), 7.34 (tt. J=10.7, 5.3 Hz, 3H), 6.70 (dd, J=7.6, 4.8 Hz, 1H), 4.98 (d, J=5.7 Hz, 2H). Chemical Formula: C₁₉H₁₅N₃S. Molecular Weight: 317.41.

### Route for ELG-000515

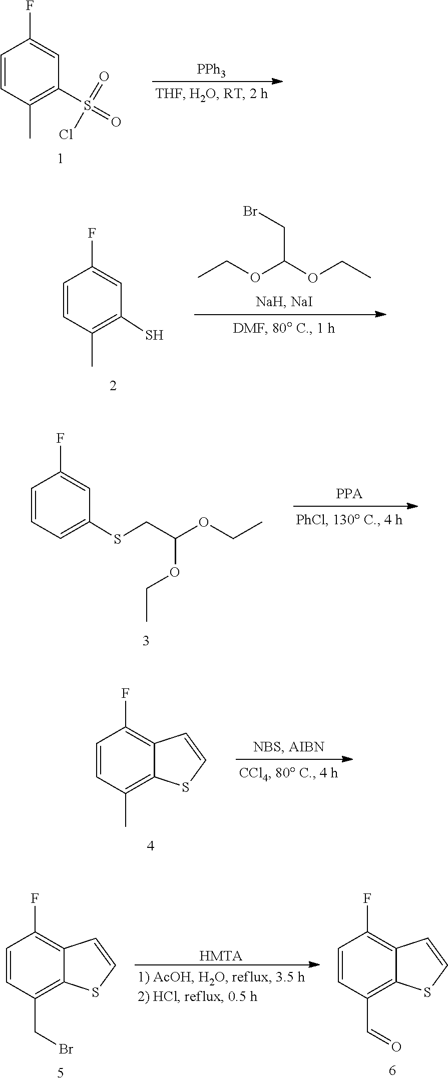

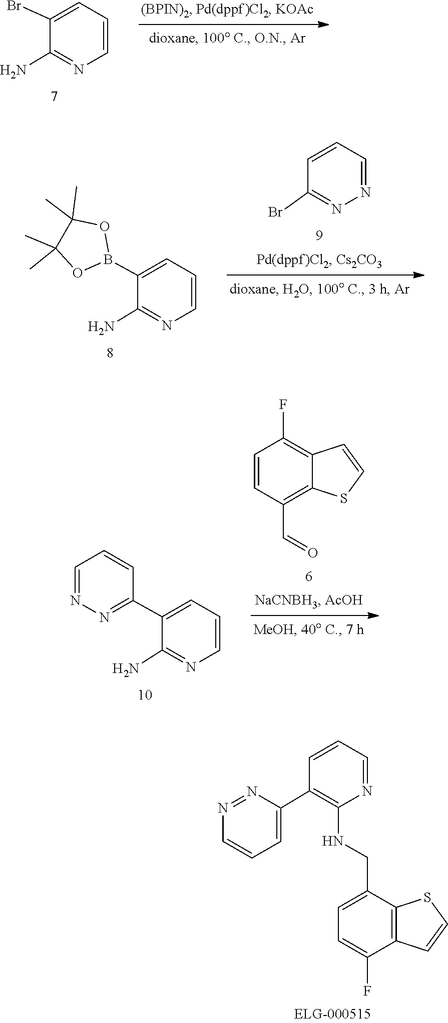 [SMILES: Nc1ncccc1Br ; CC1(C)OB(c2cccnc2N)OC1(C)C ; Brc1cccnn1 ; Nc1ncccc1-c1cccnn1 ; O=Cc1ccc(F)c2ccsc12 ; Fc1ccc(CNc2ncccc2-c2cccnn2)c2sccc12]

### The Synthesis of 5-fluoro-2-methylbenzenethiol (2)

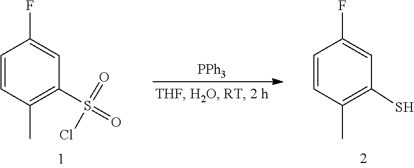

To a 100 mL round bottom flask were added 1 (5.0 g, 23.9 mmol), THF (80 mL) and triphenylphosphine (19.0 g, 71.7 mmol). The reaction mixture was stirred at room temperature for 1 hour. Then water (3.2 mL) was added and stirred for another 1 hour. Once the reaction completed, water (20 mL) was added in the reaction and extracted with EtOAc (20 mL×3), the organic phase was combined, dried over anhydrous Na₂SO₄, concentrated in vacuo and purified by silica gel chromatography using petroleum ether as eluent to afford 2 (2.2 g, yield: 65%).

### The Synthesis of (2,2-diethoxyethyl)(5-fluoro-2-methylphenyl)sulfane (3)

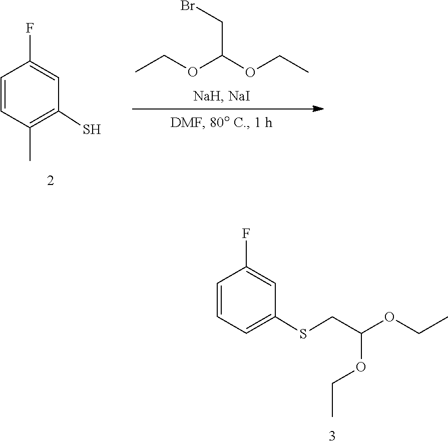

To a 100 mL round bottom flask were added 2 (2.2 g, 15.5 mmol), bromoacetaldehyde diethyl acetal (3.4 g, 17.3 mmol). NaI (1.2 g, 8.0 mmol), NaH (1.2 g, 50.0 mmol) and DMF (50 mL), the reaction mixture was stirred at 80° C. for 1 hour. Once the reaction completed, water (100 mL) was added, extracted with EtOAc (30 mL×3), the organic phase was combined, washed by brine (30 mL×3), dried over anhydrous Na₂SO₄, concentrated in vacuo and purified by silica gel chromatography using petroleum ether as eluent to afford 3 (3.8 g, yield: 95%).

### The Synthesis of 4-fluoro-7-methylbenzo[b]thiophene (4)

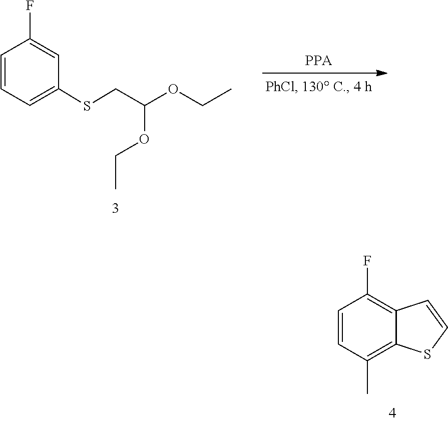 [SMILES: CCOC(CSc1cccc(F)c1)OCC ; Cc1ccc(F)c2ccsc12]

To a 100 mL round bottom flask were added paraformaldehyde (8.0 g, 88.8 mmol), chlorobenzene (60 mL) and 3 (3.8 g, 14.7 mmol). The reaction mixture and stirred at 130° C. for 4 hours. The reaction mixture was concentrated in vacuo, the residue was diluted with water (50 mL), extracted with EtOAc (30 mL×3), the combined organic phase was washed with saturated sodium bicarbonate aqueous solution (30 mL×3), dried over anhydrous Na₂SO₄, concentrated in vacuo and purified by silica gel chromatography using petroleum ether as eluent to afford 4 (1.75 g, yield: 70%).

### The Synthesis of 7-(bromomethyl)-4-fluorobenzo[b]thiophene (5)

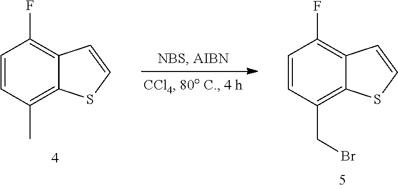

To a 100 mL round bottom flask were added 4 (1.75 g, 10.5 mmol). NBS (2.43 g, 13.6 mmol), AIBN (0.35 g, 2.1 mmol) and carbon tetrachloride (50 mL), the reaction mixture was stirred at 80° C. for 4 hours. Once the reaction completed, the reaction mixture was diluted with water (50 mL), extracted with DCM (20 mL×3), the organic phase was washed with saturated sodium bicarbonate aqueous solution (20 mL×3), the organic phase was combined, dried over anhydrous Na₂SO₄, concentrated in vacuo, and purified by silica gel chromatography using petroleum ether as eluent to afford 5 (2.3 g, yield: 91%).

### The Synthesis of 4-fluorobenzo[b]thiophene-7-carbaldehyde (6)

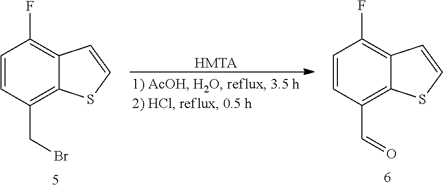

To a 100 mL round bottom flask were added 5 (114 mg, 0.81 mmol), hexamethylenetetramine (90 mg, 0.37 mmol), acetic acid (2 mL) and water (1 mL), the reaction mixture was stirred at 110° C. for 3.5 hours. Then concentrated hydrochloric acid (1 mL) was added at room temperature, keep stir at 110° C. for another 0.5 hour. Once the reaction completed, water (30 mL) was added in the reaction mixture, extracted with EA (20 mL×3), the organic phase was combined and dried over anhydrous Na₂SO₄, concentrated in vacuo, and purified by silica gel chromatography using using a mixture of petroleum ether-ethyl acetate (95:5, v/v) as eluent to afford 6 (40 mg, yield: 61%).

### The Synthesis of 3-(4,4,5,5-tetramethyl-1,3,2-dioxaborolan-2-yl)pyridin-2-amine (8)

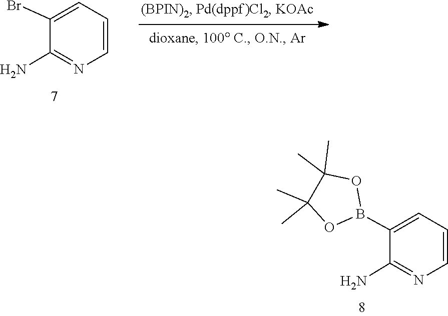 [SMILES: Nc1ncccc1Br ; CC1(C)OB(c2cccnc2N)OC1(C)C]

To a 250 mL round bottom flask were added 7 (2.5 g, 14.50 mmol). Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (10)

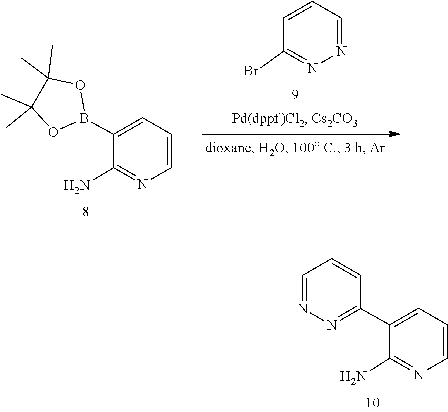 [SMILES: CC1(C)OB(c2cccnc2N)OC1(C)C ; Brc1cccnn1 ; Nc1ncccc1-c1cccnn1]

To a 250 mL round bottom flask were added 8 (3.19 g, 14.50 mmol), Pd(dppf)Cl₂ (530.6 mg, 0.73 mmol), Cs₂CO₃ (9.5 g, 29.0 mmol), 9 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 10 (1.1 g, yield: 44%).

### The Synthesis of N-((4-fluorobenzo[b]thiophen-7-yl)methyl)-3-(pyridazin-3-yl)pyridin-2-amine (ELG-000515)

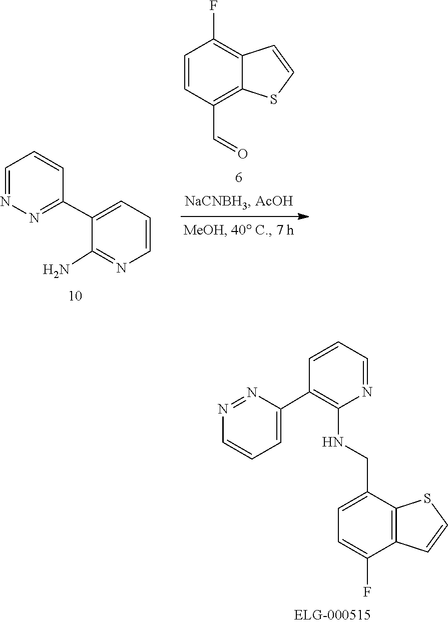 [SMILES: Nc1ncccc1-c1cccnn1 ; O=Cc1ccc(F)c2ccsc12 ; Fc1ccc(CNc2ncccc2-c2cccnn2)c2sccc12]

To a 100 mL bottom flask were added 10 (35.0 mg, 0.19 mmol), 6 (40.0 mg, 0.23 mmol). AcOH (3 drops) and methanol (3 mL), the mixture stirred at 40° C. for 2 hours. Then the reaction mixture was added NaBH₃CN (72.9 mg, 0.97 mmol) and stirred at the same temperature for another 5 hours. The mixture was directly purified by Prep-TLC (a mixture of petroleum ether and ethyl acetate, 5:1, v/v) to afford ELG-000515 (7.8 mg, yield: 12%) as a white solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.258 min; MS Calcd: 336.08; MS Found: 337.1 [M+H]⁺. HPLC (Waters UPLC, Column: ACQUITY UPLC BEH C18 1.7 μm*2.1 mm*150 mm): Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 97.53%, Rt=4.611 min. ¹H NMR (400 MHz, DMSO-d₆) δ 9.73 (t, J=5.6 Hz, 1H), 9.18 (dd, J=4.8, 1.5 Hz, 1H), 8.37 (dd, J=8.9, 1.4 Hz, 1H), 8.24-8.10 (m, 2H), 7.88-7.76 (m, 2H), 7.53 (d, J=5.5 Hz, 1H), 7.35 (dd, J=8.1, 4.9 Hz, 1H), 7.17 (dd, J=10.3, 8.1 Hz, 1H), 6.78 (dd, J=7.6, 4.8 Hz, 1H), 5.00 (d, J=5.7 Hz, 2H). Chemical Formula: C18H₁₃FN₄S. Molecular Weight: 336.39.

### Route for ELG-000522

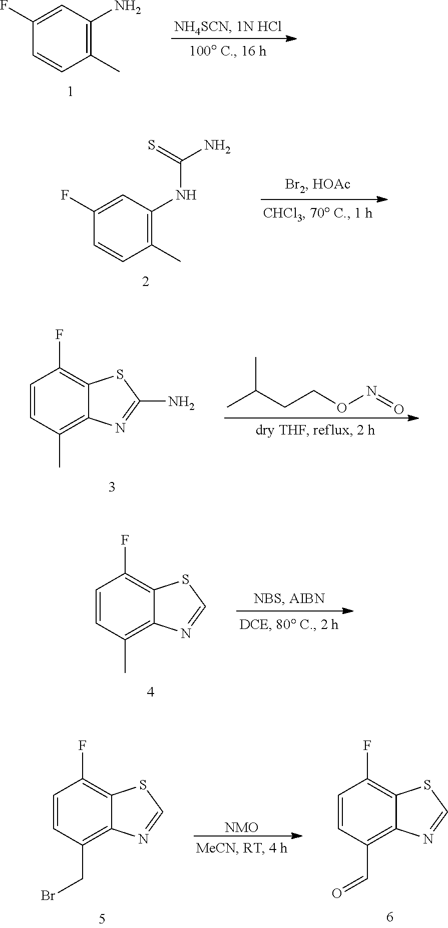

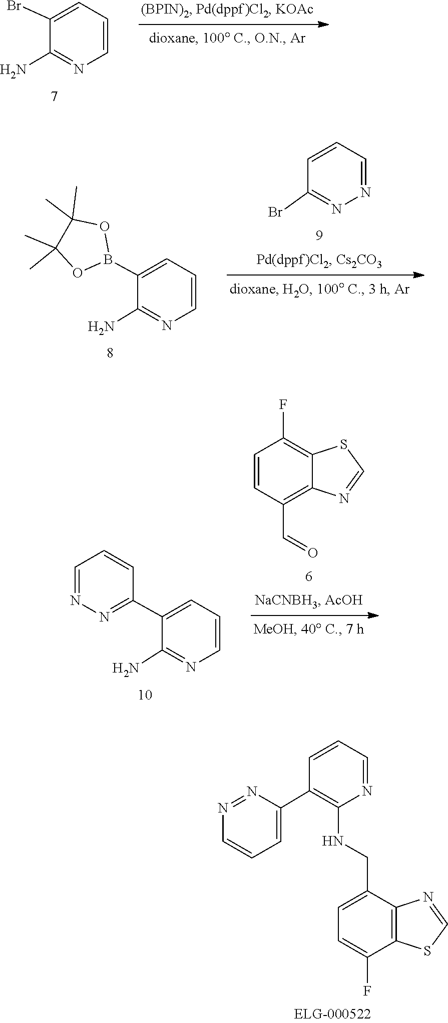 [SMILES: Nc1ncccc1Br ; CC1(C)OB(c2cccnc2N)OC1(C)C ; Brc1cccnn1 ; Nc1ncccc1-c1cccnn1 ; O=Cc1ccc(F)c2scnc12 ; Fc1ccc(CNc2ncccc2-c2cccnn2)c2ncsc12]

### The Synthesis of 1-(5-fluoro-2-methylphenyl)thiourea (2)

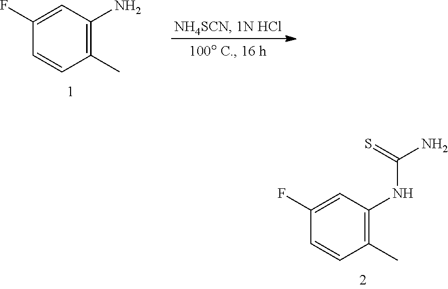

To a 100 mL round bottom flask were added 1 (650 mg, 5.19 mmol), NH₄SCN (435 mg, 5.71 mmol) and HCl (6 mL, 1 M in water), then the mixture was stirred at 100° C. for 16 hours. Then the mixture was basified by saturated ammonium hydroxide aqueous till pH>8, extracted with DCM (30 mL×5). The combined organic phase was washed with brine (30 mL×3), dried over anhydrous Na₂SO₄, concentrated under reduced pressure and purified by silica gel chromatography using petroleum ether as eluent to afford 2 (490 mg, yield: 51%).

### The Synthesis of 7-fluoro-4-methylbenzo[d]thiazol-2-amine (3)

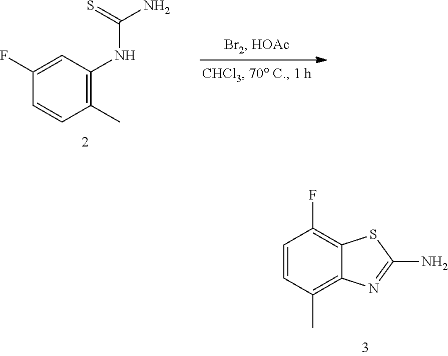

To a 100 mL round bottom flask were added 2 (490 mg, 2.66 mmol), AcOH (4.5 mL) and CHCl₃ (40 mL) and Br₂ (1.8 mL), the mixture was stirred at 70° C. for 1 hour. Then saturated NaHSO₃ aqueous was added to the reaction mixture until extra Br₂ was consumed. Then the mixture was extracted with EA (30 mL×3). The combined organic phase was washed with brine (30 mL×3), dried over anhydrous Na₂SO₄, concentrated under reduced pressure to give 3 (407 mg, yield: 84%) without further workup.

### The Synthesis of 7-fluoro-4-methylbenzo[d]thiazole (4)

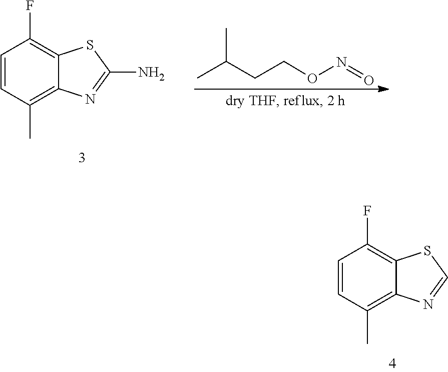

To a 100 mL bottom flask were added 3 (407 mg, 2.23 mmol) and dry THF (20 mL), the mixture was degassed under vacuum and refilled N₂ three times, then i-AmONO (721 mg, 6.15 mmol) was added. The reaction mixture was stirred at 70° C. for 2 hours. The reaction mixture was added 50 mL water to quench the reaction and concentrated to remove organic solvents. The aqueous layer was extracted with EA (30 mL×3). The combined organic phase was washed with brine (30 mL×3), dried over anhydrous Na₂SO₄, concentrated under reduced pressure and purified by silica gel chromatography using a mixture of PE-EA (98:2, v/v) as eluent to afford 4 (110 mg, yield: 29%).

### The Synthesis of 4-(bromomethyl)-7-fluorobenzo[d]thiazole (5)

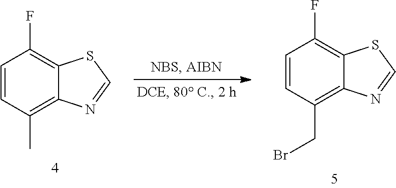

To a 50 mL bottom flask were added 4 (110 mg, 0.66 mmol), NBS (129 mg, 0.72 mmol). AIBN (12 mg, 0.07 mmol) and DCE (5 mL), the mixture was stirred at 80° C. for 2 hours. The reaction mixture was concentrated and purified by silica gel chromatography using a mixture of PE-EA (98:2, v/v) as eluent to afford 5 (109 mg, yield: 67%).

### The Synthesis of 7-fluorobenzo[d]thiazole-4-carbaldehyde (6

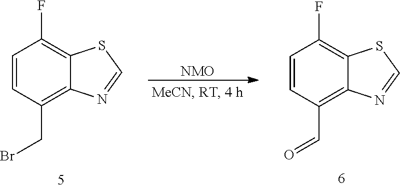

To a 100 mL round bottom flask were added 5 (109 mg, 0.44 mmol), NMO (132 mg, 1.12 mmol) and MeCN (4 mL), the mixture was stirred at room temperature for 4 hours. The reaction mixture was concentrated and purified by silica gel chromatography using a mixture of PE-EA (95:5, v/v) as eluent to afford 6 (42 mg, yield: 39%).

### The Synthesis of 3-(4,4,5,5-tetramethyl-1,3,2-dioxaborolan-2-yl)pyridin-2-amine (8)

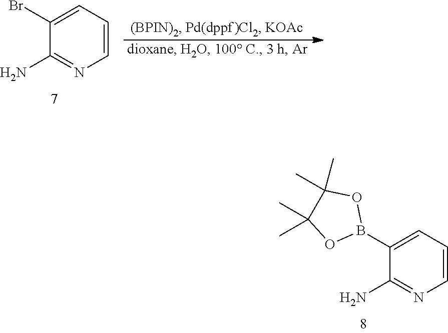

To a 250 mL round bottom flask were added 7 (2.5 g, 14.50 mmol). Pd(dppf)Cl₂ (530.6 mg, 0.73 mmol), (BPIN)₂ (4.4 g, 17.40 mmol). KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of 3-(pyridazin-3-yl)pyridin-2-amine (10)

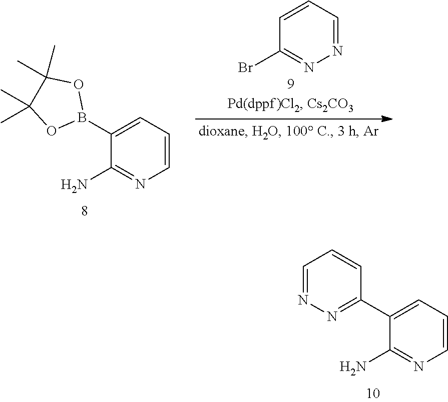 [SMILES: CC1(C)OB(c2cccnc2N)OC1(C)C ; Brc1cccnn1 ; Nc1ncccc1-c1cccnn1]

To a 250 mL round bottom flask were added 8 (3.19 g, 14.50 mmol), Pd(dppf)Cl₂ (530.6 mg, 0.73 mmol), Cs₂CO₃ (9.5 g, 29.0 mmol), 9 (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 10 (1.1 g, yield: 44%).

### The Synthesis of N-((7-fluorobenzo[d]thiazol-4-yl)methyl)-3-(pyridazin-3-yl)pyridin-2-amine (ELG-000522)

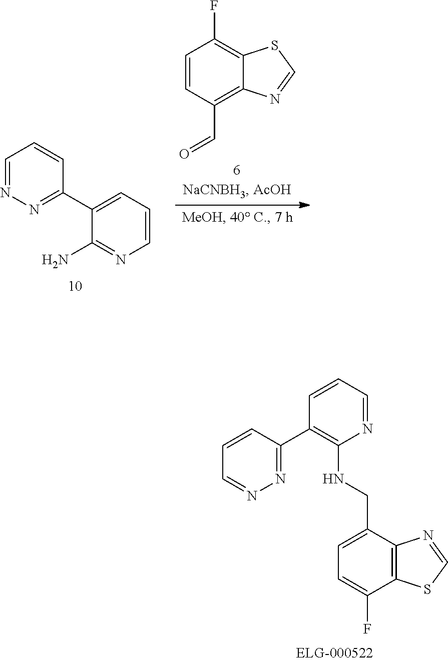 [SMILES: Nc1ncccc1-c1cccnn1 ; O=Cc1ccc(F)c2scnc12 ; Fc1ccc(CNc2ncccc2-c2cccnn2)c2ncsc12]

To a 100 mL bottom flask were added 10 (35.0 mg, 0.19 mmol), 6 (42.0 mg, 0.23 mmol). AcOH (3 drops) and methanol (3 mL), the mixture stirred at 40° C. for 2 hours. Then the reaction mixture was added NaBH₃CN (72.9 mg, 0.97 mmol) and stirred at the same temperature for another 5 hours. The mixture was directly purified by prep-TLC (a mixture of petroleum ether and ethyl acetate, 5:1, v/v) to afford ELG-000522 (13.5 mg, yield: 14%) as a yellowish solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min). Purity: 100.00%. Rt=1.131 min; MS Calcd: 337.08; MS Found: 338.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min. then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 99.37%, Rt=4.017 min. ¹H NMR (400 MHz, DMSO-d₆) δ 9.71 (t, J=5.8 Hz, 1H), 9.52 (d, J=1.3 Hz, 1H), 9.17 (dd, J=4.8, 1.3 Hz, TH), 8.34 (dd, J=8.9, 1.4 Hz, 1H), 8.20-8.14 (m, 1H), 7.83 (dd, J=8.9, 4.8 Hz, 1H), 7.48 (dd, J=8.2, 5.4 Hz, 1H), 7.39-7.24 (m, 1H), 6.76 (dd, J=7.2, 5.2 Hz, 1H), 5.25 (d, J=5.8 Hz, 2H). Chemical Formula: C₁₇H₁₂FN₅. Molecular Weight: 337.38.

### Route for ELG-000523

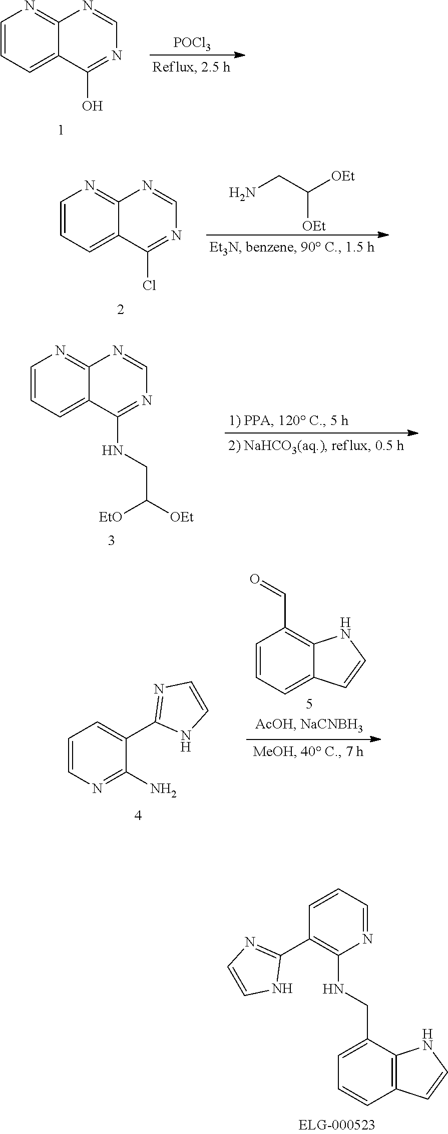

### The Synthesis of 4-chloropyrido[2,3-d]pyrimidine (2)

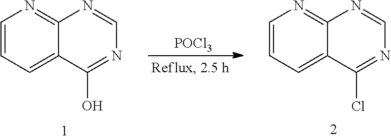

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

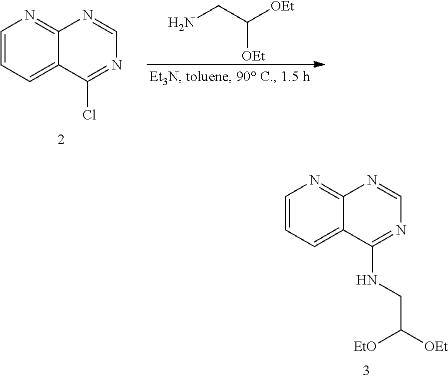 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (4)

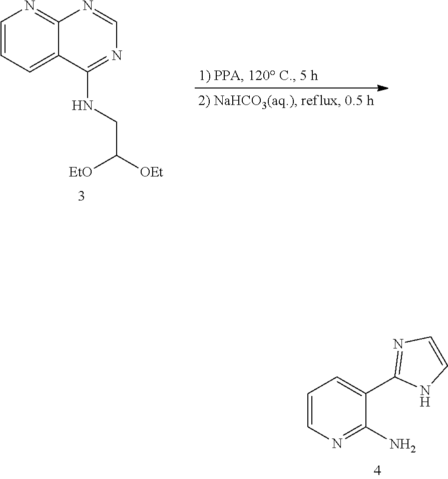 [SMILES: CCOC(CNc1ncnc2ncccc12)OCC ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 4 (0.3 g, crude). The filter cake was added saturated sodium bicarbonate solution (20 mL) and stirred at 110° C. for 0.5 hour. The reaction solution was extracted with THF (5×20 mL), combined crude product 4. The mixture was concentrated in vacuo and purified by silica gel chromatography using a mixture of petroleum ether-ethyl acetate (80:20, v/v) as eluent to afford 4 (448.0 mg, yield: 56%).

### The Synthesis of N-((1H-indol-7-yl)methyl)-3-(1H-imidazol-2-yl)pyridin-2-amine (ELG-000523)

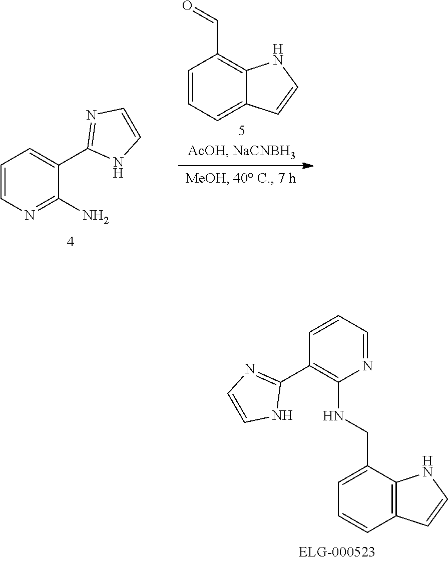 [SMILES: Nc1ncccc1-c1ncc[nH]1 ; O=Cc1cccc2cc[nH]c12 ; c1cnc(NCc2cccc3cc[nH]c23)c(-c2ncc[nH]2)c1]

To a 100 mL bottom flask were added 4 (45.1 mg, 0.28 mmol), 5 (40.6 mg, 0.28 mmol), AcOH (3 drops) and Methanol (3 mL), the mixture stirred at 40° C. for 2 hours. Then the reaction mixture was added NaBH₃CN (70.4 mg, 0.11 mmol) and stirred at the same temperature for another 5 hours. The mixture was directly purified by prep-TLC (a mixture of petroleum ether and ethyl acetate, 2:1, v/v) to afford ELG-000523 (42.5 mg, yield: 40%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.192 min: MS Calcd: 289.13; MS Found: 290.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 99.88%, Rt=4.248 min. ¹H NMR (400 MHz, DMSO-d₆) δ 12.63 (s, 1H), 11.20 (s, 1H), 9.69 (t, J=5.6 Hz, 1H), 8.07 (ddd, J=9.3, 6.2, 1.7 Hz, 2H), 7.44 (d, J=7.8 Hz, 1H), 7.34 (dd, J=10.5, 7.7 Hz, 2H), 7.07 (d, J=6.2 Hz, 2H), 6.99-6.91 (m, 1H), 6.67 (dd, J=7.6, 4.9 Hz, 1H), 6.46 (dd, J=3.0, 1.9 Hz, 1H), 5.00 (d, J=5.6 Hz, 2H). Chemical Formula: C₁₇H₁₅N₅. Molecular Weight: 289.34.

### Route for ELG-000525

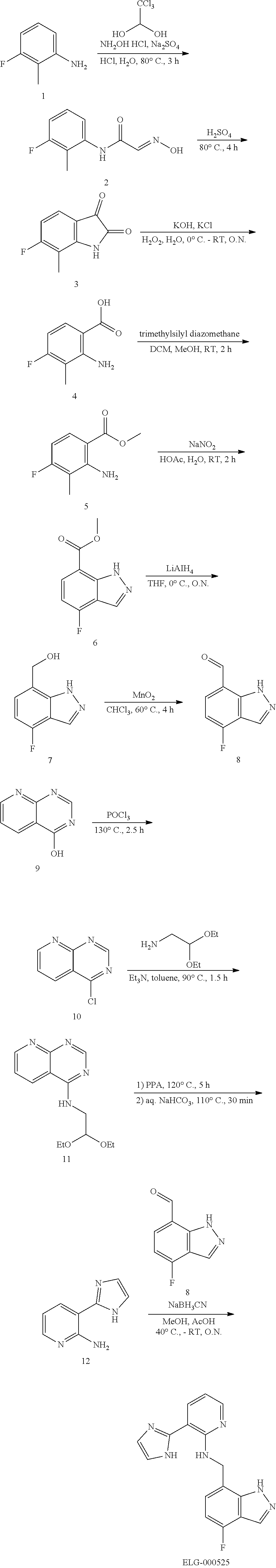

### The Synthesis of (E)-N-(3-fluoro-2-methylphenyl)-2-(hydroxyimino)acetamide (2)

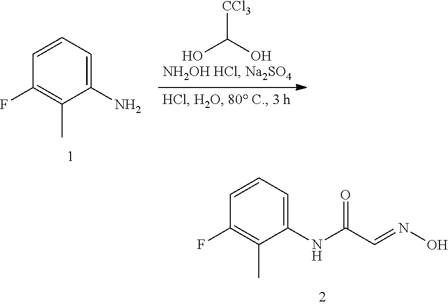

To a 1 L round bottom flask were added Na₂SO₄ (227 g, 1.59 mol), H₂O (400 mL) and 2,2,2-trichloroethane-1,1-diol (29 g, 175.6 mmol), then 1 (20.0 g, 159.8 mmol) in HCl (200 mL, 1 M in water) solution was added into the mixture, after stirred for 10 mins, NH₂OH HCl (44.4 g, 619.2 mmol) was added into the above solution and the mixture was stirred at 80° C. for 3 hours. After reaction was completed, reaction mixture was cooled down to room temperature, filtered, the filter cake was dried in vacuo to afford 2 (59 g) as crude product.

### The Synthesis of 6-fluoro-7-methylindoline-2,3-dione (3)

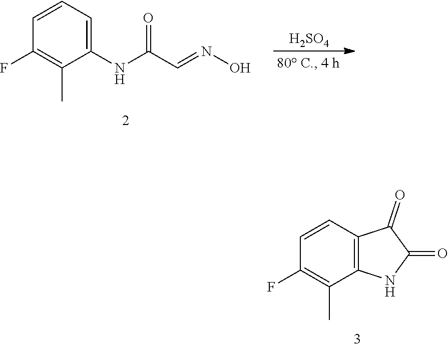

To a 1 L round bottom flask were added 2 (59 g, 310 mmol) and conc. H₂SO₄ (300 mL), the mixture was stirred at 80° C. for 4 hours. After reaction was completed, the reaction mixture was poured carefully into the ice water, extracted with EtOAc (200 mL×4), the organic phases was separated and combined, dried over anhydrous Na₂SO₄, concentrated in vacuo to afford 3 (5.34 g, yield: 17%).

### The Synthesis of 2-amino-4-fluoro-3-methylbenzoic acid (4)

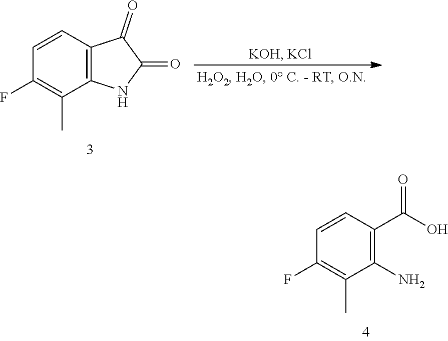

To a 250 mL three-necked flask were added 3 (5.34 g, 29.83 mmol) and H₂O (100 mL), KOH (1.84 g, 32.83 mmol) and KCl (4.66 mg, 62.52 mmol) was added at 0° C., and then slowly add H₂O₂ (5.07 g, 44.08 mmol) about 10 mins under protection of N₂, the mixture was stirred at room temperature overnight. The mixture was acidified carefully with TFA to pH=5 and diluted with H₂O (100 mL), extracted with EtOAc (100 mL×3), the organic phases was separated and combined, dried over anhydrous Na₂SO₄, concentrated in vacuo to afford 4 (4.41 g, yield: 90%).

### The Synthesis of methyl 2-amino-4-fluoro-3-methylbenzoate (5)

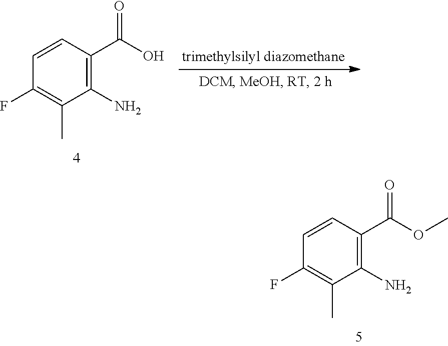

To a 250 mL round bottom flask were added 4 (4.41 g, 26.08 mmol), DCM (50 mL), MeOH (10 mL), trimethylsilyl diazomethane (4.47 g, 39.19 mmol) was added into the mixture at ice bath. Mixture was stirred at room temperature for 2 hours. Water (100 mL) was added and extracted with DCM (50 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 5 (1.36 g, yield: 84%).

### The Synthesis of methyl 4-fluoro-1H-indazole-7-carboxylate (6)

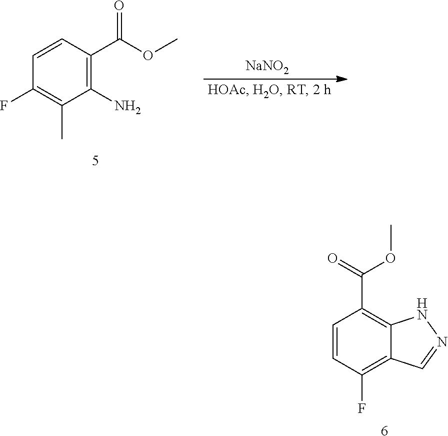

To a 100 mL round bottom flask were added 5 (1.36 g, 7.40 mmol). HOAc (15 mL), NaNO₂ (566 mg, 8.16 mmol) and H₂O (5 mL), mixture was stirred at room temperature for 2 hours. After reaction was completed, mixture was concentrated and residue was treated with a.q. NaHCO₃ solution to pH=7, mixture was extracted with EtOAc (30 mL×3), the organic phases was separated and combined, dried over anhydrous Na₂SO₄, organic phase was concentrated and purified by silica gel chromatography using a mixture of petroleum ether-ethyl acetate (80:20, v/v) as eluent to afford 6 (757 mg, yield: 90%).

### The Synthesis of (4-fluoro-1H-indazol-7-yl)methanol (7)

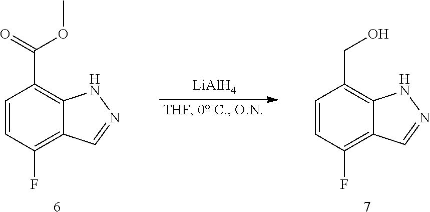

To a 100 mL three-necked flask were added 6 (757 mg, 3.94 mmol) and THF (15 mL), LiAlH₄ (2.27 mL, 5.91 mmol in THF) was added into the mixture by dropwise at 0° C. mixture was stirred at same temperature overnight. After reaction was completed, reaction mixture was quenched with 15% NaOH solution, and filtered. The filtrate was extracted with H₂O (30 mL) and EtOAc (20 mL×3), the organic phases was separated and combined, dried over anhydrous Na₂SO₄, organic phase was concentrated and purified by silica gel chromatography using a mixture of DCM-MeOH (90:10, v/v) as eluent to afford 7 (655 mg, yield: 78%).

### The Synthesis of 4-fluoro-1H-indazole-7-carbaldehyde (8)

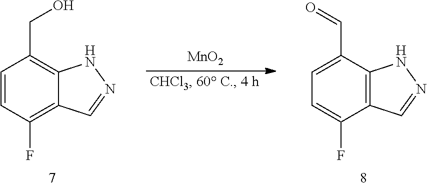

To a 100 mL three-necked flask were added 7 (655 mg, 3.93 mmol), MnO₂ (1.7 g, 19.49 mmol) and CHCl₃ (15 mL), the mixture was stirred at 60° C. for 4 hours. The mixture was filtered and filter cake was washed with DCM, the filtrate was concentrated in vacuo and purified by silica gel chromatography using a mixture of petroleum ether-ethyl acetate (80:20, v/v) as eluent to afford 8 (450 mg, yield: 90%).

### The Synthesis of 4-chloropyrido[2,3-d]pyrimidine (10)

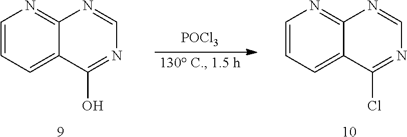

To a 250 mL round bottom flask were added 9 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 10 (1.4 g, yield: 28%) without further workup.

### The Synthesis of N-(2, 2-diethoxyethyl) pyrido [2, 3-d]pyrimidin-4-amine (11)

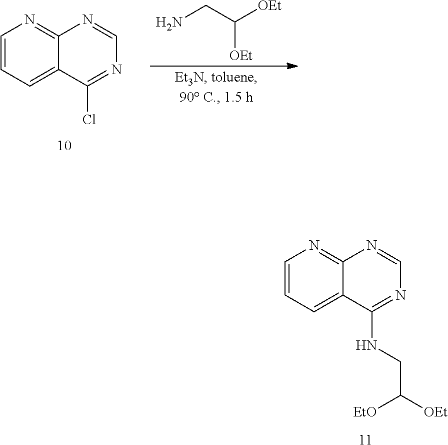

To a 250 mL round bottom flask were added 10 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 11 (1.3 g, yield: 59%).

### The Synthesis of 3-(1H-imidazol-2-yl)pyridin-2-amine (12)

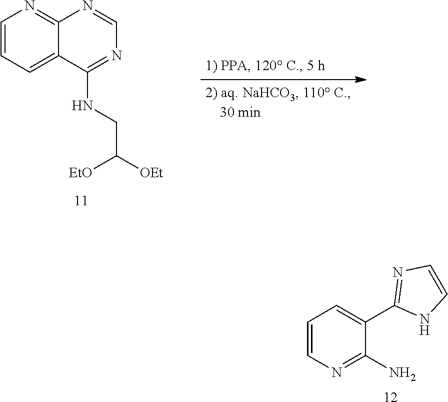

To a 250 mL round bottom flask were added 11 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 12 (0.3 g, crude). The filter cake was added saturated sodium bicarbonate solution (20 mL) and stirred at 110° C. for 0.5 hour. The reaction solution was extracted with THF (5×20 mL), combined crude product 12. The mixture was concentrated in vacuo and purified by silica gel chromatography using a mixture of petroleum ether-ethyl acetate (80:20, v/v) as eluent to afford 12 (448.0 mg, yield: 56%).

### The Synthesis of N-((4-fluoro-1H-indazol-7-yl)methyl)-3-(1H-imidazol-2-yl)pyridin-2-amine (ELG-000525)

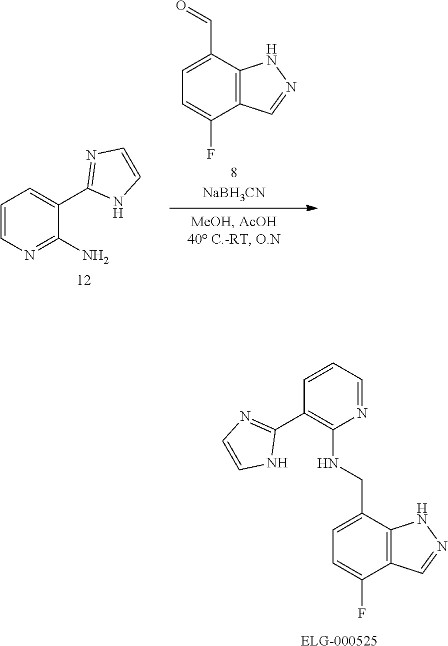

To a 50 mL round bottom flask were added 12 (40 mg, 0.25 mmol), 8 (41 mg, 0.25 mmol), MeOH (5 mL) and AcOH (5 drops), the reaction mixture was stirred at 40° C. for 2 hours, then the NaBH₃CN (79 mg, 1.25 mmol) was added into the mixture. And the reaction mixture was stirred at room temperature overnight. The reaction mixture was quenched with H₂O, then the mixture was extracted with EA (10 mL×3) and H₂O (15 mL), the combined organic layers were washed with brine, dried over anhydrous Na₂SO₄, filtered and the filtrate was concentrated. The crude product was purified by prep-TLC to afford ELG-000525 (11.6 mg, yield: 16%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.124 min; MS Calcd: 308.12; MS Found: 309.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min: Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 99.15%, Rt=3.967 min. ¹H NMR (400 MHz. DMSO-d₆) δ 13.60 (s, 1H), 12.66 (s, 1H), 9.76 (t, J=5.6 Hz, 1H), 8.20 (s, 1H), 8.05 (t, J=5.7 Hz, 2H), 7.32 (s, 1H), 7.22 (dd, J=7.5, 4.9 Hz, 1H), 7.09 (s, 1H), 6.81 (dd, J=10.2, 7.9 Hz, 1H), 6.67 (dd, J=7.4, 5.0 Hz, 1H), 5.01 (d, J=5.6 Hz, 2H). Chemical Formula: C₁₆H₁₃FN₆. Molecular Weight: 308.32.

### Route for ELG-000526

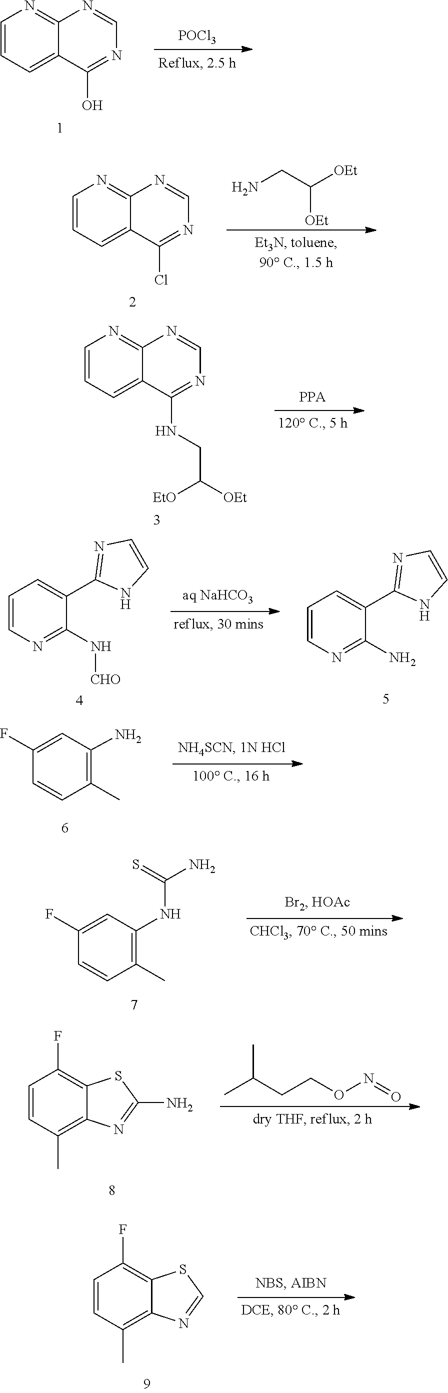

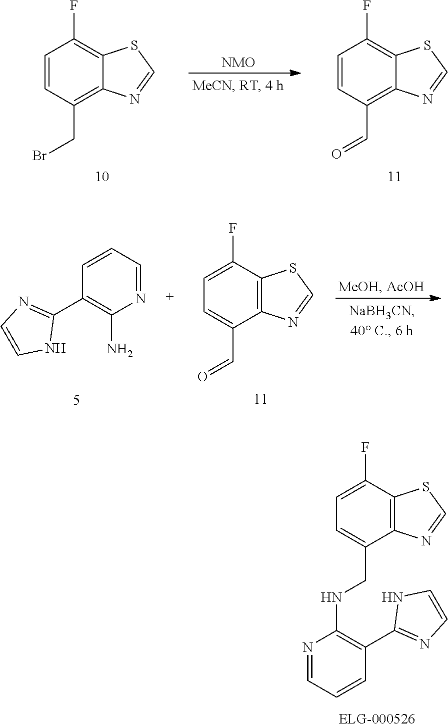

### The Synthesis of 4-chloropyrido[2,3-d]pyrimidine (2)

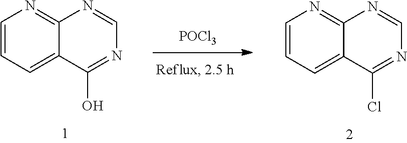

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

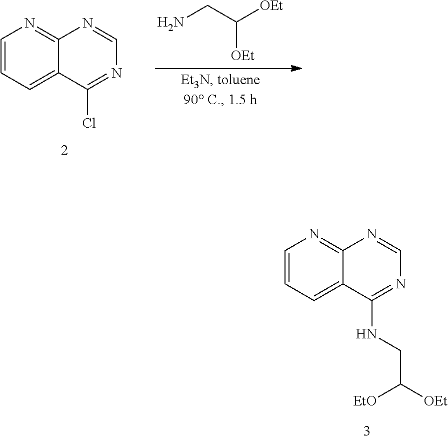 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (5)

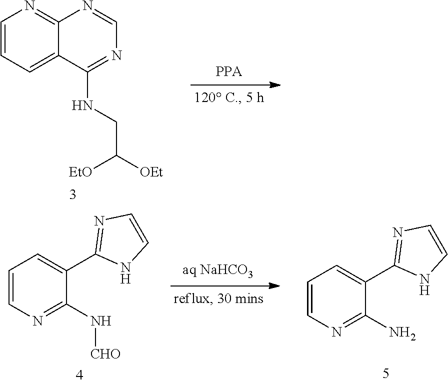

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 5 (0.3 g, crude). The filter cake was added saturated sodium bicarbonate solution (20 mL) and stirred at 110° C. for 0.5 hour. The reaction solution was extracted with THF (5×20 mL), combined crude product 5. The mixture was concentrated in vacuo and purified by silica gel chromatography using a mixture of petroleum ether-ethyl acetate (80:20, v/v) as eluent to afford 5 (448.0 mg, yield: 56%).

### The Synthesis of 1-(5-fluoro-2-methylphenyl)thiourea (7)

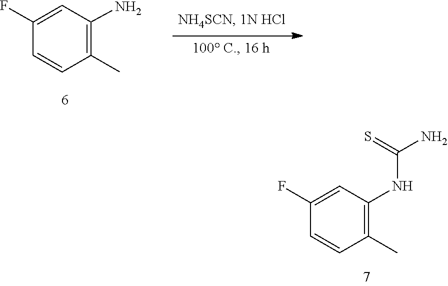

To a 100 mL round bottom flask were added 6 (650 mg, 5.19 mmol), NH₄SCN (435 mg, 5.71 mmol) and HCl (6 mL, 1 M in water), then the mixture was stirred at 100° C. for 16 hours. Then the mixture was basified by saturated ammonium hydroxide aqueous till pH>8, extracted with DCM (30 mL×5). The combined organic phase was washed with brine (30 mL×3), dried over anhydrous Na₂SO₄, concentrated under reduced pressure and purified by silica gel chromatography using petroleum ether as eluent to afford 7 (490 mg, yield: 51%).

### The Synthesis of 7-fluoro-4-methylbenzo[d]thiazol-2-amine (8)

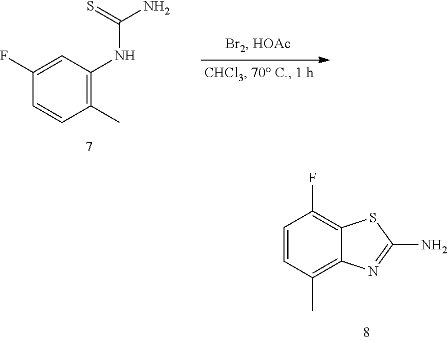

To a 100 mL round bottom flask were added 7 (490 mg, 2.66 mmol), AcOH (4.5 mL) and CHCl₃ (40 mL) and Br₂ (1.8 mL), the mixture was stirred at 70° C. for 1 hour. Then saturated NaHSO₃ aqueous was added to the reaction mixture until extra Br₂ was consumed. Then the mixture was extracted with EA (30 mL×3). The combined organic phase was washed with brine (30 mL×3), dried over anhydrous Na₂SO₄, concentrated under reduced pressure to give 8 (407 mg, yield: 84%) without further workup.

### The Synthesis of 7-fluoro-4-methylbenzo[d]thiazole (9)

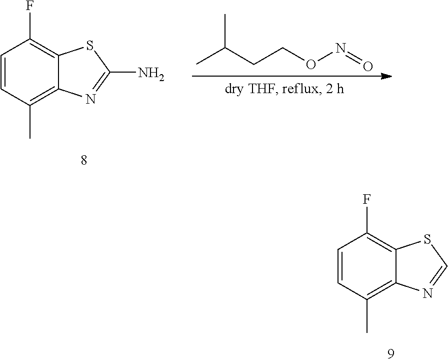

To a 100 mL bottom flask were added 8 (407 mg, 2.23 mmol) and dry THF (20 mL), the mixture was degassed under vacuum and refilled N₂ three times, then i-AmONO (721 mg, 6.15 mmol) was added. The reaction mixture was stirred at 70° C. for 2 hours. The reaction mixture was added 50 mL water to quench the reaction and concentrated to remove organic solvents. The aqueous layer was extracted with EA (30 mL×3). The combined organic phase was washed with brine (30 mL×3), dried over anhydrous Na₂SO₄, concentrated under reduced pressure and purified by silica gel chromatography using a mixture of PE-EA (98:2, v/v) as eluent to afford 9 (110 mg, yield: 29%).

### The Synthesis of 4-(bromomethyl)-7-fluorobenzo[d]thiazole (10)

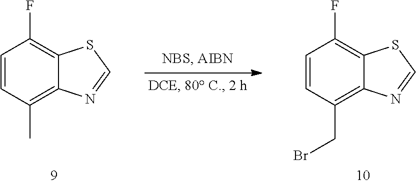

To a 50 mL bottom flask were added 9 (110 mg, 0.66 mmol). NBS (129 mg, 0.72 mmol), AIBN (12 mg, 0.07 mmol) and DCE (5 mL), the mixture was stirred at 80° C. for 2 hours. The reaction mixture was concentrated and purified by silica gel chromatography using a mixture of PE-EA (98:2, v/v) as eluent to afford 10 (109 mg, yield: 67%).

### The Synthesis of 7-fluorobenzo[d]thiazole-4-carbaldehyde (11)

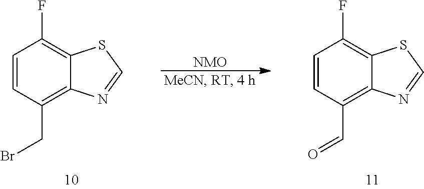

To a 100 mL round bottom flask were added 10 (109 mg, 0.44 mmol), NMO (132 mg, 1.12 mmol) and MeCN (4 mL), the mixture was stirred at room temperature for 4 hours. The reaction mixture was concentrated and purified by silica gel chromatography using a mixture of PE-EA (95:5, v/v) as eluent to afford 11 (42 mg, yield: 39%).

### The Synthesis of N-((7-fluorobenzo[d]thiazol-4-yl)methyl)-3-(1H-imidazol-2-yl)pyridin-2-amine (ELG-000526)

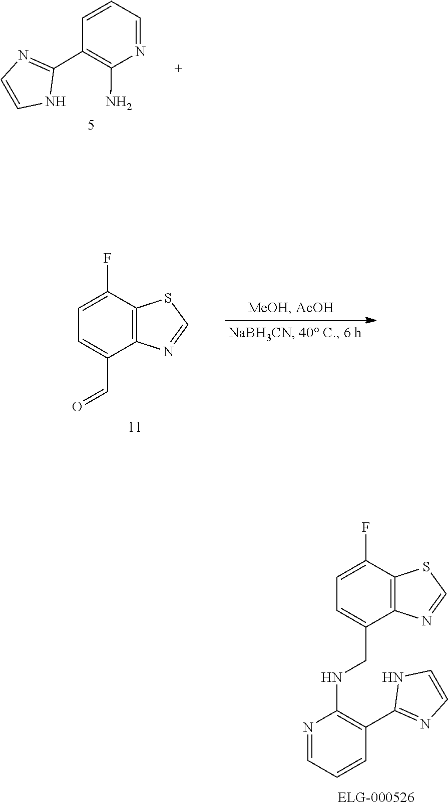 [SMILES: Nc1ncccc1-c1ncc[nH]1 ; O=Cc1ccc(F)c2scnc12 ; Fc1ccc(CNc2ncccc2-c2ncc[nH]2)c2ncsc12]

To a 100 mL bottom flask were added 5 (40.0 mg, 0.25 mmol), 11 (47.0 mg, 0.26 mmol), AcOH (5 drops) and MeOH (5 mL), the mixture stirred at 40° C. for 2 hours. Then the reaction mixture was added NaBH₃CN (47.0 mg, 0.75 mmol) and stirred at 40° C. for another 4 hours. The mixture was directly purified by prep-HPLC (a mixture of CH₃CN and H₂O with 0.5% HCO₂H as eluent) to afford ELG-000526 (22.3 mg, yield: 28%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.188 min; MS Calcd: 325.08; MS Found: 326.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 95.32%, Rt=4.339 min. ¹H NMR (400 MHz, DMSO-d₆) δ 12.63 (s, 1H), 9.83 (t, J=5.8 Hz, 1H), 9.53 (d, J=1.3 Hz, 1H), 8.08-7.96 (m, 2H), 7.45 (dd, J=8.3, 5.4 Hz, 1H), 7.32 (t, J=8.9 Hz, 2H), 7.09 (s, 1H), 6.65 (dd, J=7.6, 4.9 Hz, 1H), 5.23 (d, J=5.9 Hz, 2H) Chemical Formula: C₁₆H₁₂FN₅S. Molecular Weight: 325.37.

### Route for ELG-000530

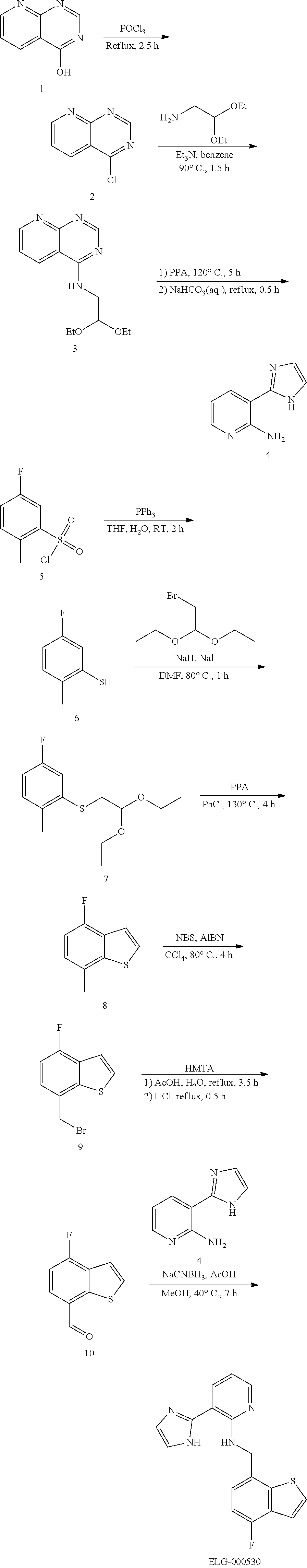

### The Synthesis of 4-chloropyrido[2,3-d]pyrimidine (2)

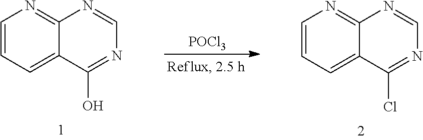

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g. yield: 28%) without further workup.

### The Synthesis of N-(2,2-diethoxyethyl)pyrido[2,3-d]pyrimidin-4-amine (3)

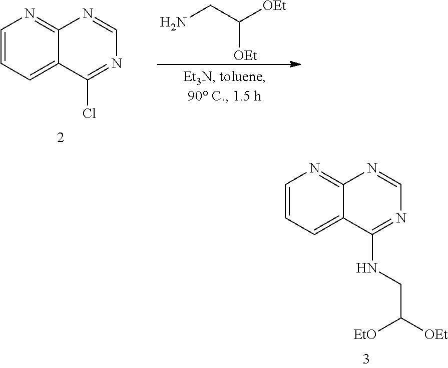 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (4)

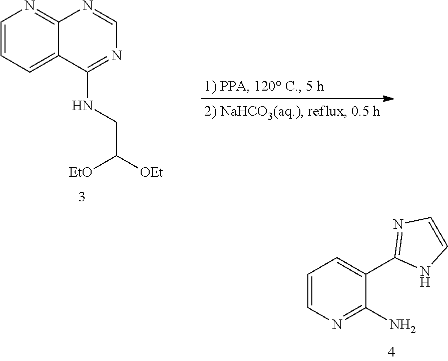 [SMILES: CCOC(CNc1ncnc2ncccc12)OCC ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 4 (0.3 g, crude). The filter cake was added saturated sodium bicarbonate solution (20 mL) and stirred at 110° C. for 0.5 hour. The reaction solution was extracted with THF (5×20 mL), combined crude product 4. The mixture was concentrated in vacuo and purified by silica gel chromatography using a mixture of petroleum ether-ethyl acetate (80:20, v/v) as eluent to afford 4 (448.0 mg, yield: 56%).

### The Synthesis of 5-fluoro-2-methylbenzenethiol (6)

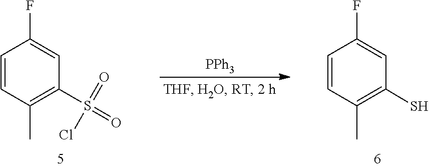

To a 100 mL round bottom flask were added 5 (5.0 g, 23.9 mmol), THF (80 mL) and triphenylphosphine (19.0 g, 71.7 mmol). The reaction mixture was stirred at room temperature for 1 hour. Then water (3.2 mL) was added and stirred for another 1 hour. Once the reaction completed, water (20 mL) was added in the reaction and extracted with EtOAc (20 mL×3), the organic phase was combined, dried over anhydrous Na₂SO₄, concentrated in vacuo and purified by silica gel chromatography using petroleum ether as eluent to afford 6 (2.2 g, yield: 65%).

### The Synthesis of (2,2-diethoxyethyl)(5-fluoro-2-methylphenyl)sulfane (7)

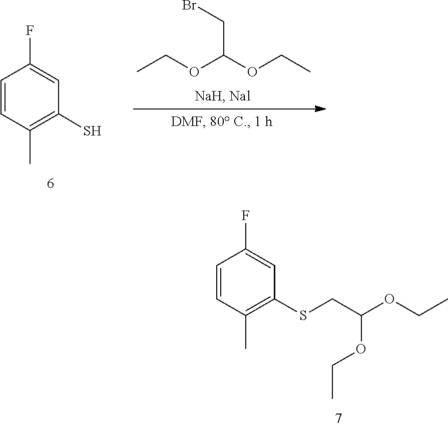

To a 100 mL round bottom flask were added 6 (2.2 g, 15.5 mmol), bromoacetaldehyde diethyl acetal (3.4 g, 17.3 mmol), NaI (1.2 g, 8.0 mmol), NaH (1.2 g, 50.0 mmol) and DMF (50 mL), the reaction mixture was stirred at 80° C. for 1 hour. Once the reaction completed, water (100 mL) was added, extracted with EtOAc (30 mL×3), the organic phase was combined, washed by brine (30 mL×3), dried over anhydrous Na₂SO₄, concentrated in vacuo and purified by silica gel chromatography using petroleum ether as eluent to afford 7 (3.8 g, yield: 95%).

### The Synthesis of 4-fluoro-7-methylbenzo[b]thiophene (8)

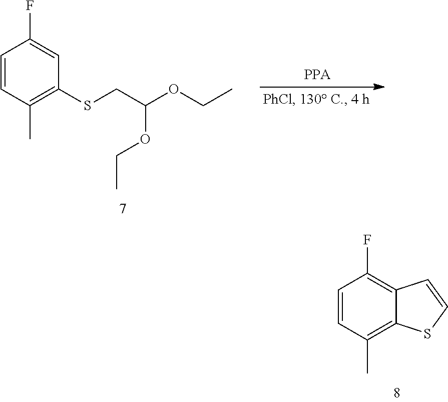 [SMILES: CCOC(CSc1cc(F)ccc1C)OCC ; Cc1ccc(F)c2ccsc12]

To a 100 mL round bottom flask were added paraformaldehyde (8.0 g, 88.8 mmol), chlorobenzene (60 mL) and 7 (3.8 g, 14.7 mmol). The reaction mixture and stirred at 130° C. for 4 hours. The reaction mixture was concentrated in vacuo, the residue was diluted with water (50 mL), extracted with EtOAc (30 mL×3), the combined organic phase was washed with saturated sodium bicarbonate aqueous solution (30 mL×3), dried over anhydrous Na₂SO₄, concentrated in vacuo and purified by silica gel chromatography using petroleum ether as eluent to afford 8 (1.75 g, yield: 70%).

### The Synthesis of 7-(bromomethyl)-4-fluorobenzo[b]thiophene (9)

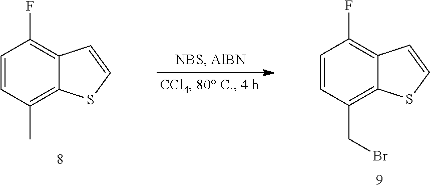

To a 100 mL round bottom flask were added 8 (1.75 g, 10.5 mmol), NBS (2.43 g, 13.6 mmol), AIBN (0.35 g, 2.1 mmol) and carbon tetrachloride (50 mL), the reaction mixture was stirred at 80° C. for 4 hours. Once the reaction completed, the reaction mixture was diluted with water (50 mL), extracted with DCM (20 mL×3), the organic phase was washed with saturated sodium bicarbonate aqueous solution (20 mL×3), the organic phase was combined, dried over anhydrous Na₂SO₄, concentrated in vacuo, and purified by silica gel chromatography using petroleum ether as eluent to afford 9 (2.3 g, yield: 91%).

### The Synthesis of 4-fluorobenzo[b]thiophene-7-carbaldehyde (10)

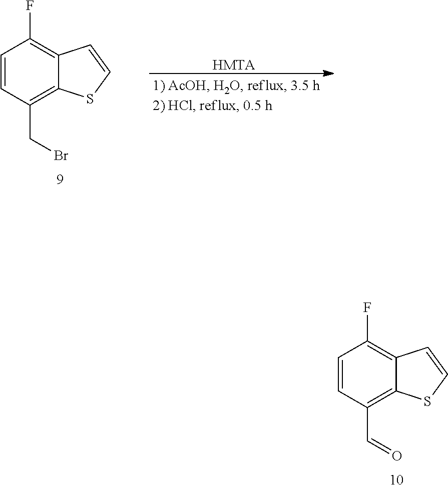

To a 100 mL round bottom flask were added 9 (114 mg, 0.81 mmol), hexamethylenetetramine (90 mg, 0.37 mmol), acetic acid (2 mL) and water (1 mL), the reaction mixture was stirred at 110° C. for 3.5 hours. Then concentrated hydrochloric acid (1 mL) was added at room temperature, keep stir at 110° C. for another 0.5 hour. Once the reaction completed, water (30 mL) was added in the reaction mixture, extracted with EA (20 mL×3), the organic phase was combined and dried over anhydrous Na₂SO₄, concentrated in vacuo, and purified by silica gel chromatography using using a mixture of petroleum ether-ethyl acetate (95:5, v/v) as eluent to afford 10 (40 mg, yield: 61%).

### The Synthesis of N-((4-fluorobenzo[b]thiophen-7-yl)methyl)-3-(1H-imidazol-2-yl)pyridin-2-amine (ELG-000530)

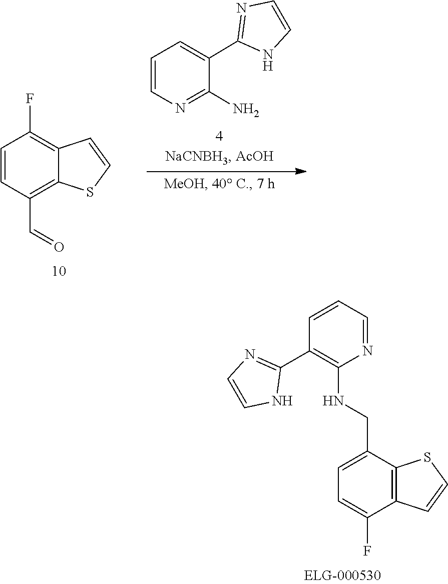 [SMILES: O=Cc1ccc(F)c2ccsc12 ; Nc1ncccc1-c1ncc[nH]1 ; Fc1ccc(CNc2ncccc2-c2ncc[nH]2)c2sccc12]

To a 100 mL bottom flask were added 10 (32.4 mg, 0.18 mmol), 4 (30.0 mg, 0.19 mmol), AcOH (3 drops) and methanol (3 mL), the mixture stirred at 40° C. for 2 hours. Then the reaction mixture was added NaBH₃CN (34.0 mg, 0.54 mmol) and stirred at the same temperature for another 5 hours. The reaction mixture was quenched by water (0.5 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000530 (7.0 mg, yield: 12%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.272 min; MS Calcd: 324.08; MS Found: 325.1 [M+H]⁺. HPLC (Waters UPLC, Column: ACQUITY UPLC BEH C18 1.7 μm*2.1 mm*150 mm); Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min). Purity: 98.66%, Rt=4.520 min. ¹H NMR (400 MHz, DMSO-d₆) δ 12.67 (brs, 1H), 9.85 (brs, 1H), 8.06 (dd, J=7.6, 1.5 Hz, 1H), 8.02 (dd, J=4.9, 1.4 Hz, 1H), 7.83 (d, J=5.5 Hz, 1H), 7.53 (d, J=5.5 Hz, 1H), 7.33 (dd, J=8.0, 4.9 Hz, 2H), 7.22-7.06 (m, 2H), 6.67 (dd, J=7.4, 4.9 Hz, 1H), 4.97 (d, J=4.5 Hz, 2H). Chemical Formula: C₁₇H₁₃FN₄S. Molecular Weight: 324.38.

### Route for ELG-000531

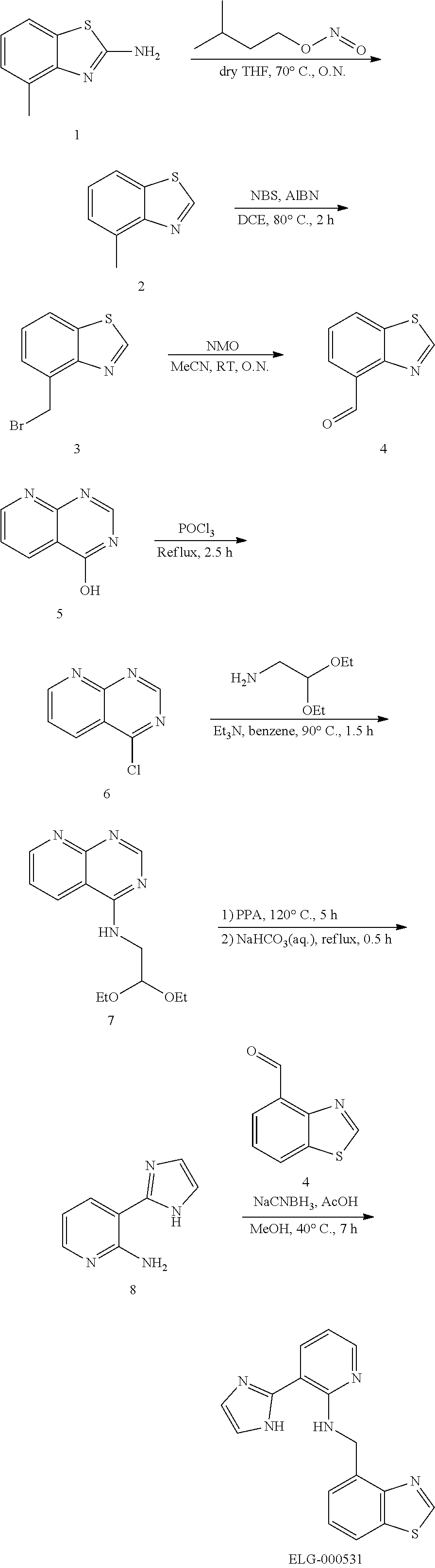

### The Synthesis of 4-methylbenzo[d]thiazole(2)

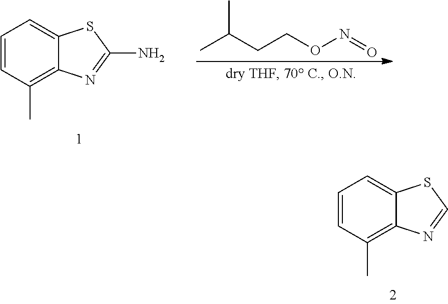

To a 250 mL round bottom flask were added 1 (2.0 g, 12.18 mmol) and dry THF(80 mL), the mixture was degassed and refilled N₂ three times, then i-AmONO (2.8 g, 24.36 mmol) was added to the mixture and stirred at 70° C. overnight. The reaction mixture was concentrated and added 50 mL water to quench the reaction. The aqueous layer was extracted with EA (8×20 mL), the combined organic phase was washed with brine (3×30 mL), dried over anhydrous Na₂SO₄, concentrated under reduced pressure and purified by silica gel chromatography using a mixture of petroleum ether-ethyl acetate (95:5, v/v) as eluent to afford 2 (1.5 g, yield: 84%).

### The Synthesis of 4-(bromomethyl)benzo[d]thiazole(3)

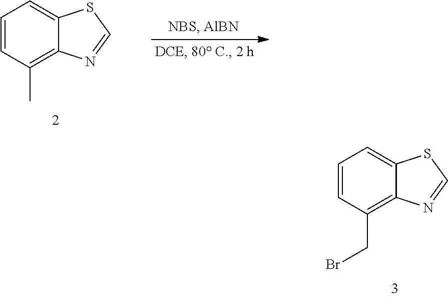

To a 250 mL round bottom flask were added 2 (1.4 g, 9.38 mmol), NBS (1.9 g, 10.32 mmol), AIBN (154.4 mg, 0.94 mmol) and DCE (40 mL), the mixture was stirred at 80° C. for 2 hours. The reaction mixture was concentrated and purified by silica gel chromatography using a mixture of petroleum ether-ethyl acetate (98:2, v/v) as eluent to afford 3 (1.5 g, yield: 71%).

### The Synthesis of benzo[d]thiazole-4-carbaldehyde(4)

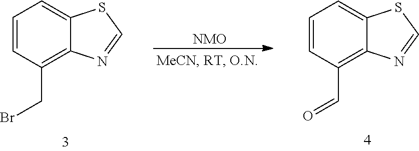

To a 100 mL round bottom flask were added 3 (700.0 mg, 3.07 mmol), NMO (1.4 g, 12.33 mmol) and MeCN (25 mL), the mixture was stirred at room temperature overnight. The reaction mixture was concentrated and purified by silica gel chromatography using a mixture of petroleum ether-ethyl acetate (97:3, v/v) as eluent to afford 4 (122.0 mg, yield: 25%).

### The Synthesis of 4-chloropyrido[2,3-d]pyrimidine (6)

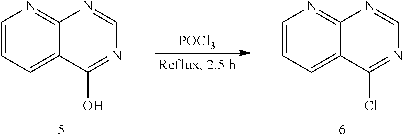

To a 250 mL round bottom flask were added 5 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 6 (1.4 g, yield: 28%) without further workup.

### The Synthesis of N-(2,2-diethoxyethyl)pyrido[2,3-d]pyrimidin-4-amine (7)

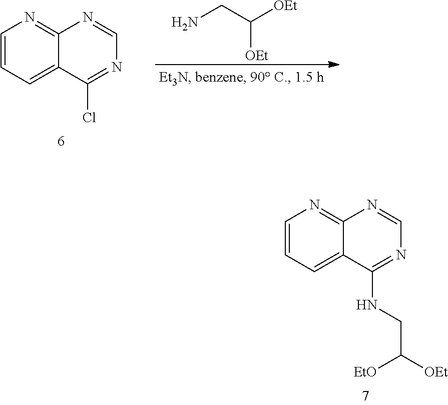 [SMILES: Clc1ncnc2ncccc12 ; CCOC(CN)OCC ; CCOC(CNc1ncnc2ncccc12)OCC]

To a 250 mL round bottom flask were added 6 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 7 (1.3 g, yield: 59%).

### The Synthesis of 3-(1H-imidazol-2-yl)pyridin-2-amine (8)

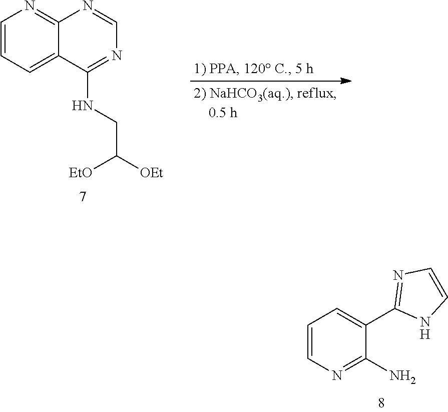 [SMILES: CCOC(CNc1ncnc2ncccc12)OCC ; Nc1ncccc1-c1ncc[nH]1]

To a 250 mL round bottom flask were added 7 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 8 (0.3 g, crude). The filter cake was added saturated sodium bicarbonate solution (20 mL) and stirred at 110° C. for 0.5 hour. The reaction solution was extracted with THF (5×20 mL), combined crude product 8. The mixture was concentrated in vacuo and purified by silica gel chromatography using a mixture of petroleum ether-ethyl acetate (80:20, v/v) as eluent to afford 8 (448.0 mg, yield: 56%).

### The Synthesis of N-(benzo[d]thiazol-4-ylmethyl)-3-(1H-imidazol-2-yl)pyridin-2-amine (ELG-000531)

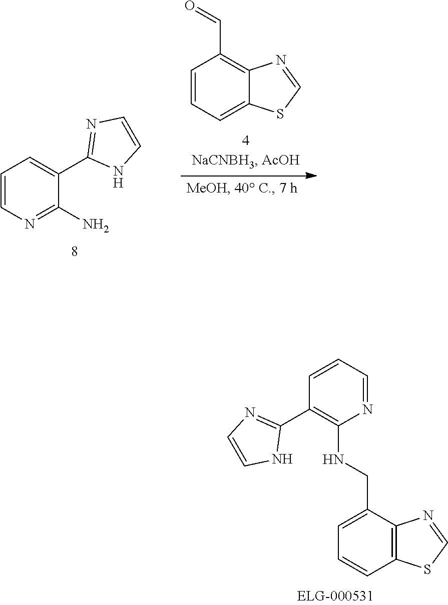 [SMILES: Nc1ncccc1-c1ncc[nH]1 ; O=Cc1cccc2scnc12 ; c1cnc(NCc2cccc3scnc23)c(-c2ncc[nH]2)c1]

To a 100 mL round bottom flask were added 8 (40.0 mg, 0.25 mmol). MeOH (3 mL), 4 (42.5 mg, 0.26 mmol) and AcOH (3 drops), the mixture was stirred at 40° C. until 4 consumed, about 2 hours. Then added NaCNBH₃ (72.3 mg, 1.15 mmol), the reaction mixture was stirred at room temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000531 (21.0 mg, yield: 28%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min: Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.130 min; MS Calcd: 307.09; MS Found: 308.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 100%, Rt=3.936 min. ¹H NMR (400 MHz, DMSO-d₆) δ 12.89 (brs, 1H), 10.00 (brs, 1H), 9.45 (s, 1H), 8.12-8.01 (m, 3H), 7.50-7.38 (m, 2H), 7.29 (brs, 2H), 6.84-6.66 (m, 1H), 5.27 (s, 2H). Chemical Formula: C₁₆H₁₃N₅S. Molecular Weight: 307.38.

### Route for ELG-000532

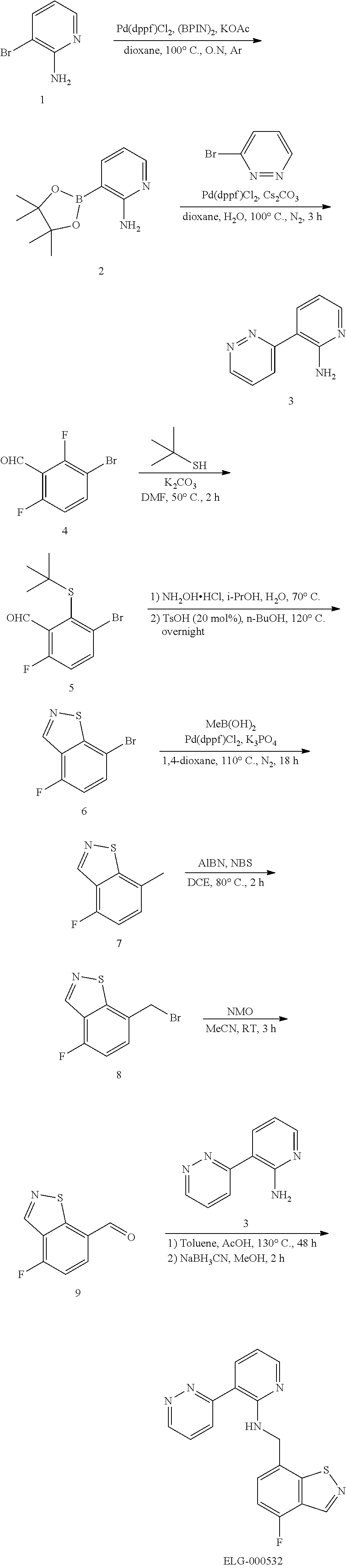

### The Synthesis of 3-(4,4,5,5-tetramethyl-1,3,2-dioxaborolan-2-yl)pyridin-2-amine (2)

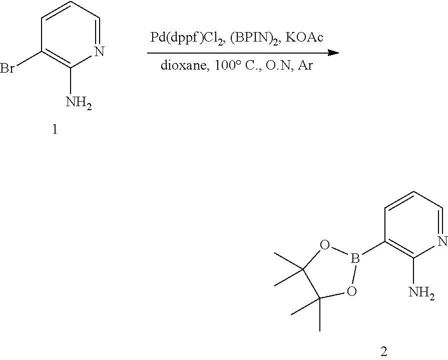 [SMILES: Nc1ncccc1Br ; CC1(C)OB(c2cccnc2N)OC1(C)C]

To a 250 mL round bottom flask were added 1 (500 mg, 2.89 mmol), (BPIN)₂ (881 mg, 3.47 mmol), AcOK (850 mg, 8.67 mmol) and Pd(dppf)Cl₂ (64 mg, 0.09 mmol). The mixture was dewatered by heated oil pump for 10 min and then the dry dioxane (20 mL) was added. The reaction mixture was degassed and refilled with Ar five times, then reaction mixture was stirred at 100° C. overnight. The reaction mixture (2, crude) was used directly for the next step without additional purification.

### The Synthesis of 3-(pyridazin-3-yl)pyridin-2-amine (3)

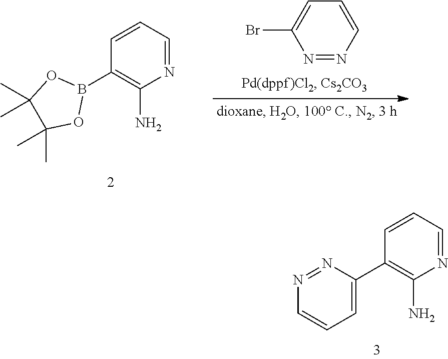 [SMILES: CC1(C)OB(c2cccnc2N)OC1(C)C ; Brc1cccnn1 ; Nc1ncccc1-c1cccnn1]

To the above reaction mixture 2 were added 3-bromopyridazine (0.53 g, 3.34 mmol). Cs₂CO₃ (2.07 g, 6.36 mmol), Pd(dppf)Cl₂ (0.07 g, 0.10 mmol) and H₂O (5 mL). The reaction mixture was degassed and refilled with N₂ five times, then stirred at 100° C. for 3 hours. The reaction mixture was concentrated in vacuo, extracted with EA (40 mL×3) and H₂O (60 mL). The combined organic layers were washed with brine, dried over anhydrous Na₂SO₄, filtered and the filtrate was concentrated. The crude product was purified by silica gel chromatography using a mixture of petroleum ether-ethyl acetate (0:100, v/v) as eluent to afford 3 (260 mg, yield: 48%).

### The Synthesis of 3-bromo-2-(tert-butylthio)-6-fluorobenzaldehyde (5)

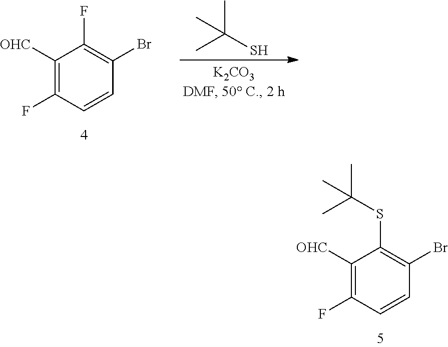

To a 250 mL round bottom flask were added 4 (5.0 g, 22.62 mmol), 2-methylpropane-2-thiol (2.0 g, 21.55 mmol), K₂CO₃ (4.5 g, 32.33 mmol) and DMF (30 mL). Then the reaction mixture was stirred at 50° C. for 2 hours. Waters (100 mL) was added and the mixture was extracted with EA (50 mL×3), the combined organic layers were washed with brine, dried over anhydrous Na₂SO₄, filtered and the filtrate was concentrated. The crude product was purified by silica gel chromatography using a mixture of petroleum ether-ethyl acetate (90:10, v/v) as eluent to afford 5 (6.06 g, yield: 96%).

### The Synthesis of 7-bromo-4-fluorobenzo[d]isothiazole (6)

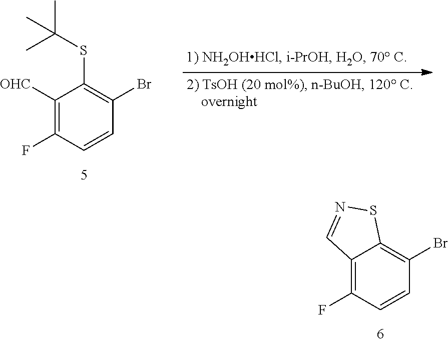

To a 250 mL round bottom flask were added 5 (6.06 g, 20.81 mmol), NH₂OH HCl (7.28 g, 104.06 mmol), i-PrOH (80 mL) and H₂O (15 mL). The reaction mixture was stirred at 70° C. for 20 mins. The organic solvent was removed in vacuo and saturated NaHCO₃ solution was added till pH>8, then the mixture was extracted with DCM (30 mL×3) and H₂O (60 mL), the organic layer was concentrated in vacuo, the residue was treated with TsOH (0.72 g, 4.16 mmol) in n-BuOH (80 mL), and the mixture was stirred at 120° C. overnight. The reaction mixture was concentrated and purified by silica gel chromatography using a mixture of petroleum ether-ethyl acetate (95:5, v/v) as eluent to afford 6 (3.3 g, yield: 69%).

### The Synthesis of 4-fluoro-7-methylbenzo[d]isothiazole (7)

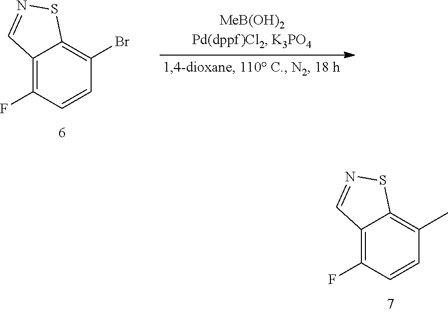

To a 250 mL round bottom flask were added 6 (3.3 g, 14.29 mmol). MeB(OH)₂ (2.57 g, 42.87 mmol), K₃PO₄ (9.10 g, 42.87 mmol). Pd(dppf)Cl₂ (0.21 g, 0.29 mmol), dioxane (50 mL), H₂O (10 mL). The reaction mixture was degassed and refilled with N₂ five times, stirred at 100° C. for 3 hours. Waters (100 mL) was added and the mixture was extracted with EA (50 mL×3), the combined organic layers were washed with brine, dried over anhydrous Na₂SO₄, filtered and the filtrate was concentrated. The crude product was purified by silica gel chromatography by using a mixture of petroleum ether-ethyl acetate (98:2, v/v) as eluent to afford 7 (1.9 g, yield: 79%).

### The Synthesis of 7-(bromomethyl)-4-fluorobenzo[d]isothiazole (8)

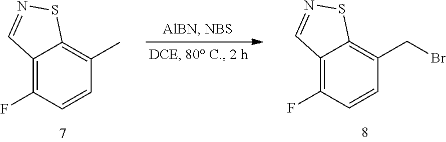

To a 100 mL round bottom flask were added 7 (1.82 g, 10.09 mmol), NBS (1.98 g, 11.12 mmol), AIBN (0.17 g, 1.01 mmol) and DCE (20 mL). The reaction mixture was stirred at 80° C. for 2 hours. The reaction mixture was concentrated and purified by silica gel chromatography using a mixture of petroleum ether-ethyl acetate (95:5, v/v) as eluent to afford 8 (1.8 g, yield: 73%).

### The Synthesis of 4-fluorobenzo[d]isothiazole-7-carbaldehyde (9)

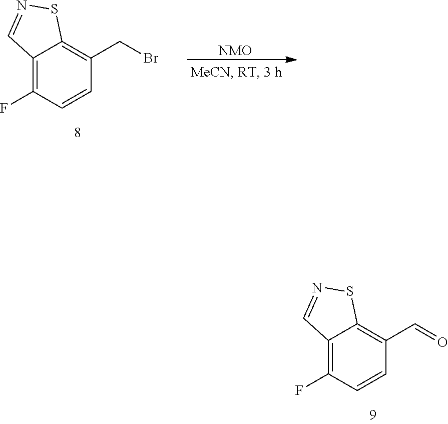

To a 100 mL round bottom flask were added 8 (1.30 g, 5.31 mmol). NMO (4.97 g, 42.45 mmol), MeCN (30 mL). Then the reaction mixture was stirred at room temperature for 3 hours. The reaction mixture was concentrated and purified by silica gel chromatography by using a mixture of petroleum ether-ethyl acetate (95:5, v/v) as eluent to afford 9 (700 mg, yield: 70%).

### The Synthesis of N-((4-fluorobenzo[d]isothiazol-7-yl)methyl)-3-(pyridazin-3-yl)pyridin-2-amine (ELG-000532)

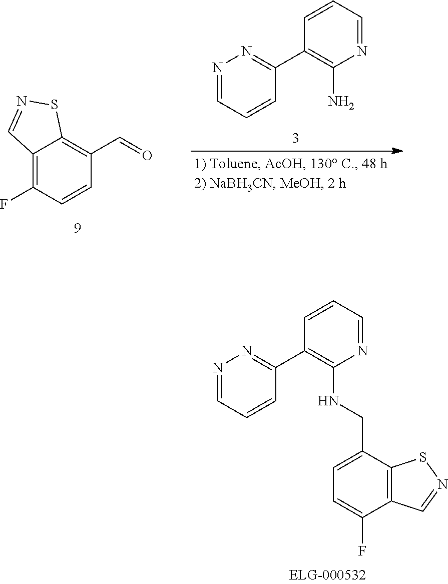 [SMILES: O=Cc1ccc(F)c2cnsc12 ; Nc1ncccc1-c1cccnn1 ; Fc1ccc(CNc2ncccc2-c2cccnn2)c2sncc12]

To a 50 mL round bottom flask were added 9 (22 mg, 0.12 mmol), 3 (21 mg, 0.12 mmol), Toluene (10 mL) and AcOH (8 drops), the reaction mixture was stirred at 130° C. for 48 hours with Dean-Stark equipment. After the reaction was completed, the organic solvent was removed in vacuo. Then to the residue was added MeOH (5 mL) and NaBH₃CN (79 mg, 1.25 mmol), stirred at room temperature for 2 hours. The reaction mixture was quenched with H₂O and extracted with EA (10 mL×3), the combined organic layers were washed with brine, dried over anhydrous Na₂SO₄, filtered and the filtrate was concentrated. The crude product was purified by prep-TLC to afford ELG-000532 (5.8 mg, yield: 16%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 95.93%, Rt=1.231 min: MS Calcd: 337.08: MS Found: 338.0 [M+H]⁺. HPLC (Waters UPLC. Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 93.58%, Rt=4.358 min. ¹H NMR (400 MHz, DMSO-d₆) δ 9.75 (t, J=5.7 Hz, 1H), 9.24 (dd, J=4.9, 1.5 Hz, 1H), 9.13 (s, 1H), 8.39 (dd, J=8.9, 1.5 Hz, 1H), 8.21 (dd, J=7.8, 1.7 Hz, 1H), 8.12 (dd, J=4.8, 1.7 Hz, 1H), 7.89 (dd, J=8.9, 4.9 Hz, 1H), 7.62 (dd, J=8.0, 4.7 Hz, 1H), 7.31 (dd, J=10.0, 8.0 Hz, 1H), 6.80 (dd, J=7.7, 4.8 Hz, 1H), 5.07 (d, J=5.7 Hz, 2H) Chemical Formula: C₁₇H₁₂FN₅S. Molecular Weight: 337.38.

### Route for ELG-000534

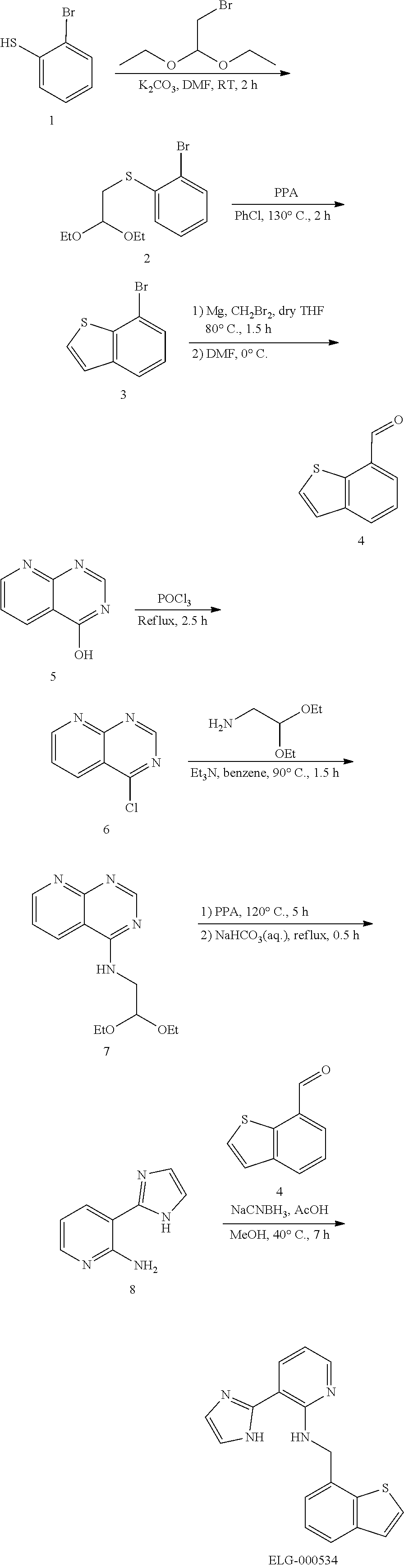

### The Synthesis of (2-bromophenyl)(2,2-diethoxyethyl)sulfane (2)

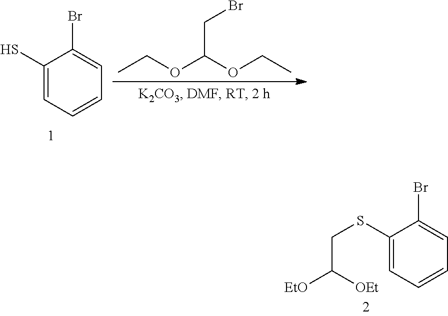

To a 100 mL round bottom flask were added 1 (4.4 g, 23.3 mmol), potassium carbonate (5.1 g, 36.9 mmol), Bromoacetaldehyde diethyl acetal (5.0 g, 25.4 mmol) and DMF (40 mL), the reaction mixture was stirred at room temperature for 4 hours. Once the reaction completed, water (100 mL) was added in the reaction and extracted with EtOAc (30 mL×4), the organic phase was washed with saturated brine (30 mL×4) and dried over Na₂SO₄ and concentrated in vacuo. The crude product was purified by silica gel chromatography using a mixture of petroleum ether-ethyl acetate (90:10, v/v) as eluent to afford 2 (6.6 g. yield: 94%).

### The Synthesis of 7-bromobenzo[b]thiophene (3)

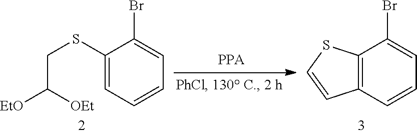 [SMILES: CCOC(CSc1ccccc1Br)OCC ; Brc1cccc2ccsc12]

To a 100 mL round bottom flask were added paraformaldehyde (15.0 g, 166.5 mmol) and chlorobenzene (50 mL) and 2 (6.6 g, 21.7 mmol). The reaction mixture was stirred at 130° C. for 2 hours. The reaction mixture was concentrated in vacuo, the residue was diluted with water (100 mL), extracted with EtOAc (30 mL×5), the combined organic phase was washed with saturated sodium bicarbonate aqueous solution (30 mL×3), dried over anhydrous Na₂SO₄ and concentrated in vacuo. The crude product was purified by silica gel chromatography using petroleum ether as eluent to afford 3 (3.7 g, yield: 79%).

### The Synthesis of benzo[b]thiophene-7-carbaldehyde (4)

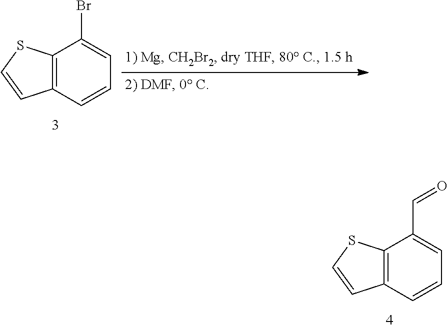 [SMILES: Brc1cccc2ccsc12 ; O=Cc1cccc2ccsc12]

To a 100 mL round bottom flask were added magnesium bars (616.8 mg, 25.7 mmol) and THF (14 mL), the reaction mixture was degassed and refilled with Ar three times, then stirred at 80° C. for 1.5 hours. The reaction mixture was cooled to 0° C., DMF (2.8 mL, 32.8 mmol) was slowly added and stirred at room temperature overnight. Once the reaction completed, the reaction mixture was concentrated in vacuo and purified by silica gel chromatography using a mixture of petroleum ether-ethyl acetate (95:5, v/v) as eluent to afford 4 (1.6 g, yield: 59%).

### The Synthesis of 4-chloropyrido[2,3-d]pyrimidine (6)

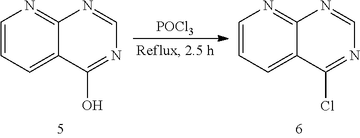

To a 250 mL round bottom flask were added 5 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 6 (1.4 g, yield: 28%) without further workup.

### The Synthesis of N-(2,2-diethoxyethyl)pyrido[2,3-d]pyrimidin-4-amine (7)

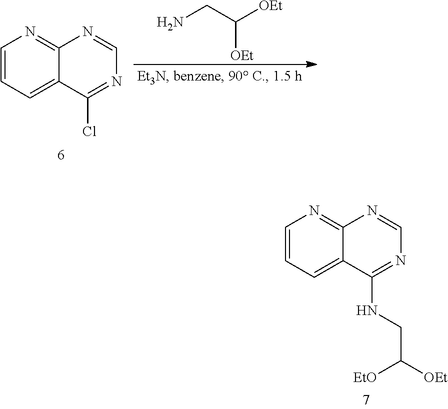 [SMILES: Clc1ncnc2ncccc12 ; CCOC(CN)OCC ; CCOC(CNc1ncnc2ncccc12)OCC]

To a 250 mL round bottom flask were added 6 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 7 (1.3 g, yield: 59%).

### The Synthesis of 3-(1H-imidazol-2-yl)pyridin-2-amine (8)

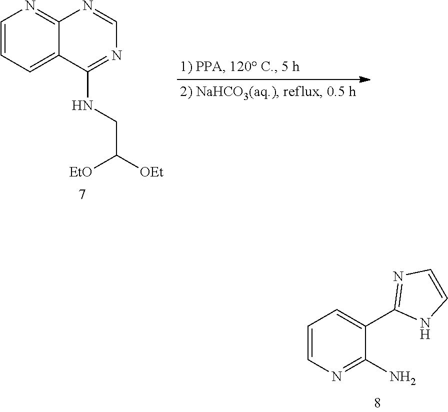 [SMILES: CCOC(CNc1ncnc2ncccc12)OCC ; Nc1ncccc1-c1ncc[nH]1]

To a 250 mL round bottom flask were added 7 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 8 (0.3 g, crude). The filter cake was added saturated sodium bicarbonate solution (20 mL) and stirred at 110° C. for 0.5 hour. The reaction solution was extracted with THF (5×20 mL), combined crude product 8. The mixture was concentrated in vacuo and purified by silica gel chromatography using a mixture of petroleum ether-ethyl acetate (80:20, v/v) as eluent to afford 8 (448.0 mg, yield: 56%).

### The Synthesis of N-(benzo[b]thiophen-7-ylmethyl)-3-(1H-imidazol-2-yl)pyridin-2-amine (ELG-000534)

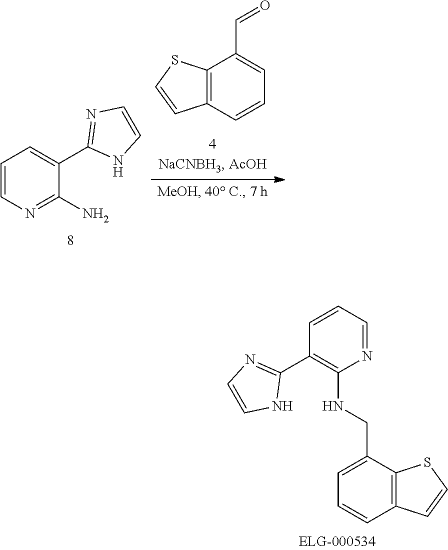

To a 100 mL round bottom flask were added 8 (29.9 mg, 0.19 mmol), MeOH (3 mL), 4 (31.8 mg, 0.20 mmol) and AcOH (3 drops), the mixture was stirred at 40° C. until 4 consumed, about 2 hours. Then added NaCNBH₃ (72.3 mg, 1.15 mmol), the reaction mixture was stirred at room temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000534 (9.9 mg, yield: 17%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%. Rt=1.211 min; MS Calcd: 306.09; MS Found: 307.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 98.71%, Rt=4.350 min. ¹H NMR (400 MHz, DMSO-d₆) δ 12.65 (s, 1H), 9.85 (t, J=5.6 Hz, 1H), 8.08-8.04 (m, 1H), 8.02 (d, J=4.8 Hz, 1H), 7.79 (d, J=7.5 Hz, 1H), 7.74 (d, J=5.4 Hz, 1H), 7.49 (d, J=5.4 Hz, 1H), 7.38-7.30 (m, 3H), 7.08 (s, 1H), 6.67 (dd. J=7.5, 4.9 Hz, 1H), 4.99 (d, J=5.7 Hz, 2H). Chemical Formula: C₁₇H₁₄N₄S. Molecular Weight: 306.39.

### Route for ELG-000536

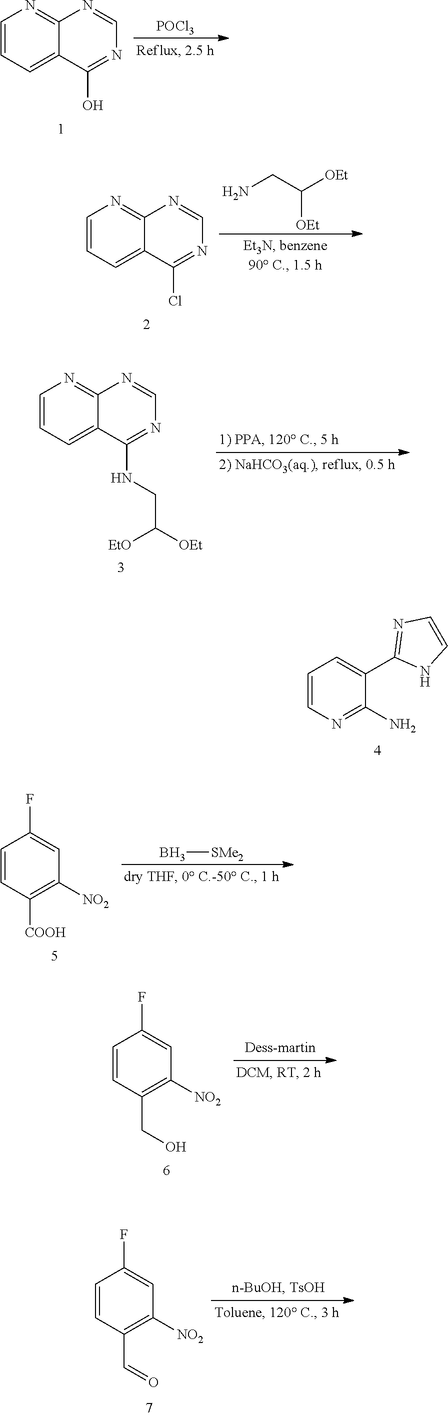

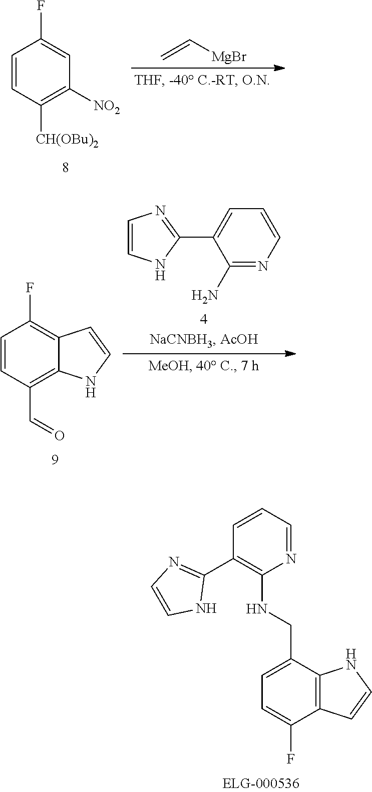

### The Synthesis of 4-chloropyrido[2,3-d]pyrimidine (2)

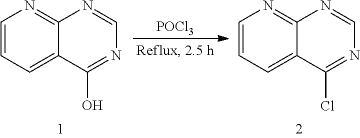

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g. yield: 28%) without further workup.

### The Synthesis of N-(2,2-diethoxyethyl)pyrido[2,3-d]pyrimidin-4-amine (3)

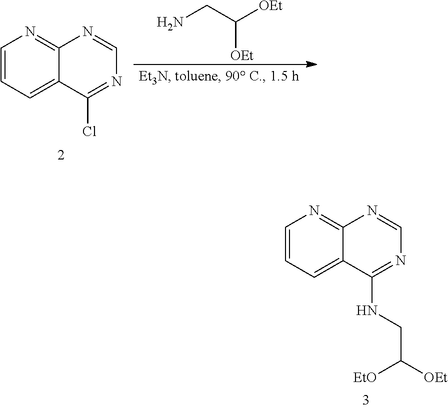 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (4)

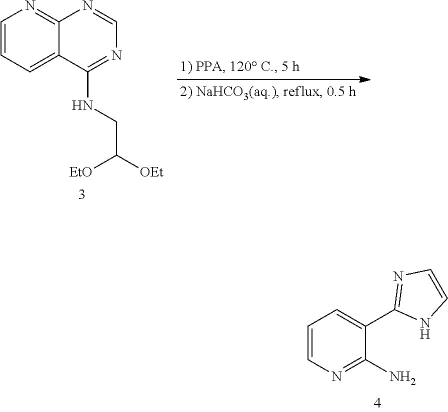 [SMILES: CCOC(CNc1ncnc2ncccc12)OCC ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 4 (0.3 g, crude). The filter cake was added saturated sodium bicarbonate solution (20 mL) and stirred at 110° C. for 0.5 hour. The reaction solution was extracted with THF (5×20 mL), combined crude product 4. The mixture was concentrated in vacuo and purified by silica gel chromatography using a mixture of petroleum ether-ethyl acetate (80:20, v/v) as eluent to afford 4 (448.0 mg, yield: 56%).

### The Synthesis of (4-fluoro-2-nitrophenyl) methanol (6)

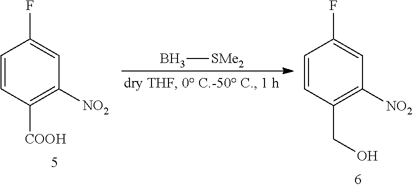

To a 100 mL three-necked flask were added 5 (2.0 g, 10.8 mmol) and THF (30 mL). The mixture was cooled to 0° C. in an ice-water bath, slowly added BH₃—SMe₂ (16.2 mL, 32.0 mmol) dropwise, then heated to 50° C. and stirred for 1 hour. After the reaction is complete, MeOH (20 mL) and water (50 mL) were added. The mixture was concentrated to remove organic solvents, extracted by EA (30 mL×5), combined organic layer, washed with brine (30 mL×3), dried over anhydrous sodium sulfate, filtered and concentrated the filtrate to afford a light yellow solid 6 (1.9 g, yield: 98%) without further purification.

### The Synthesis of 4-fluoro-2-nitrobenzaldehyde (7)

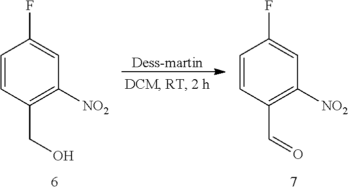 [SMILES: O=[N+]([O-])c1cc(F)ccc1CO ; "Dess-martin" ; O=Cc1ccc(F)cc1[N+](=O)[O-]]

To a 250 mL round bottom flask were added 6 (1.9 g, 11.1 mmol), DCM (40 mL) and Dess-Martin (7.1 g, 16.7 mmol) at 0° C., the mixture was stirred at room temperature for 2 hours. After the reaction is complete, water (50 mL) was added. The mixture was concentrated to remove organic solvents, extracted by EA (30 mL×3), combined organic layer, washed with brine (30 mL×2), dried over anhydrous sodium sulfate, filtered and concentrated the filtrate, purified by silica gel chromatography using a mixture of PE-EA (95:5, v/v) as eluent to afford to afford 7 (1.3 g, yield: 69%).

### The Synthesis of 1-(dibutoxymethyl)-4-fluoro-2-nitrobenzene (8)

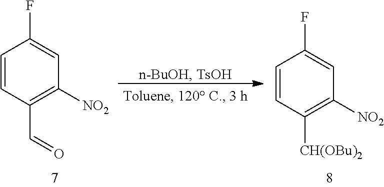

To a 100 mL round bottom flask were added 7 (500.0 mg, 2.96 mmol), n-butanol (548.2 mg, 7.39 mmol), p-toluenesulfonic acid monohydrate (11.0 mg, 0.06 mmol) and toluene (20 mL). The mixture was stirred at 120° C. and equipped with Dean-Stark equipment for 3 hours. Once the reaction completed, the reaction mixture was concentrated and purify by silica gel chromatography using a mixture of PE-EA (95:5, v/v) as eluent to afford to afford 8 (679.5 mg, yield: 76%).

### The Synthesis of 4-fluoro-1H-indole-7-carbaldehyde (9)

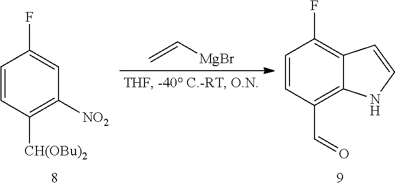

To a 100 mL three-necked flask were added 8 (450.0 mg, 1.5 mmol) and THF (15 mL), the solution was degassed and refilled Ar three times, then cooled to −40° C. and slowly added vinylmagnesium bromide (7 mL, 1 M in THF) by syringe over 1 hour in stirring. The reaction was warmed slowly to room temperature and stirred overnight. The reaction solution was quenched carefully by saturated aqueous ammonium chloride solution (20 mL) and water (30 mL), extracted by EA (30 mL×3), combined the organic layer, washed with brine (30 mL×2), dried over anhydrous sodium sulfate, filtered, concentrated the filtrate to give a yellow oil. The residue was dissolved in THF (5 mL) and hydrochloric acid (5 mL, 0.5 M in water), stirred at room temperature for 1 hour, then basified by saturated sodium bicarbonate solution till pH~8. The mixture was extracted by EA (20 mL×3), combined the organic layer, washed with brine (20 mL×2), dried over anhydrous sodium sulfate, filtered, concentrated the filtrate and purified by silica gel chromatography using a mixture of PE-EA (60:40, v/v) as eluent to afford 9 (185 mg, yield: 66%).

### The Synthesis of N-((4-fluoro-1H-indol-7-yl)methyl)-3-(1H-imidazol-2-yl)pyridine-2-amine (ELG-000536)

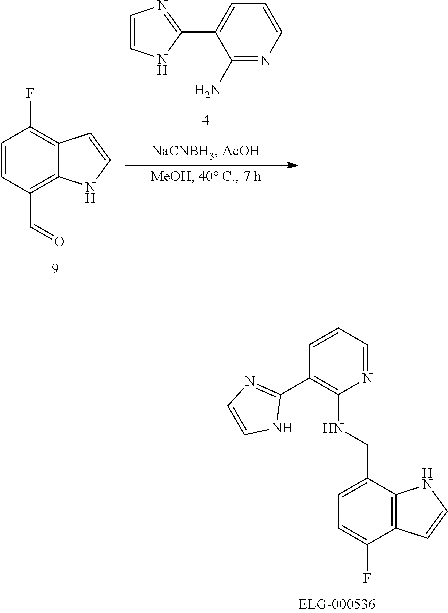 [SMILES: Nc1ncccc1-c1ncc[nH]1 ; O=Cc1ccc(F)c2cc[nH]c12 ; Fc1ccc(CNc2ncccc2-c2ncc[nH]2)c2[nH]ccc12]

To a 100 mL round bottom flask were added 4 (50.0 mg, 0.31 mmol), MeOH (3 mL), 9 (51.0 mg, 0.31 mmol) and AcOH (3 drops), the mixture was stirred at 40° C. until 9 consumed, about 2 hours. Then added NaCNBH₃ (72.3 mg, 1.15 mmol), the reaction mixture was stirred at room temperature for another 5 hours. The reaction mixture was quenched by water (1 mL), after prep. HPLC (a mixture of CH₃CN and H₂O with 0.5% HCO₂H as eluent) purification, the eluent was concentrated to remove organic solvents. The residual aqueous solution was lyophilized to afford ELG-000536 (16.0 mg, yield: 16%) as a yellow solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 96.96%, Rt=1.240 min: MS Calcd: 307.12: MS Found: 308.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 96.74%, Rt=4.577 min. ¹H NMR (400 MHz, DMSO-d₆) δ 12.71 (brs, 1H), 11.54 (s, 1H), 9.69 (brs, 1H), 8.08 (dt, J=7.4, 3.6 Hz, 2H), 7.41 (t, J=2.8 Hz, 1H), 7.35-6.95 (m, 3H), 6.71 (dd, J=10.5, 8.0 Hz, 2H), 6.52 (dd, J=3.1, 1.9 Hz, 1H), 4.95 (s, 2H). Chemical Formula: C₁₇H₁₄N₅F. Molecular Weight: 307.33.

### Route for ELG-000537

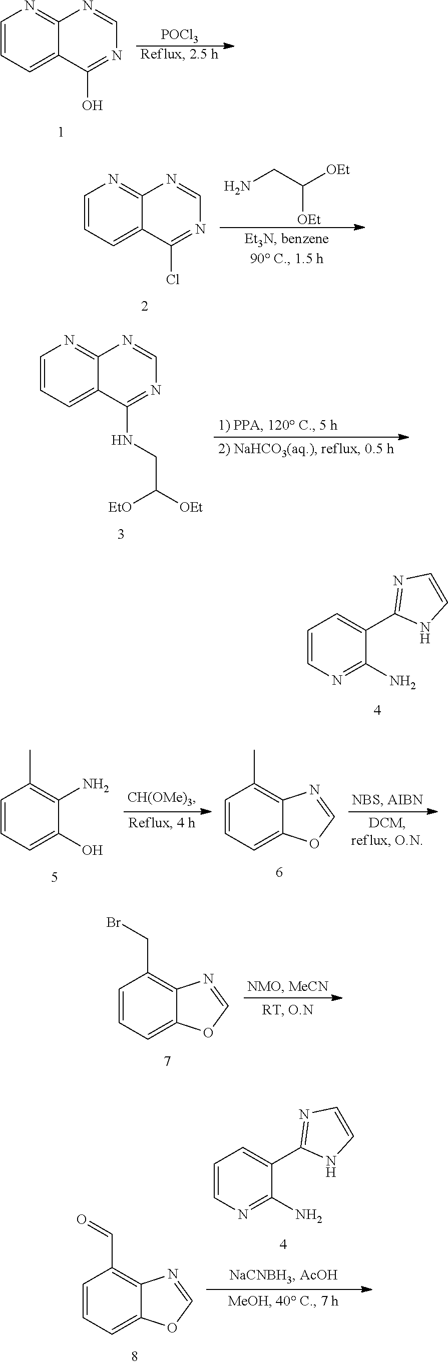

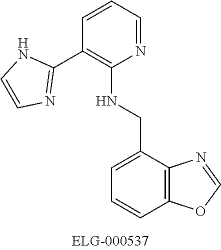 [SMILES: c1cnc(NCc2cccc3ocnc23)c(-c2ncc[nH]2)c1]

### The Synthesis of 4-chloropyrido[2,3-cd]pyrimidine (2)

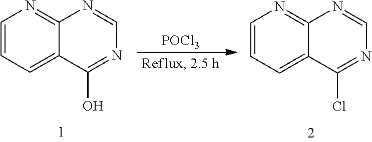

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

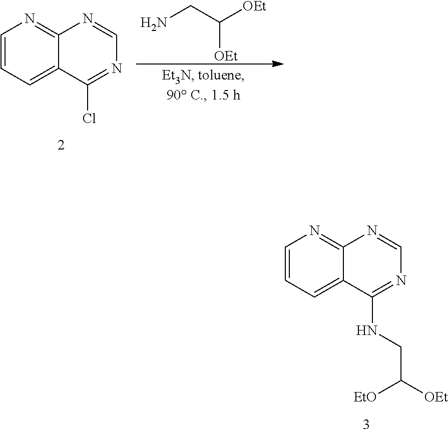 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (4)

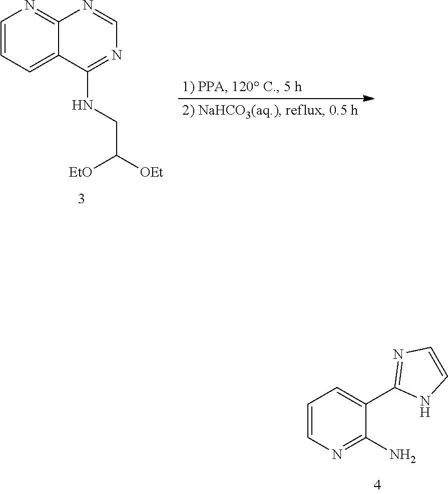 [SMILES: CCOC(CNc1ncnc2ncccc12)OCC ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 4 (0.3 g, crude). The filter cake was added saturated sodium bicarbonate solution (20 mL) and stirred at 110° C. for 0.5 hour. The reaction solution was extracted with THF (5×20 mL), combined crude product 4. The mixture was concentrated in vacuo and purified by silica gel chromatography using a mixture of petroleum ether-ethyl acetate (80:20, v/v) as eluent to afford 4 (448.0 mg, yield: 56%).

### The Synthesis of 4-methylbenzo[d]oxazole (6)

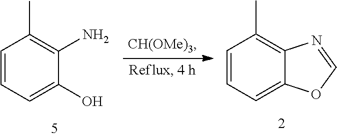

To a 100 mL round bottom flask were added 5 (616 mg, 5.0 mmol) and trimethoxymethane (15 mL), the reaction mixture was stirred at 110° C. for 4 hours. The reaction mixture was concentrated in vacuo and purified by silica gel chromatography using a mixture of petroleum ether-ethyl acetate (80:20, v/v) as eluent to afford 6 (483 mg, yield: 73%).

### The Synthesis of 4-(bromomethyl)benzo[d]oxazole (7)

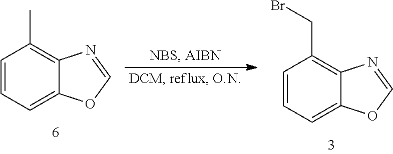

To a 100 mL round bottom flask were added 6 (483 mg, 3.6 mmol), NBS (840 mg, 4.7 mmol), AIBN (12 mg, 0.1 mmol) and DCM (40 mL), the reaction mixture was stirred at 45° C. overnight. Once the reaction completed, the reaction mixture was concentrated in vacuo and extracted with EA (20 mL×3), the organic phase was washed with saturated sodium bicarbonate aqueous solution (20 mL×3), the organic phase was combined and dried over anhydrous Na₂SO₄ and concentrated in vacuo. The crude product was purified by silica gel chromatography using a mixture of petroleum ether-ethyl acetate (95:5, v/v) as eluent to afford 7 (571 mg, yield: 74%).

### The Synthesis of benzo[d]oxazole-4-carbaldehyde (8)

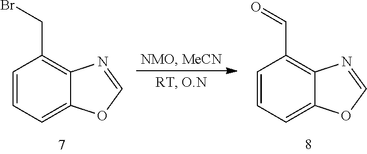 [SMILES: BrCc1cccc2ocnc12 ; O=Cc1cccc2ocnc12]

To a 100 mL round bottom flask were added 7 (250 mg, 1.18 mmol), NMO (213 mg, 1.81 mmol), acetonitrile (4 mL), the reaction mixture was stirred at room temperature for 1 hour. Once the reaction completed, water (20 mL) was added in the reaction mixture and extracted with EA (15 mL×3), the organic phase was combined, dried over anhydrous Na₂SO₄ and concentrated in vacuo. The crude product was purified by silica gel chromatography using a mixture of petroleum ether-ethyl acetate (90:10, v/v) as eluent to afford 8 (80 mg, yield: 42%).

### The Synthesis of N-(benzo[d]oxazol-4-ylmethyl)-3-(1H-imidazol-2-yl)pyridin-2-amine (ELG-000537)

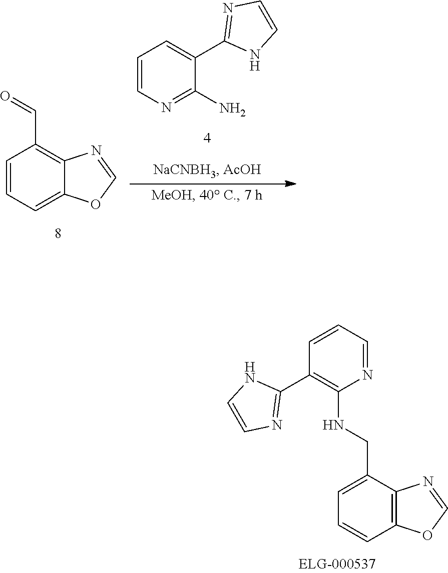 [SMILES: O=Cc1cccc2ocnc12 ; Nc1ncccc1-c1ncc[nH]1 ; c1cnc(NCc2cccc3ocnc23)c(-c2ncc[nH]2)c1]

To a 100 mL bottom flask were added 4 (30.0 mg, 0.19 mmol), 8 (27.9 mg, 0.19 mmol), AcOH (3 drops) and Methanol (2 mL), the mixture stirred at 40° C. for 2 hours. Then the reaction mixture was added NaBH₃CN (72.1 mg, 0.97 mmol) and stirred at the same temperature for another 5 hours. The mixture was directly purified by prep-TLC (a mixture of petroleum ether and ethyl acetate, 1:1, v/v) to afford ELG-000537 (24.8 mg, yield: 45%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 99.69%, Rt=1.059 min; MS Calcd: 291.11; MS Found: 292.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 98.85%, Rt=3.680 min. ¹H NMR (400 MHz, DMSO-d₆) δ 12.64 (brs, 1H), 9.79 (brs, 1H), 8.75 (s, 1H), 8.08-7.98 (m, 2H), 7.64 (dd, J=8.0, 1.0 Hz, 1H), 7.40-7.27 (m, 3H), 7.09 (brs, 1H), 6.66 (dd, J=7.5, 4.9 Hz, 1H), 5.10 (d, J=5.5 Hz, 2H). Chemical Formula: C₁₆H₁₃N₅O. Molecular Weight: 291.31.

### Route for ELG-000538

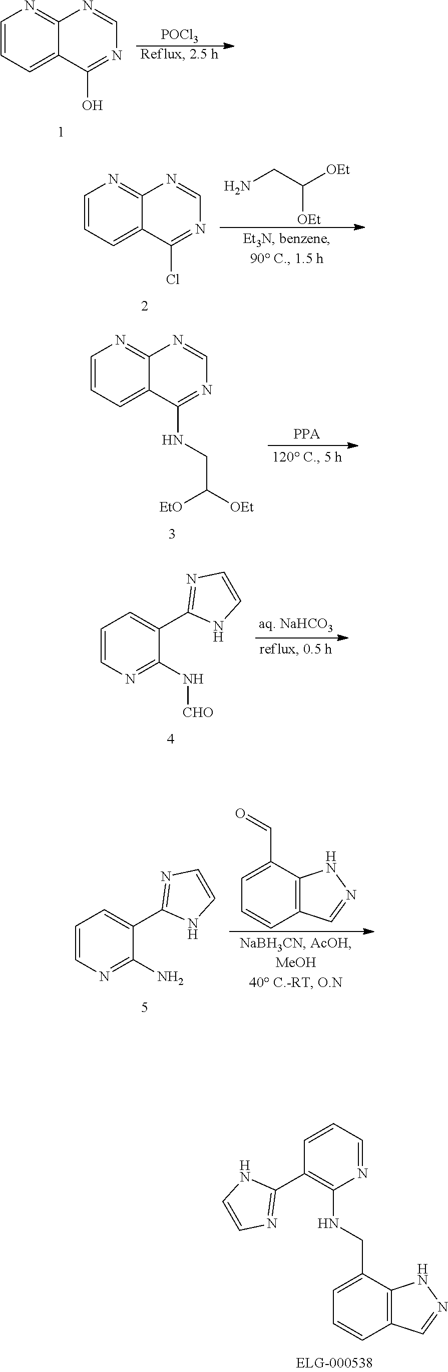

### The Synthesis of 4-chloropyrido[2,3-d]pyrimidine (2)

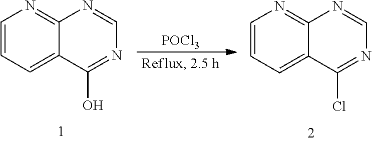

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

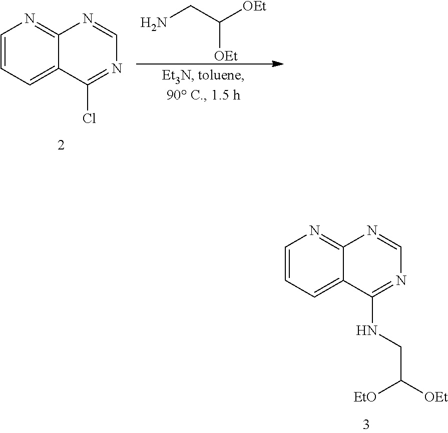 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (5)

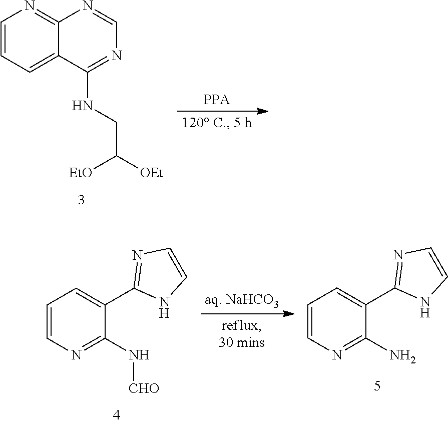 [SMILES: CCOC(CNc1ncnc2ncccc12)OCC ; O=CNc1ncccc1-c1ncc[nH]1 ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 5 (0.3 g, crude). The filter cake was added saturated sodium bicarbonate solution (20 mL) and stirred at 110° C. for 0.5 hour. The reaction solution was extracted with THF (5×20 mL), combined crude product 5. The mixture was concentrated in vacuo and purified by silica gel chromatography using a mixture of petroleum ether-ethyl acetate (80:20, v/v) as eluent to afford 5 (448.0 mg, yield: 56%).

### The Synthesis of N-((1H-indazol-7-yl)methyl)-3-(1H-imidazol-2-yl)pyridin-2-amine (ELG-000538)

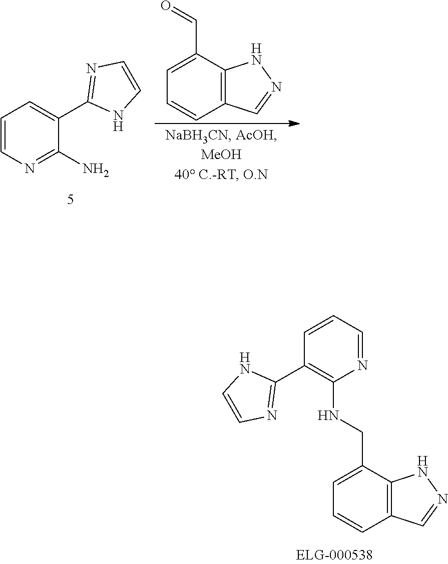

To a 100 mL bottom flask were added 5 (40.0 mg, 0.25 mmol), 1H-indazole-7-carbaldehyde (37.0 mg, 0.25 mmol). AcOH (5 drops) and Methanol (3 mL), the mixture stirred at 40° C. for 2 hours. Then the reaction mixture was added NaBH₃CN (62.8 mg, 1.00 mmol) and stirred at room temperature overnight. The mixture was directly purified by prep-TLC (a mixture of petroleum ether and ethyl acetate, 1:1, v/v) to afford ELG-000538 (10.8 mg, yield: 14%) as a white solid. LC-MS (Agilent LCMS 1260-6125B. Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100.00%, Rt=1.064 min; MS Calcd: 290.13; MS Found: 291.1 [M+H]⁺. HPLC (Waters UPLC. Column: ACQUITY UPLC BEH C18 1.7 μm*2.1 mm*150 mm); Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 100.00%, Rt=3.696 min. ¹H NMR (400 MHz, DMSO-d₆) δ 13.23 (s, 1H), 12.64 (s, 1H), 9.77 (t, J=5.5 Hz, 1H), 8.09 (s, 1H), 8.08-8.02 (m, 2H), 7.63 (d, J=8.0 Hz, 1H), 7.33 (s, 1H), 7.25 (d, J=6.7 Hz, 1H), 7.09 (s, 1H), 7.07-7.00 (m, 1H), 6.67 (dd, J=7.5, 5.0 Hz, 1H), 5.05 (d, J=5.6 Hz, 2H). Chemical Formula: C₁₆H₁₄N₆. Molecular Weight: 290.33.

### Route for ELG-000543

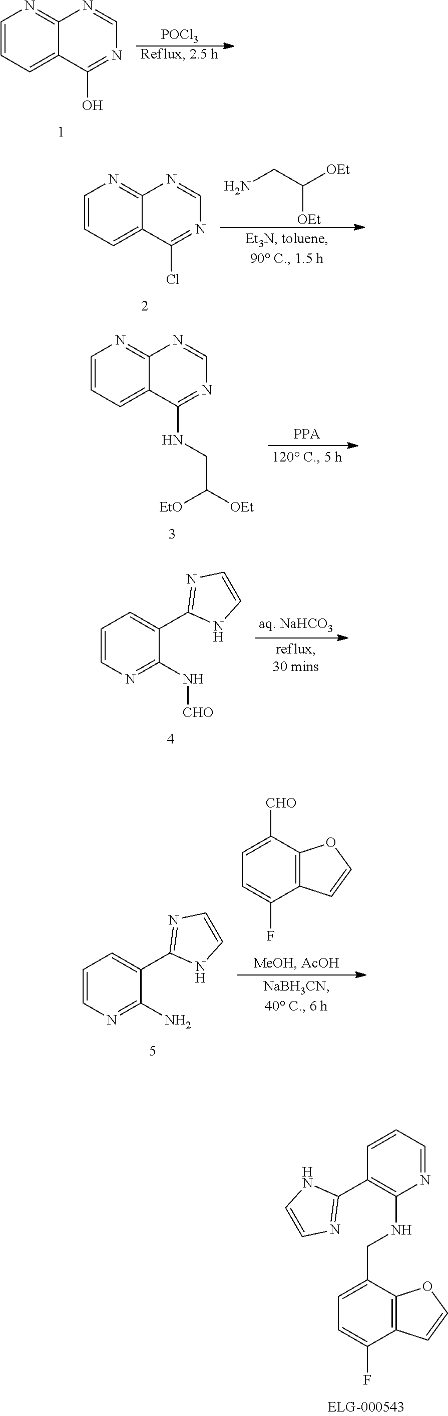

### The Synthesis of 4-chloropyrido[2,3-d]pyrimidine (2)

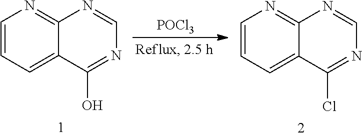

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

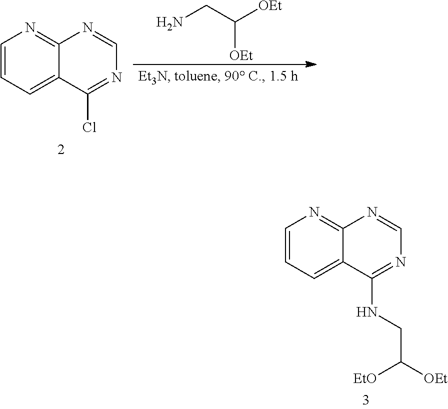 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (5)

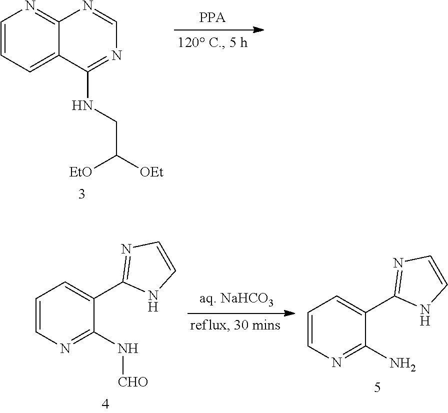 [SMILES: CCOC(CNc1ncnc2ncccc12)OCC ; O=CNc1ncccc1-c1ncc[nH]1 ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 5 (0.3 g, crude). The filter cake was added saturated sodium bicarbonate solution (20 mL) and stirred at 110° C. for 0.5 hour. The reaction solution was extracted with THF (5×20 mL), combined crude product 5. The mixture was concentrated in vacuo and purified by silica gel chromatography using a mixture of petroleum ether-ethyl acetate (80:20, v/v) as eluent to afford 5 (448.0 mg, yield: 56%).

### The Synthesis of N-((4-fluorobenzofuran-7-yl)methyl)-3-(1H-imidazol-2-yl)pyridin-2-amine (ELG-000543)

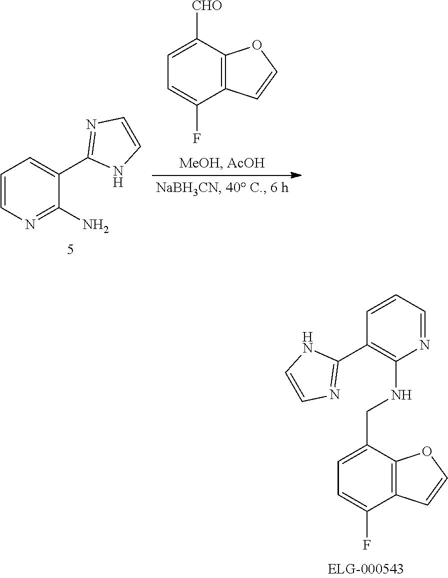

To a 100 mL round bottom flask were added 5 (29.3 mg, 0.18 mmol), 4-fluorobenzofuran-7-carbaldehyde (30 mg, 0.18 mmol), AcOH (5 drops) and MeOH (5 mL), the mixture stirred at 40° C. for 2 hours. Then the reaction mixture was added NaBH₃CN (47.0 mg, 0.75 mmol) and stirred at 40° C. for another 4 hours. The mixture was directly purified by prep-HPLC (a mixture of CH₃CN and H₂O with 0.5% HCO₂H as eluent) to afford ELG-000543 (13.7 mg, yield: 24%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%, Rt=1.170 min; MS Calcd: 308.11; MS Found: 309.2 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 98.69%, Rt=4.605 min. ¹H NMR (400 MHz, DMSO-d₆) δ 12.63 (s, 1H), 9.76 (t, J=5.7 Hz, 1H), 8.10 (d, J=2.2 Hz, 1H), 8.03 (ddd, J=6.7, 6.2, 1.7 Hz, 2H), 7.31 (s, 1H), 7.23 (dd, J=8.2, 5.3 Hz, 1H), 7.11-7.06 (m, 2H), 7.03 (dd, J=9.7, 8.3 Hz, 1H), 6.66 (dd, J=7.5, 4.9 Hz, 1H), 5.00 (d, J=5.7 Hz, 2H). Chemical Formula: C₁₇H₁₃FN₄O. Molecular Weight: 308.32.

### Route for ELG-000549

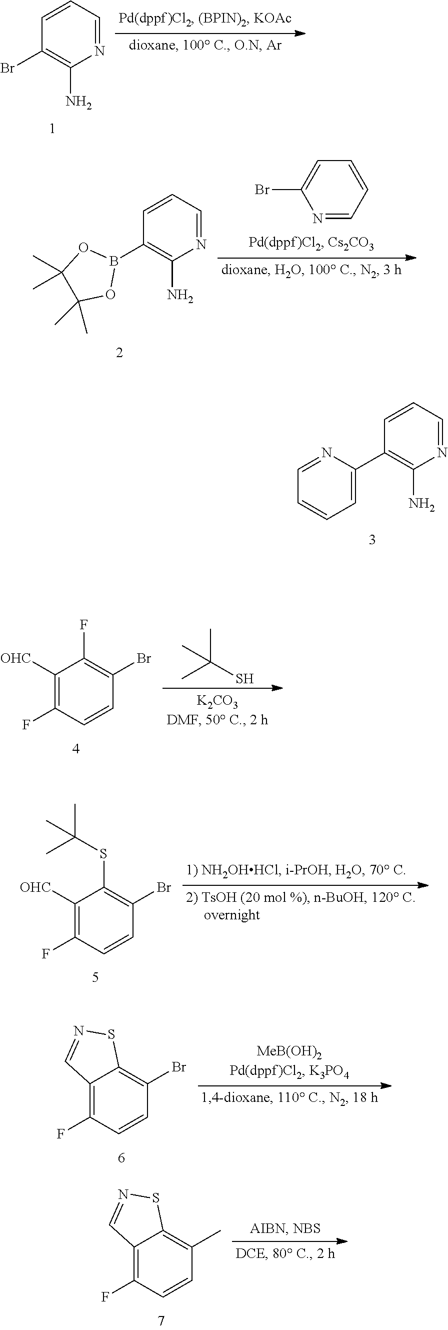

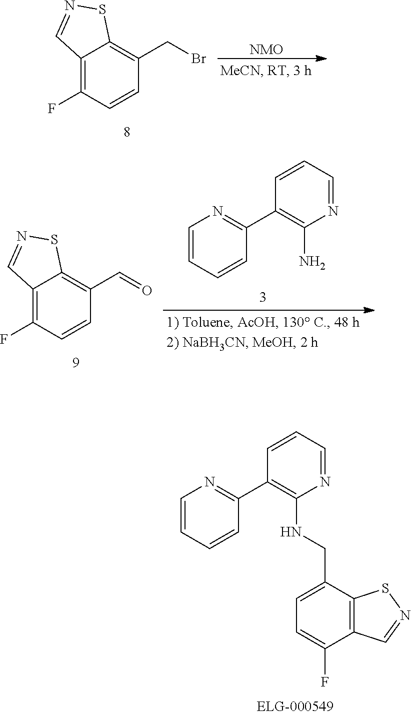

The synthesis of 3-(4,4,5,5-tetramethyl-1,3,2-dioxaborolan-2-yl)pyridin-2-amine (2)

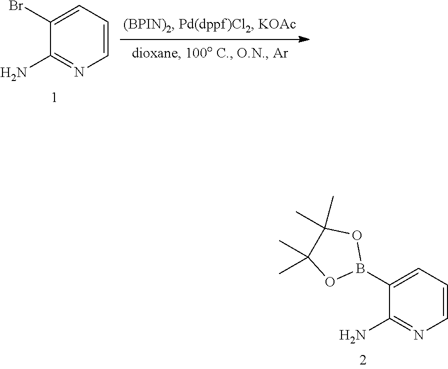 [SMILES: Nc1ncccc1Br ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

The synthesis of [2,3′-bipyridin]-2′-amine (3)

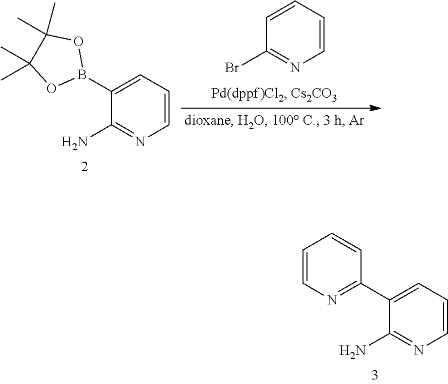 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 2-bromopyridine (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 3 (1.0 g, yield: 40%).

The synthesis of 3-bromo-2-(tert-butylthio)-6-fluorobenzaldehyde (5).

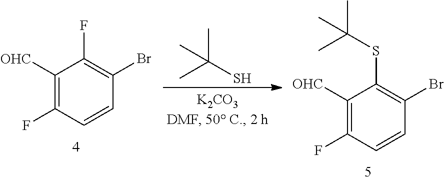

To a 250 mL round bottom flask were added 4 (5.0 g, 22.62 mmol), 2-methylpropane-2-thiol (2.0 g, 21.55 mmol), K₂CO₃ (4.5 g, 32.33 mmol) and DMF (30 mL). Then the reaction mixture was stirred at 50° C. for 2 hours. Waters (100 mL) was added and the mixture was extracted with EA (50 mL×3), the combined organic layers were washed with brine, dried over anhydrous Na₂SO₄, filtered and the filtrate was concentrated. The crude product was purified by silica gel chromatography using a mixture of petroleum ether-ethyl acetate (90:10, v/v) as eluent to afford 5 (6.06 g, yield: 96%).

The synthesis of 7-bromo-4-fluorobenzo[d]isothiazole (6).

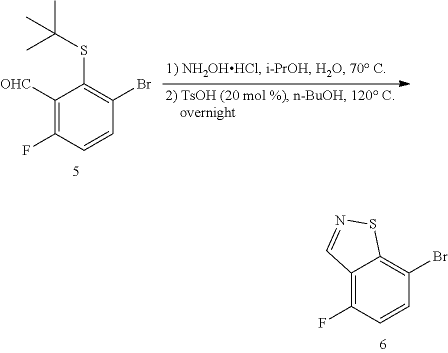

To a 250 mL round bottom flask were added 5 (6.06 g, 20.81 mmol), NH₂OH HCl (7.28 g, 104.06 mmol), i-PrOH (80 mL) and H₂O (15 mL). The reaction mixture was stirred at 70° C. for 20 mins. The organic solvent was removed in vacuo and saturated NaHCO₃ solution was added till pH>8, then the mixture was extracted with DCM (30 mL×3) and H₂O (60 mL), the organic layer was concentrated in vacuo, the residue was treated with TsOH (0.72 g, 4.16 mmol) in n-BuOH (80 mL), and the mixture was stirred at 120° C. overnight. The reaction mixture was concentrated and purified by silica gel chromatography using a mixture of petroleum ether-ethyl acetate (95:5, v/v) as eluent to afford 6 (3.3 g, yield: 69%).

The synthesis of 4-fluoro-7-methylbenzo[d]isothiazole (7).

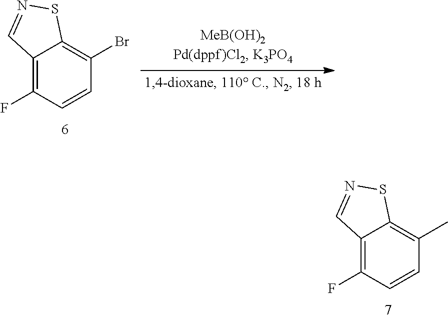

To a 250 mL round bottom flask were added 6 (3.3 g, 14.29 mmol), MeB(OH)₂ (2.57 g, 42.87 mmol), K₃PO₄ (9.10 g, 42.87 mmol), Pd(dppf)Cl₂ (0.21 g, 0.29 mmol), dioxane (50 mL), H₂O (10 mL). The reaction mixture was degassed and refilled with N₂ five times, stirred at 100° C. for 3 hours. Waters (100 mL) was added and the mixture was extracted with EA (50 mL×3), the combined organic layers were washed with brine, dried over anhydrous Na₂SO₄, filtered and the filtrate was concentrated. The crude product was purified by silica gel chromatography by using a mixture of petroleum ether-ethyl acetate (98:2, v/v) as eluent to afford 7 (1.9 g. yield: 79%).

### The Synthesis of 7-(bromomethyl)-4-fluorobenzo[d]isothiazole (8)

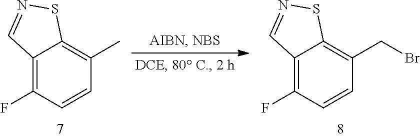

To a 100 mL round bottom flask were added 7 (1.82 g, 10.09 mmol), NBS (1.98 g, 11.12 mmol), AIBN (0.17 g, 1.01 mmol) and DCE (20 mL). The reaction mixture was stirred at 80° C. for 2 hours. The reaction mixture was concentrated and purified by silica gel chromatography using a mixture of petroleum ether-ethyl acetate (95:5, v/v) as eluent to afford 8 (1.8 g, yield: 73%).

### The Synthesis of 4-fluorobenzo[cd]isothiazole-7-carbaldehyde (9)

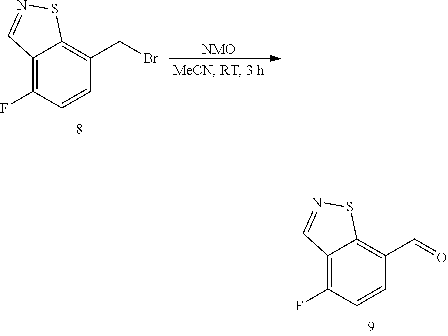

To a 100 mL round bottom flask were added 8 (1.30 g, 5.31 mmol). NMO (4.97 g, 42.45 mmol), MeCN (30 mL). Then the reaction mixture was stirred at room temperature for 3 hours. The reaction mixture was concentrated and purified by silica gel chromatography by using a mixture of petroleum ether-ethyl acetate (95:5, v/v) as eluent to afford 9 (700 mg, yield: 70%).

### The Synthesis of N-((4-fluorobenzo[d]isothiazol-7-yl)methyl)-[2,3′-bipyridin]-2′-amine (ELG-000549)

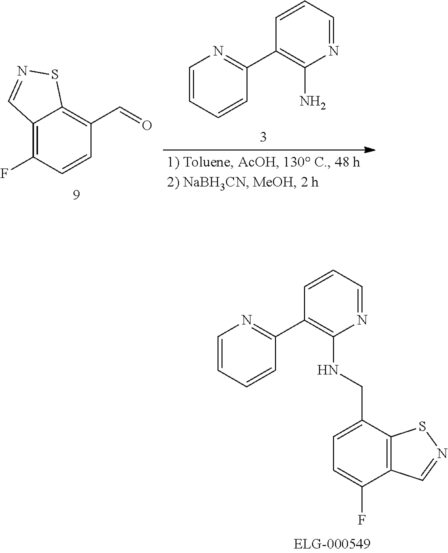

To a 50 mL round bottom flask were added 9 (60 mg, 0.33 mmol), 3 (56.5 mg, 0.33 mmol), Toluene (10 mL) and AcOH (8 drops), the reaction mixture was stirred at 130° C. for 48 hours with Dean-Stark equipment. After the reaction was completed, the organic solvent was removed in vacuo. Then to the residue was added MeOH (5 mL) and NaBH₃CN (79 mg, 1.25 mmol), stirred at room temperature for 2 hours. The reaction mixture was quenched with H₂O and extracted with EA (10 mL×3), the combined organic layers were washed with brine, dried over anhydrous Na₂SO₄, filtered and the filtrate was concentrated. The crude product was purified by prep-TLC to afford ELG-000549 (26.8 mg, yield: 24%) as a white solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 98.10%, Rt=1.264 min: MS Calcd: 336.08: MS Found: 337.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 96.23%, Rt=4.656 min. ¹H NMR (400 MHz, DMSO-d₆) δ 9.93 (t, J=5.6 Hz, 1H), 9.12 (s, 1H), 8.68 (ddd, J=4.9, 1.8, 0.9 Hz, 1H), 8.14 (dd, J=7.7, 1.7 Hz, 1H), 8.10-7.92 (m, 3H), 7.60 (dd, J=8.0, 4.7 Hz, 1H), 7.41 (ddd, J=7.4, 4.9, 1.1 Hz, 1H), 7.30 (dd, J=10.0, 8.0 Hz, 1H), 6.73 (dd, J=7.6, 4.8 Hz, 1H), 5.02 (d, J=5.7 Hz, 2H). Chemical Formula: C18H₁₃FN₄S. Molecular Weight: 336.39.

### Route for ELG-000552

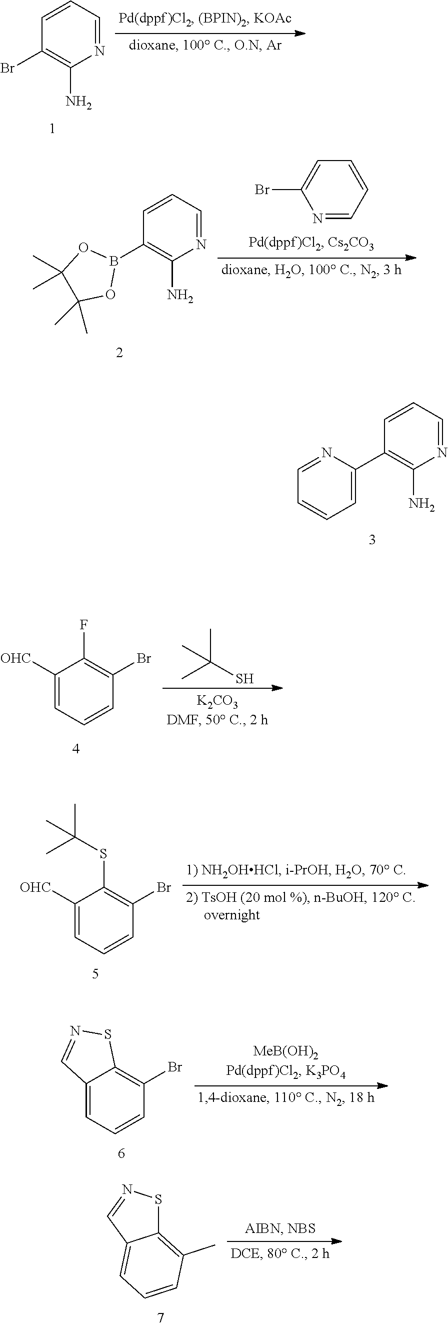

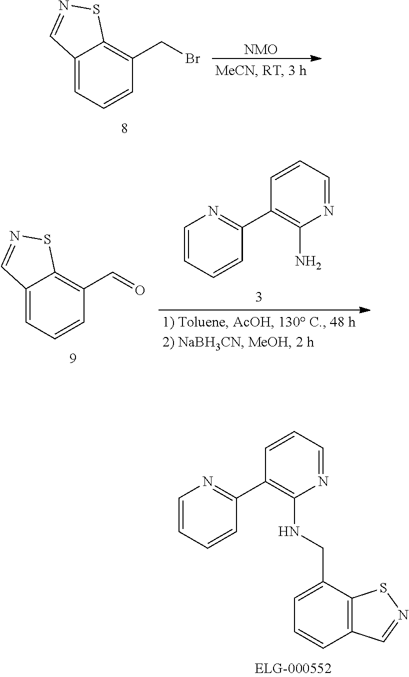

### The Synthesis of 3-(4,4,5,5-tetramethyl-1,3,2-dioxaborolan-2-yl)pyridin-2-amine (2)

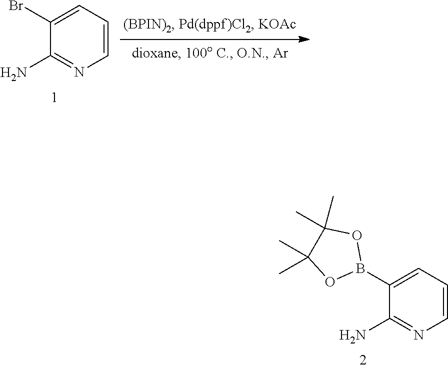 [SMILES: Nc1ncccc1Br ; CC1(C)OB(c2cccnc2N)OC1(C)C]

To a 250 mL round bottom flask were added 1 (2.5 g, 14.50 mmol), Pd(dppf)Cl₂ (530.6 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (4)

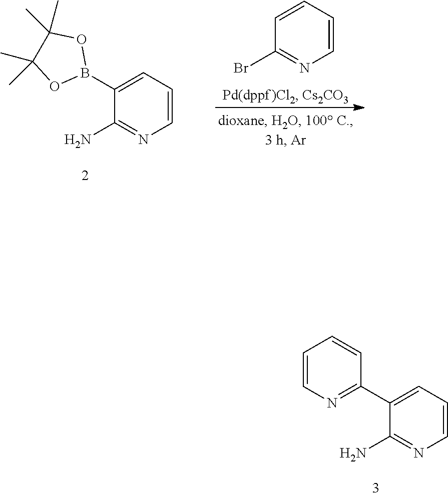 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol), Pd(dppf)Cl₂ (530.6 mg, 0.73 mmol), Cs₂CO₃ (9.5 g, 29.0 mmol), 2-bromopyridine (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 3 (1.0 g, yield: 40%).

### The Synthesis of 3-bromo-2-(tert-butylthio)benzaldehyde (5)

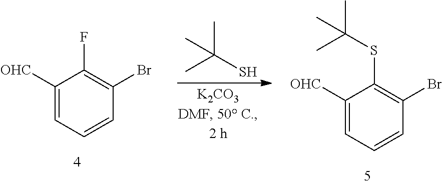

To a 250 mL round bottom flask were added 4 (3.5 g, 17.24 mmol), 2-methylpropane-2-thiol (1.6 g, 18.10 mmol), K₂CO₃ (3.6 g, 25.86 mmol) and DMF (30 mL). Then the reaction mixture was stirred at 50° C. for 2 hours. Waters (100 mL) was added and the mixture was extracted with EA (50 mL×3), the combined organic layers were washed with brine, dried over anhdrous Na₂SO₄, filtered and the filtrate was concentrated. The crude product was purified by silica gel chromatography using a mixture of petroleum ether-ethyl acetate (90:10, v/v) as eluent to afford 5 (3.3 g, yield: 70%).

### The Synthesis of 7-bromobenzo[d]isothiazole (6)

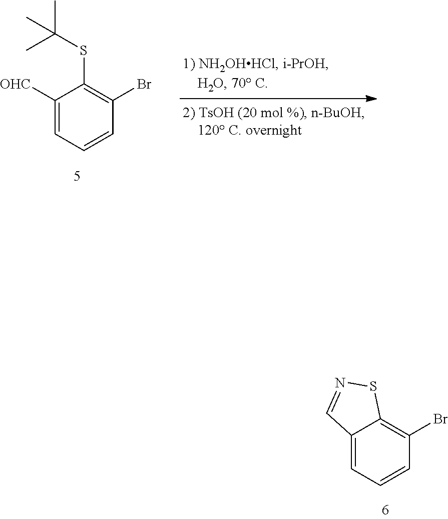

To a 250 mL round bottom flask were added 5 (3.3 g, 12.07 mmol). NH₂OH HCl (4.2 g, 60.40 mmol), i-PrOH (80 mL) and H₂O (15 mL). The reaction mixture was stirred at 70° C. for 20 mins. The organic solvent was removed in vacuo and saturated NaHCO₃ solution was added till pH>8, then the mixture was extracted with DCM (30 mL×3) and H₂O (60 mL), the organic layer was concentrated in vacuo, the residue was treated with TsOH (0.42 g, 2.41 mmol) in n-BuOH (80 mL), and the mixture was stirred at 120° C. overnight. The reaction mixture was concentrated and purified by silica gel chromatography using a mixture of petroleum ether-ethyl acetate (95:5, v/v) as eluent to afford 6 (1.1 g, yield: 42%).

### The Synthesis of 7-methylbenzo[d]isothiazole (7)

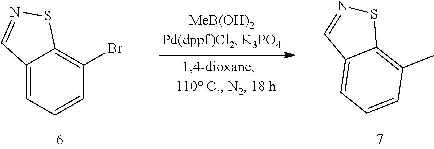

To a 250 mL round bottom flask were added 6 (1.1 g, 5.07 mmol), MeB(OH)₂ (0.9 g, 15.21 mmol), K₃PO₄ (3.2 g, 15.21 mmol), Pd(dppf)Cl₂ (0.2 g, 0.25 mmol), dioxane (50 mL), H₂O (10 mL). The reaction mixture was degassed and refilled with N₂ five times, stirred at 100° C. for 3 hours. Waters (100 mL) was added and the mixture was extracted with EA (50 mL×3), the combined organic layers were washed with brine, dried over anhydrous Na₂SO₄, filtered and the filtrate was concentrated. The crude product was purified by silica gel chromatography by using a mixture of petroleum ether-ethyl acetate (98:2, v/v) as eluent to afford 7 (460 mg, yield: 60%).

### The Synthesis of 7-(bromomethyl)benzo[d]isothiazole (8)

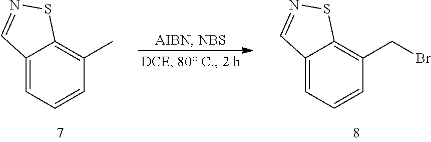

To a 100 mL round bottom flask were added 7 (460 mg, 3.08 mmol), NBS (603 mg, 3.39 mmol), AIBN (51 mg, 0.31 mmol) and DCE (10 mL). The reaction mixture was stirred at 80° C. for 2 hours. The reaction mixture was concentrated and purified by silica gel chromatography using a mixture of petroleum ether-ethyl acetate (95:5, v/v) as eluent to afford 8 (240 mg, yield: 34%).

### The Synthesis of benzo[d]isothiazole-7-carbaldehyde (9)

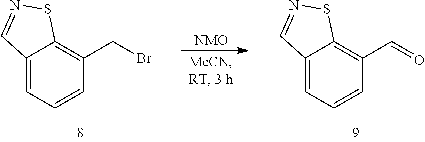

To a 100 mL round bottom flask were added 8 (240 mg, 1.05 mmol), NMO (984 mg, 8.40 mmol), MeCN (10 mL), Then the reaction mixture was stirred at room temperature for 3 hours. The reaction mixture was concentrated and purified by silica gel chromatography by using a mixture of petroleum ether-ethyl acetate (95:5, v/v) as eluent to afford 9 (120 mg, yield: 70%).

### The Synthesis of N-(benzo[d]isothiazol-7-ylmethyl)-[2,3′-bipyridin]-2′-amine (ELG-000552)

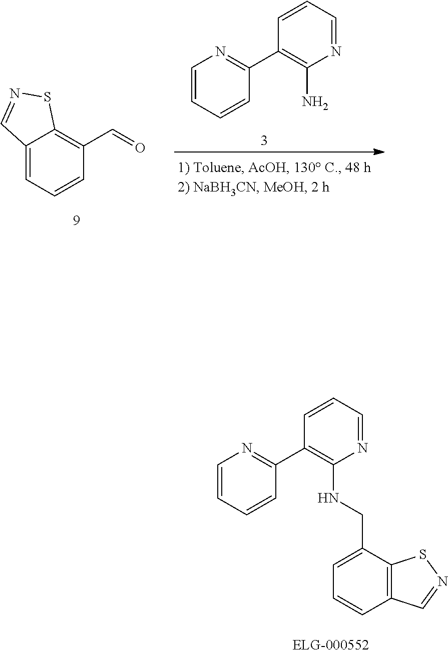

To a 50 mL round bottom flask were added 9 (30 mg, 0.18 mmol), 3 (30 mg, 0.17 mmol), Toluene (5 mL) and AcOH (8 drops), the reaction mixture was stirred at 130° C. for 48 hours with Dean-Stark equipment. After the reaction was completed, the organic solvent was removed in vacuo. Then to the residue was added MeOH (5 mL) and NaBH₃CN (33 mg, 0.53 mmol), stirred at room temperature for 2 hours. The reaction mixture was quenched with H₂O and extracted with EA (10 mL×3), the combined organic layers were washed with brine, dried over anhydrous Na₂SO₄, filtered and the filtrate was concentrated. The crude product was purified by prep-TLC to afford ELG-000552 (2.1 mg, yield: 4%) as a white solid. LC-MS (Agilent LCMS 1260-6125B, Column: Boltimate EXT C18 CoreShell (50 mm*4.6 mm*2.7 μm); Column Temperature: 45° C.: Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 100%. Rt=1.171 min; MS Calcd: 318.09; MS Found: 319.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 95.77%, Rt=3.942 min. ¹H NMR (400 MHz, DMSO-d₆) δ 11.61 (brs, 1H), 9.17 (s, 1H), 8.76-8.66 (m, 2H), 8.29 (d, J=8.2 Hz, 1H), 8.23-8.12 (m, 2H), 8.10-8.03 (m, 1H), 7.75 (d, J=7.1 Hz, 1H), 7.65-7.52 (m, 2H), 7.11 (dd. J=7.4, 6.2 Hz, 1H), 5.34 (s, 2H). Chemical Formula: C18H₁₄N₄S. Molecular Weight: 318.40.

### Route for ELG-000560

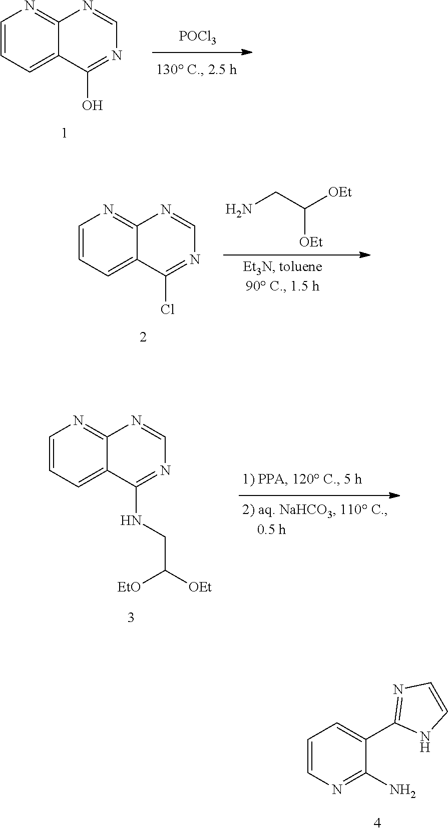

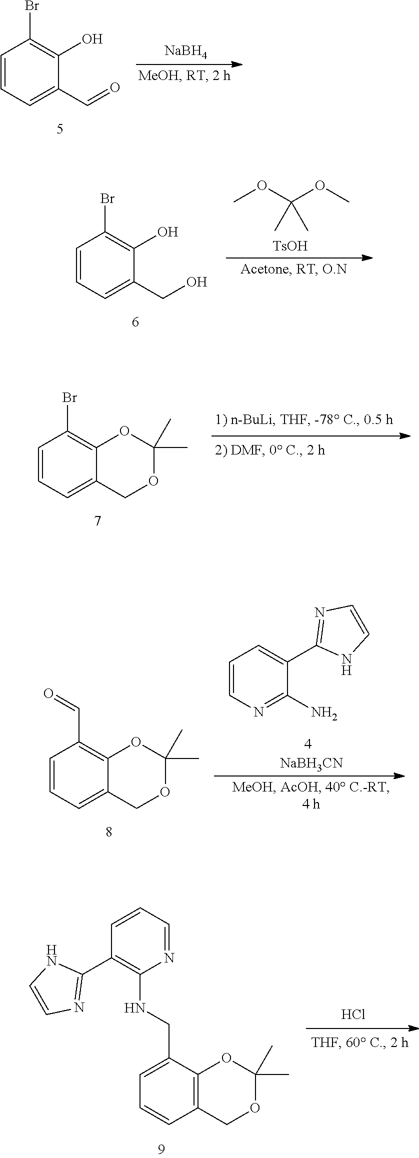

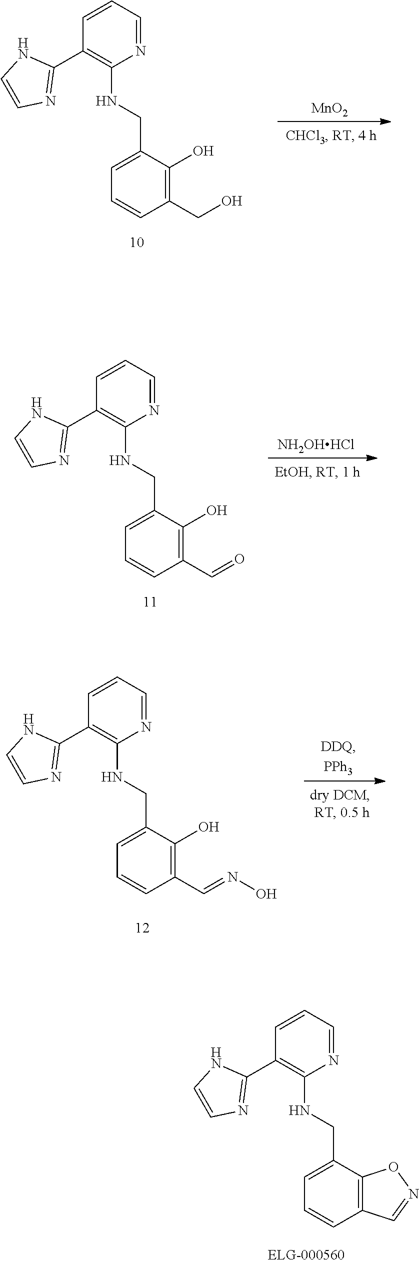

### The Synthesis of 4-chloropyrido[2,3-d]pyrimidine (2)

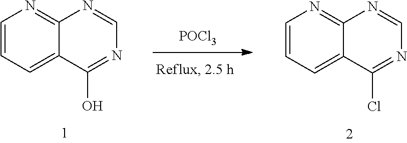

To a 250 mL round bottom flask were added 1 (4.5 g, 30.5 mmol) and POCl₃ (80 mL), the mixture was stirred at 130° C. for 2.5 hours. The reaction solution was concentrated in vacuo, the residue was diluted by ice water (200 mL) and THF (50 mL), basified carefully by saturated sodium carbonate aqueous till pH>8, extracted with THF (5×40 mL), the combined organic phase was dried over anhydrous Na₂SO₄ and concentrated in vacuo to afford 2 (1.4 g, yield: 28%) without further workup.

### The Synthesis of N-(2,2-diethoxyethyl)pyrido[2,3-d]pyrimidin-4-amine (3)

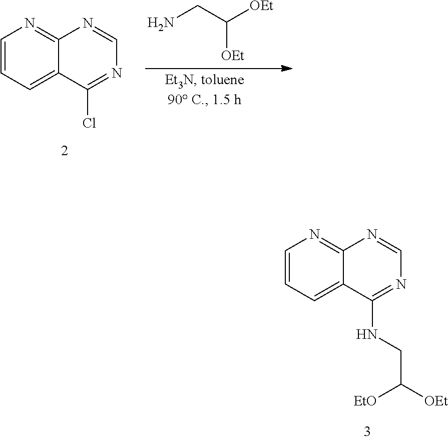 [SMILES: Clc1ncnc2ncccc12 ; CCOC(CN)OCC ; CCOC(CNc1ncnc2ncccc12)OCC]

To a 250 mL round bottom flask were added 2 (1.4 g, 8.38 mmol), 2,2-diethoxyethan-1-amine (1.6 g, 12.53 mmol), triethylamine (1.1 g, 10.9 mmol) and toluene (70 mL), the mixture was stirred at 90° C. for 1.5 hours. The reaction solution was concentrated in vacuo and the residue was purified by silica gel chromatography using a mixture of dichloromethane-methanol (95:5, v/v) as eluent to afford 3 (1.3 g, yield: 59%).

### The Synthesis of 3-(1H-imidazol-2-yl)pyridin-2-amine (4)

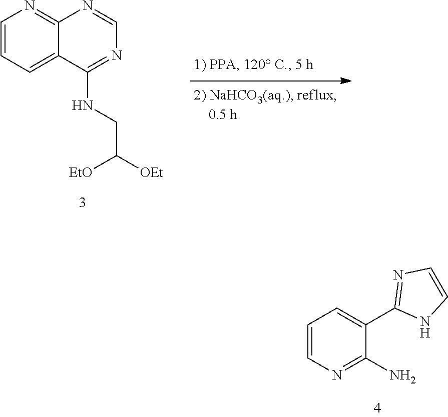 [SMILES: CCOC(CNc1ncnc2ncccc12)OCC ; Nc1ncccc1-c1ncc[nH]1]

To a 250 mL round bottom flask were added 3 (1.3 g, 4.96 mmol) and polyphosphoric acid (8 mL), the mixture was stirred at 120° C. for 5 hours. The reaction solution was quenched carefully by ice water (40.0 mL), then the mixture was neutralized by saturated aqueous sodium carbonate and filtered, the filtrate was extracted with THF (5×30 mL), the combined organic layer was concentrated in vacuo to afford 4 (0.3 g, crude). The filter cake was added saturated sodium bicarbonate solution (20 mL) and stirred at 110° C. for 0.5 hour. The reaction solution was extracted with THF (5×20 mL), combined crude product 4. The mixture was concentrated in vacuo and purified by silica gel chromatography using a mixture of petroleum ether-ethyl acetate (80:20, v/v) as eluent to afford 4 (448.0 mg, yield: 56%).

### The Synthesis of 2-bromo-6-(hydroxymethyl)phenol (6)

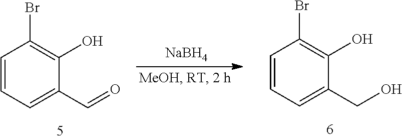 [SMILES: O=Cc1cccc(Br)c1O ; "NaBH4" ; OCc1cccc(Br)c1O]

To a 100 mL round bottom flask were added 5 (2.85 g, 14.25 mmol) and MeOH (30 mL), then slowly add NaBH₄ (0.83 g, 21.38 mmol) at 0° C., and the reaction mixture was stirred at room temperature for 2 hours. The reaction mixture was quenched with H₂O, then the mixture was extracted with EA (30 mL×3), the combined organic layers were washed with brine, dried over anhydrous Na₂SO₄, filtered and the filtrate was concentrated in vacuo to afford 6 (1.8 g) as crude product for the next step without additional purification.

### The Synthesis of 8-bromo-2,2-dimethyl-4H-benzo[d][1,3]dioxine (7)

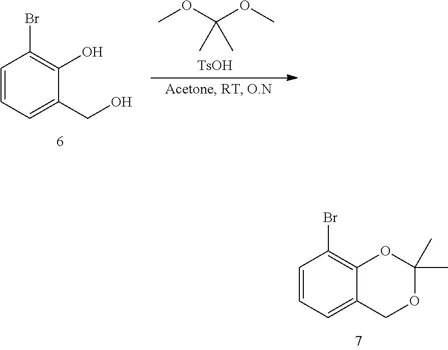

To a 100 mL round bottom flask were added 6 (1.80 g, 8.91 mmol), 2,2-dimethoxypropane (3.25 g, 31.19 mmol), TsOH (310 mg, 1.78 mmol) and acetone (30 mL), the mixture was stirred at room temperature overnight. The reaction mixture was quenched with saturated NaHCO₃ aqueous, then the mixture was extracted with EA (20 mL×3), the combined organic layers were washed with brine, dried over anhydrous Na₂SO₄, filtered and the filtrate was concentrated. The residue was purified by silica gel chromatography by using a mixture of petroleum ether-ethyl acetate (98:2, v/v) as eluent to afford 7 (1.5 g, yield: 70%).

### The Synthesis of 2,2-dimethyl-4H-benzo[d][1,3]dioxine-8-carbaldehyde (8)

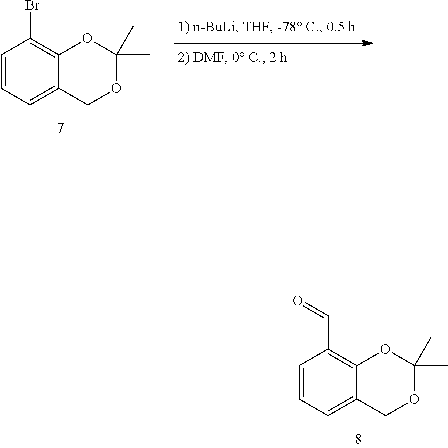

To a 100 mL three-necked flask were added 7 (1.0 g, 4.14 mmol) and THF (10 mL), then slowly add n-BuLi (2.0 mL, 4.96 mmol in THF) to the mixture at −78° C., the mixture was stirred at the same temperature for 0.5 hour. Then the dry DMF (0.60 g, 8.28 mmol) was added and the reaction temperature was warmed to room temperature, stirred for 2 hours. The reaction mixture was quenched by H₂O (30 mL), then the mixture was extracted with DCM (20 mL×3), the combined organic layers were washed with brine, dried over anhydrous Na₂SO₄, filtered and the filtrate was concentrated. The residue was purified by silica gel chromatography using a mixture of petroleum ether-ethyl acetate (90:10, v/v) as eluent to afford 8 (655 mg, yield: 82%).

### The Synthesis of N-((2,2-dimethyl-4H-benzo[d][1.3]dioxin-8-yl)methyl)-3-(1H-imidazol-2-yl)pyridin-2-amine (9)

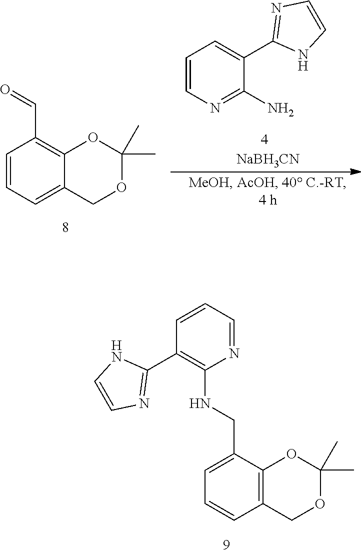 [SMILES: CC1(C)OCc2cccc(C=O)c2O1 ; Nc1ncccc1-c1ncc[nH]1 ; "NaBH3CN" ; CC1(C)OCc2cccc(CNc3ncccc3-c3ncc[nH]3)c2O1]

To a 50 mL round bottom flask were added 8 (98 mg, 0.51 mmol), 4 (80 mg, 0.50 mmol), MeOH (5 mL) and AcOH (5 drops), the reaction mixture was stirred at 40° C. for 2 hours. After the reaction was completed, NaBH₃CN (158 mg, 2.5 mmol) was added. The reaction mixture was stirred at room temperature for 2 hours. The reaction mixture was quenched with H₂O (1 mL), the mixture was concentrated in vacuo and purified by silica gel chromatography by using a mixture of petroleum ether-ethyl acetate (70:30, v/v) as eluent to afford 9 (95 mg, yield: 49%).

### The Synthesis of 2-(((3-(1H-imidazol-2-yl)pyridin-2-yl)amino)methyl)-6-(hydroxymethyl)phenol (10)

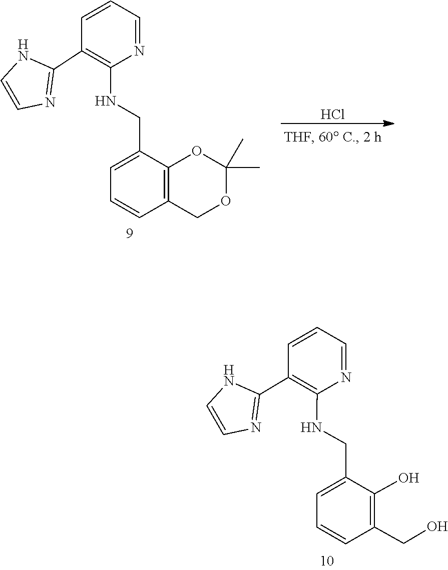 [SMILES: CC1(C)OCc2cccc(CNc3ncccc3-c3ncc[nH]3)c2O1 ; Cl ; OCc1cccc(CNc2ncccc2-c2ncc[nH]2)c1O]

To a 50 mL round bottom flask were added 9 (95 mg, 0.25 mmol), THF (10 mL), H₂O (1 mL) and HCl (6 N in water, 10 drops). The reaction mixture was stirred at 60° C. for 2 hours. After the reaction was completed, the reaction mixture was treated with saturated NaHCO₃ solution, the mixture was filtered and the filtrate was concentrated, the residue was dissolved with THF and then filtered, the filtrate was concentrated again to afford 10 (105 mg) as crude product for the next step without additional purification.

### The Synthesis of 3-(((3-(1H-imidazol-2-yl)pyridin-2-yl)amino)methyl)-2-hydroxybenzaldehyde (11)

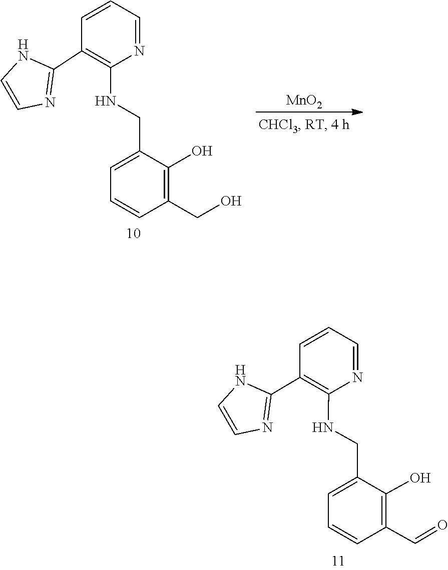 [SMILES: OCc1cccc(CNc2ncccc2-c2ncc[nH]2)c1O ; "MnO2" ; O=Cc1cccc(CNc2ncccc2-c2ncc[nH]2)c1O]

To a 50 mL round bottom flask were added 10 (105 mg, 0.36 mmol), MnO₂ (370 mg, 4.26 mmol) and CHCl₃ (10 mL), the reaction mixture was stirred at 70° C. for 4 hours. The reaction mixture was filtered and the filtrate was concentrated in vacuo to afford 11 (90 mg) as crude product for the next step without additional purification.

### The Synthesis of (E)-3-(((3-(1H-imidazol-2-yl)pyridin-2-yl)amino)methyl)-2-hydroxybenzaldehyde oxime (12)

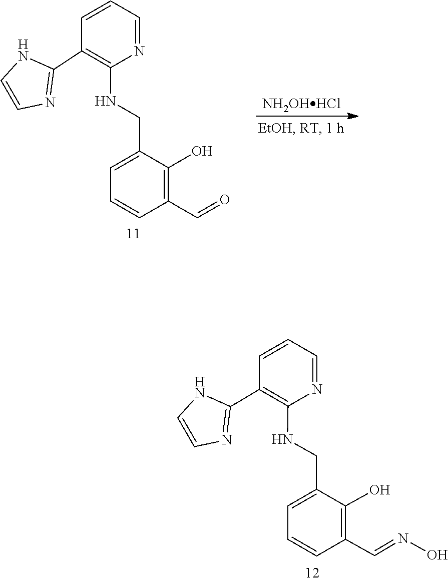 [SMILES: O=Cc1cccc(CNc2ncccc2-c2ncc[nH]2)c1O ; "NH2OH·HCl" ; O/N=C/c1cccc(CNc2ncccc2-c2ncc[nH]2)c1O]

To a 50 mL round bottom flask were added 11 (90 mg, 0.31 mmol), NH₂OH HCl (24 mg, 0.34 mmol) and EtOH (5 mL), the reaction mixture was stirred at room temperature for 1 hour. Then the reaction mixture was concentrated in vacuo to afford 12 (133 mg) as crude product for the next step without additional purification.

### The Synthesis of N-(benzo[d]isoxazol-7-ylmethyl)-3-(1H-imidazol-2-yl)pyridin-2-amine (ELG-000560)

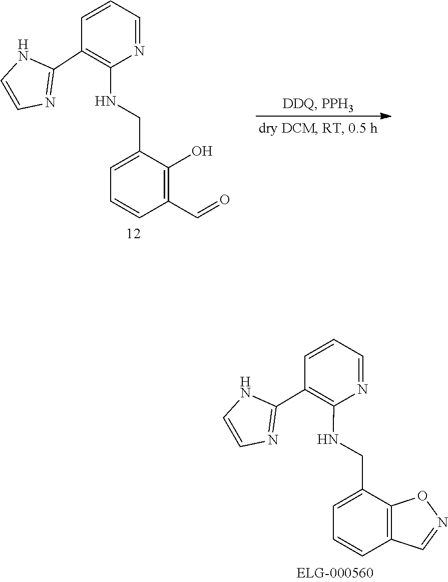

To a 50 mL round bottom flask were added DDQ (147.0 mg, 0.65 mmol), PPh₃ (170.0 mg, 0.65 mmol) and dry DCM (10 mL), the mixture was stirred at room temperature for 10 mins, then 12 (133.0 mg, 0.43 mmol) was added. The reaction mixture was stirred at room temperature for 0.5 hour. The reaction mixture was diluted with H₂O (30 mL), extracted by DCM (15 mL×3), the combined organic layers were washed with brine, dried over anhydrous Na₂SO₄, filtered and the filtrate was concentrated. The residue was purified by silica gel chromatography by using a mixture of petroleum ether-ethyl acetate (50:50, v/v) as eluent to afford a white solid (30 mg) as crude. Then the crude product was further purified by prep-TLC (a mixture of petroleum ether-ethyl acetate, 1:1, v/v) to afford ELG-000560 (2.2 mg, yield: 1%) as a white solid. LC-MS (Agilent LCMS 1260-6125B, Column: Boltimate EXT C18 CoreShell (50 mm*4.6 mm*2.7 μm): Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min, then under this condition for 1.0 min, finally changed to 90% [water+0.05% FA] and 10% [CH₃CN+0.05% FA] in 0.5 min), Purity: 94.60%, Rt=1.189 min: MS Calcd: 291.11: MS Found: 292.1 [M+H]⁺. HPLC (Waters UPLC, Column: ACQUITY UPLC BEH C18 1.7 μm*2.1 mm*150 mm): Column Temperature: 40° C.: Flow Rate: 0.3 mL/min; Mobile Phase: keep 90% [water+0.02% FA] and 10% [CH₃CN+0.02% FA] for 0.5 min, then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 94.39%, Rt=3.362 min. ¹H NMR (400 MHz, DMSO-d₆) δ 12.66 (s, 1H), 9.85 (t, J=5.9 Hz, 1H), 9.24 (s, 1H), 8.06 (dd, J=7.6, 1.7 Hz, 1H), 8.01 (dd, J=4.9, 1.7 Hz, 1H), 7.76 (d, J=7.9 Hz, 1H), 7.52 (dd, J=7.2, 0.9 Hz, 1H), 7.33 (dd, J=9.2, 5.9 Hz, 2H), 7.11 (s, 1H), 6.67 (dd, J=7.6, 4.9 Hz, 1H), 5.08 (d, J=5.9 Hz, 2H). Chemical Formula: C₁₆H₁₃N₅O. Molecular Weight: 291.31.

### Route for ELG-000567

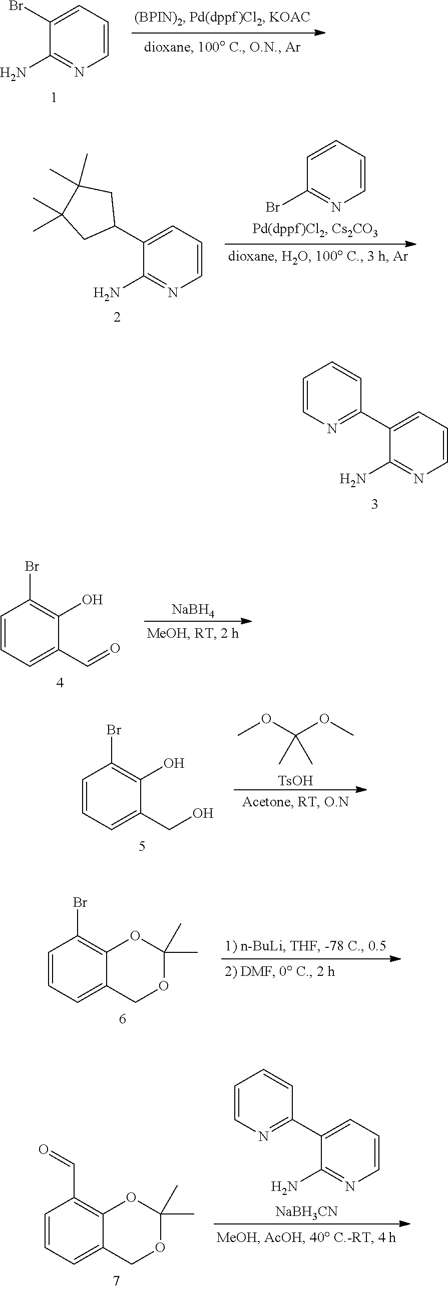

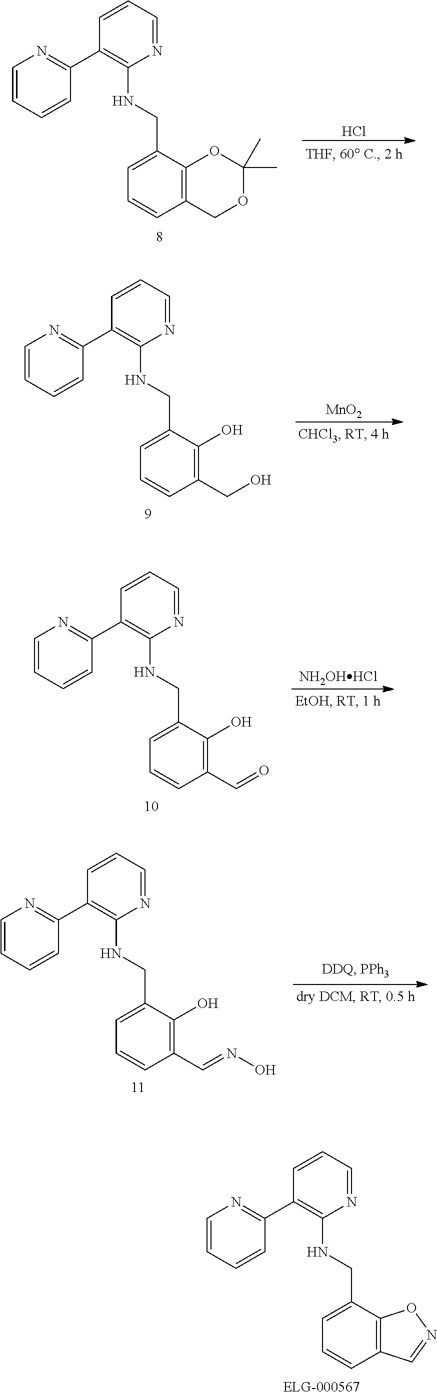

### The Synthesis of 3-(4,4,5,5-tetramethyl-1,3,2-dioxaborolan-2-yl)pyridin-2-amine (2)

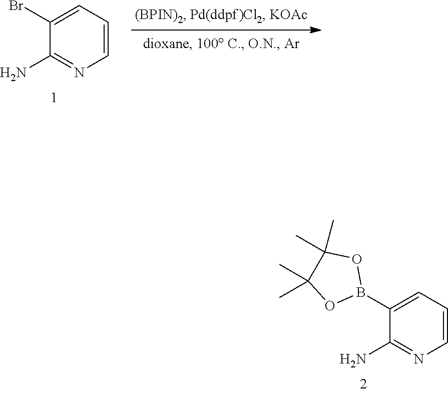 [SMILES: Nc1ncccc1Br ; CC1(C)OB(c2cccnc2N)OC1(C)C]

To a 250 mL round bottom flask were added 1 (2.5 g, 14.50 mmol). Pd(dppf)Cl₂ (531 mg, 0.73 mmol), (BPIN)₂ (4.4 g, 17.40 mmol), KOAc (4.26 g, 43.50 mmol) and dry dioxane (70 mL), the mixture was degassed and refilled with Ar three times. Then the reaction mixture was stirred at 100° C. overnight and used directly without further workup.

### The Synthesis of [2,3′-bipyridin]-2′-amine (3)

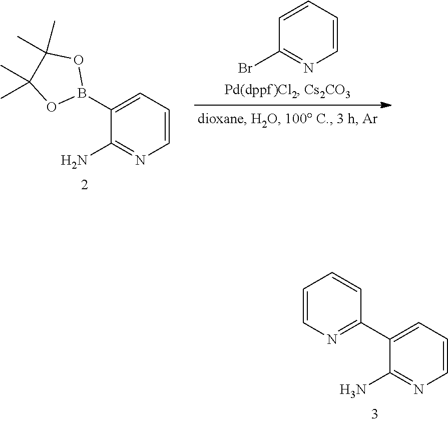 [SMILES: CC1(C)OB(c2cccnc2N)OC1(C)C ; Brc1ccccn1 ; Nc1ncccc1-c1ccccn1]

To a 250 mL round bottom flask were added 2 (3.19 g, 14.50 mmol), Pd(dppf)Cl₂ (531 mg, 0.73 mmol), Cs₂CO₃ (9.5 g, 29.0 mmol), 2-bromopyridine (2.3 g, 14.50 mmol), dioxane (70 mL) and H₂O (17.5 mL), the mixture was degassed and refilled with Ar three times. Then the reaction mixture was stirred at 100° C. for 3 hours. The reaction mixture was concentrated and purified by silica gel chromatography using DCM as eluent to afford 3 (1.0 g, yield: 40%).

### The Synthesis of 2-bromo-6-(hydroxymethyl)phenol (5)

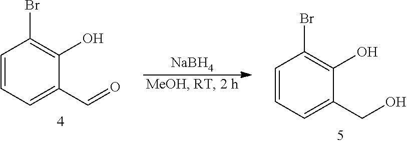

To a 100 mL round bottom flask were added 4 (2.85 g, 14.25 mmol) and MeOH (30 mL), then slowly add NaBH₄ (0.83 g, 21.38 mmol) at 0° C., and the reaction mixture was stirred at room temperature for 2 hours. The reaction mixture was quenched with H₂O, then the mixture was extracted with EA (30 mL×3), the combined organic layers were washed with brine, dried over anhydrous Na₂SO₄, filtered and the filtrate was concentrated in vacuo to afford 5 (1.8 g) as crude product for the next step without additional purification.

### The Synthesis of 8-bromo-2,2-dimethyl-4H-benzo[d][1.3]dioxine (6)

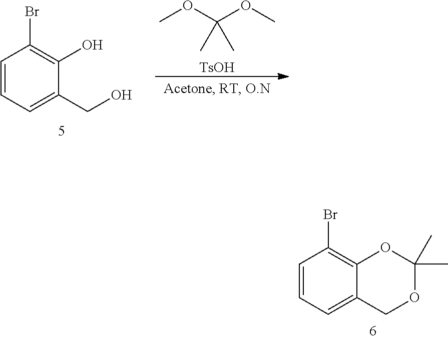

To a 100 mL round bottom flask were added 5 (1.80 g, 8.91 mmol), 2,2-dimethoxypropane (3.25 g, 31.19 mmol), TsOH (310 mg, 1.78 mmol) and acetone (30 mL), the mixture was stirred at room temperature overnight. The reaction mixture was quenched with saturated NaHCO₃ aqueous, then the mixture was extracted with EA (20 mL×3), the combined organic layers were washed with brine, dried over anhydrous Na₂SO₄, filtered and the filtrate was concentrated. The residue was purified by silica gel chromatography by using a mixture of petroleum ether-ethyl acetate (98:2, v/v) as eluent to afford 6 (1.5 g, yield: 70%).

### The Synthesis of 2,2-dimethyl-4H-benzo[d][1,3]dioxine-8-carbaldehyde (7)

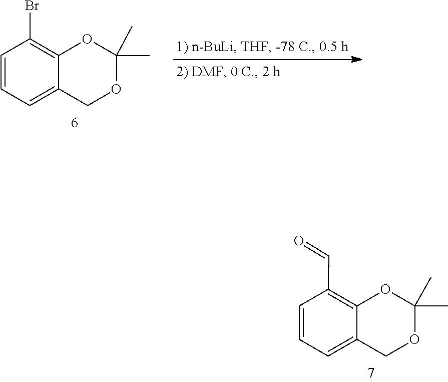

To a 100 mL three-necked flask were added 6 (1.0 g, 4.14 mmol) and THF (10 mL), then slowly add n-BuLi (2.0 mL, 4.96 mmol in THF) to the mixture at −78° C., the mixture was stirred at the same temperature for 0.5 hour. Then the dry DMF (0.60 g, 8.28 mmol) was added and the reaction temperature was warmed to room temperature, stirred for 2 hours. The reaction mixture was quenched by H₂O (30 mL), then the mixture was extracted with DCM (20 mL×3), the combined organic layers were washed with brine, dried over anhydrous Na₂SO₄, filtered and the filtrate was concentrated. The residue was purified by silica gel chromatography using a mixture of petroleum ether-ethyl acetate (90:10, v/v) as eluent to afford 7 (655 mg, yield: 82%).

The synthesis of N-((2,2-dimethyl-4H-benzo[d][1.3]dioxin-8-yl)methyl)-[2,3′-bipyridin]-2′-amine (8).

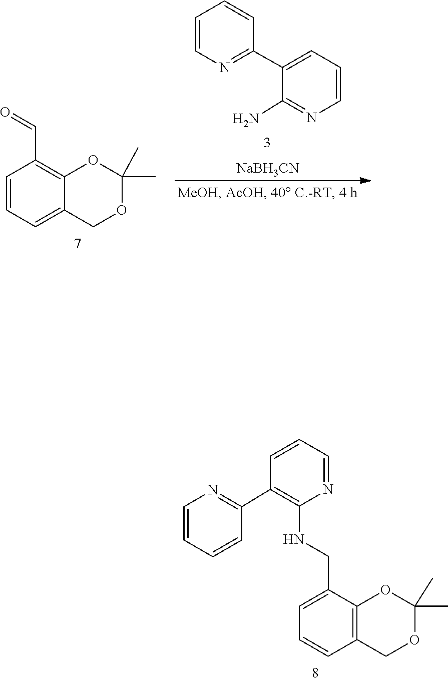 [SMILES: CC1(C)OCc2cccc(C=O)c2O1 ; Nc1ncccc1-c1ccccn1 ; "NaBH3CN" ; CC1(C)OCc2cccc(CNc3ncccc3-c3ccccn3)c2O1]

To a 50 mL round bottom flask were added 7 (336 mg, 1.75 mmol), 3 (300 mg, 1.75 mmol), MeOH (10 mL) and AcOH (5 drops), the reaction mixture was stirred at 40° C. for 2 hours. After the reaction was completed, NaBH₃CN (228 mg, 3.5 mmol) was added. The reaction mixture was stirred at room temperature for 2 hours. The reaction mixture was quenched with H₂O (1 mL), the mixture was concentrated in vacuo and purified by silica gel chromatography by using a mixture of petroleum ether-ethyl acetate (70:30, v/v) as eluent to afford 8 (80 mg, yield: 13%).

The synthesis of 2-(([2,3′-bipyridin]-2′-ylamino)methyl)-6-(hydroxymethyl)phenol (9).

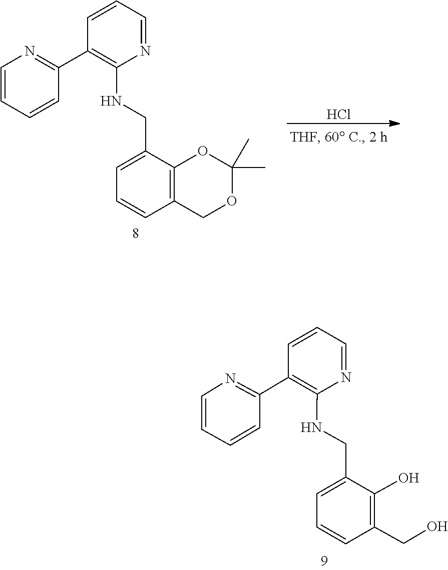 [SMILES: CC1(C)OCc2cccc(CNc3ncccc3-c3ccccn3)c2O1 ; Cl ; OCc1cccc(CNc2ncccc2-c2ccccn2)c1O]

To a 50 mL round bottom flask were added 8 (80 mg, 0.23 mmol), THF (3 mL), H₂O (0.3 mL) and HCl (6 N in water, 10 drops). The reaction mixture was stirred at 60° C. for 2 hours. After the reaction was completed, the reaction mixture was treated with saturated NaHCO₃ solution, the mixture was filtered and the filtrate was concentrated, the residue was dissolved with THF and then filtered, the filtrate was concentrated again to afford 9 (81 mg) as crude product for the next step without additional purification.

### The Synthesis of 3-(([2,3′-bipyridin]-2′-ylamino)methyl)-2-hydroxybenzaldehyde (10)

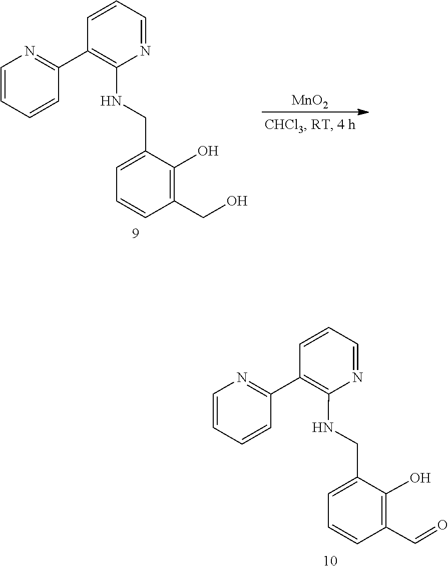 [SMILES: OCc1cccc(CNc2ncccc2-c2ccccn2)c1O ; "MnO2" ; O=Cc1cccc(CNc2ncccc2-c2ccccn2)c1O]

To a 50 mL round bottom flask were added 9 (81 mg, 0.26 mmol), MnO₂ (226 mg, 2.6 mmol) and CHCl₃ (5 mL), the reaction mixture was stirred at 70° C. for 4 hours. The reaction mixture was filtered and the filtrate was concentrated in vacuo to afford 10 (70 mg) as crude product for the next step without additional purification.

### The Synthesis of (E)-3-(([2,3′-bipyridin]-2′-ylamino)methyl)-2-hydroxybenzaldehyde oxime (11)

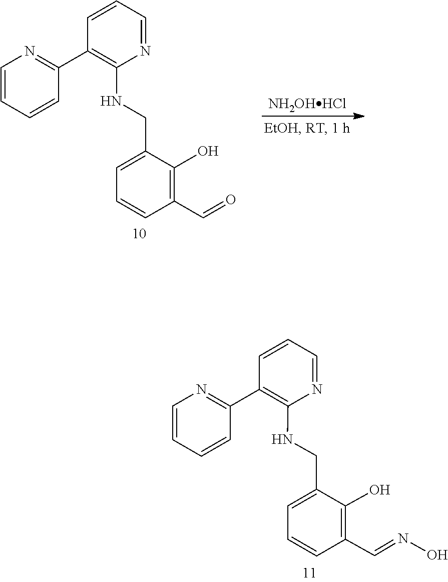 [SMILES: O=Cc1cccc(CNc2ncccc2-c2ccccn2)c1O ; "NH2OH·HCl" ; O/N=C/c1cccc(CNc2ncccc2-c2ccccn2)c1O]

To a 50 mL round bottom flask were added 10 (70 mg, 0.23 mmol), NH₂OH HCl (24 mg, 0.34 mmol) and EtOH (3 mL), the reaction mixture was stirred at room temperature for 1 hour. Then the reaction mixture was concentrated in vacuo to afford 11 (108 mg) as crude product for the next step without additional purification.

### The Synthesis of N-(benzo[d]isoxazol-7-ylmethyl)-[2,3′-bipyridin]-2′-amine (ELG-000567)

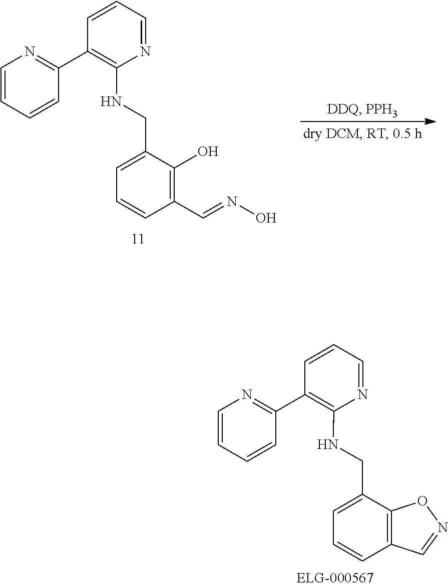

To a 50 mL round bottom flask were added DDQ (147.0 mg, 0.65 mmol), PPh₃ (170.0 mg, 0.65 mmol) and dry DCM (10 mL), the mixture was stirred at room temperature for 10 mins, then 11 (108 mg, 0.34 mmol) was added. The reaction mixture was stirred at room temperature for 0.5 hour. The reaction mixture was diluted with water (20 mL), extracted with DCM (10 mL×3), the combined organic layers were washed with brine, dried over anhdrous Na₂SO₄, filtered and the filtrate was concentrated. The residue was purified by prep-TLC to afford ELG-000567 (2.5 mg, yield: 2%) as a white solid. LC-MS (Agilent LCMS 1260-6125B, Column: Boltimate EXT C18 CoreShell (50 mm*4.6 mm*2.7 μm); Column Temperature: 45° C.; Flow Rate: 2.0 mL/min; Mobile Phase: from 90% [water+0.05% FA] and 10% [CH₃CN+0.05% FA] to 5% [water+0.05% FA] and 95% [CH₃CN+0.05% FA] in 1.5 min. then under this condition for 1.0 min, finally changed to 90% [water+0.05% FA] and 10% [CH₃CN+0.05% FA] in 0.5 min), Purity: 78.98%, Rt=1.119 min; MS Calcd: 302.12; MS Found: 303.1 [M+H]⁺. HPLC (Waters UPLC, Column: ACQUITY UPLC BEH C18 1.7 μm*2.1 mm*150 mm); Column Temperature: 40° C.; Flow Rate: 0.3 mL/min; Mobile Phase: keep 90% [water+0.02% FA] and 10% [CH₃CN+0.02% FA] for 0.5 min. then changed to 5% [water+0.02% FA] and 95% [CH₃CN+0.02% FA] in 8.5 min and under this condition for 2.5 min, finally changed to 90% [water+0.02% FA] and 10% [CH₃CN+0.02% FA] in 1.5 min and under this condition for 2 min), Purity: 89.92%, Rt=3.742 min. ¹H NMR (400 MHz, CD₃OD) δ 8.92 (s, 1H), 8.62-8.56 (m, 1H), 8.05 (s, 1H), 8.03 (dd, J=4.2, 1.8 Hz, 1H), 7.90 (dd, J=6.3, 1.7 Hz, 2H), 7.71 (d, J=7.9 Hz, 1H), 7.57 (dd. J=7.3, 0.9 Hz, 1H), 7.32-7.27 (m, 2H), 6.72 (dd, J=7.3, 5.3 Hz, 1H), 5.08 (s, 2H). Chemical Formula: C18H₁₄N₄O. Molecular Weight: 302.34.

### Example 3: Biophysical Data

TABLE 1
Binding affinities of select compounds for procaspase-6.

| | | K_{D} (μM) |
|---|---|---|
| | | A: <10 μM; B: 10-<50 μM; |
| Compound ID | Structure | C: 50-100 μM; D: >100 μM |
| ELG-000507 | 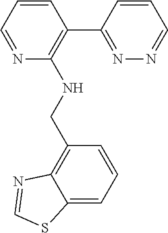 [SMILES: c1cnnc(-c2cccnc2NCc2cccc3scnc23)c1] | A |
| ELG-000504 | 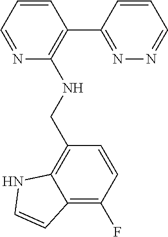 [SMILES: Fc1ccc(CNc2ncccc2-c2cccnn2)c2[nH]ccc12] | A |
| ELG-000506 | 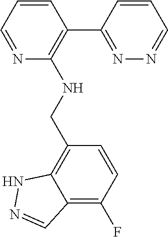 [SMILES: Fc1ccc(CNc2ncccc2-c2cccnn2)c2[nH]ncc12] | A |
| ELG-000496 | 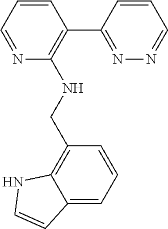 [SMILES: c1cnnc(-c2cccnc2NCc2cccc3cc[nH]c23)c1] | A |
| ELG-000480 | 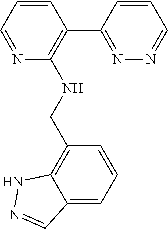 [SMILES: c1cnnc(-c2cccnc2NCc2cccc3cn[nH]c23)c1] | A |
| ELG-000522 | 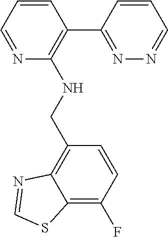 [SMILES: Fc1ccc(CNc2ncccc2-c2cccnn2)c2ncsc12] | A |
| ELG-000499 | 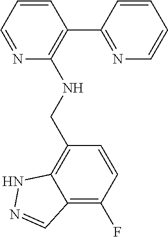 [SMILES: Fc1ccc(CNc2ncccc2-c2ccccn2)c2[nH]ncc12] | A |
| ELG-000491 | 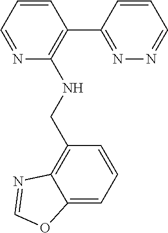 [SMILES: c1cnnc(-c2cccnc2NCc2cccc3ocnc23)c1] | A |
| ELG-000515 | 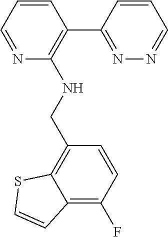 [SMILES: Fc1ccc(CNc2ncccc2-c2cccnn2)c2sccc12] | A |
| ELG-000525 | 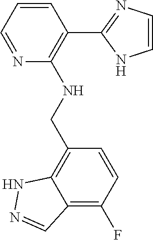 [SMILES: Fc1ccc(CNc2ncccc2-c2ncc[nH]2)c2[nH]ncc12] | A |
| ELG-000481 | 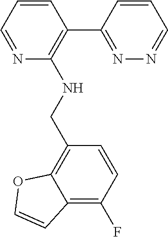 [SMILES: Fc1ccc(CNc2ncccc2-c2cccnn2)c2occc12] | B |
| ELG-000502 | 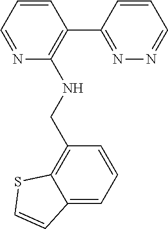 [SMILES: c1cnnc(-c2cccnc2NCc2cccc3ccsc23)c1] | B |
| ELG-000526 | 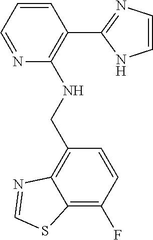 [SMILES: Fc1ccc(CNc2ncccc2-c2ncc[nH]2)c2ncsc12] | B |
| ELG-000536 | 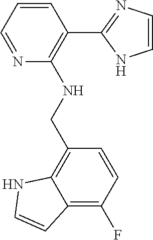 [SMILES: Fc1ccc(CNc2ncccc2-c2ncc[nH]2)c2[nH]ccc12] | B |
| ELG-000488 | 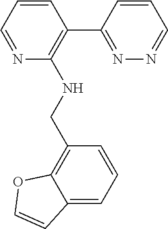 [SMILES: c1cnnc(-c2cccnc2NCc2cccc3ccoc23)c1] | B |
| ELG-000530 | 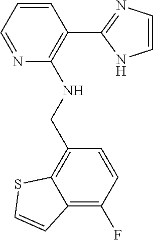 [SMILES: Fc1ccc(CNc2ncccc2-c2ncc[nH]2)c2sccc12] | B |
| ELG-000543 | 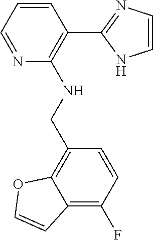 [SMILES: Fc1ccc(CNc2ncccc2-c2ncc[nH]2)c2occc12] | B |
| ELG-000538 | 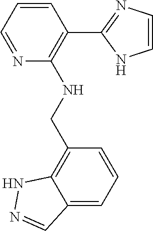 [SMILES: c1cnc(NCc2cccc3cn[nH]c23)c(-c2ncc[nH]2)c1] | B |
| ELG-000523 | 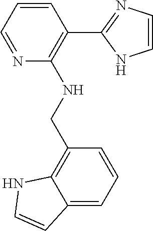 [SMILES: c1cnc(NCc2cccc3cc[nH]c23)c(-c2ncc[nH]2)c1] | B |
| ELG-000532 | 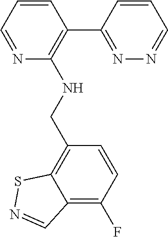 [SMILES: Fc1ccc(CNc2ncccc2-c2cccnn2)c2sncc12] | B |
| ELG-000560 | 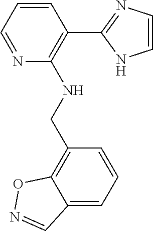 [SMILES: c1cnc(NCc2cccc3cnoc23)c(-c2ncc[nH]2)c1] | B |
| ELG-000548 | 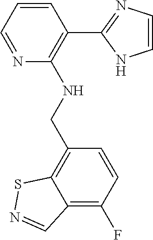 [SMILES: Fc1ccc(CNc2ncccc2-c2ncc[nH]2)c2sncc12] | C |
| ELG-000483 | 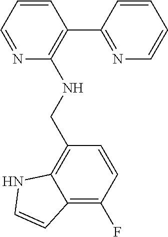 [SMILES: Fc1ccc(CNc2ncccc2-c2ccccn2)c2[nH]ccc12] | C |
| ELG-000537 | 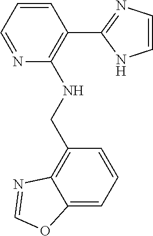 [SMILES: c1cnc(NCc2cccc3ocnc23)c(-c2ncc[nH]2)c1] | C |
| ELG-000535 | 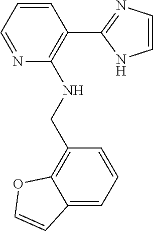 [SMILES: c1cnc(NCc2cccc3ccoc23)c(-c2ncc[nH]2)c1] | C |
| ELG-000559 | 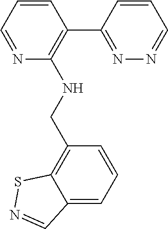 [SMILES: c1cnnc(-c2cccnc2NCc2cccc3cnsc23)c1] | C |
| ELG-000531 | 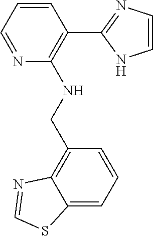 [SMILES: c1cnc(NCc2cccc3scnc23)c(-c2ncc[nH]2)c1] | C |
| ELG-000558 | 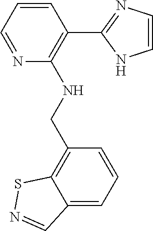 [SMILES: c1cnc(NCc2cccc3cnsc23)c(-c2ncc[nH]2)c1] | C |
| ELG-000463 | 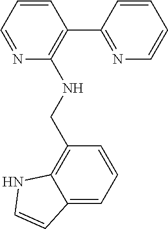 [SMILES: c1ccc(-c2cccnc2NCc2cccc3cc[nH]c23)nc1] | C |
| ELG-000468 | 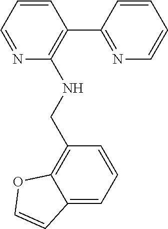 [SMILES: c1ccc(-c2cccnc2NCc2cccc3ccoc23)nc1] | C |
| ELG-000469 | 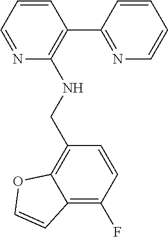 [SMILES: Fc1ccc(CNc2ncccc2-c2ccccn2)c2occc12] | C |
| ELG-000472 | 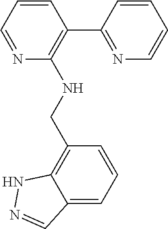 [SMILES: c1ccc(-c2cccnc2NCc2cccc3cn[nH]c23)nc1] | C |
| ELG-000497 | 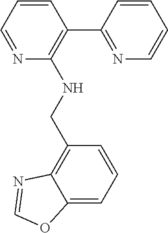 [SMILES: c1ccc(-c2cccnc2NCc2cccc3ocnc23)nc1] | C |
| ELG-000510 | 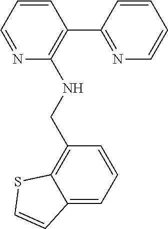 [SMILES: c1ccc(-c2cccnc2NCc2cccc3ccsc23)nc1] | C |
| ELG-000511 | 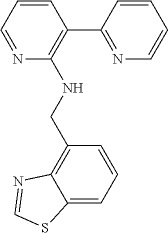 [SMILES: c1ccc(-c2cccnc2NCc2cccc3scnc23)nc1] | C |
| ELG-000518 | 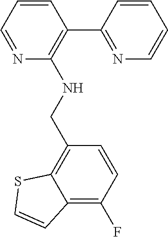 [SMILES: Fc1ccc(CNc2ncccc2-c2ccccn2)c2sccc12] | C |
| ELG-000552 | 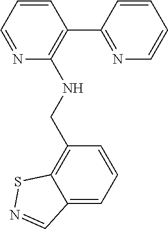 [SMILES: c1ccc(-c2cccnc2NCc2cccc3cnsc23)nc1] | C |
| ELG-000563 | 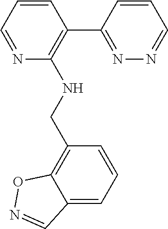 [SMILES: c1cnnc(-c2cccnc2NCc2cccc3cnoc23)c1] | C |
| ELG-000567 | 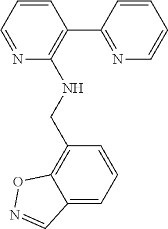 [SMILES: c1ccc(-c2cccnc2NCc2cccc3cnoc23)nc1] | C |
| ELG-000549 | 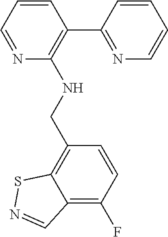 [SMILES: Fc1ccc(CNc2ncccc2-c2ccccn2)c2sncc12] | D |
| ELG-000534 | 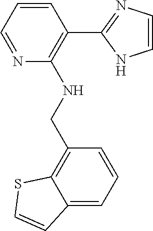 [SMILES: c1cnc(NCc2cccc3ccsc23)c(-c2ncc[nH]2)c1] | D |

### REFERENCES

1. Murray, J.; Giannetti, A. M.; Steffek, M.; Gibbons, P.; Hearn, B. R.; Cohen, F.; Tam. C.; Pozniak, C.; Bravo, B.: Lewcock, J.: Jaishankar. P.; Ly, C. Q.; Zhao, X.; Tang, Y.; Chugha, P.: Arkin, M. R.; Flygare, J.; Renslo, A. R. ChemMedChem 2014, 9, 73-77, 2. Ehrnhoefer, D. E. et al. Cell Chem. Biol. 2019, 26, 1295-1305, 3. Zhao, P. et al. Science 2020, 367, 652-660, 4. Chu, H. et al. Nature 2022, 609, 785-792.

What is claimed is:

1. A compound, or a pharmaceutically acceptable salt thereof, having the formula: 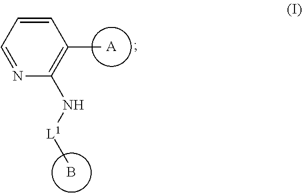
wherein
Ring A is a substituted or unsubstituted 5 to 6 membered heteroaryl or substituted or unsubstituted 8 to 10 membered fused ring heteroaryl;
Ring B is a substituted or unsubstituted 9 to 10 membered fused ring aryl or substituted or unsubstituted 9 to 10 membered fused ring heteroaryl;
L¹ is a bond or substituted or unsubstituted C₁-C₃ allylene.

2. The compound of claim 1, wherein Ring B is substituted or unsubstituted benzoisoxazolyl, substituted or unsubstituted benzoisothiazolyl, substituted or unsubstituted benzofuranyl, substituted or unsubstituted indazolyl, substituted or unsubstituted benzoxazolyl, substituted or unsubstituted benzothiazolyl, substituted or unsubstituted benzothiophenyl, or substituted or unsubstituted indolyl.

3. The compound of claim 1, wherein Ring B is 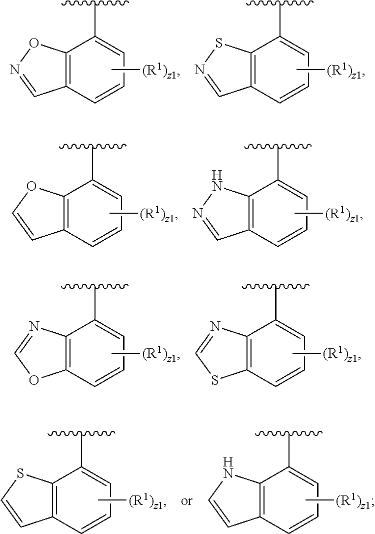
wherein
R¹ is independently halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NR^{1C}C(O)NR^{1A}R^{1B}, —N(O)ₘ₁, —NR^{1A}R^{1B}, —C(O)R^{1C}, —C(O)OR^{1C}, —OC(O)R^{1C}, —OC(O)OR^{1C}, —C(O)NR^{1A}R^{1B}, —OC(O)NR^{1A}R^{1B}, —OR^{1D}, —SR^{1D}, —NR^{1A}SO₂R^{1D}, —NR^{1A}C(O)R^{1C}, —NR^{1A}C(O)OR^{1C}, —NR^{1A}OR^{1C}, —B(OR^{1C})(OR^{1D}), —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A}, R^{1B}, R^{1C}, and R^{1D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{1A} and R^{1B} substituents bonded to the same nitrogen atom may optionally be joined to form a substituted or unsubstituted heterocycloalkyl or substituted or unsubstituted heteroaryl;
each X¹ is independently —F, —Cl, —Br, or —I;
n1 is an integer from 0 to 4;
m1 and v1 are independently 1 or 2; and
z1 is an integer from 0 to 5.

4. The compound of claim 3, wherein Ring B is

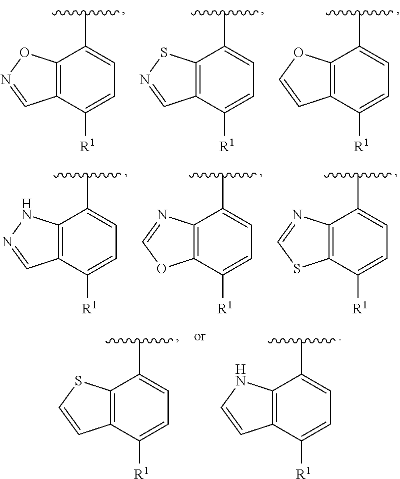

5. The compound of claim 3, wherein R¹ is independently halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —B(OH)₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

6. The compound of claim 3, wherein R′ is independently halogen.

7. The compound of claim 3, wherein R¹ is independently —F.

8. The compound of claim 3, wherein z1 is 0.

9. The compound of claim 3, wherein z1 is 1.

10. The compound of claim 1, wherein Ring B is

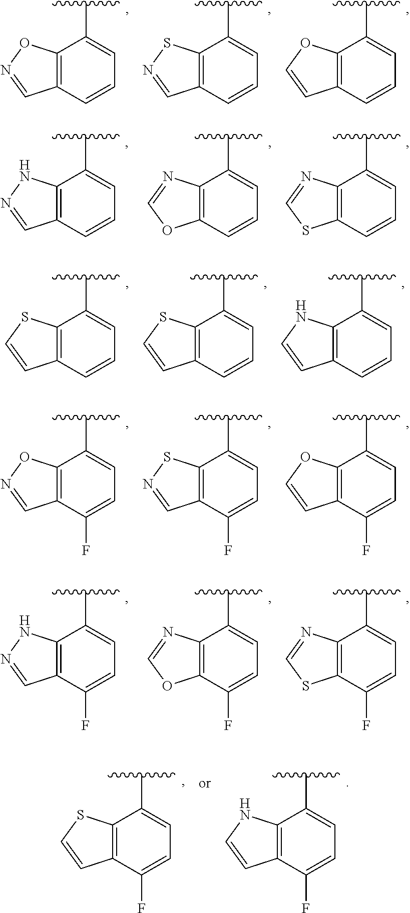

11. The compound of claim 1, wherein Ring A is a substituted or unsubstituted nitrogen-containing 5 to 6 membered heteroaryl or substituted or unsubstituted nitrogen-containing 8 to 10 membered fused ring heteroaryl.

12. The compound of claim 1, wherein Ring A is substituted or unsubstituted pyridyl, substituted or unsubstituted pyridazinyl, substituted or unsubstituted pyrimidinyl, substituted or unsubstituted pyrazinyl, substituted or unsubstituted triazinyl, substituted or unsubstituted imidazolyl, substituted or unsubstituted pyrazolyl, substituted or unsubstituted oxazolyl, substituted or unsubstituted isoxazolyl, substituted or unsubstituted furanyl, substituted or unsubstituted thienyl, substituted or unsubstituted thiazolyl, substituted or unsubstituted isothiazolyl, substituted or unsubstituted triazolyl, substituted or unsubstituted oxadiazolyl, substituted or unsubstituted tetrazolyl, substituted or unsubstituted imidazopyridinyl, substituted or unsubstituted benzothiazolyl, or substituted or unsubstituted benzoimidazolyl.

13. The compound of claim 1, wherein Ring A is 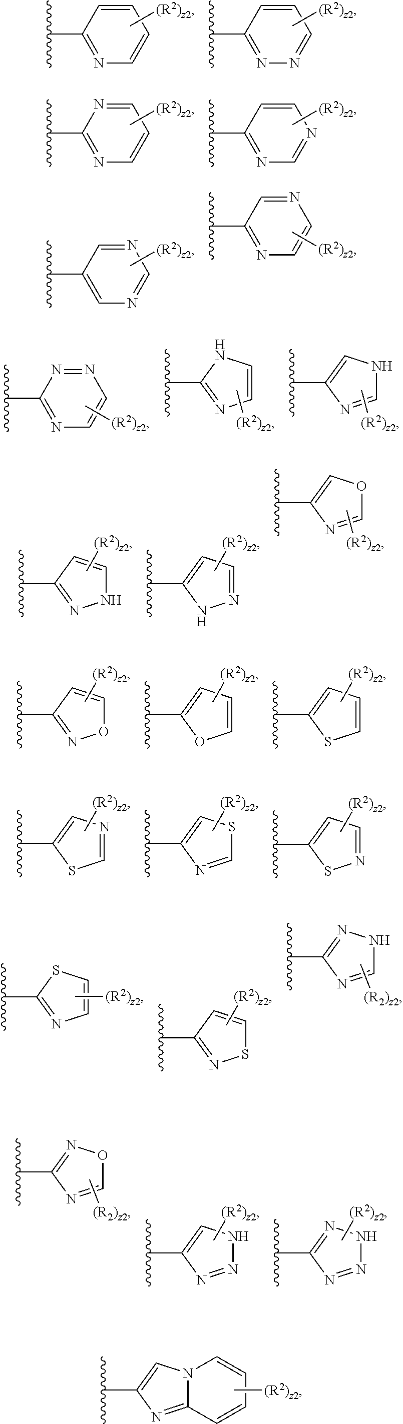 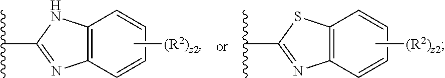
wherein
R² is independently halogen, —CC₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —OSO₃H, —SO₂NH₂, —NHNH₂, —ONH₂, —NHC(O)NH₂, —NHSO₂H, —NHC(O)H, —NHC(O)OH, —NHOH, —OCCl₃, —OCBr₃, —OCF₃, —OCI₃, —OCH₂Cl, —OCH₂Br, —OCH₂F, —OCH₂I, —OCHCl₂, —OCHBr₂, —OCHF₂, —OCHI₂, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
z2 is an integer from 0 to 5.

14. The compound of claim 13, wherein R² is independently —NH₂ or unsubstituted C₁-C₄ alkyl.

15. The compound of claim 13, wherein R² is independently —NH₂ or unsubstituted methyl.

16. The compound of claim 13, wherein z2 is 0.

17. The compound of claim 13, wherein z2 is 1.

18. The compound of claim 1, wherein Ring A is

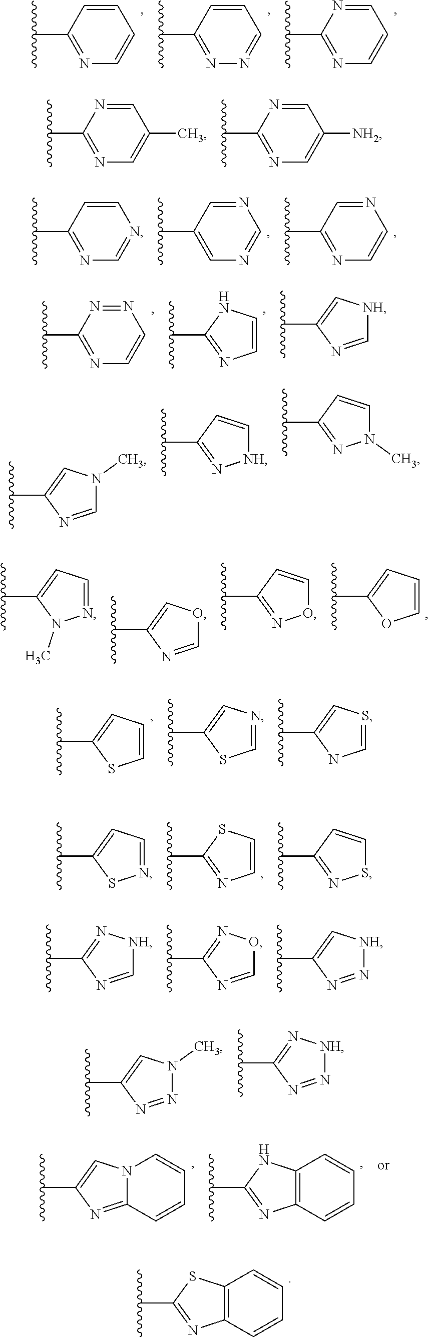

19. The compound of claim 1, wherein L¹ is unsubstituted C₁-C₃ alkylene.

20. The compound of claim 1, wherein L¹ is unsubstituted methylene.

21. A pharmaceutical composition comprising a compound of one of claims 1 to 20, or a pharmaceutically acceptable salt thereof, and a pharmaceutically acceptable excipient.

22. A method of treating a neurodegenerative disease in a subject in need thereof, said method comprising administering to the subject in need thereof a therapeutically effective amount of a compound of one of claims 1 to 20, or a pharmaceutically acceptable salt thereof.

23. The method of claim 22, wherein the neurodegenerative disease is a tauopathy.

24. The method of claim 22, wherein the neurodegenerative disease is Alzheimer's disease, Huntington's disease, amyotrophic lateral sclerosis, Lewy body disease, progressive supranuclear palsy, or Parkinson's disease.

25. The method of claim 22, wherein the neurodegenerative disease is Alzheimer's disease.

26. A method of treating a liver disease in a subject in need thereof, said method comprising administering to the subject in need thereof a therapeutically effective amount of a compound of one of claims 1 to 20, or a pharmaceutically acceptable salt thereof.

27. The method of claim 26, wherein the liver disease is nonalcoholic steatohepatitis.

28. A method of treating a fibrotic disease in a subject in need thereof, said method comprising administering to the subject in need thereof a therapeutically effective amount of a compound of one of claims 1 to 20, or a pharmaceutically acceptable salt thereof.

29. A method of treating a coronavirus infection in a subject in need thereof, said method comprising administering to the subject in need thereof a therapeutically effective amount of a compound of one of claims 1 to 20, or a pharmaceutically acceptable salt thereof.

30. A method of reducing the level of activity of caspase-6 protein in a cell, said method comprising contacting the cell with an effective amount of a compound of one of claims 1 to 20, or a pharmaceutically acceptable salt thereof.

31. A method of reducing the level of activation of procaspase-6 protein in a cell, said method comprising contacting the cell with an effective amount of a compound of one of claims 1 to 20, or a pharmaceutically acceptable salt thereof.


SEQUENCE LISTING (1 sequence)

SEQ ID NO: 1 - protein, 293 aa, synthetic construct
  REGION 1..293: note = Synthetic polypeptide
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN